(19) United States
(12) Patent Application Publication
(10) Pub. No.: US 2024/0254214 A1
(43) Pub. Date: Aug. 1, 2024

(54) TREATMENT OF OCULAR DISEASES WITH FULLY-HUMAN POST-TRANSLATIONALLY MODIFIED ANTI-VEGF Fab

(71) Applicants: REGENXBIO Inc., Rockville, MD (US); The Johns Hopkins University, Baltimore, MD (US)

(72) Inventors: Stephen Yoo, Newton, MA (US); Rickey Robert Reinhardt, Silver Spring, MD (US); Sherri Van Everen, Menlo Park, CA (US); Karen Fran Kozarsky, Bala Cynwyd, PA (US); Curran Matthew Simpson, Frederick, MD (US); Zhuchun Wu, Potomac, MD (US); Peter Anthony Campochiaro, Baltimore, MD (US); Jikui Shen, Dundalk, MD (US); Kun Ding, Baltimore, MD (US)

(21) Appl. No.: 18/360,073

(22) Filed: Jul. 27, 2023

Related U.S. Application Data
(63) Continuation of application No. 16/645,877, filed on Mar. 10, 2020, now abandoned.
(60) Provisional application No. 62/564,095, filed on Sep. 27, 2017.
(60) Provisional application No. 62/574,657, filed on Oct. 19, 2017.
(60) Provisional application No. 62/579,682, filed on Oct. 31, 2017.
(60) Provisional application No. 62/632,812, filed on Feb. 20, 2018.

(51) Int. Cl. C07K 16/22; A61F 9/00; A61K 9/00; A61M 25/00; A61M 25/01; A61P 27/02; C12N 7/00; C12N 15/86; A61K 39/00; A61K 48/00

(52) U.S. Cl. CPC C07K 16/22; A61F 9/0017; A61K 9/0019; A61K 9/0048; A61M 25/0084; A61M 25/0194; A61P 27/02; C12N 7/00; C12N 15/86; A61K 2039/505; A61K 48/00; A61M 2025/0089; A61M 2210/0612; C07K 2317/24; C07K 2317/40; C07K 2317/41; C07K 2317/54; C07K 2317/55; C07K 2317/565; C07K 2317/622; C12N 2750/14143; C12N 2750/14171

(57) ABSTRACT

Compositions and methods are described for the delivery of a fully human post-translationally modified (HuPTM) monoclonal antibody (“mAb”) or the antigen-binding fragment of a mAb against human vascular endothelial growth factor (“hVEGF”) —such as, e.g., a fully human-glycosylated (HuGly) anti-h VEGF antigen-binding fragment—to the retina/vitreal humour in the eye(s) of human subjects diagnosed with ocular diseases caused by increased neovascularization, for example, neovascular age-related macular degeneration (“nAMD”), also known as “wet” age-related macular degeneration (“WAMD”), age-related macular degeneration (“AMD”), and diabetic retinopathy.

### CROSS-REFERENCE TO RELATED APPLICATIONS

This application claims the benefit of U.S. Provisional Patent Application Nos. 62/564,095, filed Sep. 27, 2017, 62/574,657, filed Oct. 19, 2017, 62/579,682, filed Oct. 31, 2017, and 62/632,812, filed Feb. 20, 2018, which are incorporated by reference herein in their entireties.

### REFERENCE TO SEQUENCE LISTING SUBMITTED ELECTRONICALLY

This application incorporates by reference a Sequence Listing submitted with this application as text file entitled “Sequence_Listing_12656-110-228.TXT” created on Sep. 19, 2018 and having a size of 97,512 bytes.

### 1. INTRODUCTION

Compositions and methods are described for the delivery of a fully human post-translationally modified (HuPTM) monoclonal antibody (“mAb”) or the antigen-binding fragment of a mAb against vascular endothelial growth factor (“VEGF”)—such as, e.g., a fully human-glycosylated (HuGly) anti-VEGF antigen-binding fragment—to the retina/vitreal humour in the eye(s) of human subjects diagnosed with ocular diseases, in particular an ocular disease caused by increased neovascularization, for example, neovascular age-related macular degeneration (“nAMD”), also known as “wet” age-related macular degeneration (“WAMD” or “wet AMD”), age-related macular degeneration (“AMD”), and diabetic retinopathy.

### 2. BACKGROUND OF THE INVENTION

Age-related macular degeneration (AMD) is a degenerative retinal eye disease that causes a progressive, irreversible, severe loss of central vision. The disease impairs the macula—the region of highest visual acuity (VA)—and is the leading cause of blindness in Americans 60 years or older (NIH 2008).

The “wet,” neovascular form of AMD (“WAMD” or “wet AMD”), also known as neovascular age-related macular degeneration (nAMD), accounts for 15-20% of AMD cases, and is characterized by abnormal neovascularization in and under the neuroretina in response to various stimuli. This abnormal vessel growth leads to formation of leaky vessels and often haemorrhage, as well as distortion and destruction of the normal retinal architecture. Visual function is severely impaired in nAMD, and eventually inflammation and scarring cause permanent loss of visual function in the affected retina. Ultimately, photoreceptor death and scar formation result in a severe loss of central vision and the inability to read, write, and recognize faces or drive. Many patients can no longer maintain gainful employment, carry out daily activities and consequently report a diminished quality of life (Mitchell, 2006).

Diabetic retinopathy is an ocular complication of diabetes, characterized by microaneurysms, hard exudates, hemorrhages, and venous abnormalities in the non-proliferative form and neovascularization, preretinal or vitreous hemorrhages, and fibrovascular proliferation in the proliferative form. Hyperglycemia induces microvascular retinal changes, leading to blurred vision, dark spots or flashing lights, and sudden loss of vision (Cai & McGinnis, 2016).

Preventative therapies have demonstrated little effect, and therapeutic strategies have focused primarily on treating the neovascular lesion. Available treatments for nAMD include laser photocoagulation, photodynamic therapy with verteporfin, and intravitreal (“IVT”) injections with agents aimed at binding to and neutralizing vascular endothelial growth factor (“VEGF”)—a cytokine implicated in stimulating angiogenesis and targeted for intervention. Such anti-VEGF agents used include, e.g., bevacizumab (a humanized monoclonal antibody (mAb) against VEGF produced in CHO cells), ranibizumab (the Fab portion of an affinity-improved variant of bevacizumab made in prokaryotic E. coli), aflibercept (a recombinant fusion protein consisting of VEGF-binding regions of the extracellular domains of the human VEGF-receptor fused to the Fc portion of human IgG1), or pegaptanib (a pegylated aptamer (a single-stranded nucleic acid molecule) that binds to VEGF). Each of these therapies has some effect on best-corrected visual acuity; however, their effects appear limited in restoring visual acuity and in duration.

Anti-VEGF IVT injections have been shown to be effective in reducing leakage and sometimes restoring visual loss. However, because these agents are effective for only a short period of time, repeated injections for long durations are often required, thereby creating considerable treatment burden for patients. While long term therapy with either monthly ranibizumab or monthly/every 8 week aflibercept may slow the progression of vision loss and improve vision, none of these treatments prevent neovascularization from recurring (Brown 2006; Rosenfeld, 2006; Schmidt-Erfurth, 2014). Each has to be re-administered to prevent the disease from worsening. The need for repeat treatments can incur additional risk to patients and is inconvenient for both patients and treating physicians.

### 3. SUMMARY OF THE INVENTION

Compositions and methods are described for the delivery of a fully human post-translationally modified (HuPTM) antibody against VEGF to the retina/vitreal humour in the eye(s) of patients (human subjects) diagnosed with an ocular disease, in particular an ocular disease caused by increased neovascularization, for example, nAMD (also known as “wet” AMD), dry AMD, retinal vein occlusion (RVO), diabetic macular edema (DME), or diabetic retinopathy (DR) (in particular, wet AMD). Antibodies include, but are not limited to, monoclonal antibodies, polyclonal antibodies, recombinantly produced antibodies, human antibodies, humanized antibodies, chimeric antibodies, synthetic antibodies, tetrameric antibodies comprising two heavy chain and two light chain molecules, antibody light chain monomers, antibody heavy chain monomers, antibody light chain dimers, antibody heavy chain dimers, antibody light chain-heavy chain pairs, intrabodies, heteroconjugate antibodies, monovalent antibodies, antigen-binding fragments of full-length antibodies, and fusion proteins of the above. Such antigen-binding fragments include, but are not limited to, single-domain antibodies (variable domain of heavy chain antibodies (VHHs) or nanobodies), Fabs, F(ab′)₂s. and scFvs (single-chain variable fragments) of full-length anti-VEGF antibodies (preferably, full-length anti-VEGF monoclonal antibodies (mAbs) (collectively referred to herein as antigen-binding fragments”). In a preferred embodiment, the fully human post-translationally modified antibody against VEGF is a fully human post-translationally modified antigen-binding fragment of a monoclonal antibody (mAb) against VEGF (“HuPTMFab VEGFi”). In a further preferred embodiment, the HuPTMFab VEGFi is a fully human glycosylated antigen-binding fragment of an anti-VEGF mAb (“HuGlyFabVEGFi”). In an alternative embodiment, full-length mAbs can be used. Delivery may be accomplished via gene therapy—e.g., by administering a viral vector or other DNA expression construct encoding an anti-VEGF antigen-binding fragment or mAb (or a hyperglycosylated derivative) to the suprachoroidal space, subretinal space (from a transvitreal approach or with a catheter through the suprachoroidal space), intraretinal space, and/or outer surface of the sclera (i.e., juxtascleral administration) in the eye(s) of patients (human subjects) diagnosed with wet AMD, dry AMD, retinal vein occlusion (RVO), diabetic macular edema (DME), or diabetic retinopathy (DR) (in particular, wet AMD), to create a permanent depot in the eye that continuously supplies the human PTM, e.g., human-glycosylated, transgene product. In a preferred embodiment, the methods provided herein are used in patients (human subjects) diagnosed with wet AMD.

Described herein are anti-human vascular endothelial growth factor (hVEGF) antibodies, for example, anti-hVEGF antigen-binding fragments, produced by human retinal cells. Human VEGF (hVEGF) is a human protein encoded by the VEGF (VEGFA, VEGEB, VEGFC, or VEGED) gene. An exemplary amino acid sequence of h VEGF may be found at GenBank Accession No. AAA35789.1. An exemplary nucleic acid sequence of h VEGF may be found at GenBank Accession No. M32977.1.

In certain aspects, described herein are methods of treating a human subject diagnosed with neovascular age-related macular degeneration (nAMD) (also known as wet AMD or WAMD), dry AMD, retinal vein occlusion (RVO), diabetic macular edema (DME), or diabetic retinopathy (DR) (in particular, wet AMD), comprising delivering to the retina of said human subject a therapeutically effective amount of anti-hVEGF antigen-binding fragment produced by human retinal cells. In a specific aspect, described herein are methods of treating a human subject diagnosed with nAMD, dry AMD, retinal vein occlusion (RVO), diabetic macular edema (DME), or diabetic retinopathy (DR) (in particular, wet AMD), comprising delivering to the retina of said human subject a therapeutically effective amount of anti-hVEGF antigen-binding fragment produced by human retinal cells, by administering to the suprachoroidal space, subretinal space, or outer surface of the sclera in the eye of said human subject (e.g., by suprachoroidal injection (for example, via a suprachoroidal drug delivery device such as a microinjector with a microneedle), subretinal injection via transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), or a posterior juxtascleral depot procedure (for example, via a juxtascleral drug delivery device comprising a cannula whose tip can be inserted and kept in direct apposition to the scleral surface)) an expression vector encoding the anti-hVEGF antigen-binding fragment. In a specific aspect, described herein are methods of treating a human subject diagnosed with nAMD, dry AMD, retinal vein occlusion (RVO), diabetic macular edema (DME), or diabetic retinopathy (DR) (in particular, wet AMD), comprising delivering to the retina of said human subject a therapeutically effective amount of anti-hVEGF antigen-binding fragment produced by human retinal cells, by the use of a suprachoroidal drug delivery device such as a microinjector. In a specific aspect, described herein are methods of treating a human subject diagnosed with neovascular age-related macular degeneration (nAMD), dry AMD, retinal vein occlusion (RVO), diabetic macular edema (DME), or diabetic retinopathy (DR) (in particular, wet AMD), comprising delivering to the retina of said human subject a therapeutically effective amount of anti-hVEGF antigen-binding fragment produced by human retinal cells, wherein the human subject has a Best-Corrected Visual Acuity (BCVA) that is ≤20/20 and ≥20/400.

In certain aspects, described herein are methods of treating a human subject diagnosed with wet AMD, dry AMD, retinal vein occlusion (RVO), diabetic macular edema (DME), or diabetic retinopathy (DR) (in particular, wet AMD), comprising delivering to the retina of said human subject a therapeutically effective amount of anti-h VEGF antigen-binding fragment produced by human photoreceptor cells (e.g., cone cells and/or rod cells), horizontal cells, bipolar cells, amacrine cells, retina ganglion cells (e.g., midget cells, parasol cells, bistratified cells, giant retina ganglion cells, photosensitive ganglion cells, and/or Müller glia), and/or retinal pigment epithelial cells in the external limiting membrane. In a specific aspect, described herein are methods of treating a human subject diagnosed with wet AMD, dry AMD, retinal vein occlusion (RVO), diabetic macular edema (DME), or diabetic retinopathy (DR) (in particular, wet AMD), comprising delivering to the retina of said human subject a therapeutically effective amount of anti-hVEGF antigen-binding fragment produced by human photoreceptor cells (e.g., cone cells and/or rod cells), horizontal cells, bipolar cells, amacrine cells, retina ganglion cells (e.g., midget cells, parasol cells, bistratified cells, giant retina ganglion cells, photosensitive ganglion cells, and/or Müller glia), and/or retinal pigment epithelial cells in the external limiting membrane, by administering to the suprachoroidal space, subretinal space, or outer surface of the sclera in the eye of said human subject (e.g., by suprachoroidal injection (for example, via a suprachoroidal drug delivery device such as a microinjector with a microneedle), subretinal injection via the transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), or a posterior juxtascleral depot procedure (for example, via a juxtascleral drug delivery device comprising a cannula whose tip can be inserted and kept in direct apposition to the scleral surface)) an expression vector encoding the anti-hVEGF antigen-binding fragment. In a specific aspect, described herein are methods of treating a human subject diagnosed with wet AMD, dry AMD, retinal vein occlusion (RVO), diabetic macular edema (DME), or diabetic retinopathy (DR) (in particular, wet AMD), comprising delivering to the retina of said human subject a therapeutically effective amount of anti-hVEGF antigen-binding fragment produced by human photoreceptor cells (e.g., cone cells and/or rod cells), horizontal cells, bipolar cells, amacrine cells, retina ganglion cells (e.g., midget cells, parasol cells, bistratified cells, giant retina ganglion cells, photosensitive ganglion cells, and/or Müller glia), and/or retinal pigment epithelial cells in the external limiting membrane, by the use of a suprachoroidal drug delivery device such as a microinjector. In a specific aspect, described herein are methods of treating a human subject diagnosed with wet AMD, dry AMD, retinal vein occlusion (RVO), diabetic macular edema (DME), or diabetic retinopathy (DR) (in particular, wet AMD), comprising delivering to the retina of said human subject a therapeutically effective amount of anti-h VEGF antigen-binding fragment produced by human photoreceptor cells (e.g., cone cells and/or rod cells), horizontal cells, bipolar cells, amacrine cells, retina ganglion cells (e.g., midget cells, parasol cells, bistratified cells, giant retina ganglion cells, photosensitive ganglion cells, and/or Müller glia), and/or retinal pigment epithelial cells in the external limiting membrane, wherein the human subject has a BCVA that is ≤20/20 and ≥20/400.

In certain aspects, described herein are methods of treating a human subject diagnosed with wet AMD, dry AMD, retinal vein occlusion (RVO), diabetic macular edema (DME), or diabetic retinopathy (DR) (in particular, wet AMD), comprising delivering to the eye of said human subject a therapeutically effective amount of anti-hVEGF antigen-binding fragment produced by human retinal cells. In a specific aspect, described herein are methods of treating a human subject diagnosed with wet AMD, dry AMD, retinal vein occlusion (RVO), diabetic macular edema (DME), or diabetic retinopathy (DR) (in particular, wet AMD), comprising delivering to the eye of said human subject a therapeutically effective amount of anti-h VEGF antigen-binding fragment produced by human retinal cells, by administering to the suprachoroidal space, subretinal space, or outer surface of the sclera in the eye of said human subject (e.g., by suprachoroidal injection (for example, via a suprachoroidal drug delivery device such as a microinjector with a microneedle), subretinal injection via the transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), or a posterior juxtascleral depot procedure (for example, via a juxtascleral drug delivery device comprising a cannula whose tip can be inserted and kept in direct apposition to the scleral surface)) an expression vector encoding the anti-h VEGF antigen-binding fragment. In a specific aspect, described herein are methods of treating a human subject diagnosed with wet AMD, dry AMD, retinal vein occlusion (RVO), diabetic macular edema (DME), or diabetic retinopathy (DR) (in particular, wet AMD), comprising delivering to the eye of said human subject a therapeutically effective amount of anti-h VEGF antigen-binding fragment produced by human retinal cells, by the use of a suprachoroidal drug delivery device such as a microinjector. In a specific aspect, described herein are methods of treating a human subject diagnosed with wet AMD, dry AMD, retinal vein occlusion (RVO), diabetic macular edema (DME), or diabetic retinopathy (DR) (in particular, wet AMD)), comprising delivering to the eye of said human subject a therapeutically effective amount of anti-hVEGF antigen-binding fragment produced by human retinal cells, wherein the human subject has a BCVA that is ≤20/20 and ≥20/400.

In certain aspects, described herein are methods of treating a human subject diagnosed with wet AMD, dry AMD, retinal vein occlusion (RVO), diabetic macular edema (DME), or diabetic retinopathy (DR) (in particular, wet AMD), comprising delivering to the eye of said human subject a therapeutically effective amount of anti-hVEGF antigen-binding fragment produced by human photoreceptor cells (e.g., cone cells and/or rod cells), horizontal cells, bipolar cells, amacrine cells, retina ganglion cells (e.g., midget cells, parasol cells, bistratified cells, giant retina ganglion cells, photosensitive ganglion cells, and/or Müller glia), and/or retinal pigment epithelial cells in the external limiting membrane. In a specific aspect, described herein are methods of treating a human subject diagnosed with wet AMD, dry AMD, retinal vein occlusion (RVO), diabetic macular edema (DME), or diabetic retinopathy (DR) (in particular, wet AMD), comprising delivering to the eye of said human subject a therapeutically effective amount of anti-hVEGF antigen-binding fragment produced by human photoreceptor cells (e.g., cone cells and/or rod cells), horizontal cells, bipolar cells, amacrine cells, retina ganglion cells (e.g., midget cells, parasol cells, bistratified cells, giant retina ganglion cells, photosensitive ganglion cells, and/or Müller glia), and/or retinal pigment epithelial cells in the external limiting membrane, by administering to the suprachoroidal space, subretinal space, or outer surface of the sclera in the eye of said human subject (e.g., by suprachoroidal injection (for example, via a suprachoroidal drug delivery device such as a microinjector with a microneedle), subretinal injection via the transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), or a posterior juxtascleral depot procedure (for example, via a juxtascleral drug delivery device comprising a cannula whose tip can be inserted and kept in direct apposition to the scleral surface)) an expression vector encoding the anti-h VEGF antigen-binding fragment. In a specific aspect, described herein are methods of treating a human subject diagnosed with wet AMD, dry AMD, retinal vein occlusion (RVO), diabetic macular edema (DME), or diabetic retinopathy (DR) (in particular, wet AMD), comprising delivering to the eye of said human subject a therapeutically effective amount of anti-hVEGF antigen-binding fragment produced by human photoreceptor cells (e.g., cone cells and/or rod cells), horizontal cells, bipolar cells, amacrine cells, retina ganglion cells (e.g., midget cells, parasol cells, bistratified cells, giant retina ganglion cells, photosensitive ganglion cells, and/or Müller glia), and/or retinal pigment epithelial cells in the external limiting membrane, by the use of a suprachoroidal drug delivery device such as a microinjector. In a specific aspect, described herein are methods of treating a human subject diagnosed with wet AMD, dry AMD, retinal vein occlusion (RVO), diabetic macular edema (DME), or diabetic retinopathy (DR) (in particular, wet AMD), comprising delivering to the eye of said human subject a therapeutically effective amount of anti-hVEGF antigen-binding fragment produced by human photoreceptor cells (e.g., cone cells and/or rod cells), horizontal cells, bipolar cells, amacrine cells, retina ganglion cells (e.g., midget cells, parasol cells, bistratified cells, giant retina ganglion cells, photosensitive ganglion cells, and/or Müller glia), and/or retinal pigment epithelial cells in the external limiting membrane, wherein the human subject has a BCVA that is ≤20/20 and ≥20/400.

In certain aspects of the methods described herein, the antigen-binding fragment comprises a heavy chain comprising the amino acid sequence of SEQ ID NO. 1 or SEQ ID NO. 3, and a light chain comprising the amino acid sequence of SEQ ID NO. 2, or SEQ ID NO. 4.

In certain aspects of the methods described herein, the antigen-binding fragment comprises light chain CDRs 1-3 of SEQ ID NOs: 14-16 and heavy chain CDRs 1-3 of SEQ ID NOs:17-19 or SEQ ID NOs: 20, 18, and 21.

In a specific embodiment of the methods described herein, the antigen-binding fragment comprises light chain CDRs 1-3 of SEQ ID NOs: 14-16 and heavy chain CDRs 1-3 of SEQ ID NOs: 20, 18, and 21, wherein the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the second amino acid residue of the light chain CDR3 (i.e., the second Q in QQYSTVPWTF (SEQ ID NO. 16)) is not acetylated. In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) is not acetylated. In a preferred embodiment, the chemical modification(s) or lack of chemical modification(s) (as the case may be) described herein is determined by mass spectrometry.

In a specific embodiment of the methods described herein, the antigen-binding fragment comprises light chain CDRs 1-3 of SEQ ID NOs: 14-16 and heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the last amino acid residue of the heavy chain CDR1 (i.e., the Nin GYDFTHYGMN (SEQ ID NO. 20)) is not acetylated. In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) is not acetylated. In a preferred embodiment, the chemical modification(s) or lack of chemical modification(s) (as the case may be) described herein is determined by mass spectrometry.

In a specific embodiment of the methods described herein, the antigen-binding fragment comprises light chain CDRs 1-3 of SEQ ID NOs: 14-16 and heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein: (1) the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu); and (2) the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) is not acetylated, and the second amino acid residue of the light chain CDR3 (i.e., the second Q in QQYSTVPWTF (SEQ ID NO. 16)) is not acetylated. In a specific embodiment, the antigen-binding fragment comprises light chain CDRs 1-3 of SEQ ID NOs: 14-16 and heavy chain CDRs 1-3 of SEQ ID NOs: 20, 18, and 21, wherein: (1) the ninth amino acid residue of the heavy chain CDR1 (i.e., the M in GYDFTHYGMN (SEQ ID NO. 20)) carries one or more of the following chemical modifications: acctylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) is not acetylated; and (2) the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) is not acetylated. In a preferred embodiment, the chemical modification(s) or lack of chemical modification(s) (as the case may be) described herein is determined by mass spectrometry.

In certain aspects, described herein are methods of treating a human subject diagnosed with wet AMD, dry AMD, retinal vein occlusion (RVO), diabetic macular edema (DME), or diabetic retinopathy (DR) (in particular, wet AMD), comprising delivering to the eye of said human subject a therapeutically effective amount of anti-hVEGF antibody produced by human retinal cells. In a specific aspect, described herein are methods of treating a human subject diagnosed with wet AMD, dry AMD, retinal vein occlusion (RVO), diabetic macular edema (DME), or diabetic retinopathy (DR) (in particular, wet AMD), comprising delivering to the eye of said human subject a therapeutically effective amount of anti-hVEGF antibody produced by human retinal cells, by administering to the suprachoroidal space, subretinal space, or outer surface of the sclera in the eye of said human subject (e.g., by suprachoroidal injection (for example, via a suprachoroidal drug delivery device such as a microinjector with a microneedle), subretinal injection via the transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), or a posterior juxtascleral depot procedure (for example, via a juxtascleral drug delivery device comprising a cannula whose tip can be inserted and kept in direct apposition to the scleral surface)) an expression vector encoding the anti-hVEGF antibody. In a specific aspect, described herein are methods of treating a human subject diagnosed with wet AMD, dry AMD, retinal vein occlusion (RVO), diabetic macular edema (DME), or diabetic retinopathy (DR) (in particular, wet AMD), comprising delivering to the eye of said human subject a therapeutically effective amount of anti-hVEGF antibody produced by human retinal cells, by the use of a suprachoroidal drug delivery device such as a microinjector. In a specific aspect, described herein are methods of treating a human subject diagnosed with wet AMD, dry AMD, retinal vein occlusion (RVO), diabetic macular edema (DME), or diabetic retinopathy (DR) (in particular, wet AMD), comprising delivering to the eye of said human subject a therapeutically effective amount of anti-hVEGF antibody produced by human retinal cells, wherein the human subject has a BCVA that is ≤20/20 and ≥20/400.

In certain aspects, described herein are methods of treating a human subject diagnosed with wet AMD, dry AMD, retinal vein occlusion (RVO), diabetic macular edema (DME), or diabetic retinopathy (DR) (in particular, wet AMD), comprising delivering to the eye of said human subject a therapeutically effective amount of anti-hVEGF antibody produced by human photoreceptor cells (e.g., cone cells and/or rod cells), horizontal cells, bipolar cells, amacrine cells, retina ganglion cells (e.g., midget cells, parasol cells, bistratified cells, giant retina ganglion cells, photosensitive ganglion cells, and/or Müller glia), and/or retinal pigment epithelial cells in the external limiting membrane. In a specific aspect, described herein are methods of treating a human subject diagnosed with wet AMD, dry AMD, retinal vein occlusion (RVO), diabetic macular edema (DME), or diabetic retinopathy (DR) (in particular, wet AMD), comprising delivering to the eye of said human subject a therapeutically effective amount of anti-h VEGF antibody produced by human photoreceptor cells (e.g., cone cells and/or rod cells), horizontal cells, bipolar cells, amacrine cells, retina ganglion cells (e.g., midget cells, parasol cells, bistratified cells, giant retina ganglion cells, photosensitive ganglion cells, and/or Müller glia), and/or retinal pigment epithelial cells in the external limiting membrane, by administering to the suprachoroidal space, subretinal space, or outer surface of the sclera in the eye of said human subject (e.g., by suprachoroidal injection (for example, via a suprachoroidal drug delivery device such as a microinjector with a microneedle), subretinal injection via the transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), or a posterior juxtascleral depot procedure (for example, via a juxtascleral drug delivery device comprising a cannula whose tip can be inserted and kept in direct apposition to the scleral surface) an expression vector encoding the anti-hVEGF antibody. In a specific aspect, described herein are methods of treating a human subject diagnosed with wet AMD, dry AMD, retinal vein occlusion (RVO), diabetic macular edema (DME), or diabetic retinopathy (DR) (in particular, wet AMD), comprising delivering to the eye of said human subject a therapeutically effective amount of anti-h VEGF antibody produced by human photoreceptor cells (e.g., cone cells and/or rod cells), horizontal cells, bipolar cells, amacrine cells, retina ganglion cells (e.g., midget cells, parasol cells, bistratified cells, giant retina ganglion cells, photosensitive ganglion cells, and/or Müller glia), and/or retinal pigment epithelial cells in the external limiting membrane, by the use of a suprachoroidal drug delivery device such as a microinjector. In a specific aspect, described herein are methods of treating a human subject diagnosed with wet AMD, dry AMD, retinal vein occlusion (RVO), diabetic macular edema (DME), or diabetic retinopathy (DR) (in particular, wet AMD), comprising delivering to the eye of said human subject a therapeutically effective amount of anti-hVEGF antibody produced by human photoreceptor cells (e.g., cone cells and/or rod cells), horizontal cells, bipolar cells, amacrine cells, retina ganglion cells (e.g., midget cells, parasol cells, bistratified cells, giant retina ganglion cells, photosensitive ganglion cells, and/or Müller glia), and/or retinal pigment epithelial cells in the external limiting membrane, wherein the human subject has a BCVA that is ≤20/20 and ≥20/400.

In certain aspects, described herein are methods of treating a human subject diagnosed with wet AMD, dry AMD, retinal vein occlusion (RVO), diabetic macular edema (DME), or diabetic retinopathy (DR) (in particular, wet AMD), comprising delivering to the retina of said human subject a therapeutically effective amount of anti-hVEGF antibody produced by human retinal cells. In a specific aspect, described herein are methods of treating a human subject diagnosed with wet AMD, dry AMD, retinal vein occlusion (RVO), diabetic macular edema (DME), or diabetic retinopathy (DR) (in particular, wet AMD), comprising delivering to the retina of said human subject a therapeutically effective amount of anti-hVEGF antibody produced by human retinal cells, by administering to the suprachoroidal space, subretinal space, or outer surface of the sclera in the eye of said human subject (e.g., by suprachoroidal injection (for example, via a suprachoroidal drug delivery device such as a microinjector with a microneedle), subretinal injection via the transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), or a posterior juxtascleral depot procedure (for example, via a juxtascleral drug delivery device comprising a cannula whose tip can be inserted and kept in direct apposition to the scleral surface)) an expression vector encoding the anti-hVEGF antibody. In a specific aspect, described herein are methods of treating a human subject diagnosed with wet AMD, dry AMD, retinal vein occlusion (RVO), diabetic macular edema (DME), or diabetic retinopathy (DR) (in particular, wet AMD), comprising delivering to the retina of said human subject a therapeutically effective amount of anti-hVEGF antibody produced by human retinal cells, by the use of a suprachoroidal drug delivery device such as a microinjector. In a specific aspect, described herein are methods of treating a human subject diagnosed with wet AMD, dry AMD, retinal vein occlusion (RVO), diabetic macular edema (DME), or diabetic retinopathy (DR) (in particular, wet AMD), comprising delivering to the retina of said human subject a therapeutically effective amount of anti-hVEGF antibody produced by human retinal cells, wherein the human subject has a BCVA that is <20/20 and ≥20/400.

In certain aspects, described herein are methods of treating a human subject diagnosed with wet AMD, dry AMD, retinal vein occlusion (RVO), diabetic macular edema (DME), or diabetic retinopathy (DR) (in particular, wet AMD), comprising delivering to the retina of said human subject a therapeutically effective amount of anti-h VEGF antibody produced by human photoreceptor cells (e.g., cone cells and/or rod cells), horizontal cells, bipolar cells, amacrine cells, retina ganglion cells (e.g., midget cells, parasol cells, bistratified cells, giant retina ganglion cells, photosensitive ganglion cells, and/or Müller glia), and/or retinal pigment epithelial cells in the external limiting membrane. In a specific aspect, described herein are methods of treating a human subject diagnosed with wet AMD, dry AMD, retinal vein occlusion (RVO), diabetic macular edema (DME), or diabetic retinopathy (DR) (in particular, wet AMD), comprising delivering to the retina of said human subject a therapeutically effective amount of anti-h VEGF antibody produced by human photoreceptor cells (e.g., cone cells and/or rod cells), horizontal cells, bipolar cells, amacrine cells, retina ganglion cells (e.g., midget cells, parasol cells, bistratified cells, giant retina ganglion cells, photosensitive ganglion cells, and/or Müller glia), and/or retinal pigment epithelial cells in the external limiting membrane, by administering to the suprachoroidal space, subretinal space, or outer surface of the sclera in the cyc of said human subject (e.g., by suprachoroidal injection (for example, via a suprachoroidal drug delivery device such as a microinjector with a microneedle), subretinal injection via the transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), or a posterior juxtascleral depot procedure (for example, via a juxtascleral drug delivery device comprising a cannula whose tip can be inserted and kept in direct apposition to the scleral surface)) an expression vector encoding the anti-hVEGF antibody. In a specific aspect, described herein are methods of treating a human subject diagnosed with wet AMD, dry AMD, retinal vein occlusion (RVO), diabetic macular edema (DME), or diabetic retinopathy (DR) (in particular, wet AMD), comprising delivering to the retina of said human subject a therapeutically effective amount of anti-h VEGF antibody produced by human photoreceptor cells (e.g., cone cells and/or rod cells), horizontal cells, bipolar cells, amacrine cells, retina ganglion cells (e.g., midget cells, parasol cells, bistratified cells, giant retina ganglion cells, photosensitive ganglion cells, and/or Müller glia), and/or retinal pigment epithelial cells in the external limiting membrane, by the use of a suprachoroidal drug delivery device such as a microinjector. In a specific aspect, described herein are methods of treating a human subject diagnosed with wet AMD, dry AMD, retinal vein occlusion (RVO), diabetic macular edema (DME), or diabetic retinopathy (DR) (in particular, wet AMD), comprising delivering to the retina of said human subject a therapeutically effective amount of anti-hVEGF antibody produced by human photoreceptor cells (e.g., cone cells and/or rod cells), horizontal cells, bipolar cells, amacrine cells, retina ganglion cells (e.g., midget cells, parasol cells, bistratified cells, giant retina ganglion cells, photosensitive ganglion cells, and/or Müller glia), and/or retinal pigment epithelial cells in the external limiting membrane, wherein the human subject has a BCVA that is ≤20/20 and ≥20/400.

In certain aspects of the methods described herein, the antibody comprises a heavy chain comprising the amino acid sequence of SEQ ID NO. 1 or SEQ ID NO. 3, and a light chain comprising the amino acid sequence of SEQ ID NO. 2, or SEQ ID NO. 4.

In certain aspects of the methods described herein, the antibody comprises light chain CDRs 1-3 of SEQ ID NOs: 14-16 and heavy chain CDRs 1-3 of SEQ ID NOs: 17-19 or SEQ ID NOs: 20, 18, and 21.

In a specific embodiment of the methods described herein, the antigen-binding fragment comprises light chain CDRs 1-3 of SEQ ID NOs: 14-16 and heavy chain CDRs 1-3 of SEQ ID NOs: 20, 18, and 21, wherein the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the second amino acid residue of the light chain CDR3 (i.e., the second Q in QQYSTVPWTF (SEQ ID NO. 16)) is not acetylated. In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) is not acetylated. In a preferred embodiment, the chemical modification(s) or lack of chemical modification(s) (as the case may be) described herein is determined by mass spectrometry.

In a specific embodiment of the methods described herein, the antigen-binding fragment comprises light chain CDRs 1-3 of SEQ ID NOs: 14-16 and heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) is not acetylated. In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) is not acetylated. In a preferred embodiment, the chemical modification(s) or lack of chemical modification(s) (as the case may be) described herein is determined by mass spectrometry.

In a specific embodiment of the methods described herein, the antigen-binding fragment comprises light chain CDRs 1-3 of SEQ ID NOs: 14-16 and heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein: (1) the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the Nin WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu); and (2) the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) is not acetylated, and the second amino acid residue of the light chain CDR3 (i.e., the second Q in QQYSTVPWTF (SEQ ID NO. 16)) is not acetylated. In a specific embodiment, the antigen-binding fragment comprises light chain CDRs 1-3 of SEQ ID NOs: 14-16 and heavy chain CDRs 1-3 of SEQ ID NOs: 20, 18, and 21, wherein: (1) the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) is not acetylated; and (2) the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) is not acetylated. In a preferred embodiment, the chemical modification(s) or lack of chemical modification(s) (as the case may be) described herein is determined by mass spectrometry.

In certain aspects, described herein are methods of treating a human subject diagnosed with wet AMD, dry AMD, retinal vein occlusion (RVO), diabetic macular edema (DME), or diabetic retinopathy (DR) (in particular, wet AMD), comprising: delivering to the eye of said human subject, a therapeutically effective amount of an antigen-binding fragment of a mAb against hVEGF, said antigen-binding fragment containing a a2,6-sialylated glycan. In a specific aspect, described herein are methods of treating a human subject diagnosed with wet AMD, dry AMD, retinal vein occlusion (RVO), diabetic macular edema (DME), or diabetic retinopathy (DR) (in particular, wet AMD), comprising: delivering to the eye of said human subject, a therapeutically effective amount of an antigen-binding fragment of a mAb against hVEGF, said antigen-binding fragment containing a a2,6-sialylated glycan, by administering to the suprachoroidal space, subretinal space, or outer surface of the sclera in the eye of said human subject (e.g., by suprachoroidal injection (for example, via a suprachoroidal drug delivery device such as a microinjector with a microneedle), subretinal injection via the transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), or a posterior juxtascleral depot procedure (for example, via a juxtascleral drug delivery device comprising a cannula whose tip can be inserted and kept in direct apposition to the scleral surface)) an expression vector encoding the antigen-binding fragment of a mAb against hVEGF. In a specific aspect, described herein are methods of treating a human subject diagnosed with wet AMD, dry AMD, retinal vein occlusion (RVO), diabetic macular edema (DME), or diabetic retinopathy (DR) (in particular, wet AMD), comprising: delivering to the eye of said human subject, a therapeutically effective amount of an antigen-binding fragment of a mAb against hVEGF, said antigen-binding fragment containing a a2,6-sialylated glycan, by the use of a suprachoroidal drug delivery device such as a microinjector. In a specific aspect, described herein are methods of treating a human subject diagnosed with wet AMD, dry AMD, retinal vein occlusion (RVO), diabetic macular edema (DME), or diabetic retinopathy (DR) (in particular, wet AMD), comprising: delivering to the cyc of said human subject, a therapeutically effective amount of an antigen-binding fragment of a mAb against hVEGF, said antigen-binding fragment containing a a2,6-sialylated glycan, wherein the human subject has a BCVA that is ≤20/20 and ≥20/400.

In certain aspects, described herein are methods of treating a human subject diagnosed with wet AMD, dry AMD, retinal vein occlusion (RVO), diabetic macular edema (DME), or diabetic retinopathy (DR) (in particular, wet AMD), comprising: delivering to the eye of said human subject, a therapeutically effective amount of a glycosylated antigen-binding fragment of a mAb against hVEGF, wherein said antigen-binding fragment does not contain detectable NeuGc and/or α-Gal antigen (i.e., as used herein, “detectable” means levels detectable by standard assays described infra). In a specific embodiment, described herein are methods of treating a human subject diagnosed with wet AMD, dry AMD, retinal vein occlusion (RVO), diabetic macular edema (DME), or diabetic retinopathy (DR) (in particular, wet AMD), comprising: delivering to the eye of said human subject, a therapeutically effective amount of a glycosylated antigen-binding fragment of a mAb against hVEGF, by administering to the suprachoroidal space, subretinal space, or outer surface of the sclera in the eye of said human subject (e.g., by suprachoroidal injection (for example, via a suprachoroidal drug delivery device such as a microinjector with a microneedle), subretinal injection via the transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), or a posterior juxtascleral depot procedure (for example, via a juxtascleral drug delivery device comprising a cannula whose tip can be inserted and kept in direct apposition to the scleral surface)) an expression vector encoding the glycosylated antigen-binding fragment of a mAb against hVEGF, wherein said antigen-binding fragment does not contain detectable NeuGc and/or α-Gal antigen. In a specific embodiment, described herein are methods of treating a human subject diagnosed with wet AMD, dry AMD, retinal vein occlusion (RVO), diabetic macular edema (DME), or diabetic retinopathy (DR) (in particular, wet AMD), comprising: delivering to the eye of said human subject, a therapeutically effective amount of a glycosylated antigen-binding fragment of a mAb against hVEGF, by the use of a suprachoroidal drug delivery device such as a microinjector, wherein said antigen-binding fragment does not contain detectable NeuGc and/or α-Gal antigen. In a specific aspect, described herein are methods of treating a human subject diagnosed with wet AMD, dry AMD, retinal vein occlusion (RVO), diabetic macular edema (DME), or diabetic retinopathy (DR) (in particular, wet AMD), comprising: delivering to the eye of said human subject, a therapeutically effective amount of a glycosylated antigen-binding fragment of a mAb against h VEGF, wherein said antigen-binding fragment does not contain detectable NeuGc and/or α-Gal antigen, and wherein the human subject has a BCVA that is ≤20/20 and ≥20/400.

In certain aspects, described herein are methods of treating a human subject diagnosed with wet AMD, dry AMD, retinal vein occlusion (RVO), diabetic macular edema (DME), or diabetic retinopathy (DR) (in particular, wet AMD), wherein the method comprises: administering to the suprachoroidal space, subretinal space, or outer surface of the sclera in the eye of said human subject an expression vector encoding an antigen-binding fragment of a mAb against hVEGF (e.g., by suprachoroidal injection, subretinal injection via the transvitreal approach (a surgical procedure), subretinal administration via the suprachoroidal space, or a posterior juxtascleral depot procedure), wherein expression of said antigen-binding fragment is a2,6-sialylated upon expression from said expression vector in a human, immortalized retina-derived cell. In a specific embodiment, the administering step comprises the use of a suprachoroidal drug delivery device such as a microinjector. In a specific aspect, described herein are methods of treating a human subject diagnosed with wet AMD, dry AMD, retinal vein occlusion (RVO), diabetic macular edema (DME), or diabetic retinopathy (DR) (in particular, wet AMD), wherein the method comprises: administering to the suprachoroidal space, subretinal space, or outer surface of the sclera in the eye of said human subject an expression vector encoding an antigen-binding fragment of a mAb against hVEGF (e.g., by suprachoroidal injection, subretinal injection via the transvitreal approach (a surgical procedure), subretinal administration via the suprachoroidal space), or a posterior juxtascleral depot procedure, wherein expression of said antigen-binding fragment is a2,6-sialylated upon expression from said expression vector in a human, immortalized retina-derived cell, and wherein the human subject has a BCVA that is ≤20/20 and ≥20/400. In a specific embodiment, the administering step comprises the use of a suprachoroidal drug delivery device such as a microinjector.

In certain aspects, described herein are methods of treating a human subject diagnosed with wet AMD, dry AMD, retinal vein occlusion (RVO), diabetic macular edema (DME), or diabetic retinopathy (DR) (in particular, wet AMD), wherein the method comprises: administering or delivering to the retina of said human subject via the suprachoroidal space in the eye of said human subject (e.g., via a suprachoroidal drug delivery device such as a microinjector with a microneedle) an expression vector encoding an antigen-binding fragment of a mAb against hVEGF, wherein expression of said antigen-binding fragment is a2,6-sialylated upon expression from said expression vector in a human, immortalized retina-derived cell. In a specific aspect, described herein are methods of treating a human subject diagnosed with wet AMD, dry AMD, retinal vein occlusion (RVO), diabetic macular edema (DME), or diabetic retinopathy (DR) (in particular, wet AMD), wherein the method comprises: administering or delivering to the retina of said human subject via the suprachoroidal space in the eye of said human subject (e.g., via a suprachoroidal drug delivery device such as a microinjector with a microneedle) an expression vector encoding an antigen-binding fragment of a mAb against hVEGF, wherein expression of said antigen-binding fragment is a2,6-sialylated upon expression from said expression vector in a human, immortalized retina-derived cell, and wherein the human subject has a BCVA that is ≤20/20 and ≥20/400.

In certain aspects, described herein are methods of treating a human subject diagnosed with wet AMD, dry AMD, retinal vein occlusion (RVO), diabetic macular edema (DME), or diabetic retinopathy (DR) (in particular, wet AMD), wherein the method comprises: administering to the subretinal and/or intraretinal space of said human subject via the suprachoroidal space in the eye of said human subject (e.g., via a subretinal drug delivery device comprising a catheter that can be inserted and tunneled through the suprachoroidal space) an expression vector encoding an antigen-binding fragment of a mAb against hVEGF, wherein expression of said antigen-binding fragment is a2,6-sialylated upon expression from said expression vector in a human, immortalized retina-derived cell. In a specific aspect, described herein are methods of treating a human subject diagnosed with wet AMD, dry AMD, retinal vein occlusion (RVO), diabetic macular edema (DME), or diabetic retinopathy (DR) (in particular, wet AMD), wherein the method comprises: administering to the subretinal and/or intraretinal space of said human subject via the suprachoroidal space in the eye of said human subject (e.g., via a subretinal drug delivery device comprising a catheter that can be inserted and tunneled through the suprachoroidal space toward the posterior pole, where a small needle injects into the subretinal space) an expression vector encoding an antigen-binding fragment of a mAb against hVEGF, wherein expression of said antigen-binding fragment is a2,6-sialylated upon expression from said expression vector in a human, immortalized retina-derived cell, and wherein the human subject has a BCVA that is ≤20/20 and ≥20/400.

In certain aspects, described herein are methods of treating a human subject diagnosed with wet AMD, dry AMD, retinal vein occlusion (RVO), diabetic macular edema (DME), or diabetic retinopathy (DR) (in particular, wet AMD), wherein the method comprises: administering to the suprachoroidal space, subretinal space, or outer surface of the sclera in the eye of said human subject an expression vector encoding an antigen-binding fragment against hVEGF (e.g., by suprachoroidal injection, subretinal injection via the transvitreal approach (a surgical procedure), subretinal administration via the suprachoroidal space, or a posterior juxtascleral depot procedure), wherein expression of said antigen-binding fragment is a2,6-sialylated upon expression from said expression vector in a human, immortalized retina-derived cell, wherein said antigen-binding fragment does not contain detectable NeuGc and/or α-Gal antigen. In a specific embodiment, the administering step comprises the use of a suprachoroidal drug delivery device such as a microinjector. In a specific aspect, described herein are methods of treating a human subject diagnosed with wet AMD, dry AMD, retinal vein occlusion (RVO), diabetic macular edema (DME), or diabetic retinopathy (DR) (in particular, wet AMD), wherein the method comprises: administering to the suprachoroidal space, subretinal space, or outer surface of the sclera in the eye of said human subject an expression vector encoding an antigen-binding fragment against hVEGF (e.g., by suprachoroidal injection, subretinal injection via the transvitreal approach (a surgical procedure), subretinal administration via the suprachoroidal space, or a posterior juxtascleral depot procedure), wherein expression of said antigen-binding fragment is a2,6-sialylated upon expression from said expression vector in a human, immortalized retina-derived cell, wherein said antigen-binding fragment does not contain detectable NeuGc and/or α-Gal antigen, and wherein the human subject has a BCVA that is ≤20/20 and ≥20/400. In a specific embodiment, the administering step comprises the use of a suprachoroidal drug delivery device such as a microinjector.

In certain aspects, described herein are methods of treating a human subject diagnosed with wet AMD, dry AMD, retinal vein occlusion (RVO), diabetic macular edema (DME), or diabetic retinopathy (DR) (in particular, wet AMD), wherein the method comprises: administering or delivering to the retina of said human subject via the suprachoroidal space in the eye of said human subject (e.g., via a suprachoroidal drug delivery device such as a microinjector with a microneedle) an expression vector encoding an antigen-binding fragment against hVEGF, wherein expression of said antigen-binding fragment is a2,6-sialylated upon expression from said expression vector in a human, immortalized retina-derived cell, wherein said antigen-binding fragment does not contain detectable NeuGc and/or α-Gal antigen. In a specific aspect, described herein are methods of treating a human subject diagnosed with wet AMD, dry AMD, retinal vein occlusion (RVO), diabetic macular edema (DME), or diabetic retinopathy (DR) (in particular, wet AMD), wherein the method comprises: administering or delivering to the retina of said human subject via the suprachoroidal space in the eye of said human subject (e.g., via a suprachoroidal drug delivery device such as a microinjector with a microneedle) an expression vector encoding an antigen-binding fragment against hVEGF, wherein expression of said antigen-binding fragment is a2,6-sialylated upon expression from said expression vector in a human, immortalized retina-derived cell, wherein said antigen-binding fragment does not contain detectable NeuGc and/or α-Gal antigen, and wherein the human subject has a BCVA that is ≤20/20 and ≥20/400.

In certain aspects, described herein are methods of treating a human subject diagnosed with wet AMD, dry AMD, retinal vein occlusion (RVO), diabetic macular edema (DME), or diabetic retinopathy (DR) (in particular, wet AMD), wherein the method comprises: administering to the subretinal and/or intraretinal space of said human subject via the suprachoroidal space in the eye of said human subject (e.g., via a subretinal drug delivery device comprising a catheter that can be inserted and tunneled through the suprachoroidal space toward the posterior pole, where a small needle injects into the subretinal space) an expression vector encoding an antigen-binding fragment against hVEGF, wherein expression of said antigen-binding fragment is a2,6-sialylated upon expression from said expression vector in a human, immortalized retina-derived cell, wherein said antigen-binding fragment does not contain detectable NeuGc and/or α-Gal antigen. In a specific aspect, described herein are methods of treating a human subject diagnosed with wet AMD, dry AMD, retinal vein occlusion (RVO), diabetic macular edema (DME), or diabetic retinopathy (DR) (in particular, wet AMD), wherein the method comprises: administering to the subretinal and/or intraretinal space of said human subject via the suprachoroidal space in the eye of said human subject (e.g., via a subretinal drug delivery device comprising a catheter that can be inserted and tunneled through the suprachoroidal space toward the posterior pole, where a small needle injects into the subretinal space) an expression vector encoding an antigen-binding fragment against hVEGF, wherein expression of said antigen-binding fragment is a2,6-sialylated upon expression from said expression vector in a human, immortalized retina-derived cell, wherein said antigen-binding fragment does not contain detectable NeuGic and/or α-Gal antigen, and wherein the human subject has a BCVA that is ≤20/20 and ≥20/400.

In certain aspects, described herein are methods of treating a human subject diagnosed with wet AMD, dry AMD, retinal vein occlusion (RVO), diabetic macular edema (DME), or diabetic retinopathy (DR) (in particular, wet AMD), comprising: administering to the suprachoroidal space, subretinal space, or outer surface of the sclera in the eye of said human subject, a therapeutically effective amount of a recombinant nucleotide expression vector encoding an antigen-binding fragment of a mAb against hVEGF (e.g., by suprachoroidal injection, subretinal injection via the transvitreal approach (a surgical procedure), subretinal administration via the suprachoroidal space, or a posterior juxtascleral depot procedure), so that a depot is formed that releases said antigen-binding fragment containing a a2,6-sialylated glycan. In a specific embodiment, the administering step comprises the use of a suprachoroidal drug delivery device such as a microinjector. In certain aspects, described herein are methods of treating a human subject diagnosed with wet AMD, dry AMD, retinal vein occlusion (RVO), diabetic macular edema (DME), or diabetic retinopathy (DR) (in particular, wet AMD), comprising: administering to the suprachoroidal space, subretinal space, or outer surface of the sclera in the eye of said human subject, a therapeutically effective amount of a recombinant nucleotide expression vector encoding an antigen-binding fragment of a mAb against hVEGF (e.g., by suprachoroidal injection, subretinal injection via the transvitreal approach (a surgical procedure), subretinal administration via the suprachoroidal space, or a posterior juxtascleral depot procedure), so that a depot is formed that releases said antigen-binding fragment containing a a2,6-sialylated glycan, wherein the human subject has a BCVA that is ≤20/20 and ≥20/400. In a specific embodiment, the administering step comprises the use of a suprachoroidal drug delivery device such as a microinjector.

In certain aspects, described herein are methods of treating a human subject diagnosed with wet AMD, dry AMD, retinal vein occlusion (RVO), diabetic macular edema (DME), or diabetic retinopathy (DR) (in particular, wet AMD), comprising: administering or delivering to the retina of said human subject via the suprachoroidal space in the eye of said human subject (e.g., via a suprachoroidal drug delivery device such as a microinjector with a microneedle), a therapeutically effective amount of a recombinant nucleotide expression vector encoding an antigen-binding fragment of a mAb against hVEGF, so that a depot is formed that releases said antigen-binding fragment containing a a2,6-sialylated glycan. In certain aspects, described herein are methods of treating a human subject diagnosed with wet AMD, dry AMD, retinal vein occlusion (RVO), diabetic macular edema (DME), or diabetic retinopathy (DR) (in particular, wet AMD), comprising: administering or delivering to the retina of said human subject via the suprachoroidal space in the eye of said human subject (e.g., via a suprachoroidal drug delivery device such as a microinjector with a microneedle), a therapeutically effective amount of a recombinant nucleotide expression vector encoding an antigen-binding fragment of a mAb against hVEGF, so that a depot is formed that releases said antigen-binding fragment containing a a2,6-sialylated glycan, wherein the human subject has a BCVA that is ≤20/20 and ≥20/400.

In certain aspects, described herein are methods of treating a human subject diagnosed with wet AMD, dry AMD, retinal vein occlusion (RVO), diabetic macular edema (DME), or diabetic retinopathy (DR) (in particular, wet AMD), comprising: administering to the subretinal and/or intraretinal space of said human subject via the suprachoroidal space in the eye of said human subject (e.g., via a subretinal drug delivery device comprising a catheter that can be inserted and tunneled through the suprachoroidal space toward the posterior pole, where a small needle injects into the subretinal space), a therapeutically effective amount of a recombinant nucleotide expression vector encoding an antigen-binding fragment of a mAb against hVEGF, so that a depot is formed that releases said antigen-binding fragment containing a a2,6-sialylated glycan. In certain aspects, described herein are methods of treating a human subject diagnosed with wet AMD, dry AMD, retinal vein occlusion (RVO), diabetic macular edema (DME), or diabetic retinopathy (DR) (in particular, wet AMD), comprising: administering to the subretinal and/or intraretinal space of said human subject via the suprachoroidal space in the eye of said human subject (e.g., via a subretinal drug delivery device comprising a catheter that can be inserted and tunneled through the suprachoroidal space toward the posterior pole, where a small needle injects into the subretinal space), a therapeutically effective amount of a recombinant nucleotide expression vector encoding an antigen-binding fragment of a mAb against hVEGF, so that a depot is formed that releases said antigen-binding fragment containing a a2,6-sialylated glycan, wherein the human subject has a BCVA that is ≤20/20 and ≥20/400.

In certain aspects, described herein are methods of treating a human subject diagnosed with wet AMD, dry AMD, retinal vein occlusion (RVO), diabetic macular edema (DME), or diabetic retinopathy (DR) (in particular, wet AMD), comprising: administering to the suprachoroidal space, subretinal space, or outer surface of the sclera in the eye of said human subject, a therapeutically effective amount of a recombinant nucleotide expression vector encoding an antigen-binding fragment of a mAb against hVEGF (e.g., by suprachoroidal injection, subretinal injection via the transvitreal approach (a surgical procedure), subretinal administration via the suprachoroidal space, or a posterior juxtascleral depot procedure), so that a depot is formed that releases said antigen-binding fragment wherein said antigen-binding fragment is glycosylated but does not contain detectable NeuGc and/or α-Gal antigen. In a specific embodiment, the administering step comprises the use of a suprachoroidal drug delivery device such as a microinjector. In certain aspects, described herein are methods of treating a human subject diagnosed with wet AMD, dry AMD, retinal vein occlusion (RVO), diabetic macular edema (DME), or diabetic retinopathy (DR) (in particular, wet AMD), comprising: administering to the suprachoroidal space, subretinal space, or outer surface of the sclera in the eye of said human subject, a therapeutically effective amount of a recombinant nucleotide expression vector encoding an antigen-binding fragment of a mAb against hVEGF (e.g., by suprachoroidal injection, subretinal injection via the transvitreal approach (a surgical procedure), subretinal administration via the suprachoroidal space, or a posterior juxtascleral depot procedure), so that a depot is formed that releases said antigen-binding fragment wherein said antigen-binding fragment is glycosylated but does not contain detectable NeuGc and/or α-Gal antigen, and wherein the human subject has a BCVA that is ≤20/20 and ≥20/400. In a specific embodiment, the administering step comprises the use of a suprachoroidal drug delivery device such as a microinjector.

In certain aspects, described herein are methods of treating a human subject diagnosed with wet AMD, dry AMD, retinal vein occlusion (RVO), diabetic macular edema (DME), or diabetic retinopathy (DR) (in particular, wet AMD), comprising: administering or delivering to the retina of said human subject via the suprachoroidal space in the eye of said human subject (e.g., via a suprachoroidal drug delivery device such as a microinjector with a microneedle), a therapeutically effective amount of a recombinant nucleotide expression vector encoding an antigen-binding fragment of a mAb against hVEGF, so that a depot is formed that releases said antigen-binding fragment wherein said antigen-binding fragment is glycosylated but does not contain detectable NeuGc and/or α-Gal antigen. In certain aspects, described herein are methods of treating a human subject diagnosed with wet AMD, dry AMD, retinal vein occlusion (RVO), diabetic macular edema (DME), or diabetic retinopathy (DR) (in particular, wet AMD), comprising: administering or delivering to the retina of said human subject via the suprachoroidal space in the eye of said human subject (e.g., via a suprachoroidal drug delivery device such as a microinjector with a microneedle), a therapeutically effective amount of a recombinant nucleotide expression vector encoding an antigen-binding fragment of a mAb against hVEGF, so that a depot is formed that releases said antigen-binding fragment wherein said antigen-binding fragment is glycosylated but does not contain detectable NeuGc and/or α-Gal antigen, and wherein the human subject has a BCVA that is ≤20/20 and ≥20/400.

In certain aspects, described herein are methods of treating a human subject diagnosed with wet AMD, dry AMD, retinal vein occlusion (RVO), diabetic macular edema (DME), or diabetic retinopathy (DR) (in particular, wet AMD), comprising: administering to the subretinal and/or intraretinal space of said human subject via the suprachoroidal space in the eye of said human subject (e.g., via a subretinal drug delivery device comprising a catheter that can be inserted and tunneled through the suprachoroidal space toward the posterior pole, where a small needle injects into the subretinal space), a therapeutically effective amount of a recombinant nucleotide expression vector encoding an antigen-binding fragment of a mAb against hVEGF, so that a depot is formed that releases said antigen-binding fragment wherein said antigen-binding fragment is glycosylated but does not contain detectable NeuGe and/or α-Gal antigen. In certain aspects, described herein are methods of treating a human subject diagnosed with wet AMD, dry AMD, retinal vein occlusion (RVO), diabetic macular edema (DME), or diabetic retinopathy (DR) (in particular, wet AMD), comprising: administering to the subretinal and/or intraretinal space of said human subject via the suprachoroidal space in the eye of said human subject (e.g., via a subretinal drug delivery device comprising a catheter that can be inserted and tunneled through the suprachoroidal space toward the posterior pole, where a small needle injects into the subretinal space), a therapeutically effective amount of a recombinant nucleotide expression vector encoding an antigen-binding fragment of a mAb against hVEGF, so that a depot is formed that releases said antigen-binding fragment wherein said antigen-binding fragment is glycosylated but does not contain detectable NeuGc and/or α-Gal antigen, and wherein the human subject has a BCVA that is ≤20/20 and ≥20/400.

In certain aspects of the methods described herein, the antigen-binding fragment comprises a heavy chain comprising the amino acid sequence of SEQ ID NO. 1 or SEQ ID NO. 3, and a light chain comprising the amino acid sequence of SEQ ID NO. 2, or SEQ ID NO. 4.

In certain aspects of the methods described herein, the antigen-binding fragment further contains a tyrosine-sulfation.

In certain aspects of the methods described herein, production of said antigen-binding fragment containing a a2,6-sialylated glycan is confirmed by transducing PER.C6 or RPE cell line with said recombinant nucleotide expression vector in cell culture.

In certain aspects of the methods described herein, production of said antigen-binding fragment containing a tyrosine-sulfation is confirmed by transducing PER.C6 or RPE cell line with said recombinant nucleotide expression vector in cell culture.

In certain aspects of the methods described herein, the vector has a hypoxia-inducible promoter.

In certain aspects of the methods described herein, the antigen-binding fragment comprises light chain CDRs 1-3 of SEQ ID NOs: 14-16 and heavy chain CDRs 1-3 of SEQ ID NOs: 17-19 or SEQ ID NOs: 20, 18, and 21.

In a specific embodiment of the methods described herein, the antigen-binding fragment comprises light chain CDRs 1-3 of SEQ ID NOs: 14-16 and heavy chain CDRs 1-3 of SEQ ID NOs: 20, 18, and 21, wherein the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the second amino acid residue of the light chain CDR3 (i.e., the second Q in QQYSTVPWTF (SEQ ID NO. 16)) is not acetylated. In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) is not acetylated. In a preferred embodiment, the chemical modification(s) or lack of chemical modification(s) (as the case may be) described herein is determined by mass spectrometry.

In a specific embodiment of the methods described herein, the antigen-binding fragment comprises light chain CDRs 1-3 of SEQ ID NOs: 14-16 and heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the Nin WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) is not acetylated. In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the ninth amino acid residuc of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) is not acetylated. In a preferred embodiment, the chemical modification(s) or lack of chemical modification(s) (as the case may be) described herein is determined by mass spectrometry.

In a specific embodiment of the methods described herein, the antigen-binding fragment comprises light chain CDRs 1-3 of SEQ ID NOs: 14-16 and heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acctylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein: (1) the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu); and (2) the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) is not acetylated, and the second amino acid residue of the light chain CDR3 (i.e., the second Q in QQYSTVPWTF (SEQ ID NO. 16)) is not acetylated. In a specific embodiment, the antigen-binding fragment comprises light chain CDRs 1-3 of SEQ ID NOs: 14-16 and heavy chain CDRs 1-3 of SEQ ID NOs: 20, 18, and 21, wherein: (1) the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the Nin WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) is not acetylated; and (2) the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) is not acetylated. In a preferred embodiment, the chemical modification(s) or lack of chemical modification(s) (as the case may be) described herein is determined by mass spectrometry.

In certain aspects of the methods described herein, the antigen-binding fragment transgene encodes a leader peptide. A leader peptide may also be referred to as a signal peptide or leader sequence herein.

In certain aspects, described herein are methods of treating a human subject diagnosed with wet AMD, dry AMD, retinal vein occlusion (RVO), diabetic macular edema (DME), or diabetic retinopathy (DR) (in particular, wet AMD), comprising: administering to the suprachoroidal space, subretinal space, or outer surface of the sclera in the eye of said human subject, a therapeutically effective amount of a recombinant nucleotide expression vector encoding an antigen-binding fragment of a mAb against hVEGF (e.g., by suprachoroidal injection, subretinal injection via the transvitreal approach (a surgical procedure), subretinal administration via the suprachoroidal space, or a posterior juxtascleral depot procedure), so that a depot is formed that releases said antigen-binding fragment containing a a2,6-sialylated glycan; wherein said recombinant vector, when used to transduce PER. C6 or RPE cells in culture results in production of said antigen-binding fragment containing a a2,6-sialylated glycan in said cell culture. In a specific embodiment, the administering step comprises the use of a suprachoroidal drug delivery device such as a microinjector. In certain aspects, described herein are methods of treating a human subject diagnosed with wet AMD, dry AMD, retinal vein occlusion (RVO), diabetic macular edema (DME), or diabetic retinopathy (DR) (in particular, wet AMD), comprising: administering to the suprachoroidal space, subretinal space, or outer surface of the sclera in the eye of said human subject, a therapeutically effective amount of a recombinant nucleotide expression vector encoding an antigen-binding fragment of a mAb against hVEGF (e.g., by suprachoroidal injection, subretinal injection via the transvitreal approach (a surgical procedure), subretinal administration via the suprachoroidal space, or a posterior juxtascleral depot procedure), so that a depot is formed that releases said antigen-binding fragment containing a a2,6-sialylated glycan; wherein said recombinant vector, when used to transduce PER.C6 or RPE cells in culture results in production of said antigen-binding fragment containing a a2,6-sialylated glycan in said cell culture, and wherein the human subject has a BCVA that is ≤20/20 and ≥20/400. In a specific embodiment, the administering step comprises the use of a suprachoroidal drug delivery device such as a microinjector.

In certain aspects, described herein are methods of treating a human subject diagnosed with wet AMD, dry AMD, retinal vein occlusion (RVO), diabetic macular edema (DME), or diabetic retinopathy (DR) (in particular, wet AMD), comprising: administering or delivering to the retina of said human subject via the suprachoroidal space in the eye of said human subject (e.g., via a suprachoroidal drug delivery device such as a microinjector with a microneedle), a therapeutically effective amount of a recombinant nucleotide expression vector encoding an antigen-binding fragment of a mAb against hVEGF, so that a depot is formed that releases said antigen-binding fragment containing a a2,6-sialylated glycan; wherein said recombinant vector, when used to transduce PER.C6 or RPE cells in culture results in production of said antigen-binding fragment containing a a2,6-sialylated glycan in said cell culture. In certain aspects, described herein are methods of treating a human subject diagnosed with wet AMD, dry AMD, retinal vein occlusion (RVO), diabetic macular edema (DME), or diabetic retinopathy (DR) (in particular, wet AMD), comprising: administering or delivering to the retina of said human subject via the suprachoroidal space in the eye of said human subject (e.g., via a suprachoroidal drug delivery device such as a microinjector with a microneedle), a therapeutically effective amount of a recombinant nucleotide expression vector encoding an antigen-binding fragment of a mAb against hVEGF, so that a depot is formed that releases said antigen-binding fragment containing a a2,6-sialylated glycan; wherein said recombinant vector, when used to transduce PER.C6 or RPE cells in culture results in production of said antigen-binding fragment containing a a2,6-sialylated glycan in said cell culture, and wherein the human subject has a BCVA that is ≤20/20 and ≥20/400.

In certain aspects, described herein are methods of treating a human subject diagnosed with wet AMD, dry AMD, retinal vein occlusion (RVO), diabetic macular edema (DME), or diabetic retinopathy (DR) (in particular, wet AMD), comprising: administering to the subretinal and/or intraretinal space of said human subject via the suprachoroidal space in the eye of said human subject (e.g., via a subretinal drug delivery device comprising a catheter that can be inserted and tunneled through the suprachoroidal space toward the posterior pole, where a small needle injects into the subretinal space), a therapeutically effective amount of a recombinant nucleotide expression vector encoding an antigen-binding fragment of a mAb against hVEGF, so that a depot is formed that releases said antigen-binding fragment containing a a2,6-sialylated glycan; wherein said recombinant vector, when used to transduce PER.C6 or RPE cells in culture results in production of said antigen-binding fragment containing a a2,6-sialylated glycan in said cell culture. In certain aspects, described herein are methods of treating a human subject diagnosed with wet AMD, dry AMD, retinal vein occlusion (RVO), diabetic macular edema (DME), or diabetic retinopathy (DR) (in particular, wet AMD), comprising: administering to the subretinal and/or intraretinal space of said human subject via the suprachoroidal space in the eye of said human subject (e.g., via a subretinal drug delivery device comprising a catheter that can be inserted and tunneled through the suprachoroidal space toward the posterior pole, where a small needle injects into the subretinal space), a therapeutically effective amount of a recombinant nucleotide expression vector encoding an antigen-binding fragment of a mAb against hVEGF, so that a depot is formed that releases said antigen-binding fragment containing a a2,6-sialylated glycan; wherein said recombinant vector, when used to transduce PER.C6 or RPE cells in culture results in production of said antigen-binding fragment containing a a2,6-sialylated glycan in said cell culture, and wherein the human subject has a BCVA that is ≤20/20 and ≥20/400.

In certain aspects, described herein are methods of treating a human subject diagnosed with wet AMD, dry AMD, retinal vein occlusion (RVO), diabetic macular edema (DME), or diabetic retinopathy (DR) (in particular, wet AMD), comprising: administering to the suprachoroidal space, subretinal space, or outer surface of the sclera in the eye of said human subject, a therapeutically effective amount of a recombinant nucleotide expression vector encoding an antigen-binding fragment of a mAb against hVEGF (e.g., by suprachoroidal injection, subretinal injection via the transvitreal approach (a surgical procedure), subretinal administration via the suprachoroidal space, or a posterior juxtascleral depot procedure), so that a depot is formed that releases said antigen-binding fragment wherein said antigen-binding fragment is glycosylated but does not contain detectable NeuGc and/or α-Gal antigen; wherein said recombinant vector, when used to transduce PER.C6 or RPE cells in culture results in production of said antigen-binding fragment that is glycosylated but does not contain detectable NeuGc and/or α-Gal antigen in said cell culture. In a specific embodiment, the administering step comprises the use of a suprachoroidal drug delivery device such as a microinjector. In certain aspects, described herein are methods of treating a human subject diagnosed with wet AMD, dry AMD, retinal vein occlusion (RVO), diabetic macular edema (DME), or diabetic retinopathy (DR) (in particular, wet AMD), comprising: administering to the suprachoroidal space, subretinal space, or outer surface of the sclera in the eye of said human subject, a therapeutically effective amount of a recombinant nucleotide expression vector encoding an antigen-binding fragment of a mAb against hVEGF (e.g., by suprachoroidal injection, subretinal injection via the transvitreal approach (a surgical procedure), subretinal administration via the suprachoroidal space, or a posterior juxtascleral depot procedure), so that a depot is formed that releases said antigen-binding fragment wherein said antigen-binding fragment is glycosylated but does not contain detectable NeuGc and/or α-Gal antigen; wherein said recombinant vector, when used to transduce PER.C6 or RPE cells in culture results in production of said antigen-binding fragment that is glycosylated but does not contain detectable NeuGc and/or α-Gal antigen in said cell culture, and wherein the human subject has a BCVA that is ≤20/20 and ≥20/400. In a specific embodiment, the administering step comprises the use of a suprachoroidal drug delivery device such as a microinjector.

In certain aspects, described herein are methods of treating a human subject diagnosed with wet AMD, dry AMD, retinal vein occlusion (RVO), diabetic macular edema (DME), or diabetic retinopathy (DR) (in particular, wet AMD), comprising: administering or delivering to the retina of said human subject via the suprachoroidal space in the eye of said human subject (e.g., via a suprachoroidal drug delivery device such as a microinjector with a microneedle), a therapeutically effective amount of a recombinant nucleotide expression vector encoding an antigen-binding fragment of a mAb against hVEGF, so that a depot is formed that releases said antigen-binding fragment wherein said antigen-binding fragment is glycosylated but does not contain detectable NeuGc and/or α-Gal antigen; wherein said recombinant vector, when used to transduce PER.C6 or RPE cells in culture results in production of said antigen-binding fragment that is glycosylated but does not contain detectable NeuGc and/or α-Gal antigen in said cell culture. In certain aspects, described herein are methods of treating a human subject diagnosed with wet AMD, dry AMD, retinal vein occlusion (RVO), diabetic macular edema (DME), or diabetic retinopathy (DR) (in particular, wet AMD), comprising: administering or delivering to the retina of said human subject via the suprachoroidal space in the eye of said human subject (e.g., via a suprachoroidal drug delivery device such as a microinjector with a microneedle), a therapeutically effective amount of a recombinant nucleotide expression vector encoding an antigen-binding fragment of a mAb against hVEGF, so that a depot is formed that releases said antigen-binding fragment wherein said antigen-binding fragment is glycosylated but does not contain detectable NeuGc and/or α-Gal antigen; wherein said recombinant vector, when used to transduce PER.C6 or RPE cells in culture results in production of said antigen-binding fragment that is glycosylated but does not contain detectable NeuGc and/or α-Gal antigen in said cell culture, and wherein the human subject has a BCVA that is ≤20/20 and ≥20/400.

In certain aspects, described herein are methods of treating a human subject diagnosed with wet AMD, dry AMD, retinal vein occlusion (RVO), diabetic macular edema (DME), or diabetic retinopathy (DR) (in particular, wet AMD), comprising: administering to the subretinal and/or intraretinal space of said human subject via the suprachoroidal space in the eye of said human subject (e.g., via a subretinal drug delivery device comprising a catheter that can be inserted and tunneled through the suprachoroidal space toward the posterior pole, where a small needle injects into the subretinal space), a therapeutically effective amount of a recombinant nucleotide expression vector encoding an antigen-binding fragment of a mAb against hVEGF, so that a depot is formed that releases said antigen-binding fragment wherein said antigen-binding fragment is glycosylated but does not contain detectable NeuGc and/or α-Gal antigen; wherein said recombinant vector, when used to transduce PER.C6 or RPE cells in culture results in production of said antigen-binding fragment that is glycosylated but does not contain detectable NeuGc and/or α-Gal antigen in said cell culture. In certain aspects, described herein are methods of treating a human subject diagnosed with wet AMD, dry AMD, retinal vein occlusion (RVO), diabetic macular edema (DME), or diabetic retinopathy (DR) (in particular, wet AMD), comprising: administering to the subretinal and/or intraretinal space of said human subject via the suprachoroidal space in the eye of said human subject (e.g., via a subretinal drug delivery device comprising a catheter that can be inserted and tunneled through the suprachoroidal space toward the posterior pole, where a small needle injects into the subretinal space), a therapeutically effective amount of a recombinant nucleotide expression vector encoding an antigen-binding fragment of a mAb against hVEGF, so that a depot is formed that releases said antigen-binding fragment wherein said antigen-binding fragment is glycosylated but does not contain detectable NeuGc and/or α-Gal antigen; wherein said recombinant vector, when used to transduce PER.C6 or RPE cells in culture results in production of said antigen-binding fragment that is glycosylated but does not contain detectable NeuGc and/or α-Gal antigen in said cell culture, and wherein the human subject has a BCVA that is ≤20/20 and ≥20/400.

In certain aspects of the methods described herein, the human subject has a BCVA that is ≤20/63 and ≥20/400.

In certain aspects of the methods described herein, the BCVA is the BCVA in the eye to be treated in the human subject.

In certain aspects of the methods described herein, delivering to the eye comprises delivering to the retina, choroid, and/or vitreous humor of the eye.

In certain aspects of the methods described herein, the antigen-binding fragment comprises a heavy chain that comprises one, two, three, or four additional amino acids at the C-terminus.

In certain aspects of the methods described herein, the antigen-binding fragment comprises a heavy chain that does not comprise an additional amino acid at the C-terminus.

In certain aspects of the methods described herein produces a population of antigen-binding fragment molecules, wherein the antigen-binding fragment molecules comprise a heavy chain, and wherein 0.5%, 1%, 2%, 3%, 4%, 5%, 10%, or 20%, or less of the population of antigen-binding fragment molecules comprises one, two, three, or four additional amino acids at the C-terminus of the heavy chain. In certain aspects of the methods described herein produces a population of antigen-binding fragment molecules, wherein the antigen-binding fragment molecules comprise a heavy chain, and wherein 0.5%, 1%, 2%, 3%, 4%, 5%, 10%, or 20%, or less but more than 0% of the population of antigen-binding fragment molecules comprises one, two, three, or four additional amino acids at the C-terminus of the heavy chain.

In certain aspects of the methods described herein produces a population of antigen-binding fragment molecules, wherein the antigen-binding fragment molecules comprise a heavy chain, and wherein 0.5-1%, 0.5%-2%, 0.5%-3%, 0.5%-4%, 0.5%-5%, 0.5%-10%, 0.5%-20%, 1%-2%, 1%-3%, 1%-4%, 1%-5%, 1%-10%, 1%-20%, 2%-3%, 2%-4%, 2%-5%, 2%-10%, 2%-20%, 3%-4%, 3%-5%, 3%-10%, 3%-20%, 4%-5%, 4%-10%, 4%-20%, 5%-10%, 5%-20%, or 10%-20% of the population of antigen-binding fragment molecules comprises one, two, three, or four additional amino acids at the C-terminus of the heavy chain.

Subjects to whom such gene therapy is administered should be those responsive to anti-VEGF therapy. In particular embodiments, the methods encompass treating patients who have been diagnosed with wet AMD, dry AMD, retinal vein occlusion (RVO), diabetic macular edema (DME), or diabetic retinopathy (DR) (in particular, wet AMD) and identified as responsive to treatment with an anti-VEGF antibody. In more specific embodiments, the patients are responsive to treatment with an anti-VEGF antigen-binding fragment. In certain embodiments, the patients have been shown to be responsive to treatment with an anti-VEGF antigen-binding fragment injected intravitreally prior to treatment with gene therapy. In specific embodiments, the patients have previously been treated with LUCENTIS® (ranibizumab), EYLEA® (aflibercept), and/or AVASTIN® (bevacizumab), and have been found to be responsive to one or more of said LUCENTIS® (ranibizumab), EYLEA® (aflibercept), and/or AVASTIN® (bevacizumab).

Subjects to whom such viral vector or other DNA expression construct is delivered should be responsive to the anti-h VEGF antigen-binding fragment encoded by the transgene in the viral vector or expression construct. To determine responsiveness, the anti-VEGF antigen-binding fragment transgene product (e.g., produced in cell culture, bioreactors, etc.) may be administered directly to the subject, such as by intravitreal injection.

The HuPTMFab VEGFi, e.g., HuGlyFab VEGFi, encoded by the transgene can include, but is not limited to an antigen-binding fragment of an antibody that binds to hVEGF, such as bevacizumab; an anti-hVEGF Fab moiety such as ranibizumab; or such bevacizumab or ranibizumab Fab moieties engineered to contain additional glycosylation sites on the Fab domain (e.g., see Courtois et al., 2016, mAbs 8: 99-112 which is incorporated by reference herein in its entirety for it description of derivatives of bevacizumab that are hyperglycosylated on the Fab domain of the full length antibody).

The recombinant vector used for delivering the transgene should have a tropism for human retinal cells or photoreceptor cells. Such vectors can include non-replicating recombinant adeno-associated virus vectors (“rAAV”), particularly those bearing an AAV8 capsid are preferred. However, other viral vectors may be used, including but not limited to lentiviral vectors, vaccinia viral vectors, or non-viral expression vectors referred to as “naked DNA” constructs. Preferably, the HuPTMFab VEGFi, e.g., HuGlyFab VEGFi, transgene should be controlled by appropriate expression control elements, for example, the CB7 promoter (a chicken β-actin promoter and CMV enhancer), the RPE65 promoter, or opsin promoter to name a few, and can include other expression control elements that enhance expression of the transgene driven by the vector (e.g., introns such as the chicken β-actin intron, minute virus of mice (MVM) intron, human factor IX intron (e.g., FIX truncated intron 1), β-globin splice donor/immunoglobulin heavy chain spice acceptor intron, adenovirus splice donor /immunoglobulin splice acceptor intron, SV40 late splice donor/splice acceptor (19S/16S) intron, and hybrid adenovirus splice donor/IgG splice acceptor intron and polyA signals such as the rabbit β-globin poly A signal, human growth hormone (hGH) poly A signal, SV40 late poly A signal, synthetic polyA (SPA) signal, and bovine growth hormone (bGH) poly A signal). See, e.g., Powell and Rivera-Soto, 2015, Discov. Med., 19(102):49-57.

Gene therapy constructs are designed such that both the heavy and light chains are expressed. More specifically, the heavy and light chains should be expressed at about equal amounts, in other words, the heavy and light chains are expressed at approximately a 1:1 ratio of heavy chains to light chains. The coding sequences for the heavy and light chains can be engineered in a single construct in which the heavy and light chains are separated by a cleavable linker or IRES so that separate heavy and light chain polypeptides are expressed. See, e.g., Section 5.2.4 for specific leader sequences and Section 5.2.5 for specific IRES, 2A, and other linker sequences that can be used with the methods and compositions provided herein.

Pharmaceutical compositions suitable for suprachoroidal, subretinal, juxtascleral and/or intraretinal administration comprise a suspension of the recombinant (e.g., rHuGlyFab VEGFi) vector in a formulation buffer comprising a physiologically compatible aqueous buffer, a surfactant and optional excipients.

Therapeutically effective doses of the recombinant vector should be administered subretinally and/or intraretinally (e.g., by subretinal injection via the transvitreal approach (a surgical procedure), or subretinal administration via the suprachoroidal space) in a volume ranging from ≥0.1 mL to ≤0.5 mL, preferably in 0.1 to 0.30 mL (100-300 μl), and most preferably, in a volume of 0.25 mL (250 μl). Therapeutically effective doses of the recombinant vector should be administered suprachoroidally (e.g., by suprachoroidal injection) in a volume of 100 μl or less, for example, in a volume of 50-100 μl. Therapeutically effective doses of the recombinant vector should be administered to the outer surface of the sclera (e.g., by a posterior juxtascleral depot procedure) in a volume of 500 μl or less, for example, in a volume of 10-20 μl, 20-50 μl, 50-100 μl, 100-200 μl, 200-300 μl, 300-400 μl, or 400-500 μl. Subretinal injection is a surgical procedure performed by trained retinal surgeons that involves a vitrectomy with the subject under local anesthesia, and subretinal injection of the gene therapy into the retina (see, e.g., Campochiaro et al., 2017, Hum Gen Ther 28(1):99-111, which is incorporated by reference herein in its entirety). In a specific embodiment, the subretinal administration is performed via the suprachoroidal space using a suprachoroidal catheter which injects drug into the subretinal space, such as a subretinal drug delivery device that comprises a catheter which can be inserted and tunneled through the suprachoroidal spece to the posterior pole, where a small needle injects into the subretinal space (see, e.g., Baldassarre et al., 2017, Subretinal Delivery of Cells via the Suprachoroidal Space: Janssen Trial. In: Schwartz. et al. (eds) Cellular Therapies for Retinal Disease, Springer, Cham; International Patent Application Publication No. WO 2016/040635 A1; each of which is incorporated by reference herein in its entirety). Suprachoroidal administration procedures involve administration of a drug to the suprachoroidal space of the eye, and are normally performed using a suprachoroidal drug delivery device such as a microinjector with a microneedle (see, e.g., Hariprasad, 2016, Retinal Physician 13: 20-23; Goldstein, 2014, Retina Today 9(5): 82-87; each of which is incorporated by reference herein in its entirety). The suprachoroidal drug delivery devices that can be used to deposit the expression vector in the suprachoroidal space according to the invention described herein include, but are not limited to, suprachoroidal drug delivery devices manufactured by ClearsideR Biomedical, Inc. (see, for example, Hariprasad, 2016, Retinal Physician 13: 20-23) and MedOne suprachoroidal catheters. The subretinal drug delivery devices that can be used to deposit the expression vector in the subretinal space via the suprachoroidal space according to the invention described herein include, but are not limited to, subretinal drug delivery devices manufactured by Janssen Pharmaceuticals, Inc. (see, for example, International Patent Application Publication No. WO 2016/040635 A1). In a specific embodiment, administration to the outer surface of the sclera is performed by a juxtascleral drug delivery device comprising a cannula whose tip can be inserted and kept in direct apposition to the scleral surface. See Section 5.3.2 for more details of the different modes of administration. Suprachoroidal, subretinal, juxtascleral and/or intraretinal administration should result in delivery of the soluble transgene product to the retina, the vitreous humor, and/or the aqueous humor. The expression of the transgene product (e.g., the encoded anti-VEGF antibody) by retinal cells, e.g., rod, cone, retinal pigment epithelial, horizontal, bipolar, amacrine, ganglion, and/or Müller cells, results in delivery and maintenance of the transgene product in the retina, the vitreous humor, and/or the aqueous humor. Doses that maintain a concentration of the transgene product at a Cₘᵢₙ of at least 0.330 μg/ml. in the Vitreous humour, or 0.110 μg/mL in the Aqueous humour (the anterior chamber of the eye) for three months are desired; thereafter, Vitreous Cₘᵢₙ concentrations of the transgene product ranging from 1.70 to 6.60 μg/mL, and/or Aqueous Cₘᵢₙ concentrations ranging from 0.567 to 2.20 μg/mL should be maintained. However, because the transgene product is continuously produced, maintenance of lower concentrations can be effective. The concentration of the transgene product can be measured in patient samples of the vitreous humour and/or aqueous from the anterior chamber of the treated eye. Alternatively, vitreous humour concentrations can be estimated and/or monitored by measuring the patient's serum concentrations of the transgene product the ratio of systemic to vitreal exposure to the transgene product is about 1:90,000. (E.g., see, vitreous humor and serum concentrations of ranibizumab reported in Xu L, et al., 2013, Invest. Opthal. Vis. Sci. 54: 1616-1624, at p. 1621 and Table 5 at p. 1623, which is incorporated by reference herein in its entirety).

The invention has several advantages over standard of care treatments that involve repeated ocular injections of high dose boluses of the VEGF inhibitor that dissipate over time resulting in peak and trough levels. Sustained expression of the transgene product antibody, as opposed to injecting an antibody repeatedly, allows for a more consistent levels of antibody to be present at the site of action, and is less risky and more convenient for patients, since fewer injections need to be made, resulting in fewer doctor visits. Consitent protein production may leads to better clinical outcomes as edema rebound in the retina is less likely to occur. Furthermore, antibodies expressed from transgenes are post-translationally modified in a different manner than those that are directly injected because of the different microenvironment present during and after translation. Without being bound by any particular theory, this results in antibodies that have different diffusion, bioactivity, distribution, affinity, pharmacokinetic, and immunogenicity characteristics, such that the antibodies delivered to the site of action are “biobetters” in comparison with directly injected antibodies.

In addition, antibodies expressed from transgenes in vivo are not likely to contain degradation products associated with antibodies produced by recombinant technologies, such as protein aggregation and protein oxidation. Aggregation is an issue associated with protein production and storage due to high protein concentration, surface interaction with manufacturing equipment and containers, and purification with certain buffer systems. These conditions, which promote aggregation, do not exist in transgene expression in gene therapy. Oxidation, such as methionine, tryptophan, and histidine oxidation, is also associated with protein production and storage, and is caused by stressed cell culture conditions, metal and air contact, and impurities in buffers and excipients. The proteins expressed from transgenes in vivo may also oxidize in a stressed condition. However, humans, and many other organisms, are equipped with an antioxidation defense system, which not only reduces the oxidation stress, but sometimes also repairs and/or reverses the oxidation. Thus, proteins produced in vivo are not likely to be in an oxidized form. Both aggregation and oxidation could affect the potency, pharmacokinetics (clearance), and immunogenicity.

Without being bound by theory, the methods and compositions provided herein are based, in part, on the following principles:

(i) Human retinal cells are secretory cells that possess the cellular machinery for post-translational processing of secreted proteins—including glycosylation and tyrosine-O-sulfation, a robust process in retinal cells. (See, e.g., Wang et al., 2013, Analytical Biochem. 427: 20-28 and Adamis et al., 1993, BBRC 193: 631-638 reporting the production of glycoproteins by retinal cells; and Kanan et al., 2009, Exp. Eye Res. 89: 559-567 and Kanan & Al-Ubaidi, 2015, Exp. Eye Res. 133: 126-131 reporting the production of tyrosine-sulfated glycoproteins secreted by retinal cells, each of which is incorporated by reference in its entirety for post-translational modifications made by human retinal cells).

(ii) Contrary to the state of the art understanding, anti-VEGF antigen-binding fragments, such as ranibizumab (and the Fab domain of full length anti-VEGF mAbs such as bevacizumab) do indeed possess N-linked glycosylation sites. For example, see FIG. 1 which identifies non-consensus asparaginal (“N”) glycosylation sites in the CH domain (TVSWN165SGAL) and in the CL domain (QSGN158SQE), as well as glutamine (“Q”) residues that are glycosylation sites in the VH domain (Q115GT) and VL domain (TFQ100GT) of ranibizumab (and corresponding sites in the Fab of bevacizumab). (See, e.g., Valliere-Douglass et al., 2009, J. Biol. Chem. 284: 32493-32506, and Valliere-Douglass et al., 2010, J. Biol. Chem. 285: 16012-16022, each of which is incorporated by reference in its entirety for the identification of N-linked glycosylation sites in antibodies).

(iii) While such non-canonical sites usually result in low level glycosylation (e.g., about 1-5%) of the antibody population, the functional benefits may be significant in immunoprivileged organs, such as the eye (See, e.g., van de Bovenkamp et al., 2016, J. Immunol. 196:1435-1441). For example, Fab glycosylation may affect the stability, half-life, and binding characteristics of an antibody. To determine the effects of Fab glycosylation on the affinity of the antibody for its target, any technique known to one of skill in the art may be used, for example, enzyme linked immunosorbent assay (ELISA), or surface plasmon resonance (SPR). To determine the effects of Fab glycosylation on the half-life of the antibody, any technique known to one of skill in the art may be used, for example, by measurement of the levels of radioactivity in the blood or organs (e.g., the eye) in a subject to whom a radiolabelled antibody has been administered. To determine the effects of Fab glycosylation on the stability, for example, levels of aggregation or protein unfolding, of the antibody, any technique known to one of skill in the art may be used, for example, differential scanning calorimetry (DSC), high performance liquid chromatography (HPLC), e.g., size exclusion high performance liquid chromatography (SEC-HPLC), capillary electrophoresis, mass spectrometry, or turbidity measurement. Provided herein, the HuPTMFab VEGFi, e.g., HuGlyFab VEGFi, transgene results in production of a Fab which is 0.5%, 1%, 2%, 3%, 4%, 5%, 6%, 7%, 8%, 9%, or 10% or more glycosylated at non-canonical sites. In certain embodiments, 0.5%, 1%, 2%, 3%, 4%, 5%, 6%, 7%, 8%, 9%, or 10% or more Fabs from a population of Fabs are glycosylated at non-canonical sites. In certain embodiments, 0.5%, 1%, 2%, 3%, 4%, 5%, 6%, 7%, 8%, 9%, or 10% or more non-canonical sites are glycosylated. In certain embodiments, the glycosylation of the Fab at these non-canonical sites is 25%, 50%, 100%, 200%, 300%, 400%, 500%, or more greater than the amount of glycosylation of these non-canonical sites in a Fab produced in HEK293 cells.

(iv) In addition to the glycosylation sites, anti-VEGF Fabs such as ranibizumab (and the Fab of bevacizumab) contain tyrosine (“Y”) sulfation sites in or near the CDRs; see FIG. 1 which identifies tyrosine-O-sulfation sites in the VH (EDTAVY9+Y95) and VL (EDFATY8%) domains of ranibizumab (and corresponding sites in the Fab of bevacizumab). (See, e.g., Yang et al., 2015, Molecules 20:2138-2164, esp. at p. 2154 which is incorporated by reference in its entirety for the analysis of amino acids surrounding tyrosine residues subjected to protein tyrosine sulfation. The “rules” can be summarized as follows: Y residues with E or D within +5 to −5 position of Y, and where position −1 of Y is a neutral or acidic charged amino acid—but not a basic amino acid, e.g., R, K, or H that abolishes sulfation). Human IgG antibodies can manifest a number of other post-translational modifications, such as N-terminal modifications, C-terminal modifications, degradation or oxidation of amino acid residues, cysteine related variants, and glycation (See, e.g., Liu et al., 2014, mAbs 6(5): 1145-1154).

(v) Glycosylation of anti-VEGF Fabs, such as ranibizumab or the Fab fragment of bevacizumab by human retinal cells will result in the addition of glycans that can improve stability, half-life and reduce unwanted aggregation and/or immunogenicity of the transgene product. (See, e.g., Bovenkamp et al., 2016, J. Immunol. 196: 1435-1441 for a review of the emerging importance of Fab glycosylation). Significantly, glycans that can be added to HuPTMFabVEGFi, e.g., HuGlyFabVEGFi, provided herein, are highly processed complex-type biantennary N-glycans that contain 2,6-sialic acid (e.g., see FIG. 2 depicting the glycans that may be incorporated into HuPTMFab VEGFi, e.g., HuGlyFab VEGFi) and bisecting GlcNAc, but not NGNA (N-Glycolylneuraminic acid, Neu5Gc). Such glycans are not present in ranibizumab (which is made in E. coli and is not glycosylated at all) or in bevacizumab (which is made in CHO cells that do not have the 2,6-sialyltransferase required to make this post-translational modification, nor do CHO cells product bisecting GlcNAc, although they do add Neu5Gc (NGNA) as sialic acid not typical (and potentially immunogenic) to humans instead of Neu5Ac (NANA)) See, e.g., Dumont et al., 2015, Crit. Rev. Biotechnol. (Early Online, published online Sep. 18, 2015, pp. 1-13 at p. 5). Moreover, CHO cells can also produce an immunogenic glycan, the α-Gal antigen, which reacts with anti-α-Gal antibodies present in most individuals, and at high concentrations can trigger anaphylaxis. See, e.g., Bosques, 2010, Nat Biotech 28: 1153-1156. The human glycosylation pattern of the HuPTMFab VEGFi, e.g., HuGlyFabVEGFi, provided herein, should reduce immunogenicity of the transgene product and improve efficacy.

(vi)Tyrosine-sulfation of anti-VEGF Fabs, such as ranibizumab or the Fab fragment of bevacizumab a robust post-translational process in human retinal cells could result in transgene products with increased avidity for VEGF. Indeed, tyrosine-sulfation of the Fab of therapeutic antibodies against other targets has been shown to dramatically increase avidity for antigen and activity. (See, e.g., Loos et al., 2015, PNAS 112: 12675-12680, and Choe et al., 2003, Cell 114: 161-170). Such post-translational modifications are not present on ranibizumab (which is made in E. coli a host that does not possess the enzymes required for tyrosine-sulfation), and at best is under-represented in bevacizumab —a CHO cell product. Unlike human retinal cells, CHO cells are not secretory cells and have a limited capacity for post-translational tyrosine-sulfation. (See, e.g., Mikkelsen & Ezban, 1991, Biochemistry 30: 1533-1537, esp. discussion at p. 1537).

For the foregoing reasons, the production of HuPTMFab VEGFi, e.g., HuGlyFabVEGFi, should result in a “biobetter” molecule for the treatment of wet AMD, dry AMD, retinal vein occlusion (RVO), diabetic macular edema (DME), or diabetic retinopathy (DR) (in particular, wet AMD) accomplished via gene therapy—e.g., by administering a viral vector or other DNA expression construct encoding HuPTMFab VEGFi, e.g., HuGlyFab VEGFi, to the suprachorodial space, subretinal space, or the outer surface of the sclera in the eye(s) of patients (human subjects) diagnosed with wet AMD, dry AMD, retinal vein occlusion (RVO), diabetic macular edema (DME), or diabetic retinopathy (DR) (in particular, wet AMD) (e.g., by suprachorodial injection, subretinal injection via the transvitreal approach (a surgical procedure), subretinal administration via the suprachoroidal space, or a posterior juxtascleral depot procedure), to create a permanent depot in the eye that continuously supplies the fully-human post-translationally modified, e.g., human-glycosylated, sulfated transgene product produced by transduced retinal cells. The cDNA construct for the FabVEGFi should include a signal peptide that ensures proper co- and post-translational processing (glycosylation and protein sulfation) by the transduced retinal cells. Such signal sequences used by retinal cells may include but are not limited to:
MNFLLSWVHW SLALLLYLHH AKWSQA (VEGF-A signal peptide)
MERAAPSRRV PLPLLLLGGL ALLAAGVDA (Fibulin-1 signal peptide)
MAPLRPLLIL ALLAWVALA (Vitronectin signal peptide)
MRLLAKIICLMLWAICVA (Complement Factor H signal peptide)
MRLLAFLSLL ALVLQETGT (Opticin signal peptide)
MKWVTFISLLFLFSSAYS (Albumin signal peptide)
MAFLWLLSCWALLGTTFG (Chymotrypsinogen signal peptide)
MYRMQLLSCIALILALVTNS (Interleukin-2 signal peptide)
MNLLLILTFVAAAVA (Trypsinogen-2 signal peptide).
See, e.g., Stern et al., 2007, Trends Cell. Mol. Biol., 2:1-17 and Dalton & Barton, 2014, Protein Sci, 23: 517-525, each of which is incorporated by reference herein in its entirety for the signal peptides that can be used.

As an alternative, or an additional treatment to gene therapy, the HuPTMFab VEGFi product, e.g., HuGlyFabVEGFi glycoprotein, can be produced in human cell lines by recombinant DNA technology, and administered to patients diagnosed with wet AMD, dry AMD, retinal vein occlusion (RVO), diabetic macular edema (DME), or diabetic retinopathy (DR) (in particular, wet AMD) by intravitreal injection. The HuPTMFabVEGFi product, e.g., glycoprotein, may also be administered to patients with wet AMD, dry AMD, retinal vein occlusion (RVO), diabetic macular edema (DME), or diabetic retinopathy (DR) (in particular, wet AMD). Human cell lines that can be used for such recombinant glycoprotein production include but are not limited to human embryonic kidney 293 cells (HEK293), fibrosarcoma HT-1080, HKB-11, CAP, HuH-7, and retinal cell lines, PER.C6, or RPE to name a few (e.g., see Dumont et al., 2015, Crit. Rev. Biotechnol. (Early Online, published online Sep. 18, 2015, pp. 1-13) “Human cell lines for biopharmaceutical manufacturing: history, status, and future perspectives” which is incorporated by reference in its entirety for a review of the human cell lines that could be used for the recombinant production of the HuPTMFab VEGFi product, e.g., HuGlyFabVEGFi glycoprotein). To ensure complete glycosylation, especially sialylation, and tyrosine-sulfation, the cell line used for production can be enhanced by engineering the host cells to co-express α-2,6-sialyltransferase (or both α-2,3- and α-2,6-sialyltransferases) and/or TPST-1 and TPST-2 enzymes responsible for tyrosine-O-sulfation in retinal cells.

Combinations of delivery of the HuPTMFab VEGFi, e.g., HuGlyFab VEGFi, to the eye/retina accompanied by delivery of other available treatments are encompassed by the methods provided herein. The additional treatments may be administered before, concurrently or subsequent to the gene therapy treatment. Available treatments for wet AMD, dry AMD, retinal vein occlusion (RVO), diabetic macular edema (DME), or diabetic retinopathy (DR) (in particular, wet AMD) that could be combined with the gene therapy provided herein include but are not limited to laser photocoagulation, photodynamic therapy with verteporfin, and intravitreal (IVT) injections with anti-VEGF agents, including but not limited to pegaptanib, ranibizumab, aflibercept, or bevacizumab. Additional treatments with anti-VEGF agents, such as biologics, may be referred to as “rescue” therapy.

Unlike small molecule drugs, biologics usually comprise a mixture of many variants with different modifications or forms that have a different potency, pharmacokinetics, and safety profile. It is not essential that every molecule produced either in the gene therapy or protein therapy approach be fully glycosylated and sulfated. Rather, the population of glycoproteins produced should have sufficient glycosylation (from about 1% to about 10% of the population), including 2,6-sialylation, and sulfation to demonstrate efficacy. The goal of gene therapy treatment provided herein is to slow or arrest the progression of retinal degeneration, and to slow or prevent loss of vision with minimal intervention/invasive procedures. Efficacy may be monitored by measuring BCVA (Best-Corrected Visual Acuity), intraocular pressure, slit lamp biomicroscopy, indirect ophthalmoscopy, SD-OCT (SD-Optical Coherence Tomography), electroretinography (ERG). Signs of vision loss, infection, inflammation and other safety events, including retinal detachment may also be monitored. Retinal thickness may be monitored to determine efficacy of the treatments provided herein. Without being bound by any particular theory, thickness of the retina may be used as a clinical readout, wherein the greater reduction in retinal thickness or the longer period of time before thickening of the retina, the more efficacious the treatment. Retinal thickness may be determined, for example, by SD-OCT. SD-OCT is a three-dimensional imaging technology which uses low-coherence interferometry to determine the echo time delay and magnitude of backscattered light reflected off an object of interest. OCT can be used to scan the layers of a tissue sample (e.g., the retina) with 3 to 15 μm axial resolution, and SD-OCT improves axial resolution and scan speed over previous forms of the technology (Schuman, 2008, Trans. Am. Opthamol. Soc. 106:426-458). Retinal function may be determined, for example, by ERG. ERG is a non-invasive electrophysiologic test of retinal function, approved by the FDA for use in humans, which examines the light sensitive cells of the eye (the rods and cones), and their connecting ganglion cells, in particular, their response to a flash stimulation.

In preferred embodiments, the antigen-binding fragments do not contain detectable NeuGc and/or α-Gal. The phrase “detectable NeuGc and/or α-Gal” used herein means NeuGc and/or α-Gal moieties detectable by standard assay methods known in the art. For example, NeuGc may be detected by HPLC according to Hara et al., 1989, “Highly Sensitive Determination of N-Acetyl- and N-Glycolylneuraminic Acids in Human Serum and Urine and Rat Serum by Reversed-Phase Liquid Chromatography with Fluorescence Detection.” J. Chromatogr., B: Biomed. 377: 111-119, which is hereby incorporated by reference for the method of detecting NeuGc. Alternatively, NeuGc may be detected by mass spectrometry. The α-Gal may be detected using an ELISA, see, for example, Galili et al., 1998, “A sensitive assay for measuring alpha-Gal epitope expression on cells by a monoclonal anti-Gal antibody.” Transplantation. 65(8): 1129-32, or by mass spectrometry, see, for example, Ayoub et al., 2013, “Correct primary structure assessment and extensive glyco-profiling of cetuximab by a combination of intact, middle-up, middle-down and bottom-up ESI and MALDI mass spectrometry techniques.” Landes Bioscience. 5(5): 699-710. See also the references cited in Platts-Mills et al., 2015, “Anaphylaxis to the Carbohydrate Side-Chain Alpha-gal” Immunol Allergy Clin North Am. 35(2): 247-260.

In certain aspects, also provided herein are anti-VEGF antigen-binding fragments (i.e., antigen-binding fragments that immunospecifically binds to VEGF) comprising light chain CDRs 1-3 of SEQ ID NOs: 14-16 and heavy chain CDRs 1-3 of SEQ ID NOs: 20, 18, and 21, wherein the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the second amino acid residue of the light chain CDR3 (i.e., the second Q in QQYSTVPWTF (SEQ ID NO. 16)) is not acetylated. In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) is not acetylated. The anti-VEGF antigen-binding fragments provided herein can be used in any method according to the invention described herein. In a preferred embodiment, the chemical modification(s) or lack of chemical modification(s) (as the case may be) described herein is determined by mass spectrometry.

In certain aspects, also provided herein are anti-VEGF antigen-binding fragments comprising light chain CDRs 1-3 of SEQ ID NOs: 14-16 and heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) is not acetylated. In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) is not acetylated. The anti-VEGF antigen-binding fragments provided herein can be used in any method according to the invention described herein. In a preferred embodiment, the chemical modification(s) or lack of chemical modification(s) (as the case may be) described herein is determined by mass spectrometry.

In certain aspects, also provided herein are anti-VEGF antigen-binding fragments comprising light chain CDRs 1-3 of SEQ ID NOs: 14-16 and heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein: (1) the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR 1 (i.e., the N in GYDFTHYGMN (SEQ ID NO. 20)) does not carry one or more of the following chemical modifications: oxidation, acetylation, deamidation, and pyroglutamation (pyro Glu); and (2) the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) is not acetylated, and the second amino acid residuc of the light chain CDR3 (i.e., the second Q in QQYSTVPWTF (SEQ ID NO. 16)) is not acetylated. In a specific embodiment, the antigen-binding fragment comprises light chain CDRs 1-3 of SEQ ID NOs: 14-16 and heavy chain CDRs 1-3 of SEQ ID NOs: 20, 18, and 21, wherein: (1) the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) is not acetylated; and (2) the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) is not acetylated. The anti-VEGF antigen-binding fragments provided herein can be used in any method according to the invention described herein. In a preferred embodiment, the chemical modification(s) or lack of chemical modification(s) (as the case may be) described herein is determined by mass spectrometry.

Unexpected benefits of the invention are illustrated in the examples, infra, which demonstrate that expression of the HuPTMFabVEGFi from a rAAV8.anti-hVEGF Fab vector injected into the subretinal space (i) reduced subretinal neovascularization in transgenic mice that are models of nAMD in human subjects; and (ii) and surprisingly prevented retinal detachment in a transgenic mouse model of ocular neovascular disease that develops severe proliferative retinopathy and retinal detachment caused by ocular production of VEGF.

The examples also demonstrate that suprachoroidal administration of a rAAV8.anti-hVEGF Fab vector was equally effective at neutralizing VEGF-induced damage as subretinal injection of the vector. Suprachoroidal administrations allow for a quick and easy in-office procedure with low risk of complications.

Another contemplated administration route is subretinal administration via the suprachoroidal space, using a subretinal drug delivery device that has a catheter inserted and tunneled through the suprachoroidal space to inject into the subretinal space toward the posterior pole, where a small needle injects into the subretinal space. This route of administration allows the vitreous to remain intact and thus, there are fewer complication risks (less risk of gene therapy egress, and complications such as retinal detachments and macular holes), and without a vitrectomy, the resulting bleb may spread more diffusely allowing more of the surface area of the retina to be transduced with a smaller volume. The risk of induced cataract following this procedure is minimized, which is desirable for younger patients. Moreover, this procedure can deliver bleb under the fovea more safely than the standard transvitreal approach, which is desirable for patients with inherited retinal diseases effecting central vision where the target cells for transduction are in the macula. This procedure is also favorable for patients that have neutralizing antibodies (Nabs) to AAVs present in the systemic circulation which may impact other routes of delivery. Additionally, this method has shown to create blebs with less egress out the retinotomy site than the standard transvitreal approach.

Juxtascleral administration provides an additional administration route which avoids the risk of intraocular infection and retinal detachment, side effects commonly associated with injecting therapeutic agents directly into the cyc.

### 3.1 ILLUSTRATIVE EMBODIMENTS

### 3.1.1 Set 1

1. A method of treating a human subject diagnosed with wet AMD, dry AMD, retinal vein occlusion (RVO), diabetic macular edema (DME), or diabetic retinopathy (DR) (in particular, wet AMD), comprising delivering to the retina of said human subject a therapeutically effective amount of anti-hVEGF antigen-binding fragment produced by human retinal cells.
2. A method of treating a human subject diagnosed with wet AMD, dry AMD, retinal vein occlusion (RVO), diabetic macular edema (DME), or diabetic retinopathy (DR) (in particular, wet AMD), comprising delivering to the retina of said human subject a therapeutically effective amount of anti-hVEGF antigen-binding fragment produced by human retinal cells, by administering to the subretinal space in the eye of said human subject an expression vector encoding the anti-hVEGF antigen-binding fragment, by subretinal injection via the transvitreal approach or via the suprachoroidal space in the eye of said human subject.
3. A method of treating a human subject diagnosed with wet AMD, dry AMD, retinal vein occlusion (RVO), diabetic macular edema (DME), and diabetic retinopathy (DR), comprising delivering to the retina of said human subject a therapeutically effective amount of anti-h VEGF antigen-binding fragment produced by human retinal cells, by the use of a suprachoroidal drug delivery device such as a microinjector.
4 A method of treating a human subject diagnosed with wet AMD, dry AMD, retinal vein occlusion (RVO), diabetic macular edema (DME), or diabetic retinopathy (DR) (in particular, wet AMD), comprising delivering to the retina of said human subject a therapeutically effective amount of anti-hVEGF antigen-binding fragment produced by human photoreceptor cells (e.g., cone cells and/or rod cells), horizontal cells, bipolar cells, amacrine cells, retina ganglion cells (e.g., midget cells, parasol cells, bistratified cells, giant retina ganglion cells, photosensitive ganglion cells, and/or Müller glia), and/or retinal pigment epithelial cells in the external limiting membrane.
5 A method of treating a human subject diagnosed with wet AMD, dry AMD, retinal vein occlusion (RVO), diabetic macular edema (DME), or diabetic retinopathy (DR) (in particular, wet AMD), comprising delivering to the retina of said human subject a therapeutically effective amount of anti-hVEGF antigen-binding fragment produced by human photoreceptor cells (e.g., cone cells and/or rod cells), horizontal cells, bipolar cells, amacrine cells, retina ganglion cells (e.g., midget cells, parasol cells, bistratified cells, giant retina ganglion cells, photosensitive ganglion cells, and/or Müller glia), and/or retinal pigment epithelial cells in the external limiting membrane, by administering to the subretinal space in the eye of said human subject an expression vector encoding the anti-hVEGF antigen-binding fragment, by subretinal injection via the transvitreal approach or via the suprachoroidal space in the eye of said human subject.
6. A method of treating a human subject diagnosed with wet AMD, dry AMD, retinal vein occlusion (RVO), diabetic macular edema (DME), or diabetic retinopathy (DR) (in particular, wet AMD), comprising delivering to the retina of said human subject a therapeutically effective amount of anti-hVEGF antigen-binding fragment produced by human photoreceptor cells (e.g., cone cells and/or rod cells), horizontal cells, bipolar cells, amacrine cells, retina ganglion cells (e.g., midget cells, parasol cells, bistratified cells, giant retina ganglion cells, photosensitive ganglion cells, and/or Müller glia), and/or retinal pigment epithelial cells in the external limiting membrane, by the use of a suprachoroidal drug delivery device such as a microinjector.
7. The method of any one of paragraphs 1 to 6, in which the antigen-binding fragment is a Fab.
8 The method of any one of paragraphs 1 to 6, in which the antigen-binding fragment is an F(ab′)2.
9. The method of any one of paragraphs 1 to 6, in which the antigen-binding fragment is a single chain variable domain (scFv).
10. The method of any one of paragraphs 1 to 6, in which the antigen-binding fragment comprises a heavy chain comprising the amino acid sequence of SEQ ID NO. 1 or SEQ ID NO. 3, and a light chain comprising the amino acid sequence of SEQ ID NO. 2, or SEQ ID NO. 4.
11. The method of any one of paragraphs 1 to 6, wherein the antigen-binding fragment comprises light chain CDRs 1-3 of SEQ ID NOs: 14-16 and heavy chain CDRs 1-3 of SEQ ID NOs: 17-19 or SEQ ID NOs: 20, 18, and 21.
12. The method of paragraph 11, wherein the second amino acid residue of the light chain CDR3 does not carry one or more of the following chemical modifications: oxidation, acetylation, deamidation, and pyroglutamation (pyro Glu).
13. The method of paragraph 12, wherein the second amino acid residue of the light chain CDR3 is not acetylated.
14 The method of paragraph 12 or 13, wherein the eighth and eleventh amino acid residues of the light chain CDR 1 each carries one or more of the following chemical modifications: oxidation, acetylation, deamidation, and pyroglutamation (pyro Glu).
15. The method of any one of paragraphs 11-14, wherein the antigen-binding fragment comprises a heavy chain CDR1 of SEQ ID NO. 20 and wherein the last amino acid residue of the heavy chain CDR1 does not carry one or more of the following chemical modifications: oxidation, acetylation, deamidation, and pyroglutamation (pyro Glu).
16. The method of paragagraph 15, wherein the last amino acid residue of the heavy chain CDR 1 is not acetylated.
17. The method of paragraph 15 or 16, wherein the ninth amino acid residue of the heavy chain CDR1 carries one or more of the following chemical modifications: acetylation, deamidation, and pyroglutamation (pyro Glu), the third amino acid residue of the heavy chain CDR2 carries one or more of the following chemical modifications: acetylation, deamidation, and pyroglutamation (pyro Glu).
18. The method of any one of paragraphs 1-17, wherein the delivering step comprises administering an expression vector encoding the anti-h VEGF antigen-binding fragment at a dose ranging from 3×10⁹ genome copies to 2.5×10¹¹ genome copies.
19. The method of any one of paragraphs 1-17, wherein the delivering step comprises administering an expression vector encoding the anti-h VEGF antigen-binding fragment at a dose of about 3×10⁹genome copies.
20. The method of any one of paragraphs 1-17, wherein the delivering step comprises administering an expression vector encoding the anti-h VEGF antigen-binding fragment at a dose of about 1×10¹⁰ genome copies.
21. The method of any one of paragraphs 1-17, wherein the delivering step comprises administering an expression vector encoding the anti-h VEGF antigen-binding fragment at a dose of about 6×10¹⁰ genome copies.
22. The method of any one of paragraphs 1-17, wherein the delivering step comprises administering an expression vector encoding the anti-h VEGF antigen-binding fragment at a dose of about 1.6×10¹¹ genome copies.
23. The method of any one of paragraphs 1-17, wherein the delivering step comprises administering an expression vector encoding the anti-h VEGF antigen-binding fragment at a dose of about 2.5×10¹¹ genome copies
24. A method of treating a human subject diagnosed with neovascular age-related macular degeneration (wet AMD, dry AMD, retinal vein occlusion (RVO), diabetic macular edema (DME), or diabetic retinopathy (DR) (in particular, wet AMD)), comprising delivering to the eye of said human subject, a therapeutically effective amount of an antigen-binding fragment (a Fab, F(ab′)₂, or an scFv, collectively referred to herein as an “antigen-binding fragment”) of a mAb against hVEGF, said antigen-binding fragment containing a a2,6-sialylated glycan.
25. A method of treating a human subject diagnosed with wet AMD, dry AMD, retinal vein occlusion (RVO), diabetic macular edema (DME), or diabetic retinopathy (DR) (in particular, wet AMD), comprising delivering to the eye of said human subject, a therapeutically effective amount of an antigen-binding fragment (a Fab, F(ab′)₂, or an scFv, collectively referred to herein as an “antigen-binding fragment”) of a mAb against hVEGF, said antigen-binding fragment containing a a2,6-sialylated glycan, by administering to the subretinal space in the eye of said human subject an expression vector encoding the antigen-binding fragment of a mAb against hVEGF, by subretinal injection via the transvitreal approach or via the suprachoroidal space in the eye of said human subject.
26. A method of treating a human subject diagnosed with wet AMD, dry AMD, retinal vein occlusion (RVO), diabetic macular edema (DME), or diabetic retinopathy (DR) (in particular, wet AMD), comprising delivering to the eye of said human subject, a therapeutically effective amount of an antigen-binding fragment (a Fab, F(ab′)₂, or an scFv, collectively referred to herein as an “antigen-binding fragment”) of a mAb against hVEGF, said antigen-binding fragment containing a a2,6-sialylated glycan, by the use of a suprachoroidal drug delivery device such as a microinjector.
27. A method of treating a human subject diagnosed with wet AMD, dry AMD, retinal vein occlusion (RVO), diabetic macular edema (DME), or diabetic retinopathy (DR) (in particular, wet AMD), comprising delivering to the eye of said human subject, a therapeutically effective amount of a glycosylated antigen-binding fragment of a mAb against hVEGF, wherein said antigen-binding fragment does not contain detectable NeuGc and/or α-Gal antigen.
28. A method of treating a human subject diagnosed with wet AMD, dry AMD, retinal vein occlusion (RVO), diabetic macular edema (DME), or diabetic retinopathy (DR) (in particular, wet AMD), comprising delivering to the eye of said human subject, a therapeutically effective amount of a glycosylated antigen-binding fragment of a mAb against hVEGF, by administering to the subretinal space in the eye of said human subject an expression vector encoding the glycosylated antigen-binding fragment of a mAb against hVEGF, by subretinal injection via the transvitreal approach or via the suprachoroidal space in the eye of said human subject, wherein said antigen-binding fragment does not contain detectable NeuGc and/or α-Gal antigen.
29. A method of treating a human subject diagnosed with wet AMD, dry AMD, retinal vein occlusion (RVO), diabetic macular edema (DME), or diabetic retinopathy (DR) (in particular, wet AMD), comprising delivering to the eye of said human subject, a therapeutically effective amount of a glycosylated antigen-binding fragment of a mAb against hVEGF, by the use of a suprachoroidal drug delivery device such as a microinjector, wherein said antigen-binding fragment does not contain detectable NeuGc and/or α-Gal antigen.
30. The method of any one of paragraphs 24-29, wherein the delivering step comprises administering a recombinant nucleotide expression vector encoding the antigen-binding fragment of a mAb against hVEGF at a dose ranging from 3×10⁹ genome copies to 2.5×10¹¹ genome copies.
31. The method of any one of paragraphs 24-29, wherein the delivering step comprises administering a recombinant nucleotide expression vector encoding the antigen-binding fragment of a mAb against hVEGF at a dose of about 3×10⁹ genome copies.
32. The method of any one of paragraphs 24-29, wherein the delivering step comprises administering a recombinant nucleotide expression vector encoding the antigen-binding fragment of a mAb against hVEGF at a dose of about 1×10¹⁰ genome copies.
33. The method of any one of paragraphs 24-29, wherein the delivering step comprises administering a recombinant nucleotide expression vector encoding the antigen-binding fragment of a mAb against hVEGF at a dose of about 6×10¹⁰ genome copies.
34. The method of any one of paragraphs 24-29, wherein the delivering step comprises administering a recombinant nucleotide expression vector encoding the antigen-binding fragment of a mAb against hVEGF at a dose of about 1.6×10¹¹ genome copies.
35. The method of any one of paragraphs 24-29, wherein the delivering step comprises a recombinant nucleotide expression vector encoding the antigen-binding fragment of a mAb against h VEGF at a dose of about 2.5×10¹¹ genome copies.
36 A method of treating a human subject diagnosed with wet AMD, dry AMD, retinal vein occlusion (RVO), diabetic macular edema (DME), or diabetic retinopathy (DR) (in particular, wet AMD), wherein the method comprises: administering to the subretinal space in the eye of said human subject a expression vector encoding an antigen-binding fragment against hVEGF, wherein expression of said antigen-binding fragment is a2,6-sialylated upon expression from said expression vector in a human, immortalized retina-derived cell.
37. A method of treating a human subject diagnosed with wet AMD, dry AMD, retinal vein occlusion (RVO), diabetic macular edema (DME), or diabetic retinopathy (DR) (in particular, wet AMD), wherein the method comprises: administering to the subretinal space in the eye of said human subject a expression vector encoding an antigen-binding fragment against hVEGF, wherein expression of said antigen-binding fragment is a2,6-sialylated upon expression from said expression vector in a human, immortalized retina-derived cell, and wherein the administering step comprises the use of a subretinal drug delivery device comprising a catheter that can be inserted and tunneled through the suprachoroidal space toward the posterior pole, where a small needle injects into the subretinal space.
38. A method of treating a human subject diagnosed with wet AMD, dry AMD, retinal vein occlusion (RVO), diabetic macular edema (DME), or diabetic retinopathy (DR) (in particular, wet AMD), wherein the method comprises: administering or delivering to the retina of said human patient via the suprachoroidal space in the eye of said human subject a expression vector encoding an antigen-binding fragment against hVEGF, wherein expression of said antigen-binding fragment is a2,6-sialylated upon expression from said expression vector in a human, immortalized retina-derived cell.
39. A method of treating a human subject diagnosed with wet AMD, dry AMD, retinal vein occlusion (RVO), diabetic macular edema (DME), or diabetic retinopathy (DR) (in particular, wet AMD), wherein the method comprises: administering to the subretinal space in the eye of said human subject a expression vector encoding an antigen-binding fragment against hVEGF, wherein expression of said antigen-binding fragment is a2,6-sialylated upon expression from said expression vector in a human, immortalized retina-derived cell, wherein said antigen-binding fragment does not contain detectable NeuGc and/or α-Gal antigen.
40. A method of treating a human subject diagnosed with wet AMD, dry AMD, retinal vein occlusion (RVO), diabetic macular edema (DME), or diabetic retinopathy (DR) (in particular, wet AMD), wherein the method comprises: administering to the subretinal space in the eye of said human subject a expression vector encoding an antigen-binding fragment against hVEGF, wherein expression of said antigen-binding fragment is a2,6-sialylated upon expression from said expression vector in a human, immortalized retina-derived cell, wherein said antigen-binding fragment does not contain detectable NeuGc and/or α-Gal antigen, and wherein the administering step comprises the use of a subretinal drug delivery device comprising a catheter that can be inserted and tunneled through the suprachoroidal space toward the posterior pole, where a small needle injects into the subretinal space.
41. A method of treating a human subject diagnosed with wet AMD, dry AMD, retinal vein occlusion (RVO), diabetic macular edema (DME), or diabetic retinopathy (DR) (in particular, wet AMD), wherein the method comprises: administering or delivering to the retina of said human patient via the suprachoroidal space in the eye of said human subject a expression vector encoding an antigen-binding fragment against h VEGF, wherein expression of said antigen-binding fragment is a2,6-sialylated upon expression from said expression vector in a human, immortalized retina-derived cell, wherein said antigen-binding fragment does not contain detectable NeuGc and/or α-Gal antigen.
42. The method of any one of paragraphs 36-41, wherein the expression vector encoding the antigen-binding fragment against hVEGF is administered at a dose ranging from 3×10⁹ genome copies to 2.5×10¹¹ genome copies.
43. The method of any one of paragraphs 36-41, wherein the expression vector encoding the antigen-binding fragment against hVEGF is administered at a dose of about 3×10⁹ genome copies.
44. The method of any one of paragraphs 36-41, wherein the expression vector encoding the antigen-binding fragment against hVEGF is administered at a dose of about 1×10¹⁰ genome copies.
45. The method of any one of paragraphs 36-41, wherein the expression vector encoding the antigen-binding fragment against hVEGF is administered at a dose of about 6×10¹⁰ genome copies.
46. The method of any one of paragraphs 36-41, wherein the expression vector encoding the antigen-binding fragment against hVEGF is administered at a dose of about 1.6×10¹¹ genome copies.
47 The method of any one of paragraphs 36-41, wherein the expression vector encoding the antigen-binding fragment against hVEGF is administered at a dose of about 2.5×10¹¹ genome copies
48. A method of treating a human subject diagnosed with wet AMD, dry AMD, retinal vein occlusion (RVO), diabetic macular edema (DME), or diabetic retinopathy (DR) (in particular, wet AMD), comprising administering to the subretinal space in the eye of said human subject, a therapeutically effective amount of a recombinant nucleotide expression vector encoding an antigen-binding fragment of a mAb against hVEGF, so that a depot is formed that releases said antigen-binding fragment containing a a2,6-sialylated glycan.
49. A method of treating a human subject diagnosed with wet AMD, dry AMD, retinal vein occlusion (RVO), diabetic macular edema (DME), or diabetic retinopathy (DR) (in particular, wet AMD), comprising administering to the subretinal space in the eye of said human subject, a therapeutically effective amount of a recombinant nucleotide expression vector encoding an antigen-binding fragment of a mAb against hVEGF, so that a depot is formed that releases said antigen-binding fragment containing a a2,6-sialylated glycan, wherein the administering step comprises the use of a subretinal drug delivery device comprising a catheter that can be inserted and tunneled through the suprachoroidal space toward the posterior pole, where a small needle injects into the subretinal space.
50. A method of treating a human subject diagnosed with wet AMD, dry AMD, retinal vein occlusion (RVO), diabetic macular edema (DME), or diabetic retinopathy (DR) (in particular, wet AMD), comprising administering or delivering to the retina of said human patient via the suprachoroidal space in the eye of said human subject, a therapeutically effective amount of a recombinant nucleotide expression vector encoding an antigen-binding fragment of a mAb against hVEGF, so that a depot is formed that releases said antigen-binding fragment containing a a2,6-sialylated glycan.
51. A method of treating a human subject diagnosed with wet AMD, dry AMD, retinal vein occlusion (RVO), diabetic macular edema (DME), or diabetic retinopathy (DR) (in particular, wet AMD), comprising administering to the subretinal space in the eye of said human subject, a therapeutically effective amount of a recombinant nucleotide expression vector encoding an antigen-binding fragment of a mAb against hVEGF, so that a depot is formed that releases said antigen-binding fragment wherein said antigen-binding fragment is glycosylated but does not contain detectable NeuGc and/or α-Gal antigen.
52. A method of treating a human subject diagnosed with wet AMD, dry AMD, retinal vein occlusion (RVO), diabetic macular edema (DME), or diabetic retinopathy (DR) (in particular, wet AMD), comprising administering to the subretinal space in the eye of said human subject, a therapeutically effective amount of a recombinant nucleotide expression vector encoding an antigen-binding fragment of a mAb against hVEGF, so that a depot is formed that releases said antigen-binding fragment wherein said antigen-binding fragment is glycosylated but does not contain detectable NeuGc and/or α-Gal antigen, and wherein the administering step comprises the use of a subretinal drug delivery device comprising a catheter that can be inserted and tunneled through the suprachoroidal space toward the posterior pole, where a small needle injects into the subretinal space.
53. A method of treating a human subject diagnosed with wet AMD, dry AMD, retinal vein occlusion (RVO), diabetic macular edema (DME), or diabetic retinopathy (DR) (in particular, wet AMD), comprising administering or delivering to the retina of said human patient via the suprachoroidal space in the eye of said human subject, a therapeutically effective amount of a recombinant nucleotide expression vector encoding an antigen-binding fragment of a mAb against hVEGF, so that a depot is formed that releases said antigen-binding fragment wherein said antigen-binding fragment is glycosylated but does not contain detectable NeuGc and/or α-Gal antigen.
54. The method of any one of paragraphs 48-53, wherein the recombinant nucleotide expression vector encoding the antigen-binding fragment of a mAb against hVEGF is administered at a dose ranging from 3×10⁹ genome copies to 2.5×10¹¹ genome copies.
55. The method of any one of paragraphs 48-53, wherein the recombinant nucleotide expression vector encoding the antigen-binding fragment of a mAb against hVEGF is administered at a dose of about 3×10⁹ genome copies.
56. The method of any one of paragraphs 48-53, wherein the recombinant nucleotide expression vector encoding the antigen-binding fragment of a mAb against hVEGF is administered at a dose of about 1×10¹⁰ genome copics.
57. The method of any one of paragraphs 48-53, wherein the recombinant nucleotide expression vector encoding the antigen-binding fragment of a mAb against h VEGF is administered at a dose of about 6×10¹⁰ genome copies.
58. The method of any one of paragraphs 48-53, wherein the recombinant nucleotide expression vector encoding the antigen-binding fragment of a mAb against h VEGF is administered at a dose of about 1.6×10¹¹ genome copies.
59. The method of any one of paragraphs 48-53, wherein the recombinant nucleotide expression vector encoding the antigen-binding fragment of a mAb against hVEGF is administered at a dose of about 2.5×10¹¹ genome copies
60. The method of any one of paragraphs 24 to 59 in which the antigen-binding fragment comprises a heavy chain comprising the amino acid sequence of SEQ ID NO. 1 or SEQ ID NO. 3, and a light chain comprising the amino acid sequence of SEQ ID NO. 2, or SEQ ID NO. 4.
61. The method of any one of paragraphs 24 to 60, in which the antigen-binding fragment further contains a tyrosine-sulfation.
62. The method of any one of paragraphs 24 to 61 in which production of said antigen-binding fragment containing a a2,6-sialylated glycan is confirmed by transducing PER.C6 or RPE cell line with said recombinant nucleotide expression vector in cell culture.
63. The method of any one of paragraphs 24 to 61 in which production of said antigen-binding fragment containing a tyrosine-sulfation is confirmed by transducing PER. C6 or RPE cell line with said recombinant nucleotide expression vector in cell culture.
64. The method of any one of paragraphs 24 to 63, wherein the vector has a hypoxia-inducible promoter.
65. The method of any one of paragraphs 24 to 64, wherein the antigen-binding fragment comprises light chain CDRs 1-3 of SEQ ID NOs: 14-16 and heavy chain CDRs 1-3 of SEQ ID NOs: 17-19 or SEQ ID NOs: 20, 18, and 21.
66. The method of paragraph 65, wherein the second amino acid residue of the light chain CDR3 does not carry one or more of the following chemical modifications: oxidation, acetylation, deamidation, and pyroglutamation (pyro Glu).
67. The method of paragraph 66, wherein the second amino acid residue of the light chain CDR3 is not acetylated.
68. The method of paragraph 66 or 67, wherein the eighth and eleventh amino acid residues of the light chain CDR1 each carries one or more of the following chemical modifications: oxidation, acetylation, deamidation, and pyroglutamation (pyro Glu).
69. The method of any one of paragraphs 65-68, wherein the antigen-binding fragment comprises a heavy chain CDR1 of SEQ ID NO. 20 and wherein the last amino acid residue of the heavy chain CDR1 does not carry one or more of the following chemical modifications: oxidation, acetylation, deamidation, and pyroglutamation (pyro Glu).
70. The method of paragraph 69, wherein the last amino acid residue of the heavy chain CDR 1 is not acetylated.
71 The method of paragraph 69 or 70, wherein the ninth amino acid residue of the heavy chain CDR1 carries one or more of the following chemical modifications: acetylation, deamidation, and pyroglutamation (pyro Glu), the third amino acid residue of the heavy chain CDR2 carries one or more of the following chemical modifications: acetylation, deamidation, and pyroglutamation (pyro Glu).
72. The method of any one of paragraphs 24 to 71, wherein the antigen-binding fragment transgene encodes a leader peptide.
73. A method of treating a human subject diagnosed with wet AMD, dry AMD, retinal vein occlusion (RVO), diabetic macular edema (DME), or diabetic retinopathy (DR) (in particular, wet AMD), comprising administering to the subretinal space in the eye of said human subject, a therapeutically effective amount of a recombinant nucleotide expression vector encoding an antigen-binding fragment of a mAb against hVEGF, so that a depot is formed that releases said antigen-binding fragment containing a a2,6-sialylated glycan; wherein said recombinant vector, when used to transduce PER.C6 or RPE cells in culture results in production of said antigen-binding fragment containing a a2,6-sialylated glycan in said cell culture.
74. A method of treating a human subject diagnosed with wet AMD, dry AMD, retinal vein occlusion (RVO), diabetic macular edema (DME), or diabetic retinopathy (DR) (in particular, wet AMD), comprising administering to the subretinal space in the eye of said human subject, a therapeutically effective amount of a recombinant nucleotide expression vector encoding an antigen-binding fragment of a mAb against hVEGF, so that a depot is formed that releases said antigen-binding fragment containing a a2,6-sialylated glycan; wherein said recombinant vector, when used to transduce PER.C6 or RPE cells in culture results in production of said antigen-binding fragment containing a a2,6-sialylated glycan in said cell culture, and wherein the administering step comprises the use of a subretinal drug delivery device comprising a catheter that can be inserted and tunneled through the suprachoroidal space toward the posterior pole, where a small needle injects into the subretinal space.
75. A method of treating a human subject diagnosed with wet AMD, dry AMD, retinal vein occlusion (RVO), diabetic macular edema (DME), or diabetic retinopathy (DR) (in particular, wet AMD), comprising administering or delivering to the retina of said human patient via the suprachoroidal space in the eye of said human subject, a therapeutically effective amount of a recombinant nucleotide expression vector encoding an antigen-binding fragment of a mAb against hVEGF, so that a depot is formed that releases said antigen-binding fragment containing a a2,6-sialylated glycan; wherein said recombinant vector, when used to transduce PER.C6 or RPE cells in culture results in production of said antigen-binding fragment containing a a2,6-sialylated glycan in said cell culture.
76 A method of treating a human subject diagnosed with wet AMD, dry AMD, retinal vein occlusion (RVO), diabetic macular edema (DME), or diabetic retinopathy (DR) (in particular, wet AMD), comprising administering to the subretinal space in the eye of said human subject, a therapeutically effective amount of a recombinant nucleotide expression vector encoding an antigen-binding fragment of a mAb against hVEGF, so that a depot is formed that releases said antigen-binding fragment wherein said antigen-binding fragment is glycosylated but does not contain detectable NeuGc and/or α-Gal antigen; wherein said recombinant vector, when used to transduce PER.C6 or RPE cells in culture results in production of said antigen-binding fragment that is glycosylated but does not contain detectable NeuGc and/or α-Gal antigen in said cell culture.
77. A method of treating a human subject diagnosed with wet AMD, dry AMD, retinal vein occlusion (RVO), diabetic macular edema (DME), or diabetic retinopathy (DR) (in particular, wet AMD), comprising administering to the subretinal space in the eye of said human subject, a therapeutically effective amount of a recombinant nucleotide expression vector encoding an antigen-binding fragment of a mAb against hVEGF, so that a depot is formed that releases said antigen-binding fragment wherein said antigen-binding fragment is glycosylated but does not contain detectable NeuGc and/or α-Gal antigen; wherein said recombinant vector, when used to transduce PER.C6 or RPE cells in culture results in production of said antigen-binding fragment that is glycosylated but does not contain detectable NeuGe and/or α-Gal antigen in said cell culture, and wherein the administering step comprises the use of a subretinal drug delivery device comprising a catheter that can be inserted and tunneled through the suprachoroidal space toward the posterior pole, where a small needle injects into the subretinal space.
78. A method of treating a human subject diagnosed with wet AMD, dry AMD, retinal vein occlusion (RVO), diabetic macular edema (DME), or diabetic retinopathy (DR) (in particular, wet AMD), comprising administering or delivering to the retina of said human patient via the suprachoroidal space in the eye of said human subject, a therapeutically effective amount of a recombinant nucleotide expression vector encoding an antigen-binding fragment of a mAb against hVEGF, so that a depot is formed that releases said antigen-binding fragment wherein said antigen-binding fragment is glycosylated but does not contain detectable NeuGc and/or α-Gal antigen; wherein said recombinant vector, when used to transduce PER.C6 or RPE cells in culture results in production of said antigen-binding fragment that is glycosylated but does not contain detectable NeuGc and/or α-Gal antigen in said cell culture.
79. The method of any one of paragraphs 73-78, wherein the recombinant nucleotide expression vector encoding the antigen-binding fragment of a mAb against hVEGF is administered at a dose ranging from 3×10⁹ genome copies to 2.5×10¹¹ genome copies.
80. The method of any one of paragraphs 73-78, wherein the recombinant nucleotide expression vector encoding the antigen-binding fragment of a mAb against hVEGF is administered at a dose of about 3×10⁹ genome copies.
81. The method of any one of paragraphs 73-78, wherein the recombinant nucleotide expression vector encoding the antigen-binding fragment of a mAb against hVEGF is administered at a dose of about 1×10¹⁰ genome copies.
82. The method of any one of paragraphs 73-78, wherein the recombinant nucleotide expression vector encoding the antigen-binding fragment of a mAb against hVEGF is administered at a dose of about 6×10¹⁰ genome copies.
83. The method of any one of paragraphs 73-78, wherein the recombinant nucleotide expression vector encoding the antigen-binding fragment of a mAb against hVEGF is administered at a dose of about 1.6×10¹¹ genome copies.
84. The method of any one of paragraphs 73-78, wherein the recombinant nucleotide expression vector encoding the antigen-binding fragment of a mAb against hVEGF is administered at a dose of about 2.5×10¹¹ genome copies
85. The method of any one of paragraphs 24-35, wherein delivering to the eye comprises delivering to the retina, choroid, and/or vitreous humor of the eye.
86. The method of any one of paragraphs 1 to 85, wherein the antigen-binding fragment comprises a heavy chain that comprises one, two, three, or four additional amino acids at the C-terminus.
87. The method of any one of paragraphs 1 to 85, wherein the antigen-binding fragment comprises a heavy chain that does not comprise an additional amino acid at the C-terminus.
88 The method of any one of paragraphs 1 to 85, which produces a population of antigen-binding fragment molecules, wherein the antigen-binding fragment molecules comprise a heavy chain, and wherein 0.5%, 1%, 2%, 3%, 4%, 5%, 10%, or 20%, or less but more than 0% of the population of antigen-binding fragment molecules comprises one, two, three, or four additional amino acids at the C-terminus of the heavy chain.
89. The method of any one of paragraphs 1 to 85, which produces a population of antigen-binding fragment molecules, wherein the antigen-binding fragment molecules comprise a heavy chain, and wherein 0.5-1%, 0.5%-2%, 0.5%-3%, 0.5%-4%, 0.5%-5%, 0.5%-10%, 0.5%-20%, 1%-2%, 1%-3%, 1%-4%, 1%-5%, 1%-10%, 1%-20%, 2%-3%, 2%-4%, 2%-5%, 2%-10%, 2%-20%, 3%-4%, 3%-5%, 3%-10%, 3%-20%, 4%-5%, 4%-10%, 4%-20%, 5%-10%, 5%-20%, or 10%-20% of the population of antigen-binding fragment molecules comprises one, two, three, or four additional amino acids at the C-terminus of the heavy chain.
90. The method of any one of paragraphs 1 to 89, wherein the human subject has a BCVA that is ≤20/20 and ≥20/400 91. The method of any one of paragraphs 1 to 90, wherein the human subject has a BCVA that is ≤20/63 and ≥20/400.
92. The method of paragraph 90 or 91, wherein the BCVA is the BCVA in the eye to be treated in the human subject.
93. An antigen-binding fragment that immunospecifically binds to VEGF, wherein the antigen-binding fragment comprising light chain CDRs 1-3 of SEQ ID NOs: 14-16 and heavy chain CDRs 1-3 of SEQ ID NOs: 20, 18, and 21, and wherein the second amino acid residue of the light chain CDR3 does not carry one or more of the following chemical modifications: oxidation, acetylation, deamidation, and pyroglutamation (pyro Glu).
94. The antigen-binding fragment of paragraph 93, wherein the second amino acid residue of the light chain CDR3 is not acetylated.
95. The antigen-binding fragment of paragraph 93 or 94, wherein the eighth and eleventh amino acid residues of the light chain CDR1 each carries one or more of the following chemical modifications: oxidation, acetylation, deamidation, and pyroglutamation (pyro Glu).
96. The antigen-binding fragment of any one of paragraphs 93-95, wherein the last amino acid residue of the heavy chain CDR1 does not carry one or more of the following chemical modifications: oxidation, acetylation, deamidation, and pyroglutamation (pyro Glu).
97. The antigen-binding fragment of paragraph 96, wherein the last amino acid residue of the heavy chain CDR1 is not acetylated.
98. The antigen-binding fragment of paragraph 96 or 97, wherein the ninth amino acid residue of the heavy chain CDR1 carries one or more of the following chemical modifications: acetylation, deamidation, and pyroglutamation (pyro Glu), the third amino acid residue of the heavy chain CDR2 carries one or more of the following chemical modifications: acetylation, deamidation, and pyroglutamation (pyro Glu).

### 3.1.2 Set 2

1. A method of treating a human subject diagnosed with wet AMD, dry AMD, retinal vein occlusion (RVO), diabetic macular edema (DME), or diabetic retinopathy (DR) (in particular, wet AMD), comprising administering to the suprachoroidal space in the eye of said human subject an expression vector encoding an anti-human vascular endothelial growth factor (hVEGF) antibody.
2. The method of paragraph 1, wherein the administering is by injecting the expression vector into the suprachoroidal space using a suprachoroidal drug delivery device.
3. The method of paragraph 1 or 2, wherein the suprachoroidal drug delivery device is a microinjecor.
4. A method of treating a human subject diagnosed with wet AMD, dry AMD, retinal vein occlusion (RVO), diabetic macular edema (DME), or diabetic retinopathy (DR) (in particular, wet AMD), comprising administering to the subretinal space in the eye of said human subject an expression vector encoding an anti-hVEGF antibody via the suprachoroidal space in the eye of said human subject.
5. The method of paragraph 4, wherein the administering is by the use of a subretinal drug delivery device comprising a catheter that can be inserted and tunneled through the suprachoroidal space toward the posterior pole, where a small needle injects into the subretinal space.
6 The method of paragraph 5, wherein the administering comprises inserting and tunneling the catheter of the subretinal drug delivery device through the suprachoroidal space.
7. A method of treating a human subject diagnosed with wet AMD, dry AMD, retinal vein occlusion (RVO), diabetic macular edema (DME), or diabetic retinopathy (DR) (in particular, wet AMD), comprising administering to the outer surface of the sclera in the eye of said human subject an expression vector encoding an anti-hVEGF antibody.
8. The method of paragraph 7, wherein the administering is by the use of a juxtascleral drug delivery device that comprises a cannula whose tip can be inserted and kept in direct apposition to the scleral surface.
9. The method of paragraph 8, wherein the administering comprises inserting and keeping the tip of the cannula in direct apposition to the scleral surface.
10. The method of any one of paragraphs1-9, wherein the administering delivers a therapeutically effective amount of the anti-h VEGF antibody to the retina of said human subject.
11. The method of paragraph 10, wherein the therapeutically effective amount of the anti-h VEGF antibody is produced by human retinal cells of said human subject.
12. The method of paragraph 10, wherein the therapeutically effective amount of the anti-hVEGF antibody is produced by human photoreceptor cells, horizontal cells, bipolar cells, amacrine cells, retina ganglion cells, and/or retinal pigment epithelial cells in the external limiting membrane of said human subject.
13. The method of paragraph 12, wherein the human photoreceptor cells are cone cells and/or rod cells.
14. The method of paragraph 12, wherein the retina ganglion cells are midget cells, parasol cells, bistratified cells, giant retina ganglion cells, photosensitive ganglion cells, and/or Müller glia.
15. The method of any one of paragraphs 1-14, wherein the human subject has a Best-Corrected Visual Acuity (BCVA) that is ≤20/20 and ≥20/400.
16. The method of any one of paragraphs 1-14, wherein the human subject has a BCVA that is <20/63 and ≥20/400.
17. The method of paragraph 15 or 16, wherein the BCVA is the BCVA in the eye to be treated in the human subject.
18. The method of any one of paragraphs 1-17, wherein the anti-h VEGF antibody is an anti-hVEGF antigen-binding fragment.
19 The method of paragraph 18, in which the antigen-binding fragment is a Fab.
20 The method of paragraph 18, in which the antigen-binding fragment is a F(ab′)₂.
21. The method of paragraph 18, in which the antigen-binding fragment is a single chain variable domain (scFv).
22. The method of any one of paragraphs 1-21, in which the anti-hVEGF antibody comprises a heavy chain comprising the amino acid sequence of SEQ ID NO. 1 or SEQ ID NO. 3, and a light chain comprising the amino acid sequence of SEQ ID NO. 2, or SEQ ID NO. 4.
23. The method of any one of paragraphs 1-21, wherein the anti-h VEGF antibody comprises light chain CDRs 1-3 of SEQ ID NOs: 14-16 and heavy chain CDRs 1-3 of SEQ ID NOs: 17-19 or SEQ ID NOs: 20, 18, and 21.
24. The method of paragraph 22, wherein the second amino acid residue of the light chain CDR3 does not carry one or more of the following chemical modifications: oxidation, acetylation, deamidation, and pyroglutamation (pyro Glu).
25. The method of paragraph 23, wherein the second amino acid residue of the light chain CDR3 is not acetylated.
26. The method of paragraph 23 or 24, wherein the eighth and eleventh amino acid residues of the light chain CDR1 each carries one or more of the following chemical modifications: oxidation, acetylation, deamidation, and pyroglutamation (pyro Glu).
27. The method of any one of paragraphs 22-25, wherein the anti-hVEGF antibody comprises a heavy chain CDR1 of SEQ ID NO. 20 and wherein the last amino acid residue of the heavy chain CDR1 does not carry one or more of the following chemical modifications: oxidation, acetylation, deamidation, and pyroglutamation (pyro Glu).
28. The method of paragraph 26, wherein the last amino acid residue of the heavy chain CDR 1 is not acetylated.
29. The method of paragraph 26 or 27, wherein the ninth amino acid residue of the heavy chain CDR1 carries one or more of the following chemical modifications: acetylation, deamidation, and pyroglutamation (pyro Glu), the third amino acid residue of the heavy chain CDR2 carries one or more of the following chemical modifications: acetylation, deamidation, and pyroglutamation (pyro Glu).
30 The method of any one of paragraphs 1-29, wherein the expression vector is an AAV vector.
31 The method of paragraph 30, wherein the expression vector is an AAV8 vector.

### 4. BRIEF DESCRIPTION OF THE DRAWINGS

FIG. 1. The amino acid sequence of ranibizumab (top) showing 5 different residues in bevacizumab Fab (below). The starts of the variable and constant heavy chains (V_{H} and C_{H}) and light chains (V_{L} and V_{C}) are indicated by arrows (→), and the CDRs are underscored. Non-consensus glycosylation sites (“Gsite”) tyrosine-O-sulfation sites (“Ysite”) are indicated.

FIG. 2. Glycans that can be attached to HuGlyFab VEGFi. (Adapted from Bondt et al., 2014, Mol & Cell Proteomics 13.1: 3029-3039).

FIG. 3. The amino acid sequence of hyperglycosylated variants of ranibizumab (above) and bevacizumab Fab (below). The starts of the variable and constant heavy chains (V_{H} and C_{H}) and light chains (V_{L} and V_{C}) are indicated by arrows (→), and the CDRs are underscored. Non-consensus glycosylation sites (“Gsite”) and tyrosine-O-sulfation sites (“Ysite”) are indicated. Four hyperglycoslated variants are indicated with an asterisk (*).

FIG. 4. Dose-dependent reduction in neovascular area in Rho/VEGF Mice administered subretinal injections of Vector 1. Rho/VEGF mice were injected subretinally with the indicated doses of Vector 1 or control (PBS or empty vector at 1×10¹⁰ GC/eye), and one week later the area of retinal neovascularization was quantitated. The numbers of mice/group are designated on each bar. * indicates a p value between 0.0019 and 0.0062; ** indicates of a p value <0.0001.

FIG. 5. Reduction in the incidence and severity of retinal detachment in Tet/Opsin/VEGF mice administered subretinal injections of Vector 1. Tet/opsin/VEGF mice were injected subretinally with the indicated doses of Vector 1 or control (PBS or empty vector at 1×10¹⁰ GC/eye). Ten days later, VEGF expression was induced with the addition of doxycycline to the drinking water, and after 4 days, eyes were assessed for the presence of full retinal detachment, partial detachment, or no detachment.

FIG. 6. Protein levels after subretinal injection of expression cassettes for 3 different VEGF neutralizing proteins. The cDNAs for an anti-VEGFfab, anti-VEGF full length antibody (Ab), and soluble Fltl were inserted into the same expression cassette containing a CMV promoter and rabbit β-globin poly A signal and packaged in AAV8. Adult C57BL/6 mice were given a subretinal injection of 3×10⁹ GC in one eye. Fourteen days after injection, mice were euthanized, retinas were dissected, and the level of each transgene was measured by ELISA. Each bar represents the mean (±SEM) with n=6 for first two bars and n=22 for third bar.

FIG. 7A. Schematic of AAV8-antiVEGFfab genome.

FIG. 7B. Adult C57BL/6 mice were given a subretinal injection of 10¹⁰ genome copies (GC) of empty AAV8 vector or a dose of AAV8-antiVEGFfab between 1×10⁸ and 1×10¹⁰ GC. After 7 days, mice were euthanized, eyes were removed and frozen until assayed. Eyes were homogenized in lysis buffer and AAV8-anti VEGFfab protein was measured by ELISA. Bars represent the mean (±SEM) with n=for each bar or n≥ for each bar.

FIG. 8A. Subretinal injection of AAV8-antiVEGFfab suppresses type 3 choroidal neovascularization in rho/VEGF mice. At postnatal day (P) 14, rho/VEGF transgenic mice, in which the rhodopsin promoter drives expression of human VEGF165 in photoreceptors, were given a subretinal injection of 1×10¹⁰ GC of empty AAV8, a dose of AAV8-anti VEGFfab between 3×10⁶ and 1×10¹⁰ GC, or phosphate-buffered saline (PBS). At P21, retinal flat mounts were stained with FITC-labeled Griffonia Simplicifolia lectin which stains vascular cells. Retinal flat mounts from PBS-injected eyes showed numerous sprouts of neovascularization (NV) that at higher magnification (top row, center) were seen to originate from the deep capillary bed of the retina and extend into the subretinal space. Eyes injected with empty vector showed many sprouts of NV similar to what was seen in PBS-injected eyes (top row, right). In contrast, flat mounts from eyes injected with 10¹⁰ GC, 3×10⁹ GC, or 10⁹ GC of AAV8-antiVEGFfab showed few sprouts of NV (second row), flat mounts from eyes with doses between 3×10⁸ GC and 10⁷ GC showed an intermediate number of NV sprouts (second and third rows), and eyes injected with 3×10⁶ GC looked similar to control eyes. Scale bar=100 μm.

FIG. 8B. Image analysis was used to measure the area of NV per retina and bars show the mean (±SEM). *p<0.05; **p<0.01 for difference from empty vector by ANOVA with Bonferroni correction for multiple comparisons.

FIGS. 9A, 9B. Adult Tet/opsin/VEGF double transgenic mice had a subretinal injection of AAV8-anti VEGFfab in doses ranging from 1×10⁸ to 1×10¹⁰ GC in one eye and no injection in the fellow eye or 1×10¹⁰ GC of null vector in one eye and PBS in the fellow eye. Ten days after injection, 2 mg/ml of doxycycline was added to drinking water and after 4 days fundus photos were graded for presence of total, partial, or no retinal detachment and the percentage of detachment was measured for each eye. Representative fundus photos show total retinal detachments in mice injected with 1×10⁸ or 3×10⁹ GC of AAV8-anti VEGFfab (A, left) similar to those seen in mice injected with PBS or empty vector (B). There was a partial retinal detachment in an eye injected with 1×10⁹ GC and no retinal detachment in eyes injected with 3×10⁹ or 1×10¹⁰ GC of AAV8-antiVEGFfab (A, right 3 columns).

FIG. 9C. An ocular section stained with Hoechst from an eye injected with 3×10⁹ GC of AAV8-antiVEGFfab showed no retinal detachment while a section from the uninjected fellow eye showed a total retinal detachment.

FIG. 9D. Pie charts show a dose-dependent reduction in exudative retinal detachments in eyes injected with AAV8-antiVEGFfab.

FIG. 9E. P-values performed by Fisher's test shown for different doses whether there is a difference regarding the presence of no, partial, or total detachment from the empty vector group. The mean (±SEM) percentage retinal detachment was significantly less for eyes injected with 3×10⁹ or 1×10¹⁰ GC of AAV8-antiVEGFfab than for any of the other groups by one-way ANOVA with Bonfferoni correction for multiple comparisons (*p=0.002, **p=0.001).

FIG. 10A. Adult Tet/opsin/VEGF double transgenic mice had subretinal injection of 3×10⁹ GC AAV8-antiVEGFfab in one eye and no injection in the fellow eye or 3×10⁹ GC of null vector in one eye and no injection in the fellow eye. One month after injection, 2 mg/ml of doxycycline was added to drinking water and after 4 days fundus photos were graded for presence of total, partial, or no retinal detachment and the percentage of the retina detached was measured in each eye. Fundus photos from a representative mouse injected with 3×10⁹ GC of AAV8-antiVEGFfab showed no detachment in the injected eye and total detachment in the fellow eye (A, left 2 panels), while those from representative mouse injected with 3×10⁹ GC empty vector showed severe total retinal detachment in each eye (A, right 2 panels).

FIG. 10B. Ocular sections stained with Hoechst from a mouse injected with 3×10⁹ GC of AAV8-antiVEGFfab showed attached retina in the injected eye and total detachment in the fellow eye (B, left 2 panels). Ocular sections from a mouse injected with 3×10⁹ GC empty vector showed total retinal detachment in each eye (B, right 2 panels).

FIG. 10C. Nine of 10 eyes injected with AAV8-anti VEGFfab had no retinal detachment, while 8 of 10 fellow eyes had total retinal detachment (p<0.001 by Fisher's test). In contrast, 7 of 8 eyes injected with empty vector and fellow eyes had total retinal detachment (p=1.0). Compared to eyes injected with empty vector, there was significant prevention of retinal detachment in eyes injected with AAV8-antiVEGFfab (p=0.001 by Fisher's test).

FIG. 10D. In eyes injected with AAV8-antiVEGFfab (n=10) the mean (±SEM) percentage retinal detachment (RD) per eye was significant less than that in fellow eyes (* p<0.001) or eyes injected with empty vector (n-8; +p-0.001 by one-way ANOVA with Bonferroni correction).

FIG. 11. Target sequences (SEQ ID NO. 38 and SEQ ID NO. 39) are illustrated.

FIG. 12. 4×2.5 μg of Control and Retinal Cell Line were separated using SDS-PAGE. The bands at ˜25 kD were excised.

FIG. 13. Gel-based peptide mapping results for sample Control. Data matched to both sequences (SEQ ID NO. 38 and SEQ ID NO. 40). The boxed amino acid residues each carries one of the following chemical modifications: oxidation, acetylation, deamidation, and pyroglutamation (pyro Glu).

FIG. 14. Solution-based peptide mapping results for sample Control. Data matched to both sequences (SEQ ID NO. 38 and SEQ ID NO. 40). The boxed amino acid residues each carries one of the following chemical modifications: oxidation, acetylation, deamidation, and pyroglutamation (pyro Glu).

FIGS. 15A, 15B. Intact mass results for sample Control. The main peak in the observed chromatogram was summed to obtain a spectrum for deconvolution (A). The spectrum was deconvoluted to two components at 24,432.0 Da and 24,956.0 Da average mass. The deconvoluted spectrum and annotated raw data are illustrated (B).

FIG. 16. Gel-based peptide mapping results for sample Retinal Cell Line. Data matched to both sequences (SEQ ID NO. 38 and SEQ ID NO. 39). The boxed amino acid residues each carries one of the following chemical modifications: oxidation, acetylation, deamidation, and pyroglutamation (pyro Glu).

FIGS. 17A, 17B. Intact mass results for sample Retinal Cell Line. The main peak in the observed chromatogram was summed to obtain a spectrum for deconvolution (A). The spectrum was deconvoluted to two components at 24,428.0 Da and 24,952.0 Da average mass. The deconvoluted spectrum and annotated raw data are illustrated (B).

FIG. 18. Clustal Multiple Sequence Alignment of AAV capsids 1-9 (SEQ ID NOS: 41-51). Amino acid substitutions (shown in bold in the bottom rows) can be made to AAV9 and AAV8 capsids by “recruiting” amino acid residues from the corresponding position of other aligned AAV capsids. Sequence regions designated by “HVR”=hypervariable regions.

FIG. 19. Increased area and intensity of GFP expression between one and two weeks after suprachoroidal injection of AAV8.GFP. The mean (±SEM) level of GFP was high in homogenates of retina and RPE/choroid at 1 and 2 weeks after suprachoroidal injection.

FIGS. 20A, 20B. Measurement of albumin in vitreous samples by ELISA for eyes given suprachoroidal injection of AAV8. antiVEGFfab versus fellow eyes given suprachoroidal AAV8.GFP (A). Equally high levels of antiVEGFfab were detected in eyes injected with suprachoroidal or subretinal AAV8.antiVEGFfab (B)

FIG. 21. The mean (=SEM) level of GFP measured by ELISA was significantly higher in homogenates of RPE/choroid from eyes given 2 versus those given 1 injection. The difference was not significantly different for retinal homogenates.

FIG. 22. Vitreous albumin level of eyes which received no prior vector injection or SC or SR injection of AAV8.GFP 2 or 7 weeks before, and those that received SC or SR injection of anti VEGFfab. Compared with eyes that received no prior vector injection or SC or SR injection of AAV8.GFP 2 or 7 weeks before, those that received SC or SR injection of anti VEGFfab showed significantly less VEGF-induced increase in vitreous albumin.

FIG. 23. Measurement of AntiVEGFfab by ELISA in RPE/choroid and retinal homogenates showed no significant difference between SC and SR AAV8.antiVEGFfab at either time point.

FIG. 24. A suprachoroidal drug delivery device manufactured by ClearsideR Biomedical, Inc.

FIG. 25. A subretinal drug delivery device comprising a catheter that can be inserted and tunneled through the suprachoroidal space toward the posterior pole, where a small needle injects into the subretinal space, manufactured by Janssen Pharmaceuticals, Inc.

FIG. 26A-26D. Illustration of the posterior juxtascleral depot procedure.

### 5. DETAILED DESCRIPTION OF THE INVENTION

Compositions and methods are described for the delivery of a fully human post-translationally modified (HuPTM) antibody against VEGF to the retina/vitreal humour in the eye(s) of patients (human subjects) diagnosed with an ocular disease, in particular an ocular disease caused by increased neovascularization, for example, nAMD (also known as “wet” AMD), dry AMD, retinal vein occlusion (RVO), diabetic macular edema (DME), or diabetic retinopathy (DR) (in particular, wet AMD). Antibodies include, but are not limited to, monoclonal antibodies, polyclonal antibodies, recombinantly produced antibodies, human antibodies, humanized antibodies, chimeric antibodies, synthetic antibodies, tetrameric antibodies comprising two heavy chain and two light chain molecules, antibody light chain monomers, antibody heavy chain monomers, antibody light chain dimers, antibody heavy chain dimers, antibody light chain-heavy chain pairs, intrabodies, heteroconjugate antibodies, monovalent antibodies, and antigen-binding fragments of full-length antibodies, and fusion proteins of the above. Such antigen-binding fragments include, but are not limited to, single-domain antibodies (variable domain of heavy chain antibodies (VHHs) or nanobodies), Fabs, F(ab′)₂s. and scFvs (single-chain variable fragments) of full-length anti-VEGF antibodies (preferably, full-length anti-VEGF monoclonal antibodies (mAbs)) (collectively referred to herein as “antigen-binding fragments”). In a preferred embodiment, the fully human post-translationally modified antibody against VEGF is a fully human post-translationally modified antigen-binding fragment of a monoclonal antibody (mAb) against VEGF (“HuPTMFabVEGFi”). In a further preferred embodiment, the HuPTMFab VEGFi is a fully human glycosylated antigen-binding fragment of an anti-VEGF mAb (“HuGlyFab VEGFi”). See, also, International Patent Application Publication No. WO/2017/180936 (International Patent Application No. PCT/US2017/027529, filed Apr. 14, 2017), and International Patent Application Publication No. WO/2017/181021 (International Patent Application No. PCT/US2017/027650, filed Apr. 14, 2017), each of which is incorporated by reference herein in its entirety, for compositions and methods that can be used according to the invention described herein. In an alternative embodiment, full-length mAbs can be used. Delivery may be accomplished via gene therapy—e.g., by administering a viral vector or other DNA expression construct encoding an anti-VEGF antigen-binding fragment or mAb (or a hyperglycosylated derivative) to the suprachoroidal space, subretinal space (from a transvitreal approach or with a catheter through the suprachoroidal space), intraretinal space, and/or outer surface of the sclera (i.e., juxtascleral administration) in the eye(s) of patients (human subjects) diagnosed with wet AMD, dry AMD, retinal vein occlusion (RVO), diabetic macular edema (DME), or diabetic retinopathy (DR) (in particular, wet AMD), to create a permanent depot in the eye that continuously supplies the human PTM, e.g., human-glycosylated, transgene product. See, e.g., administration modes described in Section 5.3.2. In a preferred embodiment, the methods provided herein are used in patients (human subjects) diagnosed with wet AMD

Subjects to whom such gene therapy is administered should be those responsive to anti-VEGF therapy. In particular embodiments, the methods encompass treating patients who have been diagnosed with wet AMD, dry AMD, retinal vein occlusion (RVO), diabetic macular edema (DME), or diabetic retinopathy (DR) (in particular, wet AMD) and identified as responsive to treatment with an anti-VEGF antibody. In more specific embodiments, the patients are responsive to treatment with an anti-VEGF antigen-binding fragment. In certain embodiments, the patients have been shown to be responsive to treatment with an anti-VEGF antigen-binding fragment injected intravitreally prior to treatment with gene therapy. In specific embodiments, the patients have previously been treated with LUCENTIS® (ranibizumab), EYLEA® (aflibercept), and/or AVASTIN® (bevacizumab), and have been found to be responsive to one or more of said LUCENTIS® (ranibizumab), EYLEA® (aflibercept), and/or AVASTIN® (bevacizumab).

Subjects to whom such viral vector or other DNA expression construct is delivered should be responsive to the anti-VEGF antigen-binding fragment encoded by the transgene in the viral vector or expression construct. To determine responsiveness, the anti-h VEGF antigen-binding fragment transgene product (e.g., produced in cell culture, bioreactors, etc.) may be administered directly to the subject, such as by intravitreal injection.

The HuPTMFab VEGFi, e.g., HuGlyFabVEGFi, encoded by the transgene can include, but is not limited to an antigen-binding fragment of an antibody that binds to hVEGF, such as bevacizumab; an anti-hVEGF Fab moiety such as ranibizumab; or such bevacizumab or ranibizumab Fab moieties engineered to contain additional glycosylation sites on the Fab domain (e.g., see Courtois et al., 2016, mAbs 8: 99-112 which is incorporated by reference herein in its entirety for it description of derivatives of bevacizumab that are hyperglycosylated on the Fab domain of the full length antibody).

The recombinant vector used for delivering the transgene should have a tropism for human retinal cells or photoreceptor cells. Such vectors can include non-replicating recombinant adeno-associated virus vectors (“rAAV”), particularly those bearing an AAV8 capsid are preferred. However, other viral vectors may be used, including but not limited to lentiviral vectors, vaccinia viral vectors, or non-viral expression vectors referred to as “naked DNA” constructs. Preferably, the HuPTMFab VEGFi, e.g., HuGlyFab VEGFi, transgene should be controlled by appropriate expression control elements, for example, the CB7 promoter (a chicken β-actin promoter and CMV enhancer), the RPE65 promoter, or opsin promoter to name a few, and can include other expression control elements that enhance expression of the transgene driven by the vector (e.g., introns such as the chicken β-actin intron, minute virus of mice (MVM) intron, human factor IX intron (e.g., FIX truncated intron 1), β-globin splice donor/immunoglobulin heavy chain spice acceptor intron, adenovirus splice donor /immunoglobulin splice acceptor intron, SV40 late splice donor/splice acceptor (19S/16S) intron, and hybrid adenovirus splice donor/IgG splice acceptor intron and poly A signals such as the rabbit β-globin poly A signal, human growth hormone (hGH) polyA signal, SV40 late poly A signal, synthetic polyA (SPA) signal, and bovine growth hormone (bGH) polyA signal). See, e.g., Powell and Rivera-Soto, 2015, Discov. Med., 19(102):49-57.

In preferred embodiments, gene therapy constructs are designed such that both the heavy and light chains are expressed. More specifically, the heavy and light chains should be expressed at about equal amounts, in other words, the heavy and light chains are expressed at approximately a 1: 1 ratio of heavy chains to light chains. The coding sequences for the heavy and light chains can be engineered in a single construct in which the heavy and light chains are separated by a cleavable linker or IRES so that separate heavy and light chain polypeptides are expressed. See, e.g., Section 5.2.4 for specific leader sequences and Section 5.2.5 for specific IRES, 2A, and other linker sequences that can be used with the methods and compositions provided herein.

Pharmaceutical compositions suitable for suprachoroidal, subretinal, juxtascleral and/or intraretinal administration comprise a suspension of the recombinant (e.g., rHuGlyFab VEGFi) vector in a formulation buffer comprising a physiologically compatible aqueous buffer, a surfactant and optional excipients.

Therapeutically effective doses of the recombinant vector should be administered subretinally and/or intraretinally (e.g., by subretinal injection via the transvitreal approach (a surgical procedure), or subretinal administration via the suprachoroidal space) in a volume ranging from ≥0.1 mL to ≤0.5 ml., preferably in 0.1 to 0.30 ml. (100-300 μl), and most preferably, in a volume of 0.25 mL (250 μl). Therapeutically effective doses of the recombinant vector should be administered suprachoroidally (e.g., by suprachoroidal injection) in a volume of 100 μl or less, for example, in a volume of 50-100 μl. Therapeutically effective doses of the recombinant vector should be administered to the outer surface of the sclera in a volume of 500 μl or less, for example, in a volume of 500 μl or less, for example, in a volume of 10-20 μl, 20-50 μl, 50-100 μl, 100-200 μl, 200-300 μl, 300-400 μl, or 400-500 μl. Subretinal injection is a surgical procedure performed by trained retinal surgeons that involves a partial vitrectomy with the subject under local anesthesia, and injection of the gene therapy into the retina. (see, e.g., Campochiaro et al., 2017, Hum Gen Ther 28(1):99-111, which is incorporated by reference herein in its entirety). In a specific embodiment, the subretinal administration is performed via the suprachoroidal space using a subretinal drug delivery device that comprises a catheter which can be inserted and tunneled through the suprachoroidal spece to the posterior pole, where a small needle injects into the subretinal space (see, e.g., Baldassarre et al., 2017, Subretinal Delivery of Cells via the Suprachoroidal Space: Janssen Trial. In: Schwartz et al. (eds) Cellular Therapies for Retinal Disease, Springer, Cham; International Patent Application Publication No. WO 2016/040635 A1; each of which is incorporated by reference herein in its entirety). Suprachoroidal administration procedures involve administration of a drug to the suprachoroidal space of the eye, and are normally performed using a suprachoroidal drug delivery device such as a microinjector with a microneedle (see, e.g., Hariprasad, 2016, Retinal Physician 13: 20-23; Goldstein, 2014, Retina Today 9(5): 82-87; each of which is incorporated by reference herein in its entirety). The suprachoroidal drug delivery devices that can be used to deposit the expression vector in the suprachoroidal space according to the invention described herein include, but are not limited to, suprachoroidal drug delivery devices manufactured by ClearsideR Biomedical, Inc. (see, for example, Hariprasad, 2016, Retinal Physician 13: 20-23). The subretinal drug delivery devices that can be used to deposit the expression vector in the subretinal space via the suprachoroidal space according to the invention described herein include, but are not limited to, subretinal drug delivery devices manufactured by Janssen Pharmaceuticals, Inc. (see, for example, International Patent Application Publication No. WO 2016/040635 A1). In a specific embodiment, administration to the outer surface of the sclera is performed by a juxtascleral drug delivery device that comprises a cannula, whose tip can be inserted and kept in direct apposition to the scleral surface. See Section 5.3.2 for more details of the different modes of administration. Suprachoroidal, subretinal, juxtascleral and/or intraretinal administration should result in delivery of the soluble transgene product to the retina, the vitreous humor, and/or the aqueous humor. The expression of the transgene product (e.g., the encoded anti-VEGF antibody) by retinal cells, e.g., rod, cone, retinal pigment epithelial, horizontal, bipolar, amacrine, ganglion, and/or Müller cells, results in delivery and maintenance of the transgene product in the retina, the vitreous humor, and/or the aqueous humor. Doses that maintain a concentration of the transgene product at a Cₘᵢₙ of at least 0.330 μg/mL in the Vitreous humour, or 0.110 μg/mL in the Aqueous humour (the anterior chamber of the eye) for three months are desired; thereafter, Vitreous Cₘᵢₙ concentrations of the transgene product ranging from 1.70 to 6.60 μg/mL, and/or Aqueous Cₘᵢₙ concentrations ranging from 0.567 to 2.20 μg/mL should be maintained. However, because the transgene product is continuously produced, maintenance of lower concentrations can be effective. The concentration of the transgene product can be measured in patient samples of the vitreous humour and/or aqueous from the anterior chamber of the treated eye. Alternatively, vitreous humour concentrations can be estimated and/or monitored by measuring the patient's serum concentrations of the transgene product the ratio of systemic to vitreal exposure to the transgene product is about 1:90,000. (E.g., see, vitreous humor and serum concentrations of ranibizumab reported in Xu L, et al., 2013, Invest. Opthal. Vis. Sci. 54: 1616-1624, at p. 1621 and Table 5 at p. 1623, which is incorporated by reference herein in its entirety).

The invention has several advantages over standard of care treatments that involve repeated ocular injections of high dose boluses of the VEGF inhibitor that dissipate over time resulting in peak and trough levels. Sustained expression of the transgene product antibody, as opposed to injecting an antibody repeatedly, allows for a more consistent levels of antibody to be present at the site of action, and is less risky and more convenient for patients, since fewer injections need to be made, resulting in fewer doctor visits. Consitent protein production may leads to better clinical outcomes as edema rebound in the retina is less likely to occur. Furthermore, antibodies expressed from transgenes are post-translationally modified in a different manner than those that are directly injected because of the different microenvironment present during and after translation. Without being bound by any particular theory, this results in antibodies that have different diffusion, bioactivity, distribution, affinity, pharmacokinetic, and immunogenicity characteristics, such that the antibodies delivered to the site of action are “biobetters” in comparison with directly injected antibodies.

In addition, antibodies expressed from transgenes in vivo are not likely to contain degradation products associated with antibodies produced by recombinant technologies, such as protein aggregation and protein oxidation. Aggregation is an issue associated with protein production and storage due to high protein concentration, surface interaction with manufacturing equipment and containers, and purification with certain buffer systems. These conditions, which promote aggregation, do not exist in transgene expression in gene therapy. Oxidation, such as methionine, tryptophan, and histidine oxidation, is also associated with protein production and storage, and is caused by stressed cell culture conditions, metal and air contact, and impurities in buffers and excipients. The proteins expressed from transgenes in vivo may also oxidize in a stressed condition. However, humans, and many other organisms, are equipped with an antioxidation defense system, which not only reduces the oxidation stress, but sometimes also repairs and/or reverses the oxidation. Thus, proteins produced in vivo are not likely to be in an oxidized form. Both aggregation and oxidation could affect the potency, pharmacokinetics (clearance), and immunogenicity.

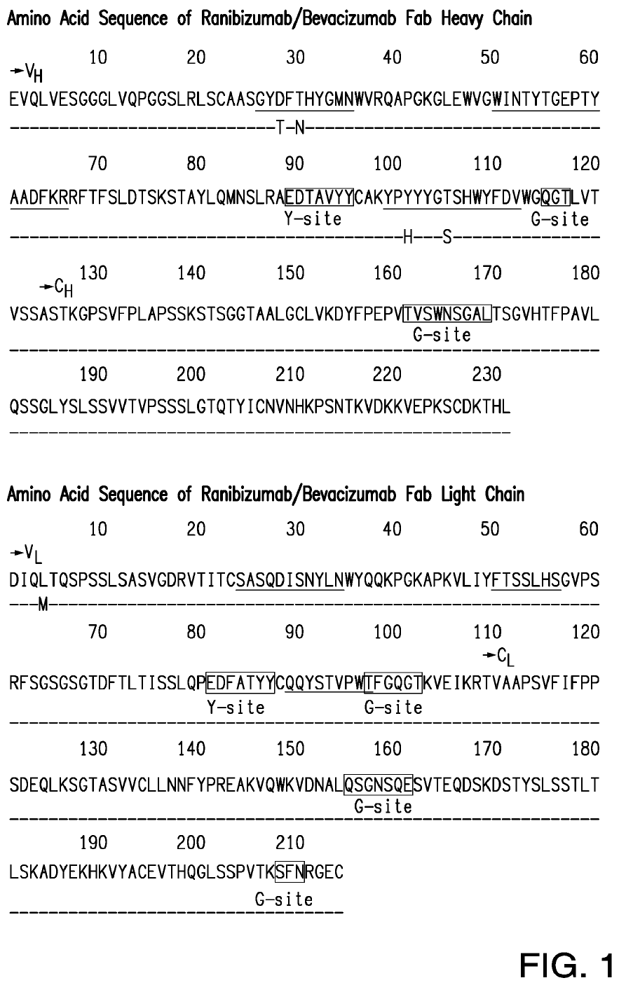

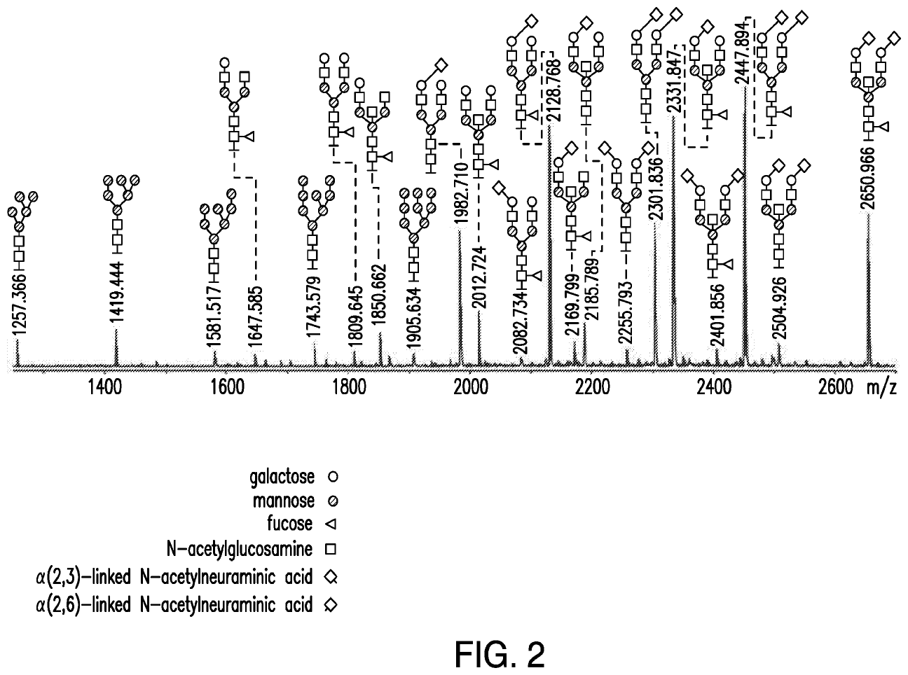

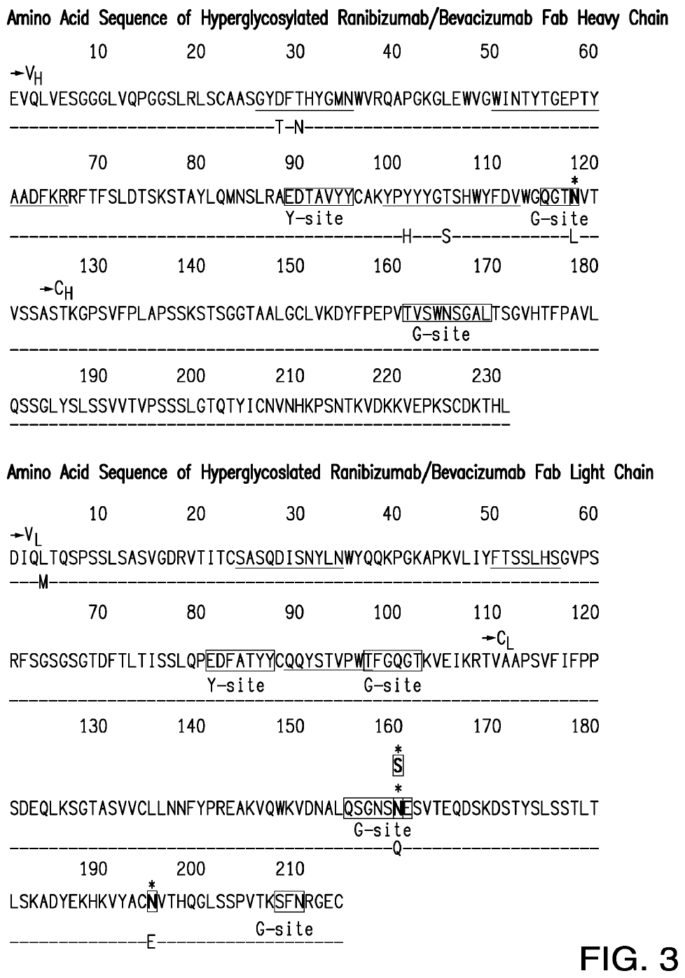

Without being bound by theory, the methods and compositions provided herein are based, in part, on the following principles:
(i) Human retinal cells are secretory cells that possess the cellular machinery for post-translational processing of secreted proteins—including glycosylation and tyrosine-O-sulfation, a robust process in retinal cells. (See, e.g., Wang et al., 2013, Analytical Biochem. 427: 20-28 and Adamis et al., 1993, BBRC 193: 631-638 reporting the production of glycoproteins by retinal cells; and Kanan et al., 2009, Exp. Eye Res. 89: 559-567 and Kanan & Al-Ubaidi, 2015, Exp. Eye Res. 133: 126-131 reporting the production of tyrosine-sulfated glycoproteins secreted by retinal cells, each of which is incorporated by reference in its entirety for post-translational modifications made by human retinal cells).
(ii) Contrary to the state of the art understanding, anti-VEGF antigen-binding fragments, such as ranibizumab (and the Fab domain of full length anti-VEGF mAbs such as bevacizumab) do indeed possess N-linked glycosylation sites. For example, see FIG. 1 which identifies non-consensus asparaginal (“N”) glycosylation sites in the C_{H} domain (TVSWN165SGAL) and in the C_{L} domain (QSGN158SQE), as well as glutamine (“Q”) residues that are glycosylation sites in the V_{H} domain (Q115GT) and V_{L} domain (TFQ100GT) of ranibizumab (and corresponding sites in the Fab of bevacizumab). (See, e.g., Valliere-Douglass et al., 2009, J. Biol. Chem. 284: 32493-32506, and Valliere-Douglass et al., 2010, J. Biol. Chem. 285: 16012-16022, each of which is incorporated by reference in its entirety for the identification of N-linked glycosylation sites in antibodies).
(iii)While such non-canonical sites usually result in low level glycosylation (e.g., about 1-5%) of the antibody population, the functional benefits may be significant in immunoprivileged organs, such as the eye (See, e.g., van de Bovenkamp et al., 2016, J. Immunol. 196:1435-1441). For example, Fab glycosylation may affect the stability, half-life, and binding characteristics of an antibody. To determine the effects of Fab glycosylation on the affinity of the antibody for its target, any technique known to one of skill in the art may be used, for example, enzyme linked immunosorbent assay (ELISA), or surface plasmon resonance (SPR). To determine the effects of Fab glycosylation on the half-life of the antibody, any technique known to one of skill in the art may be used, for example, by measurement of the levels of radioactivity in the blood or organs (e.g., the eye) in a subject to whom a radiolabelled antibody has been administered. To determine the effects of Fab glycosylation on the stability, for example, levels of aggregation or protein unfolding, of the antibody, any technique known to one of skill in the art may be used, for example, differential scanning calorimetry (DSC), high performance liquid chromatography (HPLC), e.g., size exclusion high performance liquid chromatography (SEC-HPLC), capillary electrophoresis, mass spectrometry, or turbidity measurement. Provided herein, the HuPTMFab VEGFi, e.g., HuGlyFabVEGFi, transgene results in production of a Fab which is 0.5%, 1%, 2%, 3%, 4%, 5%, 6%, 7%, 8%, 9%, or 10% or more glycosylated at non-canonical sites. In certain embodiments, 0.5%, 1%, 2%, 3%, 4%, 5%, 6%, 7%, 8%, 9%, or 10% or more Fabs from a population of Fabs are glycosylated at non-canonical sites. In certain embodiments, 0.5%, 1%, 2%, 3%, 4%, 5%, 6%, 7%, 8%, 9%, or 10% or more non-canonical sites are glycosylated. In certain embodiments, the glycosylation of the Fab at these non-canonical sites is 25%, 50%, 100%, 200%, 300%, 400%, 500%, or more greater than the amount of glycosylation of these non-canonical sites in a Fab produced in HEK293 cells.
(iv)In addition to the glycosylation sites, anti-VEGF Fabs such as ranibizumab (and the Fab of bevacizumab) contain tyrosine (“Y”) sulfation sites in or near the CDRs; see FIG. 1 which identifies tyrosine-O-sulfation sites in the V_{H} (EDTA VY⁹⁴Y⁹⁵) and V_{L} (EDFATY⁸⁶) domains of ranibizumab (and corresponding sites in the Fab of bevacizumab). (See, e.g., Yang et al., 2015, Molecules 20:2138-2164, esp. at p. 2154 which is incorporated by reference in its entirety for the analysis of amino acids surrounding tyrosine residues subjected to protein tyrosine sulfation. The “rules” can be summarized as follows: Y residues with E or D within +5 to −5 position of Y, and where position −1 of Y is a neutral or acidic charged amino acid—but not a basic amino acid, e.g., R, K, or H that abolishes sulfation). Human IgG antibodies can manifest a number of other post-translational modifications, such as N-terminal modifications, C-terminal modifications, degradation or oxidation of amino acid residues, cysteine related variants, and glycation (See, e.g., Liu et al., 2014, mAbs 6(5):1145-1154).
(v) Glycosylation of anti-VEGF Fabs, such as ranibizumab or the Fab fragment of bevacizumab by human retinal cells will result in the addition of glycans that can improve stability, half-life and reduce unwanted aggregation and/or immunogenicity of the transgene product. (See, e.g., Bovenkamp et al., 2016, J. Immunol. 196: 1435-1441 for a review of the emerging importance of Fab glycosylation). Significantly, glycans that can be added to HuPTMFab VEGFi, e.g., HuGlyFabVEGFi, provided herein, are highly processed complex-type biantennary N-glycans that contain 2,6-sialic acid (e.g., see FIG. 2 depicting the glycans that may be incorporated into HuPTMFab VEGFi, e.g., HuGlyFabVEGFi) and bisecting GlcNAc, but not NGNA (N-Glycolylneuraminic acid, Neu5Gc). Such glycans are not present in ranibizumab (which is made in E. coli and is not glycosylated at all) or in bevacizumab (which is made in CHO cells that do not have the 2,6-sialyltransferase required to make this post-translational modification, nor do CHO cells product bisecting GlcNAc, although they do add Neu5Gc (NGNA) as sialic acid not typical (and potentially immunogenic) to humans instead of Neu5Ac (NANA)). See, e.g., Dumont et al., 2015, Crit. Rev. Biotechnol. (Early Online, published online Sep. 18, 2015, pp. 1-13 at p. 5). Moreover, CHO cells can also produce an immunogenic glycan, the α-Gal antigen, which reacts with anti-α-Gal antibodies present in most individuals, and at high concentrations can trigger anaphylaxis. See, e.g., Bosques, 2010, Nat Biotech 28: 1153-1156. The human glycosylation pattern of the HuPTMFab VEGFi, e.g., HuGlyFab VEGFi, provided herein, should reduce immunogenicity of the transgene product and improve efficacy.
(vi) Tyrosine-sulfation of anti-VEGF Fabs, such as ranibizumab or the Fab fragment of bevacizumab—a robust post-translational process in human retinal cells—could result in transgene products with increased avidity for VEGF. Indeed, tyrosine-sulfation of the Fab of therapeutic antibodies against other targets has been shown to dramatically increase avidity for antigen and activity. (See, e.g., Loos et al., 2015, PNAS 112: 12675-12680, and Choe et al., 2003, Cell 114: 161-170). Such post-translational modifications are not present on ranibizumab (which is made in E. coli a host that does not possess the enzymes required for tyrosine-sulfation), and at best is under-represented in bevacizumab —a CHO cell product. Unlike human retinal cells, CHO cells are not secretory cells and have a limited capacity for post-translational tyrosine-sulfation. (See, e.g., Mikkelsen & Ezban, 1991, Biochemistry 30: 1533-1537, esp. discussion at p. 1537).

For the foregoing reasons, the production of HuPTMFab VEGFi, e.g., HuGlyFab VEGFi, should result in a “biobetter” molecule for the treatment of wet AMD, dry AMD, retinal vein occlusion (RVO), diabetic macular edema (DME), or diabetic retinopathy (DR) (in particular, wet AMD) accomplished via gene therapy—e.g., by administering a viral vector or other DNA expression construct encoding HuPTMFabVEGFi, e.g., HuGlyFab VEGFi, to the suprachoroidal space, subretinal space, or outer surface of the sclera in the eye(s) of patients (human subjects) diagnosed with wet AMD, dry AMD, retinal vein occlusion (RVO), diabetic macular edema (DME), or diabetic retinopathy (DR) (in particular, wet AMD) (e.g., by suprachoroidal injection, subretinal injection via the transvitreal approach (a surgical procedure), subretinal administration via the suprachoroidal space, or a posterior juxtascleral depot procedure), to create a permanent depot in the eye that continuously supplies the fully-human post-translationally modified, e.g., human-glycosylated, sulfated transgene product produced by transduced retinal cells. The cDNA construct for the Fab VEGFi should include a signal peptide that ensures proper co- and post-translational processing (glycosylation and protein sulfation) by the transduced retinal cells. Such signal sequences used by retinal cells may include but are not limited to:
MNFLLSWVHW SLALLLYLHH AKWSQA (VEGF-A signal peptide)
MERAAPSRRV PLPLLLLGGL ALLAAGVDA (Fibulin-1 signal peptide)
MAPLRPLLIL ALLAWVALA (Vitronectin signal peptide)
MRLLAKIICLMLWAICVA (Complement Factor H signal peptide)
MRLLAFLSLL ALVLQETGT (Opticin signal peptide)
MKWVTFISLLFLFSSAYS (Albumin signal peptide)
MAFLWLLSCWALLGTTFG (Chymotrypsinogen signal peptide)
MYRMQLLSCIALILALVTNS (Interleukin-2 signal peptide)
MNLLLILTFVAAAVA (Trypsinogen-2 signal peptide).
See, e.g., Stern et al., 2007, Trends Cell. Mol. Biol., 2:1-17 and Dalton & Barton, 2014, Protein Sci, 23: 517-525, each of which is incorporated by reference herein in its entirety for the signal peptides that can be used.

As an alternative, or an additional treatment to gene therapy, the HuPTMFab VEGFi product, e.g., HuGlyFabVEGFi glycoprotein, can be produced in human cell lines by recombinant DNA technology, and administered to patients diagnosed with wet AMD, dry AMD, retinal vein occlusion (RVO), diabetic macular edema (DME), or diabetic retinopathy (DR) (in particular, wet AMD) by intravitreal injection. The HuPTMFab VEGFi product, e.g., glycoprotein, may also be administered to patients with wet AMD, dry AMD, retinal vein occlusion (RVO), diabetic macular edema (DME), or diabetic retinopathy (DR) (in particular, wet AMD). Human cell lines that can be used for such recombinant glycoprotein production include but are not limited to human embryonic kidney 293 cells (HEK293), fibrosarcoma HT-1080, HKB-11, CAP, HuH-7, and retinal cell lines, PER.C6, or RPE to name a few (e.g., see Dumont et al., 2015, Crit. Rev. Biotechnol. (Early Online, published online Sep. 18, 2015, pp. 1-13) “Human cell lines for biopharmaceutical manufacturing: history, status, and future perspectives” which is incorporated by reference in its entirety for a review of the human cell lines that could be used for the recombinant production of the HuPTMFab VEGFi product, e.g., HuGlyFabVEGFi glycoprotein). To ensure complete glycosylation, especially sialylation, and tyrosine-sulfation, the cell line used for production can be enhanced by engineering the host cells to co-express α-2,6-sialyltransferase (or both α-2,3- and α-2,6-sialyltransferases) and/or TPST-1 and TPST-2 enzymes responsible for tyrosine-O-sulfation in retinal cells.

Combinations of delivery of the HuPTMFab VEGFi, e.g., HuGlyFab VEGFi, to the eye/retina accompanied by delivery of other available treatments are encompassed by the methods provided herein. The additional treatments may be administered before, concurrently or subsequent to the gene therapy treatment. Available treatments for wet AMD, dry AMD, retinal vein occlusion (RVO), diabetic macular edema (DME), or diabetic retinopathy (DR) (in particular, wet AMD) that could be combined with the gene therapy provided herein include but are not limited to laser photocoagulation, photodynamic therapy with verteporfin, and intravitreal (IVT) injections with anti-VEGF agents, including but not limited to pegaptanib, ranibizumab, aflibercept, or bevacizumab. Additional treatments with anti-VEGF agents, such as biologics, may be referred to as “rescue” therapy.

Unlike small molecule drugs, biologics usually comprise a mixture of many variants with different modifications or forms that have a different potency, pharmacokinetics, and safety profile. It is not essential that every molecule produced either in the gene therapy or protein therapy approach be fully glycosylated and sulfated. Rather, the population of glycoproteins produced should have sufficient glycosylation (from about 1% to about 10% of the population), including 2,6-sialylation, and sulfation to demonstrate efficacy. The goal of gene therapy treatment provided herein is to slow or arrest the progression of retinal degeneration, and to slow or prevent loss of vision with minimal intervention/invasive procedures. Efficacy may be monitored by measuring BCVA (Best-Corrected Visual Acuity), intraocular pressure, slit lamp biomicroscopy, indirect ophthalmoscopy, SD-OCT (SD-Optical Coherence Tomography), electroretinography (ERG). Signs of vision loss, infection, inflammation and other safety events, including retinal detachment may also be monitored. Retinal thickness may be monitored to determine efficacy of the treatments provided herein. Without being bound by any particular theory, thickness of the retina may be used as a clinical readout, wherein the greater reduction in retinal thickness or the longer period of time before thickening of the retina, the more efficacious the treatment. Retinal thickness may be determined, for example, by SD-OCT. SD-OCT is a three-dimensional imaging technology which uses low-coherence interferometry to determine the echo time delay and magnitude of backscattered light reflected off an object of interest. OCT can be used to scan the layers of a tissue sample (e.g., the retina) with 3 to 15 μm axial resolution, and SD-OCT improves axial resolution and scan speed over previous forms of the technology (Schuman, 2008, Trans. Am. Opthamol. Soc. 106:426-458). Retinal function may be determined, for example, by ERG. ERG is a non-invasive electrophysiologic test of retinal function, approved by the FDA for use in humans, which examines the light sensitive cells of the cyc (the rods and cones), and their connecting ganglion cells, in particular, their response to a flash stimulation.

### 5.1 N-Glycosylation, Tyrosine Sulfation, and O-Glycosylation

The amino acid sequence (primary sequence) of the anti-VEGF antigen-binding fragment of a HuPTMFabVEGFi, e.g., HuGlyFabVEGFi, used in the methods described herein comprises at least one site at which N-glycosylation or tyrosine sulfation takes place. In certain embodiments, the amino acid sequence of the anti-VEGF antigen-binding fragment comprises at least one N-glycosylation site and at least one tyrosine sulfation site. Such sites are described in detail below. In certain embodiments, the amino acid sequence of the anti-VEGF antigen-binding fragment comprises at least one O-glycosylation site, which can be in addition to one or more N-glycosylation sites and/or tyrosine sulfation sites present in said amino acid sequence.

### 5.1.1 N-Glycosylation

### Reverse Glycosylation Sites

The canonical N-glycosylation sequence is known in the art to be Asn-X-Ser(or Thr), wherein X can be any amino acid except Pro. However, it recently has been demonstrated that asparagine (Asn) residues of human antibodies can be glycosylated in the context of a reverse consensus motif, Ser(or Thr)-X-Asn, wherein X can be any amino acid except Pro. See Valliere-Douglass et al., 2009, J. Biol. Chem. 284:32493-32506; and Valliere-Douglass et al., 2010, J. Biol. Chem. 285:16012-16022. As disclosed herein, and contrary to the state of the art understanding, anti-VEGF antigen-binding fragments for use in accordance with the methods described herein, e.g., ranibizumab, comprise several of such reverse consensus sequences. Accordingly, the methods described herein comprise use of anti-VEGF antigen-binding fragments that comprise at least one N-glycosylation site comprising the sequence Ser(or Thr)-X-Asn, wherein X can be any amino acid except Pro (also referred to herein as a “reverse N-glycosylation site”).

In certain embodiments, the methods described herein comprise use of an anti-VEGF antigen-binding fragment that comprises one, two, three, four, five, six, seven, eight, nine, ten, or more than ten N-glycosylation sites comprising the sequence Ser(or Thr)-X-Asn, wherein X can be any amino acid except Pro. In certain embodiments, the methods described herein comprise use of an anti-VEGF antigen-binding fragment that comprises one, two, three, four, five, six, seven, eight, nine, ten, or more than ten reverse N-glycosylation sites, as well as one, two, three, four, five, six, seven, eight, nine, ten, or more than ten non-consensus N-glycosylation sites (as defined herein, below).

In a specific embodiment, the anti-VEGF antigen-binding fragment comprising one or more reverse N-glycosylation sites used in the methods described herein is ranibizumab, comprising a light chain and a heavy chain of SEQ ID NOs. 1 and 2, respectively. In another specific embodiment, the anti-VEGF antigen-binding fragment comprising one or more reverse N-glycosylation sites used in the methods comprises the Fab of bevacizumab, comprising a light chain and a heavy chain of SEQ ID NOs. 3 and 4, respectively.

### Non-Consensus Glycosylation Sites

In addition to reverse N-glycosylation sites, it recently has been demonstrated that glutamine (Gln) residues of human antibodies can be glycosylated in the context of a non-consensus motif, Gln-Gly-Thr. See Valliere-Douglass et al., 2010, J. Biol. Chem. 285:16012-16022. Surprisingly, anti-VEGF antigen-binding fragments for use in accordance with the methods described herein, e.g., ranibizumab, comprise several of such non-consensus sequences. Accordingly, the methods described herein comprise use of anti-VEGF antigen-binding fragments that comprise at least one N-glycosylation site comprising the sequence Gln-Gly-Thr (also referred to herein as a “non-consensus N-glycosylation site”).

In certain embodiments, the methods described herein comprise use of an anti-VEGF antigen-binding fragment that comprises one, two, three, four, five, six, seven, eight, nine, ten, or more than ten N-glycosylation sites comprising the sequence Gln-Gly-Thr.

In a specific embodiment, the anti-VEGF antigen-binding fragment comprising one or more non-consensus N-glycosylation sites used in the methods described herein is ranibizumab (comprising a light chain and a heavy chain of SEQ ID NOs. 1 and 2, respectively). In another specific embodiment, the anti-VEGF antigen-binding fragment comprising one or more non-consensus N-glycosylation sites used in the methods comprises the Fab of bevacizumab (comprising a light chain and a heavy chain of SEQ ID NOs. 3 and 4, respectively).

### Engineered N-Glycosylation Sites

In certain embodiments, a nucleic acid encoding an anti-VEGF antigen-binding fragment is modified to include 1, 2, 3, 4, 5, 6, 7, 8, 9, 10, or more N-glycosylation sites (including the canonical N-glycosylation consensus sequence, reverse N-glycosylation site, and non-consensus N-glycosylation sites) than would normally be associated with the HuGlyFabVEGFi (e.g., relative to the number of N-glycosylation sites associated with the anti-VEGF antigen-binding fragment in its unmodified state). In specific embodiments, introduction of glycosylation sites is accomplished by insertion of N-glycosylation sites (including the canonical N-glycosylation consensus sequence, reverse N-glycosylation site, and non-consensus N-glycosylation sites) anywhere in the primary structure of the antigen-binding fragment, so long as said introduction does not impact binding of the antigen-binding fragment to its antigen, VEGF. Introduction of glycosylation sites can be accomplished by, e.g., adding new amino acids to the primary structure of the antigen-binding fragment, or the antibody from which the antigen-binding fragment is derived (i.e., the glycosylation sites are added, in full or in part), or by mutating existing amino acids in the antigen-binding fragment, or the antibody from which the antigen-binding fragment is derived, in order to generate the N-glycosylation sites (i.e., amino acids are not added to the antigen-binding fragment/antibody, but selected amino acids of the antigen-binding fragment/antibody are mutated so as to form N-glycosylation sites). Those of skill in the art will recognize that the amino acid sequence of a protein can be readily modified using approaches known in the art, e.g., recombinant approaches that include modification of the nucleic acid sequence encoding the protein.

In a specific embodiment, an anti-VEGF antigen-binding fragment used in the method described herein is modified such that, when expressed in retinal cells, it can be hyperglycosylated. See Courtois et al., 2016, mAbs 8:99-112 which is incorporated by reference herein in its entirety. In a specific embodiment, said anti-VEGF antigen-binding fragment is ranibizumab (comprising a light chain and a heavy chain of SEQ ID NOs. 1 and 2, respectively). In another specific embodiment, said anti-VEGF antigen-binding fragment comprises the Fab of bevacizumab (comprising a light chain and a heavy chain of SEQ ID NOs. 3 and 4, respectively).

### N-Glycosylation of Anti-VEGF Antigen-Binding Fragments

Unlike small molecule drugs, biologics usually comprise a mixture of many variants with different modifications or forms that have a different potency, pharmacokinetics, and safety profile. It is not essential that every molecule produced either in the gene therapy or protein therapy approach be fully glycosylated and sulfated. Rather, the population of glycoproteins produced should have sufficient glycosylation (including 2,6-sialylation) and sulfation to demonstrate efficacy. The goal of gene therapy treatment provided herein is to slow or arrest the progression of retinal degeneration, and to slow or prevent loss of vision with minimal intervention/invasive procedures.

In I a specific embodiment, an anti-VEGF antigen-binding fragment, e.g., ranibizumab, used in accordance with the methods described herein, when expressed in a retinal cell, could be glycosylated at 100% of its N-glycosylation sites. However, one of skill in the art will appreciate that not every N-glycosylation site of an anti-VEGF antigen-binding fragment need be N-glycosylated in order for benefits of glycosylation to be attained. Rather, benefits of glycosylation can be realized when only a percentage of N-glycosylation sites are glycosylated, and/or when only a percentage of expressed antigen-binding fragments are glycosylated. Accordingly, in certain embodiments, an anti-VEGF antigen-binding fragment used in accordance with the methods described herein, when expressed in a retinal cell, is glycosylated at 10%-20%, 20%-30%, 30%-40%, 40%-50%, 50%-60%, 60%-70%, 70%-80%, 80% -90%, or 90%-100% of it available N-glycosylation sites. In certain embodiments, when expressed in a retinal cell, 10%-20%, 20%-30%, 30%-40%, 40%-50%, 50%-60%, 60% -70%, 70%-80%, 80%-90%, or 90%-100% of the an anti-VEGF antigen-binding fragments used in accordance with the methods described herein are glycosylated at least one of their available N-glycosylation sites.

In a specific embodiment, at least 10%, 20% 30%, 40%, 50%, 60%, 70%, 75%, 80%, 85%, 90%, 95%, or 99% of the N-glycosylation sites present in an anti-VEGF antigen-binding fragment used in accordance with the methods described herein are glycosylated at an Asn residue (or other relevant residue) present in an N-glycosylation site, when the anti-VEGF antigen-binding fragment is expressed in a retinal cell. That is, at least 50%, 60%, 70%, 75%, 80%, 85%, 90%, 95%, or 99% of the N-glycosylation sites of the resultant HuGlyFab VEGFi are glycosylated.

In another specific embodiment, at least 10%, 20% 30%, 40%, 50%, 60%, 70%, 75%, 80%, 85%, 90%, 95%, or 99% of the N-glycosylation sites present in an anti-VEGF antigen-binding fragment used in accordance with the methods described herein are glycosylated with an identical attached glycan linked to the Asn residue (or other relevant residue) present in an N-glycosylation site, when the anti-VEGF antigen-binding fragment is expressed in a retinal cell. That is, at least 50%, 60%, 70%, 75%, 80%, 85%, 90%, 95%, or 99% of the N-glycosylation sites of the resultant HuGlyFabVEGFi an identical attached glycan.

When an anti-VEGF antigen-binding fragment, e.g., ranibizumab, used in accordance with the methods described herein is expressed in a retinal cell, the N-glycosylation sites of the of the antigen-binding fragment can be glycosylated with various different glycans. N-glycans of antigen-binding fragments have been characterized in the art. For example, Bondt et al., 2014, Mol. & Cell. Proteomics 13.11:3029-3039 (incorporated by reference herein in its entirety for it disclosure of Fab-associated N-glycans) characterizes glycans associated with Fabs, and demonstrates that Fab and Fc portions of antibodies comprise distinct glycosylation patterns, with Fab glycans being high in galactosylation, sialylation, and bisection (e.g., with bisecting GlcNAc) but low in fucosylation with respect to Fc glycans. Like Bondt, Huang et al., 2006, Anal. Biochem. 349:197-207 (incorporated by reference herein in its entirety for it disclosure of Fab-associated N-glycans) found that most glycans of Fabs are sialylated. However, in the Fab of the antibody examined by Huang (which was produced in a murine cell background), the identified sialic residues were N-Glycolylneuraminic acid (“Neu5Gc” or “NeuGc”) (which is not natural to humans) instead of N-acetylneuraminic acid (“Neu5Ac,” the predominant human sialic acid). In addition, Song et al., 2014, Anal. Chem. 86:5661-5666 (incorporated by reference herein in its entirety for it disclosure of Fab-associated N-glycans) describes a library of N-glycans associated with commercially available antibodies.

Importantly, when the anti-VEGF antigen-binding fragments, e.g., ranibizumab, used in accordance with the methods described herein are expressed in human retinal cells, the need for in vitro production in prokaryotic host cells (e.g., E. coli) or eukaryotic host cells (e.g., CHO cells) is circumvented. Instead, as a result of the methods described herein (e.g., use of retinal cells to express anti-h VEGF antigen-binding fragments), N-glycosylation sites of the anti-VEGF antigen-binding fragments are advantageously decorated with glycans relevant to and beneficial to treatment of humans. Such an advantage is unattainable when CHO cells or E. coli are utilized in antibody/antigen-binding fragment production, because e.g., CHO cells (1) do not express 2,6 sialyltransferase and thus cannot add 2,6 sialic acid during N-glycosylation and (2) can add Neu5Gc as sialic acid instead of Neu5Ac; and because E. coli does not naturally contain components needed for N-glycosylation. Accordingly, in one embodiment, an anti-VEGF antigen-binding fragment expressed in a retinal cell to give rise to a HuGlyFab VEGFi used in the methods of treatment described herein is glycosylated in the manner in which a protein is N-glycosylated in human retinal cells, e.g., retinal pigment cells, but is not glycosylated in the manner in which proteins are glycosylated in CHO cells. In another embodiment, an anti-VEGF antigen-binding fragment expressed in a retinal cell to give rise to a HuGlyFab VEGFi used in the methods of treatment described herein is glycosylated in the manner in which a protein is N-glycosylated in human retinal cells, e.g., retinal pigment cells, wherein such glycosylation is not naturally possible using a prokaryotic host cell, e.g., using E. coli.

In certain embodiments, a HuGlyFabVEGFi, e.g., ranibizumab, used in accordance with the methods described herein comprises one, two, three, four, five or more distinct N-glycans associated with Fabs of human antibodies. In a specific embodiment, said N-glycans associated with Fabs of human antibodies are those described in Bondt et al., 2014, Mol. & Cell. Proteomics 13.11:3029-3039, Huang et al., 2006, Anal. Biochem. 349:197-207, and/or Song et al., 2014, Anal. Chem. 86:5661-5666. In certain embodiments, a HuGlyFabVEGFi, e.g., ranibizumab, used in accordance with the methods described herein does not comprise detectable NeuGc and/or α-Gal antigen.

In a specific embodiment, the HuGlyFab VEGFi, e.g., ranibizumab, used in accordance with the methods described herein are predominantly glycosylated with a glycan comprising 2,6-linked sialic acid. In certain embodiments, HuGlyFab VEGFi comprising 2,6-linked sialic acid is polysialylated, i.e., contains more than one sialic acid. In certain embodiments, each N-glycosylation site of said HuGlyFab VEGFi comprises a glycan comprising 2,6-linked sialic acid, i.e., 100% of the N-glycosylation site of said HuGlyFab VEGFi comprise a glycan comprising 2,6-linked sialic acid. In another specific embodiment, at least 20%, 30%, 40%, 50%, 60%, 70%, 75%, 80%, 85%, 90%, 95%, or 99% of the N-glycosylation sites of a HuGlyFab VEGFi used in accordance with the methods described herein are glycosylated with a glycan comprising 2,6-linked sialic acid. In another specific embodiment, at least 10%-20%, 20%-30%, 30%-40%, 40%-50%, 50%-60%, 60%-70%, 70%-80%, 80% 90%, or 90%-99% of the N-glycosylation sites of a HuGlyFab VEGFi used in accordance with the methods described herein are glycosylated with a glycan comprising 2,6-linked sialic acid. In another specific embodiment, at least 20%, 30%, 40%, 50%, 60%, 70%, 75%, 80%, 85%, 90%, 95%, or 99% of the antigen-binding fragments expressed in a retinal cell in accordance with methods described herein (i.e., the antigen-binding fragments that give rise to HuGlyFabVEGFi, e.g., ranibizumab) are glycosylated with a glycan comprising 2,6-linked sialic acid. In another specific embodiment, at least 10%-20%, 20%-30%, 30%-40%, 40%-50%, 50%-60%, 60%-70%, 70%-80%, 80%-90%, or 90%-99% of the antigen-binding fragments expressed in a retinal cell in accordance with methods described herein (i.e., the Fabs that give rise to HuGlyFabVEGFi, e.g., ranibizumab) are glycosylated with a glycan comprising 2,6-linked sialic acid. In another specific embodiment, said sialic acid is Neu5Ac. In accordance with such embodiments, when only a percentage of the N-glycosylation sites of a HuGlyFab VEGFi are 2,6 sialylated or polysialylated, the remaining N-glycosylation can comprise a distinct N-glycan, or no N-glycan at all (i.e., remain non-glycosylated).

When a HuGlyFabVEGFi is 2,6 polysialylated, it comprises multiple sialic acid residues, e.g., 2, 3, 4, 5, 6, 7, 8, 9, 10 or more than 10 sialic acid residues. In certain embodiments, when a HuGlyFabVEGFi is polysialylated, it comprises 2-5, 5-10, 10-20, 20-30, 30-40, or 40-50 sialic acid residues. In certain embodiments, when a HuGlyFab VEGFi is polysialylated, it comprises 2,6-linked (sialic acid)“, wherein n can be any number from 1-100.

In a specific embodiment, the HuGlyFabVEGFi, e.g., ranibizumab, used in accordance with the methods described herein are predominantly glycosylated with a glycan comprising a bisecting GlcNAc. In certain embodiments, each N-glycosylation site of said HuGlyFab VEGFi comprises a glycan comprising a bisecting GlcNAc, i.e., 100% of the N-glycosylation site of said HuGlyFabVEGFi comprise a glycan comprising a bisecting GlcNAc. In another specific embodiment, at least 20%, 30%, 40%, 50%, 60%, 70%, 75%, 80%, 85%, 90%, 95%, or 99% of the N-glycosylation sites of a HuGlyFab VEGFi used in accordance with the methods described herein are glycosylated with a glycan comprising a bisecting GlcNAc. In another specific embodiment, at least 10%-20%, 20%-30%, 30%-40%, 40%-50%, 50% -60%, 60%-70%, 70%-80%, 80%-90%, or 90%-99% of the N-glycosylation sites of a HuGlyFabVEGFi used in accordance with the methods described herein are glycosylated with a glycan comprising a bisecting GlcNAc. In another specific embodiment, at least 20%, 30%, 40%, 50%, 60%, 70%, 75%, 80%, 85%, 90%, 95%, or 99% of the antigen-binding fragments expressed in a retinal cell in accordance with methods described herein (i.e., the antigen-binding fragments that give rise to HuGlyFab VEGFi, e.g., ranibizumab) are glycosylated with a glycan comprising a bisecting GlcNAc. In another specific embodiment, at least 10%-20%, 20% -30%, 30%-40%, 40%-50%, 50%-60%, 60%-70%, 70%-80%, 80%-90%, or 90%-99% of the antigen-binding fragments expressed in a retinal cell in accordance with methods described herein (i.e., the antigen-binding fragments that give rise to HuGlyFabVEGFi, e.g., ranibizumab) are glycosylated with a glycan comprising a bisecting GlcNAc.

In certain embodiments, the HuGlyFab VEGFi, e.g., ranibizumab, used in accordance with the methods described herein are hyperglycosylated, i.e., in addition to the N-glycosylation resultant from the naturally occurring N-glycosylation sites, said HuGlyFabVEGFi comprise glycans at N-glycosylation sites engineered to be present in the amino acid sequence of the antigen-binding fragment giving rise to HuGlyFabVEGFi. In certain embodiments, the HuGlyFabVEGFi, e.g., ranibizumab, used in accordance with the methods described herein is hyperglycosylated but does not comprise detectable NeuGc and/or α-Gal antigen.

Assays for determining the glycosylation pattern of antibodies, including antigen-binding fragments are known in the art. For example, hydrazinolysis can be used to analyze glycans. First, polysaccharides are released from their associated protein by incubation with hydrazine (the Ludger Liberate Hydrazinolysis Glycan Release Kit, Oxfordshire, UK can be used). The nucleophile hydrazine attacks the glycosidic bond between the polysaccharide and the carrier protein and allows release of the attached glycans. N-acetyl groups are lost during this treatment and have to be reconstituted by re-N-acetylation. Glycans may also be released using enzymes such as glycosidases or endoglycosidases, such as PNGase F and Endo H, which cleave cleanly and with fewer side reactions than hydrazines. The free glycans can be purified on carbon columns and subsequently labeled at the reducing end with the fluorophor 2-amino benzamide. The labeled polysaccharides can be separated on a GlycoSep-N column (GL Sciences) according to the HPLC protocol of Royle et al, Anal Biochem 2002, 304(1):70-90. The resulting fluorescence chromatogram indicates the polysaccharide length and number of repeating units. Structural information can be gathered by collecting individual peaks and subsequently performing MS/MS analysis. Thereby the monosaccharide composition and sequence of the repeating unit can be confirmed and additionally in homogeneity of the polysaccharide composition can be identified. Specific peaks of low or high molecular weight can be analyzed by MALDI-MS/MS and the result used to confirm the glycan sequence. Each peak in the chromatogram corresponds to a polymer, e.g., glycan, consisting of a certain number of repeat units and fragments, e.g., sugar residues, thereof. The chromatogram thus allows measurement of the polymer, e.g., glycan, length distribution. The elution time is an indication for polymer length, while fluorescence intensity correlates with molar abundance for the respective polymer, e.g., glycan. Other methods for assessing glycans associated with antigen-binding fragments include those described by Bondt et al., 2014, Mol. & Cell. Proteomics 13.11:3029-3039, Huang et al., 2006, Anal. Biochem. 349:197-207, and/or Song et al., 2014, Anal. Chem. 86:5661-5666.

Homogeneity or heterogeneity of the glycan patterns associated with antibodies (including antigen-binding fragments), as it relates to both glycan length or size and numbers glycans present across glycosylation sites, can be assessed using methods known in the art, e.g., methods that measure glycan length or size and hydrodynamic radius. HPLC, such as Size exclusion, normal phase, reversed phase, and anion exchange HPLC, as well as capillary electrophoresis, allows the measurement of the hydrodynamic radius. Higher numbers of glycosylation sites in a protein lead to higher variation in hydrodynamic radius compared to a carrier with less glycosylation sites. However, when single glycan chains are analyzed, they may be more homogenous due to the more controlled length. Glycan length can be measured by hydrazinolysis, SDS PAGE, and capillary gel electrophoresis. In addition, homogeneity can also mean that certain glycosylation site usage patterns change to a broader/narrower range. These factors can be measured by Glycopeptide LC-MS/MS.

### Benefits of N-Glycosylation

N-glycosylation confers numerous benefits on the HuGly Fab VEGFi used in the methods described herein. Such benefits are unattainable by production of antigen-binding fragments in E. coli, because E. coli does not naturally possess components needed for N-glycosylation. Further, some benefits are unattainable through antibody production in, e.g., CHO cells, because CHO cells lack components needed for addition of certain glycans (e.g., 2,6 sialic acid and bisecting GlcNAc) and because CHO cells can add glycans, e.g., Neu5Gc not typical to humans. See, e.g., Song et al., 2014, Anal. Chem. 86:5661-5666. Accordingly, by virtue of the discovery set forth herein that anti-VEGF antigen-binding fragments, e.g., ranibizumab, comprise non-canonical N-glycosylation sites (including both reverse and non-consensus glycosylation sites), a method of expressing such anti-VEGF antigen-binding fragments in a manner that results in their glycosylation (and thus improved benefits associated with the antigen-binding fragments) has been realized. In particular, expression of anti-VEGF antigen-binding fragments in human retinal cells results in the production of HuGlyFab VEGFi (e.g., ranibizumab) comprising beneficial glycans that otherwise would not be associated with the antigen-binding fragments or their parent antibody.

While non-canonical glycosylation sites usually result in low level glycosylation (e.g., 1-5%) of the antibody population, the functional benefits may be significant in immunoprivileged organs, such as the eye (See, e.g., van de Bovenkamp et al., 2016, J. Immunol. 196:1435-1441). For example, Fab glycosylation may affect the stability, half-life, and binding characteristics of an antibody. To determine the effects of Fab glycosylation on the affinity of the antibody for its target, any technique known to one of skill in the art may be used, for example, enzyme linked immunosorbent assay (ELISA), or surface plasmon resonance (SPR). To determine the effects of Fab glycosylation on the half-life of the antibody, any technique known to one of skill in the art may be used, for example, by measurement of the levels of radioactivity in the blood or organs (e.g., the eye) in a subject to whom a radiolabelled antibody has been administered. To determine the effects of Fab glycosylation on the stability, for example, levels of aggregation or protein unfolding, of the antibody, any technique known to one of skill in the art may be used, for example, differential scanning calorimetry (DSC), high performance liquid chromatography (HPLC), e.g., size exclusion high performance liquid chromatography (SEC-HPLC), capillary electrophoresis, mass spectrometry, or turbidity measurement. Provided herein, the HuGlyFabVEGFi transgene results in production of an antigen-binding fragment which is 0.5%, 1%, 2%, 3%, 4%, 5%, 6%, 7%, 8%, 9%, or 10% or more glycosylated at non-canonical sites. In certain embodiments, 0.5%, 1%, 2%, 3%, 4%, 5%, 6%, 7%, 8%, 9%, or 10% or more antigen-binding fragments from a population of antigen-binding fragments are glycosylated at non-canonical sites. In certain embodiments, 0.5%, 1%, 2%, 3%, 4%, 5%, 6%, 7%, 8%, 9%, or 10% or more non-canonical sites are glycosylated. In certain embodiments, the glycosylation of the antigen-binding fragment at these non-canonical sites is 25%, 50%, 100%, 200%, 300%, 400%, 500%, or more greater than the amount of glycosylation of these non-canonical sites in an antigen-binding fragment produced in HEK293 cells.

The presence of sialic acid on HuGlyFab VEGFi used in the methods described herein can impact clearance rate of the HuGlyFabVEGFi, e.g., the rate of clearance from the vitreous humour. Accordingly, sialic acid patterns of a HuGlyFab VEGFi can be used to generate a therapeutic having an optimized clearance rate. Method of assessing antigen-binding fragment clearance rate are known in the art. See, e.g., Huang et al., 2006, Anal. Biochem. 349:197-207.

In another specific embodiment, a benefit conferred by N-glycosylation is reduced aggregation. Occupied N-glycosylation sites can mask aggregation prone amino acid residues, resulting in decreased aggregation. Such N-glycosylation sites can be native to an antigen-binding fragment used herein, or engineered into an antigen-binding fragment used herein, resulting in HuGlyFabVEGFi that is less prone to aggregation when expressed, e.g., expressed in retinal cells. Methods of assessing aggregation of antibodies are known in the art. See, e.g., Courtois et al., 2016, mAbs 8:99-112 which is incorporated by reference herein in its entirety.

In another specific embodiment, a benefit conferred by N-glycosylation is reduced immunogenicity. Such N-glycosylation sites can be native to an antigen-binding fragment used herein, or engineered into an antigen-binding fragment used herein, resulting in HuGlyFabVEGFi that is less prone to immunogenicity when expressed, e.g., expressed in retinal cells.

In another specific embodiment, a benefit conferred by N-glycosylation is protein stability. N-glycosylation of proteins is well-known to confer stability on them, and methods of assessing protein stability resulting from N-glycosylation are known in the art. See, e.g., Sola and Griebenow, 2009, J Pharm Sci., 98(4): 1223 1245.

In another specific embodiment, a benefit conferred by N-glycosylation is altered binding affinity. It is known in the art that the presence of N-glycosylation sites in the variable domains of an antibody can increase the affinity of the antibody for its antigen. See, e.g., Bovenkamp et al., 2016, J. Immunol. 196:1435-1441. Assays for measuring antibody binding affinity are known in the art. See, e.g., Wright et al., 1991, EMBO) J. 10:2717-2723; and Leibiger et al., 1999, Biochem. J. 338:529-538.

### 5.1.2 Tyrosine Sulfation

Tyrosine sulfation occurs at tyrosine (Y) residues with glutamate (E) or aspartate (D) within +5 to −5 position of Y, and where position −1 of Y is a neutral or acidic charged amino acid, but not a basic amino acid, e.g., arginine (R), lysine (K), or histidine (H) that abolishes sulfation. Surprisingly, anti-VEGF antigen-binding fragments for use in accordance with the methods described herein, e.g., ranibizumab, comprise tyrosine sulfation sites (see FIG. 1). Accordingly, the methods described herein comprise use of anti-VEGF antigen-binding fragments, e.g., HuPTMFabVEGFi, that comprise at least one tyrosine sulfation site, such the anti-VEGF antigen-binding fragments, when expressed in retinal cells, can be tyrosine sulfated.

Importantly, tyrosine-sulfated antigen-binding fragments, e.g., ranibizumab, cannot be produced in E. coli, which naturally does not possess the enzymes required for tyrosine-sulfation. Further, CHO cells are deficient for tyrosine sulfation-they are not secretory cells and have a limited capacity for post-translational tyrosine-sulfation. See, e.g., Mikkelsen & Ezban, 1991, Biochemistry 30: 1533-1537. Advantageously, the methods provided herein call for expression of anti-VEGF antigen-binding fragments, e.g., HuPTMFab VEGFi, for example, ranibizumab, in retinal cells, which are secretory and do have capacity for tyrosine sulfation. See Kanan et al., 2009, Exp. Eye Res. 89: 559-567 and Kanan & Al-Ubaidi, 2015, Exp. Eye Res. 133: 126-131 reporting the production of tyrosine-sulfated glycoproteins secreted by retinal cells.

Tyrosine sulfation is advantageous for several reasons. For example, tyrosine-sulfation of the antigen-binding fragment of therapeutic antibodies against targets has been shown to dramatically increase avidity for antigen and activity. See, e.g., Loos et al., 2015, PNAS 112: 12675-12680, and Choe et al., 2003, Cell 114: 161-170. Assays for detection tyrosine sulfation are known in the art. See, e.g., Yang et al., 2015, Molecules 20:2138-2164.

### 5.1.3 O-Glycosylation

O-glycosylation comprises the addition of N-acetyl-galactosamine to serine or threonine residues by the enzyme. It has been demonstrated that amino acid residues present in the hinge region of antibodies can be O-glycosylated. In certain embodiments, the anti-VEGF antigen-binding fragments, e.g., ranibizumab, used in accordance with the methods described herein comprise all or a portion of their hinge region, and thus are capable of being O-glycosylated when expressed in human retinal cells. The possibility of O-glycosylation confers another advantage to the HuPTMFab VEGFi, e.g., HuGlyFabVEGFi, provided herein, as compared to, e.g., antigen-binding fragments produced in E. coli, again because the E. coli naturally does not contain machinery equivalent to that used in human O-glycosylation. (Instead, O-glycosylation in E. coli has been demonstrated only when the bacteria is modified to contain specific O-glycosylation machinery. See, e.g., Faridmoayer et al., 2007, J. Bacteriol. 189:8088-8098.)O-glycosylated HuPTMFabVEGFi, e.g., HuGlyFab VEGFi, by virtue of possessing glycans, shares advantageous characteristics with N-glycosylated HuGlyFabVEGFi (as discussed above).

### 5.2 Constructs and Formulations

For use in the methods provided herein are viral vectors or other DNA expression constructs encoding an anti-VEGF antigen-binding fragment or a hyperglycosylated derivative of an anti-VEGF antigen-binding fragment. The viral vectors and other DNA expression constructs provided herein include any suitable method for delivery of a transgene to a target cell (e.g., retinal pigment epithelial cells). The means of delivery of a transgene include viral vectors, liposomes, other lipid-containing complexes, other macromolecular complexes, synthetic modified mRNA, unmodified mRNA, small molecules, non-biologically active molecules (e.g., gold particles), polymerized molecules (e.g., dendrimers), naked DNA, plasmids, phages, transposons, cosmids, or episomes. In some embodiments, the vector is a targeted vector, e.g., a vector targeted to retinal pigment epithelial cells.

In some aspects, the disclosure provides for a nucleic acid for use, wherein the nucleic acid encodes a HuPTMFabVEGFi, e.g., HuGlyFabVEGFi operatively linked to a promoter selected from the group consisting of: cytomegalovirus (CMV) promoter, Rous sarcoma virus (RSV) promoter, MMT promoter, EF-1 alpha promoter, UB6 promoter, chicken beta-actin promoter, CAG promoter, RPE65 promoter and opsin promoter.

In certain embodiments, provided herein are recombinant vectors that comprise one or more nucleic acids (e.g. polynucleotides). The nucleic acids may comprise DNA, RNA, or a combination of DNA and RNA. In certain embodiments, the DNA comprises one or more of the sequences selected from the group consisting of promoter sequences, the sequence of the gene of interest (the transgene, e.g., an anti-VEGF antigen-binding fragment), untranslated regions, and termination sequences. In certain embodiments, viral vectors provided herein comprise a promoter operably linked to the gene of interest.

In certain embodiments, nucleic acids (e.g., polynucleotides) and nucleic acid sequences disclosed herein may be codon-optimized, for example, via any codon-optimization technique known to one of skill in the art (see, e.g., review by Quax et al., 2015, Mol Cell 59:149-161).

In a specific embodiment, the constructs described herein comprise the following components: (1) AAV2 inverted terminal repeats that flank the expression cassette; (2) Control elements, which include a) the CB7 promoter, comprising the CMV enhancer/chicken β-actin promoter, b) a chicken β-actin intron and c) a rabbit β-globin poly A signal; and (3) nucleic acid sequences coding for the heavy and light chains of anti-VEGF antigen-binding fragment, separated by a self-cleaving furin (F)/F2A linker, ensuring expression of equal amounts of the heavy and the light chain polypeptides.

5.2.1 mRNA

In certain embodiments, the vectors provided herein are modified mRNA encoding for the gene of interest (e.g., the transgene, for example, an anti-VEGF antigen-binding fragment moiety). The synthesis of modified and unmodified mRNA for delivery of a transgene to retinal pigment epithelial cells is taught, for example, in Hansson et al., J. Biol. Chem., 2015, 290(9):5661-5672, which is incorporated by reference herein in its entirety. In certain embodiments, provided herein is a modified mRNA encoding for an anti-VEGF antigen-binding fragment moiety.

### 5.2.2 Viral Vectors

Viral vectors include adenovirus, adeno-associated virus (AAV, e.g., AAV8), lentivirus, helper-dependent adenovirus, herpes simplex virus, poxvirus, hemagglutinin virus of Japan (HVJ), alphavirus, vaccinia virus, and retrovirus vectors. Retroviral vectors include murine leukemia virus (MLV)- and human immunodeficiency virus (HIV)-based vectors. Alphavirus vectors include semliki forest virus (SFV) and sindbis virus (SIN). In certain embodiments, the viral vectors provided herein are recombinant viral vectors. In certain embodiments, the viral vectors provided herein are altered such that they are replication-deficient in humans. In certain embodiments, the viral vectors are hybrid vectors, e.g., an AAV vector placed into a “helpless” adenoviral vector. In certain embodiments, provided herein are viral vectors comprising a viral capsid from a first virus and viral envelope proteins from a second virus. In specific embodiments, the second virus is vesicular stomatitus virus (VSV). In more specific embodiments, the envelope protein is VSV-G protein.

In certain embodiments, the viral vectors provided herein are HIV based viral vectors. In certain embodiments, HIV-based vectors provided herein comprise at least two polynucleotides, wherein the gag and pol genes are from an HIV genome and the env gene is from another virus.

In certain embodiments, the viral vectors provided herein are herpes simplex virus-based viral vectors. In certain embodiments, herpes simplex virus-based vectors provided herein are modified such that they do not comprise one or more immediately early (IE) genes, rendering them non-cytotoxic.

In certain embodiments, the viral vectors provided herein are MLV based viral vectors. In certain embodiments, MLV-based vectors provided herein comprise up to 8 kb of heterologous DNA in place of the viral genes.

In certain embodiments, the viral vectors provided herein are lentivirus-based viral vectors. In certain embodiments, lentiviral vectors provided herein are derived from human lentiviruses. In certain embodiments, lentiviral vectors provided herein are derived from non-human lentiviruses. In certain embodiments, lentiviral vectors provided herein are packaged into a lentiviral capsid. In certain embodiments, lentiviral vectors provided herein comprise one or more of the following elements: long terminal repeats, a primer binding site, a polypurine tract, att sites, and an encapsidation site.

In certain embodiments, the viral vectors provided herein are alphavirus-based viral vectors. In certain embodiments, alphavirus vectors provided herein are recombinant, replication-defective alphaviruses. In certain embodiments, alphavirus replicons in the alphavirus vectors provided herein are targeted to specific cell types by displaying a functional heterologous ligand on their virion surface.

In certain embodiments, the viral vectors provided herein are AAV based viral vectors. In preferred embodiments, the viral vectors provided herein are AAV8 based viral vectors. In certain embodiments, the AAV8 based viral vectors provided herein retain tropism for retinal cells. In certain embodiments, the AAV-based vectors provided herein encode the AAV rep gene (required for replication) and/or the AAV cap gene (required for synthesis of the capsid proteins). Multiple AAV serotypes have been identified. In certain embodiments, AAV-based vectors provided herein comprise components from one or more serotypes of AAV. In certain embodiments, AAV based vectors provided herein comprise capsid components from one or more of AAV1, AAV2, AAV3, AAV4, AAV5, AAV6, AAV7, AAV8, AAV9, AAV10, AAV11, or AAVrh10. In preferred embodiments, AAV based vectors provided herein comprise components from one or more of AAV8, AAV9, AAV10, AAV11, or AAVrh10 serotypes.

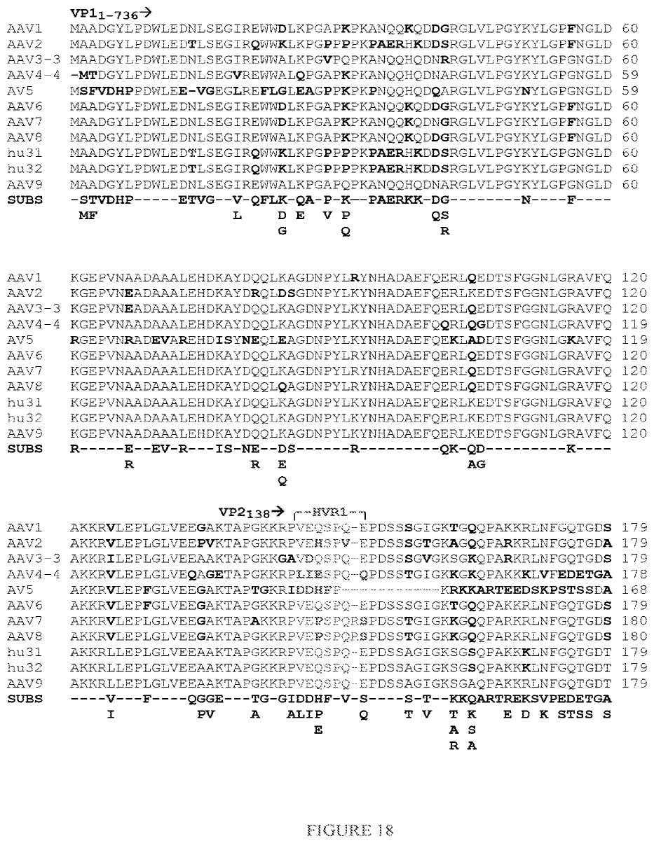

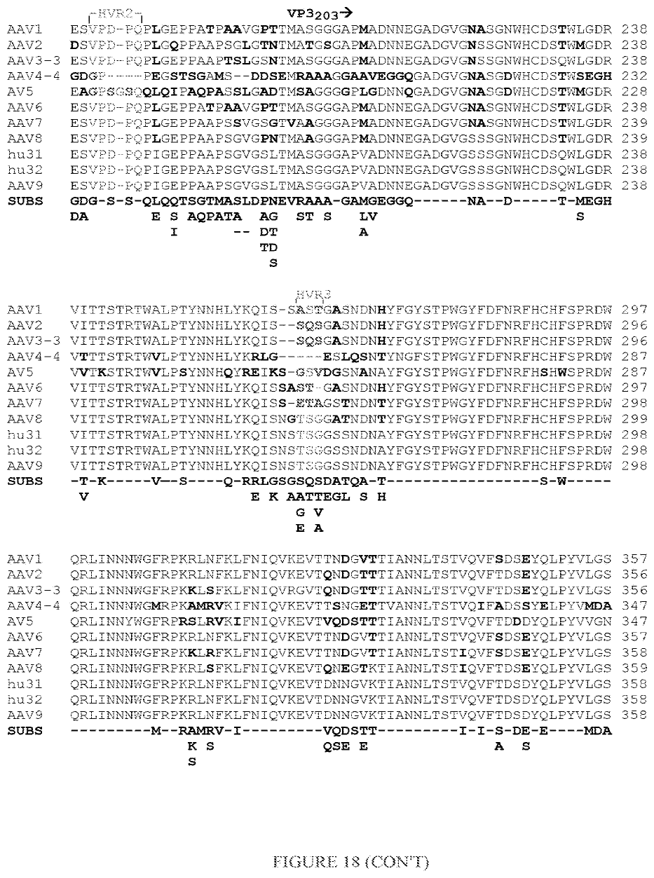

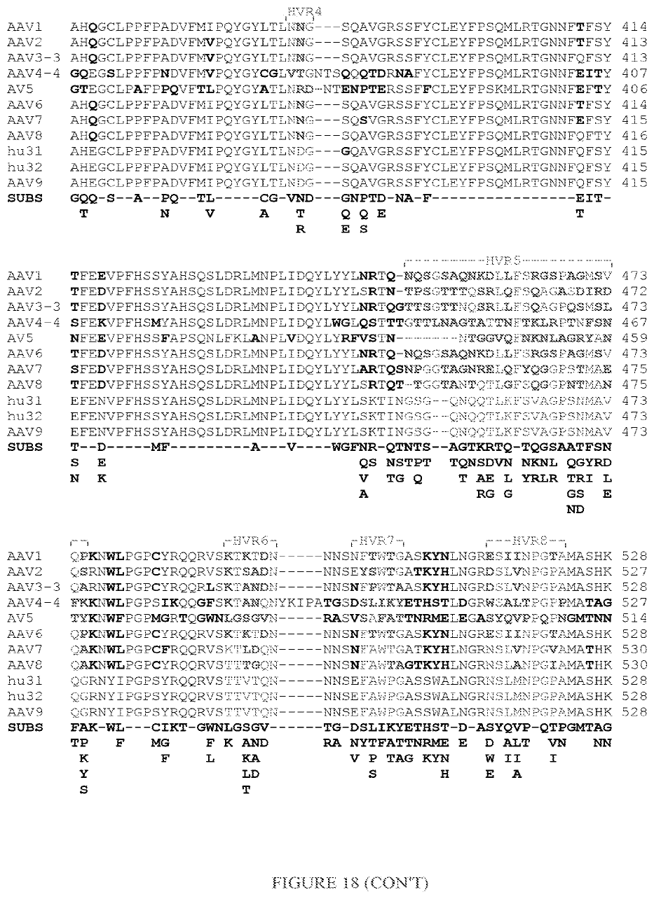

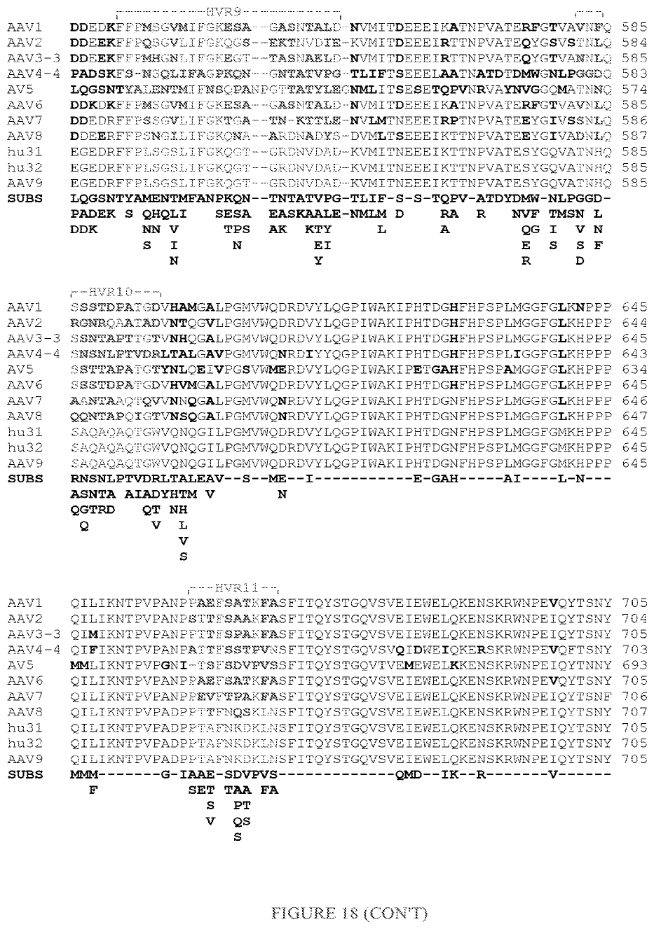

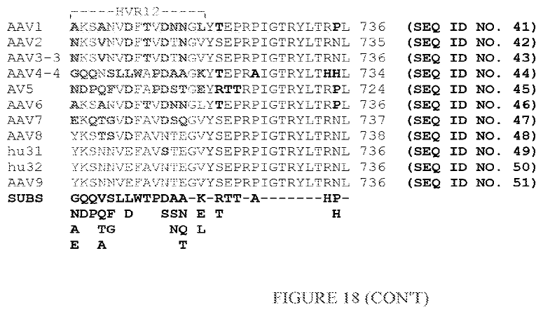

Provided in particular embodiments are AAV8 vectors comprising a viral genome comprising an expression cassette for expression of the transgene, under the control of regulatory elements and flanked by ITRs and a viral capsid that has the amino acid sequence of the AAV8 capsid protein or is at least 95%, 96%, 97%, 98%, 99% or 99.9% identical to the amino acid sequence of the AAV8 capsid protein (SEQ ID NO: 48) while retaining the biological function of the AAV8 capsid. In certain embodiments, the encoded AAV8 capsid has the sequence of SEQ ID NO: 48 with 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions and retaining the biological function of the AAV8 capsid. FIG. 18 provides a comparative alignment of the amino acid sequences of the capsid proteins of different AAV serotypes with potential amino acids that may be substituted at certain positions in the aligned sequences based upon the comparison in the row labeled SUBS. Accordingly, in specific embodiments, the AAV8 vector comprises an AAV8 capsid variant that has 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions identified in the SUBS row of FIG. 18 that are not present at that position in the native AAV8 sequence.

In certain embodiments, the AAV that is used in the methods described herein is Anc80 or Anc80L65, as described in Zinn et al., 2015, Cell Rep. 12(6): 1056-1068, which is incorporated by reference in its entirety. In certain embodiments, the AAV that is used in the methods described herein comprises one of the following amino acid insertions: LGETTRP or LALGETTRP, as described in U.S. Pat. Nos. 9,193,956; 9,458,517; and 9,587,282 and US patent application publication no. 2016/0376323, each of which is incorporated herein by reference in its entirety. In certain embodiments, the AAV that is used in the methods described herein is AAV.7m8, as described in U.S. Pat. Nos. 9,193,956; 9,458,517; and 9,587,282 and US patent application publication no. 2016/0376323, each of which is incorporated herein by reference in its entirety. In certain embodiments, the AAV that is used in the methods described herein is any AAV disclosed in U.S. Pat. No. 9,585,971, such as AAV-PHP.B. In certain embodiments, the AAV that is used in the methods described herein is an AAV disclosed in any of the following patents and patent applications, each of which is incorporated herein by reference in its entirety: United States Patent Nos. 7,906, 111; 8,524,446; 8,999,678; 8,628,966; 8,927,514; 8,734,809; U.S. Pat. Nos. 9,284,357; 9,409,953; 9,169,299; 9,193,956; 9,458,517; and 9,587,282 US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335.

AAV8-based viral vectors are used in certain of the methods described herein. Nucleic acid sequences of AAV based viral vectors and methods of making recombinant AAV and AAV capsids are taught, for example, in U.S. Pat. No. 7,282,199 B2, U.S. Pat. No. 7,790,449 B2, U.S. Pat. No. 8,318,480 B2, U.S. Pat. No. 8,962,332 B2 and International Patent Application No. PCT/EP2014/076466, each of which is incorporated herein by reference in its entirety. In one aspect, provided herein are AAV (e.g., AAV8)-based viral vectors encoding a transgene (e.g., an anti-VEGF antigen-binding fragment). In specific embodiments, provided herein are AAV8-based viral vectors encoding an anti-VEGF antigen-binding fragment. In more specific embodiments, provided herein are AAV8-based viral vectors encoding ranibizumab.

In certain embodiments, a single-stranded AAV (ssAAV) may be used supra. In certain embodiments, a self-complementary vector, e.g., scAAV, may be used (see, e.g., Wu, 2007, Human Gene Therapy, 18(2): 171-82, McCarty et al, 2001, Gene Therapy, Vol 8, Number 16, Pages 1248-1254; and U.S. Pat. Nos. 6,596,535; 7,125,717; and 7,456,683, each of which is incorporated herein by reference in its entirety).

In certain embodiments, the viral vectors used in the methods described herein are adenovirus based viral vectors. A recombinant adenovirus vector may be used to transfer in the anti-VEGF antigen-binding fragment. The recombinant adenovirus can be a first generation vector, with an E1 deletion, with or without an E3 deletion, and with the expression cassette inserted into either deleted region. The recombinant adenovirus can be a second generation vector, which contains full or partial deletions of the E2 and E4 regions. A helper-dependent adenovirus retains only the adenovirus inverted terminal repeats and the packaging signal (phi). The transgene is inserted between the packaging signal and the 3′ITR, with or without stuffer sequences to keep the genome close to wild-type size of approx. 36 kb. An exemplary protocol for production of adenoviral vectors may be found in Alba et al., 2005, “Gutless adenovirus: last generation adenovirus for gene therapy,” Gene Therapy 12:S18-S27, which is incorporated by reference herein in its entirety.

In certain embodiments, the viral vectors used in the methods described herein are lentivirus based viral vectors. A recombinant lentivirus vector may be used to transfer in the anti-VEGF antigen-binding fragment. Four plasmids are used to make the construct: Gag/pol sequence containing plasmid, Rev sequence containing plasmids, Envelope protein containing plasmid (i.e. VSV-G), and Cis plasmid with the packaging elements and the anti-VEGF antigen-binding fragment gene.

For lentiviral vector production, the four plasmids are co-transfected into cells (i.e., HEK293 based cells), whereby polyethylenimine or calcium phosphate can be used as transfection agents, among others. The lentivirus is then harvested in the supernatant (lentiviruses need to bud from the cells to be active, so no cell harvest needs/should be done). The supernatant is filtered (0.45 μm) and then magnesium chloride and benzonase added. Further downstream processes can vary widely, with using TFF and column chromatography being the most GMP compatible ones. Others use ultracentrifugation with/without column chromatography. Exemplary protocols for production of lentiviral vectors may be found in Lesch et al., 2011, “Production and purification of lentiviral vector generated in 293T suspension cells with baculoviral vectors,” Gene Therapy 18:531-538, and Ausubel et al., 2012, “Production of CGMP-Grade Lentiviral Vectors,” Bioprocess Int. 10(2):32-43, both of which are incorporated by reference herein in their entireties.

In a specific embodiment, a vector for use in the methods described herein is one that encodes an anti-VEGF antigen-binding fragment (e.g., ranibizumab) such that, upon introduction of the vector into a relevant cell (e.g., a retinal cell in vivo or in vitro), a glycosylated and or tyrosine sulfated variant of the anti-VEGF antigen-binding fragment is expressed by the cell. In a specific embodiment, the expressed anti-VEGF antigen-binding fragment comprises a glycosylation and/or tyrosine sulfation pattern as described in Section 5.1, above.

### 5.2.3 Promoters and Modifiers of Gene Expression

In certain embodiments, the vectors provided herein comprise components that modulate gene delivery or gene expression (e.g., “expression control elements”). In certain embodiments, the vectors provided herein comprise components that modulate gene expression. In certain embodiments, the vectors provided herein comprise components that influence binding or targeting to cells. In certain embodiments, the vectors provided herein comprise components that influence the localization of the polynucleotide (e.g., the transgene) within the cell after uptake. In certain embodiments, the vectors provided herein comprise components that can be used as detectable or selectable markers, e.g., to detect or select for cells that have taken up the polynucleotide.

In certain embodiments, the viral vectors provided herein comprise one or more promoters. In certain embodiments, the promoter is a constitutive promoter. In certain embodiments, the promoter is an inducible promoter. Inducible promoters may be preferred so that transgene expression may be turned on and off as desired for therapeutic efficacy. Such promoters include, for example, hypoxia-induced promoters and drug inducible promoters, such as promoters induced by rapamycin and related agents. Hypoxia-inducible promoters include promoters with HIF binding sites, see, for example, Schödel, et al., 2011, Blood 117(23):c207-e217 and Kenneth and Rocha, 2008, Biochem J. 414:19-29, each of which is incorporated by reference for teachings of hypoxia-inducible promoters. In addition, hypoxia-inducible promoters that may be used in the constructs include the erythropoietin promoter and N-WASP promoter (see, Tsuchiya, 1993, J. Biochem. 113:395 for disclosure of the erythropoietin promoter and Salvi, 2017, Biochemistry and Biophysics Reports 9:13-21 for disclosure of N-WASP promoter, both of which are incorporated by reference for the teachings of hypoxia-induced promoters). Alternatively, the constructs may contain drug inducible promoters, for example promoters inducible by administration of rapamycin and related analogs (see, for example, International Patent Application Publication Nos. WO94/18317, WO 96/20951, WO 96/41865, WO 99/10508, WO 99/10510, WO 99/36553, and WO 99/41258, and U.S. Pat. No. 7,067,526 (disclosing rapamycin analogs), which are incorporated by reference herein for their disclosure of drug inducible promoters). In certain embodiments the promoter is a hypoxia-inducible promoter. In certain embodiments, the promoter comprises a hypoxia-inducible factor (HIF) binding site. In certain embodiments, the promoter comprises a HIF-1a binding site. In certain embodiments, the promoter comprises a HIF-2a binding site. In certain embodiments, the HIF binding site comprises an RCGTG motif. For details regarding the location and sequence of HIF binding sites, see, e.g., Schödel, et al., Blood, 2011, 117(23):e207-e217, which is incorporated by reference herein in its entirety. In certain embodiments, the promoter comprises a binding site for a hypoxia induced transcription factor other than a HIF transcription factor. In certain embodiments, the viral vectors provided herein comprise one or more IRES sites that is preferentially translated in hypoxia. For teachings regarding hypoxia-inducible gene expression and the factors involved therein, see, e.g., Kenneth and Rocha, Biochem J., 2008, 414:19-29, which is incorporated by reference herein in its entirety.

In certain embodiments, the promoter is a CB7 promoter (see Dinculescu et al., 2005, Hum Gene Ther 16: 649-663, incorporated by reference herein in its entirety). In some embodiments, the CB7 promoter includes other expression control elements that enhance expression of the transgene driven by the vector. In certain embodiments, the other expression control elements include chicken β-actin intron and/or rabbit β-globin polA signal. In certain embodiments, the promoter comprises a TATA box. In certain embodiments, the promoter comprises one or more elements. In certain embodiments, the one or more promoter elements may be inverted or moved relative to one another. In certain embodiments, the elements of the promoter are positioned to function cooperatively. In certain embodiments, the elements of the promoter are positioned to function independently. In certain embodiments, the viral vectors provided herein comprise one or more promoters selected from the group consisting of the human CMV immediate early gene promoter, the SV40 early promoter, the Rous sarcoma virus (RS) long terminal repeat, and rat insulin promoter. In certain embodiments, the vectors provided herein comprise one or more long terminal repeat (LTR) promoters selected from the group consisting of AAV, MLV, MMTV, SV40, RSV, HIV-1, and HIV-2 LTRs. In certain embodiments, the vectors provided herein comprise one or more tissue specific promoters (e.g., a retinal pigment epithelial cell-specific promoter). In certain embodiments, the viral vectors provided herein comprise a RPE65 promoter. In certain embodiments, the vectors provided herein comprise a VMD2 promoter.

In certain embodiments, the viral vectors provided herein comprise one or more regulatory elements other than a promoter. In certain embodiments, the viral vectors provided herein comprise an enhancer. In certain embodiments, the viral vectors provided herein comprise a repressor. In certain embodiments, the viral vectors provided herein comprise an intron or a chimeric intron. In certain embodiments, the viral vectors provided herein comprise a polyadenylation sequence.

### 5.2.4 Signal Peptides

In certain embodiments, the vectors provided herein comprise components that modulate protein delivery. In certain embodiments, the viral vectors provided herein comprise one or more signal peptides. Signal peptides may also be referred to herein as “leader sequences” or “leader peptides”. In certain embodiments, the signal peptides allow for the transgene product (e.g., the anti-VEGF antigen-binding fragment moiety) to achieve the proper packaging (e.g. glycosylation) in the cell. In certain embodiments, the signal peptides allow for the transgene product (e.g., the anti-VEGF antigen-binding fragment moiety) to achieve the proper localization in the cell. In certain embodiments, the signal peptides allow for the transgene product (e.g., the anti-VEGF antigen-binding fragment moiety) to achieve secretion from the cell. Examples of signal peptides to be used in connection with the vectors and transgenes provided herein may be found in Table 1.

TABLE 1
Signal peptides for use with the vectors provided herein.

| SEQ | | |
|---|---|---|
| ID NO. | Signal Peptide | Sequence |
| 5 | VEGF-A signal peptide | MNFLLSWVHW SLALLLYLHH |
| | | AKWSQA |
| 6 | Fibulin-1 signal peptide | MERAAPSRRV PLPLLLLGGL |
| | | ALLAAGVDA |
| 7 | Vitronectin signal peptide | MAPLRPLLIL ALLAWVALA |
| 8 | Complement Factor H signal peptide | MRLLAKIICLMLWAICVA |
| 9 | Opticin signal peptide | MRLLAFLSLL ALVLQETGT |
| 22 | Albumin signal peptide | MKWVTFISLLFLFSSAYS |
| 23 | Chymotrypsinogen signal peptide | MAFLWLLSCWALLGTTFG |
| 24 | Interleukin-2 signal peptide | MYRMQLLSCIALILALVTNS |
| 25 | Trypsinogen-2 signal peptide | MNLLLILTFVAAAVA |

### 5.2.5 Polycistronic Messages—IRES and F2A Linkers

Internal ribosome entry sites. A single construct can be engineered to encode both the heavy and light chains separated by a cleavable linker or IRES so that separate heavy and light chain polypeptides are expressed by the transduced cells. In certain embodiments, the viral vectors provided herein provide polycistronic (e.g., bicistronic) messages. For example, the viral construct can encode the heavy and light chains separated by an internal ribosome entry site (IRES) elements (for examples of the use of IRES elements to create bicistronic vectors see, e.g., Gurtu et al., 1996, Biochem. Biophys. Res. Comm. 229(1):295-8, which is herein incorporated by reference in its entirety). IRES elements bypass the ribosome scanning model and begin translation at internal sites. The use of IRES in AAV is described, for example, in Furling et al., 2001, Gene Ther 8(11): 854-73, which is herein incorporated by reference in its entirety. In certain embodiments, the bicistronic message is contained within a viral vector with a restraint on the size of the polynucleotide(s) therein. In certain embodiments, the bicistronic message is contained within an AAV virus-based vector (e.g., an AAV8-based vector).

Furin-F2A linkers. In other embodiments, the viral vectors provided herein encode the heavy and light chains separated by a cleavable linker such as the self-cleaving furin/F2A (F/F2A) linkers (Fang et al., 2005, Nature Biotechnology 23: 584-590, and Fang, 2007, Mol Ther 15: 1153-9, each of which is incorporated by reference herein in its entirety).

For example, a furin-F2A linker may be incorporated into an expression cassette to separate the heavy and light chain coding sequences, resulting in a construct with the structure:
Leader—Heavy chain—Furin site—F2A site—Leader—Light chain—PolyA.

The F2A site, with the amino acid sequence LLNFDLLKLAGDVESNPGP (SEQ ID NO: 26) is self-processing, resulting in “cleavage” between the final G and P amino acid residues. Additional linkers that could be used include but are not limited to:

T2A:
(SEQ ID NO: 27)
(GSG)EGRGSLLTCGDVEENPGP;
P2A:
(SEQ ID NO: 28)
(GSG)ATNFSLLKQAGDVEENPGP;
E2A:
(SEQ ID NO: 29)
(GSG)QCTNYALLKLAGDVESNPGP;
F2A:
(SEQ ID NO: 30)
(GSG)VKQTLNFDLLKLAGDVESNPGP.

A peptide bond is skipped when the ribosome encounters the F2A sequence in the open reading frame, resulting in the termination of translation, or continued translation of the downstream sequence (the light chain). This self-processing sequence results in a string of additional amino acids at the end of the C-terminus of the heavy chain. However, such additional amino acids are then cleaved by host cell Furin at the furin sites, located immediately prior to the F2A site and after the heavy chain sequence, and further cleaved by carboxypeptidases. The resultant heavy chain may have one, two, three, or more additional amino acids included at the C-terminus, or it may not have such additional amino acids, depending on the sequence of the Furin linker used and the carboxypeptidase that cleaves the linker in vivo (See, e.g., Fang et al., 17 Apr. 2005, Nature Biotechnol. Advance Online Publication; Fang et al., 2007, Molecular Therapy 15(6): 1153-1159; Luke, 2012, Innovations in Biotechnology, Ch. 8, 161-186). Furin linkers that may be used comprise a series of four basic amino acids, for example, RKRR, RRRR, RRKR, or RKKR. Once this linker is cleaved by a carboxypeptidase, additional amino acids may remain, such that an additional zero, one, two, three or four amino acids may remain on the C-terminus of the heavy chain, for example, R, RR, RK, RKR, RRR, RRK, RKK, RKRR, RRRR, RRKR, or RKKR. In certain embodiments, one the linker is cleaved by an carboxypeptidase, no additional amino acids remain. In certain embodiments, 0.5%, 1%, 2%, 3%, 4%, 5%, 10%, 15%, or 20%, or less but more than 0% of the antibody, e.g., antigen-binding fragment, population produced by the constructs for use in the methods described herein has one, two, three, or four amino acids remaining on the C-terminus of the heavy chain after cleavage. In certain embodiments, 0.5-1%, 0.5%-2%, 0.5%-3%, 0.5%-4%, 0.5%-5%, 0.5%-10%, 0.5%-20%, 1%-2%, 1%-3%, 1%-4%, 1%-5%, 1%-10%, 1%-20%, 2%-3%, 2%-4%, 2%-5%, 2%-10%, 2%-20%, 3%-4%, 3%-5%, 3%-10%, 3%-20%, 4%-5%, 4%-10%, 4%-20%, 5%-10%, 5%-20%, or 10%-20% of the antibody, e.g., antigen-binding fragment, population produced by the constructs for use in the methods described herein has one, two, three, or four amino acids remaining on the C-terminus of the heavy chain after cleavage. In certain embodiments, the furin linker has the sequence R—X—K/R—R, such that the additional amino acids on the C-terminus of the heavy chain are R, RX, RXK, RXR, RXKR, or RXRR, where X is any amino acid, for example, alanine (A). In certain embodiments, no additional amino acids may remain on the C-terminus of the heavy chain.

In certain embodiments, an expression cassette described herein is contained within a viral vector with a restraint on the size of the polynucleotide(s) therein. In certain embodiments, the expression cassette is contained within an AAV virus-based vector (e.g., an AAV8-based vector).

### 5.2.6 Untranslated Regions

In certain embodiments, the viral vectors provided herein comprise one or more untranslated regions (UTRs), e.g., 3′ and/or 5′ UTRs. In certain embodiments, the UTRs are optimized for the desired level of protein expression. In certain embodiments, the UTRs are optimized for the mRNA half life of the transgene. In certain embodiments, the UTRs are optimized for the stability of the mRNA of the transgene. In certain embodiments, the UTRs are optimized for the secondary structure of the mRNA of the transgene.

### 5.2.7 Inverted Terminal Repeats

In certain embodiments, the viral vectors provided herein comprise one or more inverted terminal repeat (ITR) sequences. ITR sequences may be used for packaging the recombinant gene expression cassette into the virion of the viral vector. In certain embodiments, the ITR is from an AAV, e.g., AAV8 or AAV2 (see, e.g., Yan et al., 2005, J. Virol., 79(1): 364-379; U.S. Pat. No. 7,282,199 B2, U.S. Pat. No. 7,790,449 B2, U.S. Pat. No. 8,318,480 B2, U.S. Pat. No. 8,962,332 B2 and International Patent Application No. PCT/EP2014/076466, each of which is incorporated herein by reference in its entirety).

### 5.2.8 Transgenes

The HuPTMFab VEGFi, e.g., HuGlyFabVEGFi encoded by the transgene can include, but is not limited to an antigen-binding fragment of an antibody that binds to VEGF, such as bevacizumab; an anti-VEGF Fab moiety such as ranibizumab; or such bevacizumab or ranibizumab Fab moieties engineered to contain additional glycosylation sites on the Fab domain (e.g., see Courtois et al., 2016, mAbs 8: 99-112 which is incorporated by reference herein in its entirety for it description of derivatives of bevacizumab that are hyperglycosylated on the Fab domain of the full length antibody).

In certain embodiments, the vectors provided herein encode an anti-VEGF antigen-binding fragment transgene. In specific embodiments, the anti-VEGF antigen-binding fragment transgene is controlled by appropriate expression control elements for expression in retinal cells: In certain embodiments, the anti-VEGF antigen-binding fragment transgene comprises bevacizumab Fab portion of the light and heavy chain cDNA sequences (SEQ ID NOs. 10 and 11, respectively). In certain embodiments, the anti-VEGF antigen-binding fragment transgene comprises ranibizumab light and heavy chain cDNA sequences (SEQ ID NOs. 12 and 13, respectively). In certain embodiments, the anti-VEGF antigen-binding fragment transgene encodes a bevacizumab Fab, comprising a light chain and a heavy chain of SEQ ID NOs: 3 and 4, respectively. In certain embodiments, the anti-VEGF antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 3. In certain embodiments, the anti-VEGF antigen-binding fragment transgene encodes an antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 4. In certain embodiments, the anti-VEGF antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 3 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 4. In certain embodiments, the anti-VEGF antigen-binding fragment transgene encodes a hyperglycosylated ranibizumab, comprising a light chain and a heavy chain of SEQ ID NOs: 1 and 2, respectively. In certain embodiments, the anti-VEGF antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 1. In certain embodiments, the anti-VEGF antigen-binding fragment transgene encodes an antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 2. In certain embodiments, the anti-VEGF antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 1 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 2.

In certain embodiments, the anti-VEGF antigen-binding fragment transgene encodes a hyperglycosylated bevacizumab Fab, comprising a light chain and a heavy chain of SEQ ID NOs: 3 and 4, with one or more of the following mutations: L118N (heavy chain), E195N (light chain), or Q160N or Q160S (light chain). In certain embodiments, the anti-VEGF antigen-binding fragment transgene encodes a hyperglycosylated ranibizumab, comprising a light chain and a heavy chain of SEQ ID NOs: 1 and 2, with one or more of the following mutations: L118N (heavy chain), E195N (light chain), or Q160N or Q160S (light chain). The sequences of the antigen-binding fragment transgene cDNAs may be found, for example, in Table 2. In certain embodiments, the sequence of the antigen-binding fragment transgene cDNAs is obtained by replacing the signal sequence of SEQ ID NOs: 10 and 11 or SEQ ID NOs: 12 and 13 with one or more signal sequences listed in Table 1.

In certain embodiments, the anti-VEGF antigen-binding fragment transgene encodes an antigen-binding fragment and comprises the nucleotide sequences of the six bevacizumab CDRs. In certain embodiments, the anti-VEGF antigen-binding fragment transgene encodes an antigen-binding fragment and comprises the nucleotide sequences of the six ranibizumab CDRs. In certain embodiments, the anti-VEGF antigen-binding fragment transgene encodes an antigen-binding fragment comprising a heavy chain variable region comprising heavy chain CDRs 1-3 of ranibizumab (SEQ ID NOs: 20, 18, and 21). In certain embodiments, the anti-VEGF antigen-binding fragment transgene encodes an antigen-binding fragment comprising a light chain variable region comprising light chain CDRs 1-3 of ranibizumab (SEQ ID NOs: 14-16). In certain embodiments, the anti-VEGF antigen-binding fragment transgene encodes an antigen-binding fragment comprising a heavy chain variable region comprising heavy chain CDRs 1-3 of bevacizumab (SEQ ID NOs: 17-19). In certain embodiments, the anti-VEGF antigen-binding fragment transgene encodes an antigen-binding fragment comprising a light chain variable region comprising light chain CDRs 1-3 of bevacizumab (SEQ ID NOs: 14-16). In certain embodiments, the anti-VEGF antigen-binding fragment transgene encodes an antigen-binding fragment comprising a heavy chain variable region comprising heavy chain CDRs 1-3 of ranibizumab (SEQ ID NOs: 20, 18, and 21) and a light chain variable region comprising light chain CDRs 1-3 of ranibizumab (SEQ ID NOs: 14-16). In certain embodiments, the anti-VEGF antigen-binding fragment transgene encodes an antigen-binding fragment comprising a heavy chain variable region comprising heavy chain CDRs 1-3 of bevacizumab (SEQ ID NOs: 17-19) and a light chain variable region comprising light chain CDRs 1-3 of bevacizumab (SEQ ID NOs: 14-16).

In certain embodiments, the anti-VEGF antigen-binding fragment transgene encodes an antigen-binding fragment comprising a light chain variable region comprising light chain CDRs 1-3 of SEQ ID NOs: 14-16, wherein the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a light chain variable region comprising light chain CDRs 1-3 of SEQ ID NOs: 14-16, wherein the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a light chain variable region comprising light chain CDRs 1-3 of SEQ ID NOs: 14-16, wherein the second amino acid residue of the light chain CDR3 (i.e., the second Q in QQYSTVPWTF (SEQ ID NO. 16)) is not acetylated. In a specific embodiment, the anti-VEGF antigen-binding fragment transgene encodes an antigen-binding fragment comprising a light chain variable region comprising light chain CDRs 1-3 of SEQ ID NOs: 14-16, wherein the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) is not acetylated. In a preferred embodiment, the chemical modification(s) or lack of chemical modification(s) (as the case may be) described herein is determined by mass spectrometry.

In certain embodiments, the anti-VEGF antigen-binding fragment transgene encodes an antigen-binding fragment comprising a heavy chain variable region comprising heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a heavy chain variable region comprising heavy chain CDRs 1-3 of SEQ ID NOs: 20, 18, and 21, wherein the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a heavy chain variable region comprising heavy chain CDRs 1-3 of SEQ ID NOs: 20, 18, and 21, wherein the last amino acid residue of the heavy chain CDR1 (i.e., the Nin GYDFTHYGMN (SEQ ID NO. 20)) is not acetylated. In a specific embodiment, the anti-VEGF antigen-binding fragment transgene encodes an antigen-binding fragment comprising a heavy chain variable region comprising heavy chain CDRs 1-3 of SEQ ID NOs: 20, 18, and 21, wherein the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) is not acetylated. In a preferred embodiment, the chemical modification(s) or lack of chemical modification(s) (as the case may be) described herein is determined by mass spectrometry.

In certain embodiments, the anti-VEGF antigen-binding fragment transgene encodes an antigen-binding fragment comprising a light chain variable region comprising light chain CDRs 1-3 of SEQ ID NOs: 14-16 and a heavy chain variable region comprising heavy chain CDRs 1-3 of SEQ ID NOs: 20, 18, and 21, wherein the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu), and wherein the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a light chain variable region comprising light chain CDRs 1-3 of SEQ ID NOs: 14-16 and a heavy chain variable region comprising heavy chain CDRs 1-3 of SEQ ID NOs: 20, 18, and 21, wherein: (1) the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acctylation, deamidation, and pyroglutamation (pyro Glu), and the last amino acid residuc of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu); and (2) the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a light chain variable region comprising light chain CDRs 1-3 of SEQ ID NOs: 14-16 and a heavy chain variable region comprising heavy chain CDRs 1-3 of SEQ ID NOs: 20, 18, and 21, wherein the second amino acid residue of the light chain CDR3 (i.e., the second Q in QQYSTVPWTF (SEQ ID NO. 16)) is not acetylated, and wherein the last amino acid residue of the heavy chain CDR1 (i.e., the N in GYDFTHYGMN (SEQ ID NO. 20)) is not acetylated. In a specific embodiment, the antigen-binding fragment comprises a heavy chain CDR1 of SEQ ID NO. 20, wherein: (1) the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) is not acetylated; and (2) the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) is not acetylated. In a preferred embodiment, the chemical modification(s) or lack of chemical modification(s) (as the case may be) described herein is determined by mass spectrometry.

In certain aspects, also provided herein are anti-VEGF antigen-binding fragments comprising light chain CDRs 1-3 of SEQ ID NOs: 14-16 and heavy chain CDRs 1-3 of SEQ ID NOs: 20, 18, and 21, and transgenes encoding such antigen-VEGF antigen-binding fragments, wherein the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the second amino acid residue of the light chain CDR3 (i.e., the second Q in QQYSTVPWTF (SEQ ID NO. 16)) is not acetylated. In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) is not acetylated. The anti-VEGF antigen-binding fragments and transgenes provided herein can be used in any method according to the invention described herein. In a preferred embodiment, the chemical modification(s) or lack of chemical modification(s) (as the case may be) described herein is determined by mass spectrometry.

In certain aspects, also provided herein are anti-VEGF antigen-binding fragments comprising light chain CDRs 1-3 of SEQ ID NOs: 14-16 and heavy chain CDRs 1-3 of SEQ ID NOs: 20, 18, and 21, and transgenes encoding such antigen-VEGF antigen-binding fragments, wherein the last amino acid residue of the heavy chain CDR1 (i.e., the Nin GYDFTHYGMN (SEQ ID NO. 20)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the last amino acid residue of the heavy chain CDR1 (i.e., the Nin GYDFTHYGMN (SEQ ID NO. 20)) is not acetylated. In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) is not acetylated. The anti-VEGF antigen-binding fragments and transgenes provided herein can be used in any method according to the invention described herein. In a preferred embodiment, the chemical modification(s) or lack of chemical modification(s) (as the case may be) described herein is determined by mass spectrometry.

In certain aspects, also provided herein are anti-VEGF antigen-binding fragments comprising light chain CDRs 1-3 of SEQ ID NOs: 14-16 and heavy chain CDRs 1-3 of SEQ ID NOs: 20, 18, and 21, and transgenes encoding such antigen-VEGF antigen-binding fragments, wherein the last amino acid residue of the heavy chain CDR1 (i.e., the Nin GYDFTHYGMN (SEQ ID NO. 20)) does not carry one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein: (1) the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu); and (2) the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) is not acetylated, and the second amino acid residue of the light chain CDR3 (i.e., the second Q in QQYSTVPWTF (SEQ ID NO. 16)) is not acetylated. In a specific embodiment, the antigen-binding fragment comprises light chain CDRs 1-3 of SEQ ID NOs: 14-16 and heavy chain CDRs 1-3 of SEQ ID NOs: 20, 18, and 21, wherein: (1) the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) is not acetylated; and (2) the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) is not acetylated. The anti-VEGF antigen-binding fragments and transgenes provided herein can be used in any method according to the invention described herein. In a preferred embodiment, the chemical modification(s) or lack of chemical modification(s) (as the case may be) described herein is determined by mass spectrometry.

TABLE 2
Exemplary transgene sequences

| VEGF antigen- | |
|---|---|
| binding fragment | |
| (SEQ ID NO.) | Sequence |
| bevacizumab cDNA | gctagcgcca ccatgggctg gtcctgcatc atcctgttcc tggtggccac |
| (Light chain) | cgccaccggc gtgcactccg acatccagat gacccagtcc ccctcctccc |
| (10) | tgtccgcctc cgtgggcgac cgggtgacca tcacctgctc cgcctcccag |
| | gacatctcca actacctgaa ctggtaccag cagaagcccg gcaaggcccc |
| | caaggtgctg atctacttca cctcctccct gcactccggc gtgccctccc |
| | ggttctccgg ctccggctcc ggcaccgact tcaccctgac catctcctcc |
| | ctgcagcccg aggacttcgc cacctactac tgccagcagt actccaccgt |
| | gccctggacc ttcggccagg gcaccaaggt ggagatcaag cggaccgtgg |
| | ccgccccctc cgtgttcatc ttccccccct ccgacgagca gctgaagtcc |
| | ggcaccgcct ccgtggtgtg cctgctgaac aacttctacc cccgggaggc |
| | caaggtgcag tggaaggtgg acaacgccct gcagtccggc aactcccagg |
| | agtccgtgac cgagcaggac tccaaggact ccacctactc cctgtcctcc |
| | accctgaccc tgtccaaggc cgactacgag aagcacaagg tgtacgcctg |
| | cgaggtgacc caccagggcc tgtcctcccc cgtgaccaag tccttcaacc |
| | ggggcgagtg ctgagcggcc gcctcgag |
| bevacizumab cDNA | gctagcgcca ccatgggctg gtcctgcatc atcctgttcc tggtggccac |
| (Heavy chain) | cgccaccggc gtgcactccg aggtgcagct ggtggagtcc ggcggcggcc |
| (11) | tggtgcagcc cggcggctcc ctgcggctgt cctgcgccgc ctccggctac |
| | accttcacca actacggcat gaactgggtg cggcaggccc ccggcaaggg |
| | cctggagtgg gtgggctgga tcaacaccta caccggcgag cccacctacg |
| | ccgccgactt caagcggcgg ttcaccttct ccctggacac ctccaagtcc |
| | accgcctacc tgcagatgaa ctccctgcgg gccgaggaca ccgccgtgta |
| | ctactgcgcc aagtaccccc actactacgg ctcctcccac tggtacttcg |
| | acgtgtgggg ccagggcacc ctggtgaccg tgtcctccgc ctccaccaag |
| | ggcccctccg tgttccccct ggccccctcc tccaagtcca cctccggcgg |
| | caccgccgcc ctgggctgcc tggtgaagga ctacttcccc gagcccgtga |
| | ccgtgtcctg gaactccggc gccctgacct ccggcgtgca caccttcccc |
| | gccgtgctgc agtcctccgg cctgtactcc ctgtcctccg tggtgaccgt |
| | gccctcctcc tccctgggca cccagaccta catctgcaac gtgaaccaca |
| | agccctccaa caccaaggtg gacaagaagg tggagcccaa gtcctgcgac |
| | aagacccaca cctgcccccc ctgccccgcc cccgagctgc tgggcggccc |
| | ctccgtgttc ctgttccccc ccaagcccaa ggacaccctg atgatctccc |
| | ggacccccga ggtgacctgc gtggtggtgg acgtgtccca cgaggacccc |
| | gaggtgaagt tcaactggta cgtggacggc gtggaggtgc acaacgccaa |
| | gaccaagccc cgggaggagc agtacaactc cacctaccgg gtggtgtccg |
| | tgctgaccgt gctgcaccag gactggctga acggcaagga gtacaagtgc |
| | aaggtgtcca acaaggccct gcccgccccc |
| | atcgagaaga ccatctccaa ggccaagggc cagccccggg agccccaggt |
| | gtacaccctg cccccctccc gggaggagat gaccaagaac caggtgtccc |
| | tgacctgcct ggtgaagggcttctacccct ccgacatcgc cgtggagtgg |
| | gagtccaacg gccagcccga gaacaactac aagaccaccc cccccgtgct |
| | ggactccgac ggctccttct tcctgtactc caagctgaccgtggacaagt |
| | cccggtggca gcagggcaac gtgttctcct gctccgtgat gcacgaggcc |
| | ctgcacaacc actacaccca gaagtccctg tccctgtccc ccggcaagtg |
| | agcggccgcc |
| bevacizumab Fab | DIQMTQSPSSLSASVGDRVTITCSASQDISNYLNWYQQKPGKAPKVLIYFTSSLH |
| Amino Acid | SGVPSRFSGSGSGTDFTLTISSLQPEDFATYYCQQYSTVPWTFGQGTKVEIKRTV |
| Sequence (Light | AAPSVFIFPPSDEQLKSGTASVVCLLNNFYPREAKVQWKVDNALQSGNSQESVTE |
| chain) | QDSKDSTYSLSSTLTLSKADYEKHKVYACEVTHQGLSSPVTKSFNRGEC |
| (3) | |
| bevacizumab Fab | EVQLVESGGGLVQPGGSLRLSCAASGYTFTNYGMNWVRQAPGKGLEWVGWINTYT |
| Amino Acid | GEPTYAADFKRRFTFSLDTSKSTAYLQMNSLRAEDTAVYYCAKYPHYYGSSHWYF |
| Sequence (Heavy | DVWGQGTLVTVSSASTKGPSVFPLAPSSKSTSGGTAALGCLVKDYFPEPVTVSWN |
| chain) | SGALTSGVHTFPAVLQSSGLYSLSSVVTVPSSSLGTQTYICNVNHKPSNTKVDKK |
| (4) | VEPKSCDKTHL |
| ranibizumab cDNA | gagctccatg gagtttttca aaaagacggc acttgccgca ctggttatgg |
| (Light chain | gttttagtgg tgcagcattg gccgatatcc agctgaccca gagcccgagc |
| comprising a | agcctgagcg caagcgttgg tgatcgtgtt accattacct gtagcgcaag |
| signal sequence) | ccaggatatt agcaattatc tgaattggta tcagcagaaa ccgggtaaag |
| (12) | caccgaaagt tctgatttat tttaccagca gcctgcatag cggtgttccg |
| | agccgtttta gcggtagcgg tagtggcacc gattttaccc tgaccattag |
| | cagcctgcag ccggaagatt ttgcaaccta ttattgtcag cagtatagca |
| | ccgttccgtg gacctttggt cagggcacca aagttgaaat taaacgtacc |
| | gttgcagcac cgagcgtttt tatttttccg cctagtgatg aacagctgaa |
| | aagcggcacc gcaagcgttg tttgtctgct gaataatttt tatccgcgtg |
| | aagcaaaagt gcagtggaaa gttgataatg cactgcagag cggtaatagc |
| | caagaaagcg ttaccgaaca ggatagcaaa gatagcacct atagcctgag |
| | cagcaccctg accctgagca aagcagatta tgaaaaacac aaagtgtatg |
| | cctgcgaagt tacccatcag ggtctgagca gtccggttac caaaagtttt |
| | aatcgtggcg aatgctaata gaagcttggt acc |
| ranibizumab cDNA | gagctcatat gaaatacctg ctgccgaccg ctgctgctgg tctgctgctc |
| (Heavy chain | ctcgctgccc agccggcgat ggccgaagtt cagctggttg aaagcggtgg |
| comprising a | tggtctggtt cagcctggtg gtagcctgcg tctgagctgt gcagcaagcg |
| signal sequence) | gttatgattt tacccattat ggtatgaatt gggttcgtca ggcaccgggt |
| (13) | aaaggtctgg aatgggttgg ttggattaat acctataccg gtgaaccgac |
| | ctatgcagca gattttaaac gtcgttttac ctttagcctg gataccagca |
| | aaagcaccgc atatctgcag atgaatagcc tgcgtgcaga agataccgca |
| | gtttattatt gtgccaaata tccgtattac tatggcacca gccactggta |
| | tttcgatgtt tggggtcagg gcaccctggt taccgttagc agcgcaagca |
| | ccaaaggtcc gagcgttttt ccgctggcac cgagcagcaa aagtaccagc |
| | ggtggcacag cagcactggg ttgtctggtt aaagattatt ttccggaacc |
| | ggttaccgtg agctggaata gcggtgcact gaccagcggt gttcatacct |
| | ttccggcagt tctgcagagc agcggtctgt atagcctgag cagcgttgtt |
| | accgttccga gcagcagcct gggcacccag acctatattt gtaatgttaa |
| | tcataaaccg agcaatacca aagtggataa aaaagttgag ccgaaaagct |
| | gcgataaaac ccatctgtaa tagggtacc |
| ranibizumab Fab | DIQLTQSPSSLSASVGDRVTITCSASQDISNYLNWYQQKPGKAPKVLIYFTSSLH |
| Amino Acid | SGVPSRFSGSGSGTDFTLTISSLQPEDFATYYCQQYSTVPWTFGQGTKVEIKRTV |
| Sequence (Light | AAPSVFIFPPSDEQLKSGTASVVCLLNNFYPREAKVQWKVDNALQSGNSQESVTE |
| chain) | QDSKDSTYSLSSTLTLSKADYEKHKVYACEVTHQGLSSPVTKSFNRGEC |
| (1) | |
| ranibizumab Fab | EVQLVESGGGLVQPGGSLRLSCAASGYDFTHYGMNWVRQAPGKGLEWVGWINTYT |
| Amino Acid | GEPTYAADFKRRFTFSLDTSKSTAYLQMNSLRAEDTAVYYCAKYPYYYGTSHWYF |
| Sequence (Heavy | DVWGQGTLVTVSSASTKGPSVFPLAPSSKSTSGGTAALGCLVKDYFPEPVTVSWN |
| chain) | SGALTSGVHTFPAVLQSSGLYSLSSVVTVPSSSLGTQTYICNVNHKPSNTKVDKK |
| (2) | VEPKSCDKTHL |
| bevacizumab Light | SASQDISNYLN |
| Chain CDRs | FTSSLHS |
| (14, 15, and 16) | QQYSTVPWT |
| bevacizumab Heavy | GYTFTNYGMN |
| Chain CDRs | WINTYTGEPTYAADFKR |
| (17, 18, and 19) | YPHYYGSSHWYFDV |
| ranibizumab Light | SASQDISNYLN |
| Chain CDRs | FTSSLHS |
| (14, 15, and 16) | QQYSTVPWT |
| ranibizumab Heavy | GYDFTHYGMN |
| Chain CDRs | WINTYTGEPTYAADFKR |
| (20, 18, and 21) | YPYYYGTSHWYFDV |

### 5.2.9 Constructs

In certain embodiments, the viral vectors provided herein comprise the following elements in the following order: a) a constitutive or a hypoxia-inducible promoter sequence, and b) a sequence encoding the transgene (e.g., an anti-VEGF antigen-binding fragment moiety). In certain embodiments, the sequence encoding the transgene comprises multiple ORFs separated by IRES elements. In certain embodiments, the ORFs encode the heavy and light chain domains of the anti-VEGF antigen-binding fragment. In certain embodiments, the sequence encoding the transgene comprises multiple subunits in one ORF separated by F/F2A sequences. In certain embodiments, the sequence comprising the transgene encodes the heavy and light chain domains of the anti-VEGF antigen-binding fragment separated by an F/F2A sequence. In certain embodiments, the viral vectors provided herein comprise the following elements in the following order: a) a constitutive or a hypoxia-inducible promoter sequence, and b) a sequence encoding the transgene (e.g., an anti-VEGF antigen-binding fragment moiety), wherein the transgene comprises the signal peptide of VEGF (SEQ ID NO: 5), and wherein the transgene encodes a light chain and a heavy chain sequence separated by an IRES element. In certain embodiments, the viral vectors provided herein comprise the following elements in the following order: a) a constitutive or a hypoxia-inducible promoter sequence, and b) a sequence encoding the transgene (e.g., an anti-VEGF antigen-binding fragment moiety), wherein the transgene comprises the signal peptide of VEGF (SEQ ID NO: 5), and wherein the transgene encodes a light chain and a heavy chain sequence separated by a cleavable F/F2A sequence.

In certain embodiments, the viral vectors provided herein comprise the following elements in the following order: a) a first ITR sequence, b) a first linker sequence, c) a constitutive or a hypoxia-inducible promoter sequence, d) a second linker sequence, e) an intron sequence, f) a third linker sequence, g) a first UTR sequence, h) a sequence encoding the transgene (e.g., an anti-VEGF antigen-binding fragment moiety), i) a second UTR sequence, j) a fourth linker sequence, k) a poly A sequence, 1) a fifth linker sequence, and m) a second ITR sequence.

In certain embodiments, the viral vectors provided herein comprise the following elements in the following order: a) a first ITR sequence, b) a first linker sequence, c) a constitutive or a hypoxia-inducible promoter sequence, d) a second linker sequence, e) an intron sequence, f) a third linker sequence, g) a first UTR sequence, h) a sequence encoding the transgene (e.g., an anti-VEGF antigen-binding fragment moiety), i) a second UTR sequence, j) a fourth linker sequence, k) a poly A sequence, 1) a fifth linker sequence, and m) a second ITR sequence, wherein the transgene comprises the signal peptide of VEGF (SEQ ID NO: 5), and wherein the transgene encodes a light chain and a heavy chain sequence separated by a cleavable F/F2A sequence.

### 5.2.10 Manufacture and Testing of Vectors

The viral vectors provided herein may be manufactured using host cells. The viral vectors provided herein may be manufactured using mammalian host cells, for example, A549, WEHI, 10T1/2, BHK, MDCK, COSI, COS7, BSC 1, BSC 40, BMT 10, VERO, W138, HeLa, 293, Saos, C2C12, L, HT1080, HepG2, primary fibroblast, hepatocyte, and myoblast cells. The viral vectors provided herein may be manufactured using host cells from human, monkey, mouse, rat, rabbit, or hamster.

The host cells are stably transformed with the sequences encoding the transgene and associated elements (i.e., the vector genome), and the means of producing viruses in the host cells, for example, the replication and capsid genes (e.g., the rep and cap genes of AAV). For a method of producing recombinant AAV vectors with AAV8 capsids, see Section IV of the Detailed Description of U.S. Pat. No. 7,282,199 B2, which is incorporated herein by reference in its entirety. Genome copy titers of said vectors may be determined, for example, by TAQMAN® analysis. Virions may be recovered, for example, by CsCl2 sedimentation.

In vitro assays, e.g., cell culture assays, can be used to measure transgene expression from a vector described herein, thus indicating, e.g., potency of the vector. For example, the PER.C6® Cell Line (Lonza), a cell line derived from human embryonic retinal cells, or retinal pigment epithelial cells, e.g., the retinal pigment epithelial cell line hTERT RPE-1 (available from ATCC®), can be used to assess transgene expression. Once expressed, characteristics of the expressed product (i.e., HuGlyFab VEGFi) can be determined, including determination of the glycosylation and tyrosine sulfation patterns associated with the HuGlyFab VEGFi. Glycosylation patterns and methods of determining the same are discussed in Section 5.1.1, while tyrosine sulfation patterns and methods of determining the same are discussed in Section 5.1.2. In addition, benefits resulting from glycosylation/sulfation of the cell-expressed HuGlyFab VEGFi can be determined using assays known in the art, e.g., the methods described in Sections 5.1.1 and 5.1.2.

### 5.2.11 Compositions

Compositions are described comprising a vector encoding a transgene described herein and a suitable carrier. A suitable carrier (e.g., for suprachoroidal, subretinal, juxtascleral, and/or intraretinal administration) would be readily selected by one of skill in the art.

### 5.3 Gene Therapy

Methods are described for the administration of a therapeutically effective amount of a transgene construct to human subjects having an ocular disease, in particular an ocular disease caused by increased neovascularization. More particularly, methods for administration of a therapeutically effective amount of a transgene construct to patients having wet AMD, dry AMD, retinal vein occlusion (RVO), diabetic macular edema (DME), or diabetic retinopathy (DR) (in particular, wet AMD), in particular, for suprachoroidal, subretinal, juxtascleral and/or intraretinal administration (e.g., by suprachoroidal injection, subretinal injection via the transvitreal approach (a surgical procedure), subretinal administration via the suprachoroidal space, or a posterior juxtascleral depot procedure) are described. In particular embodiments, such methods for suprachoroidal, subretinal, juxtascleral and/or intraretinal administration of a therapeutically effective amount of a transgene construct can be used to treat to patients having wet AMD, dry AMD, retinal vein occlusion (RVO), diabetic macular edema (DME), or diabetic retinopathy (DR) (in particular, wet AMD) (e.g., by suprachorodial inject, subretinal injection via the transvitreal approach (a surgical procedure), subretinal administration via the suprachoroidal space, or a posterior juxtascleral depot procedure).

Methods are described for suprachoroidal, subretinal, juxtascleral and/or intraretinal administration of a therapeutically effective amount of a transgene construct to patients diagnosed with an ocular disease, in particular an ocular disease caused by increased neovascularization (e.g., by suprachoroidal injection, subretinal injection via the transvitreal approach (a surgical procedure), subretinal administration via the suprachoroidal space, or a posterior juxtascleral depot procedure). In particular embodiments, such methods for suprachoroidal, subretinal, juxtascleral and/or intraretinal administration of a therapeutically effective amount of a transgene construct to can be used to treat patients diagnosed with wet AMD, dry AMD, retinal vein occlusion (RVO), diabetic macular edema (DME), or diabetic retinopathy (DR) (in particular, wet AMD); and in particular, wet AMD (neovascular AMD), or diabetic retinopathy (e.g., by suprochoroidal injection, subretinal injection via the transvitreal approach (a surgical procedure), subretinal administration via the suprachoroidal space, or a posterior juxtascleral depot procedure).

Also provided herein are methods for suprachoroidal, subretinal, juxtascleral and/or intraretinal administration of a therapeutically effective amount of a transgene construct (e.g., by suprachoroidal injection, subretinal injection via the transvitreal approach (a surgical procedure), subretinal administration via the suprachoroidal space, or a posterior juxtascleral depot procedure) and methods of administration of a therapeutically effective amount of a transgene construct to the retinal pigment epithelium.

### 5.3.1 Target Patient Populations

In certain embodiments, the methods provided herein are for the administration to patients diagnosed with an ocular disease (for example, wet AMD, dry AMD, retinal vein occlusion (RVO), diabetic macular edema (DME), or diabetic retinopathy (DR) (in particular, wet AMD)), in particular an ocular disease caused by increased neovascularization.

In certain embodiments, the methods provided herein are for the administration to patients diagnosed with severe AMD. In certain embodiments, the methods provided herein are for the administration to patients diagnosed with attenuated AMD.

In certain embodiments, the methods provided herein are for the administration to patients diagnosed with severe wet AMD. In certain embodiments, the methods provided herein are for the administration to patients diagnosed with attenuated wet AMD.

In certain embodiments, the methods provided herein are for the administration to patients diagnosed with severe diabetic retinopathy. In certain embodiments, the methods provided herein are for the administration to patients diagnosed with attenuated diabetic retinopathy.

In certain embodiments, the methods provided herein are for the administration to patients diagnosed with AMD who have been identified as responsive to treatment with an anti-VEGF antibody.

In certain embodiments, the methods provided herein are for the administration to patients diagnosed with AMD who have been identified as responsive to treatment with an anti-VEGF antigen-binding fragment.

In certain embodiments, the methods provided herein are for the administration to patients diagnosed with AMD who have been identified as responsive to treatment with an anti-VEGF antigen-binding fragment injected intravitreally prior to treatment with gene therapy.

In certain embodiments, the methods provided herein are for the administration to patients diagnosed with AMD who have been identified as responsive to treatment with LUCENTIS® (ranibizumab), EYLEA® (aflibercept), and/or AVASTIN® (bevacizumab).

### 5.3.2 Dosage and Mode of Administration

Therapeutically effective doses of the recombinant vector should be administered subretinally, and/or intraretinally (e.g., by subretinal injection via the transvitreal approach (a surgical procedure), or via the suprachoroidal space) in a volume ranging from ≥0.1 ml. to ≤0.5 mL, preferably in 0.1 to 0.30 mL (100-300 μl), and most preferably, in a volume of 0.25 mL (250 μl). Therapeutically effective doses of the recombinant vector should be administered suprachoroidally (e.g., by suprachoroidal injection) in a volume of 100 μl or less, for example, in a volume of 50-100 μl. Therapeutically effective doses of the recombinant vector should be administered to the ourter surface of the sclera in a volume of 500 μl or less, for example, in a volume of 500 μl or less, for example, in a volume of 10-20 μl, 20-50 μl, 50-100 μl, 100-200 μl, 200-300 μl, 300-400 μl, or 400-500 μl.

In certain embodiments, the recombinant vector is administered suprachoroidally (e.g., by suprachoroidal injection). In a specific embodiment, suprachorodial administration (e.g., an injection into the suprachoroidal space) is performed using a suprachoroidal drug delivery device. Suprachoroidal drug delivery devices are often used in suprachoroidal administration procedures, which involve administration of a drug to the suprachoroidal space of the eye (see, e.g., Hariprasad, 2016, Retinal Physician 13: 20-23; Goldstein, 2014, Retina Today 9(5): 82-87; Baldassarre et al., 2017; each of which is incorporated by reference herein in its entirety). The suprachoroidal drug delivery devices that can be used to deposit the expression vector in the subretinal space according to the invention described herein include, but are not limited to, suprachoroidal drug delivery devices manufactured by Clearside® Biomedical, Inc. (see, for example, Hariprasad, 2016, Retinal Physician 13: 20-23) and MedOne suprachoroidal catheters.

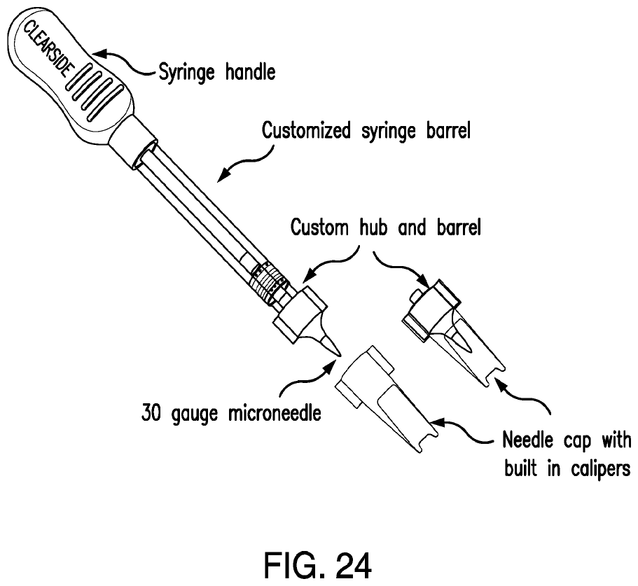

In a specific embodiment, the suprachoroidal drug delivery device is a syringe with a 1 millimeter 30 gauge needle (see FIG. 24). During an injection using this device, the needle pierces to the base of the sclera and fluid containing drug enters the suprachoroidal space, leading to expansion of the suprachoroidal space. As a result, there is tactile and visual feedback during the injection. Following the injection, the fluid flows posteriorly and absorbs dominantly in the choroid and retina. This results in the production of transgene protein from all retinal cell layers and choroidal cells. Using this type of device and procedure allows for a quick and easy in-office procedure with low risk of complications. A max volume of 100 μl can be injected into the suprachoroidal space.

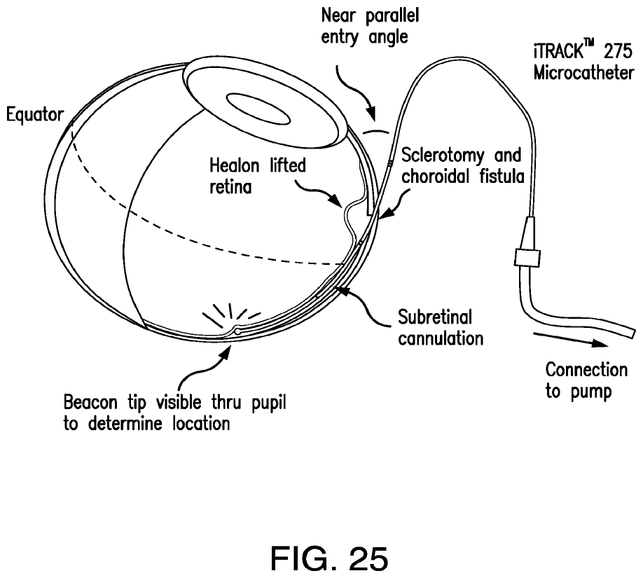

In certain embodiments, the recombinant vector is administered subretinally via the suprachoroidal space by use of a subretinal drug delivery device. In certain embodiments, the subretinal drug delivery device is a catheter which is inserted and tunneled through the suprachoroidal space around to the back of the eye during a surgical procedure to deliver drug to the subretinal space (see FIG. 25). This procedure allows the vitreous to remain intact and thus, there are fewer complication risks (less risk of gene therapy egress, and complications such as retinal detachments and macular holes), and without a vitrectomy, the resulting bleb may spread more diffusely allowing more of the surface area of the retina to be transduced with a smaller volume. The risk of induced cataract following this procedure is minimized, which is desirable for younger patients. Moreover, this procedure can deliver bleb under the fovea more safely than the standard transvitreal approach, which is desirable for patients with inherited retinal diseases effecting central vision where the target cells for transduction are in the macula. This procedure is also favorable for patients that have neutralizing antibodies (Nabs) to AAVs present in the systemic circulation which may impact other routes of delivery (such as surpachoroidal and intravitreal). Additionally, this method has shown to create blebs with less egress out the retinotomy site than the standard transvitreal approach. The subretinal drug delivery device originally manufactured by Janssen Pharmaceuticals, Inc. now by Orbit Biomedical Inc. (see, for example, Subretinal Delivery of Cells via the Suprachoroidal Space: Janssen Trial. In: Schwartz et al. (eds) Cellular Therapies for Retinal Disease, Springer, Cham; International Patent Application Publication No. WO 2016/040635 A1) can be used for such purpose.

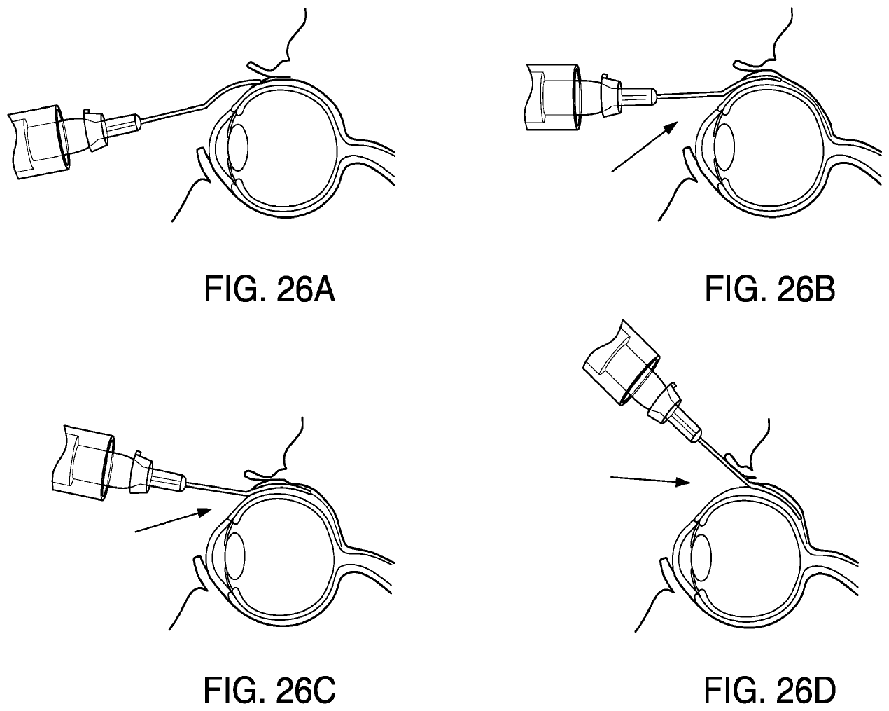

In certain embodiments, the recombinant vector is administered to the outer surface of the sclera (for example, by the use of a juxtascleral drug delivery device that comprises a cannula, whose tip can be inserted and kept in direct apposition to the scleral surface). In a specific embodiment, administration to the outer surface of the sclera is performed using a posterior juxtascleral depot procedure, which involves drug being drawn into a blunt-tipped curved cannula and then delivered in direct contact with the outer surface of the sclera without puncturing the eyeball. In particular, following the creation of a small incision to bare sclera, the cannula tip is inserted (see FIG. 26A). The curved portion of the cannula shaft is inserted, keeping the cannula tip in direct apposition to the scleral surface (see FIGS. 26B-26D). After complete insertion of the cannula (FIG. 26D), the drug is slowly injected while gentle pressure is maintained along the top and sides of the cannula shaft with sterile cotton swabs. This method of delivery avoids the risk of intraocular infection and retinal detachment, side effects commonly associated with injecting therapeutic agents directly into the eye.

Doses that maintain a concentration of the transgene product at a Cₘᵢₙ of at least 0.330 μg/mL in the Vitreous humour, or 0.110 μg/mL in the Aqueous humour (the anterior chamber of the eye) for three months are desired; thereafter, Vitreous Cₘᵢₙ concentrations of the transgene product ranging from 1.70 to 6.60 μg/mL, and/or Aqueous Cₘᵢₙ concentrations ranging from 0.567 to 2.20 μg/ml. should be maintained. However, because the transgene product is continuously produced (under the control of a constitutive promoter or induced by hypoxic conditions when using an hypoxia-inducible promoter), maintenance of lower concentrations can be effective. Vitreous humour concentrations can be measured directly in patient samples of fluid collected from the vitreous humour or the anterior chamber, or estimated and/or monitored by measuring the patient's serum concentrations of the transgene product—the ratio of systemic to vitreal exposure to the transgene product is about 1:90,000. (E.g., see, vitreous humor and serum concentrations of ranibizumab reported in Xu L, et al., 2013, Invest. Opthal. Vis. Sci. 54: 1616-1624, at p. 1621 and Table 5 at p. 1623, which is incorporated by reference herein in its entirety).

In certain embodiments, dosages are measured by genome copies per ml or the number of genome copies administered to the eye of the patient (e.g., administered suprachoroidally, subretinally, juxtasclerally and/or intraretinally (e.g., by suprachoroidal injection, subretinal injection via the transvitreal approach (a surgical procedure), subretinal administration via the suprachoroidal space, or a posterior juxtascleral depot procedure)). In certain embodiments, 2.4×10¹¹ genome copies per ml to 1×10¹³ genome copies per ml are administered. In a specific embodiment, 2.4×10¹¹ genome copies per ml to 5 ×10¹¹ genome copies per ml are administered. In another specific embodiment, 5×10¹¹ genome copies per ml to 1×10¹² genome copies per ml are administered. In another specific embodiment, 1×10¹² genome copies per ml to 5×10¹² genome copies per ml are administered. In another specific embodiment, 5×10¹² genome copies per ml to 1×10¹³ genome copies per ml are administered. In another specific embodiment, about 2.4×10¹¹ genome copies per ml are administered. In another specific embodiment, about 5×10¹¹ genome copies per ml are administered. In another specific embodiment, about 1×10¹² genome copies per ml are administered. In another specific embodiment, about 5×10¹² genome copies per ml are administered. In another specific embodiment, about 1×10¹³ genome copies per ml are administered. In certain embodiments, 1×10⁹ to 1×10¹² genome copies are administered. In specific embodiments, 3×10⁹ to 2.5×10¹¹ genome copies are administered. In specific embodiments, 1×10⁹ to 2.5×10¹¹ genome copies are administered. In specific embodiments, 1×10⁹ to 1×10¹¹ genome copies are administered. In specific embodiments, 1×10⁹ to 5×10⁹ genome copies are administered. In specific embodiments, 6×10⁹ to 3×10¹⁰ genome copies are administered. In specific embodiments, 4×10¹⁰ to 1×10¹¹ genome copies are administered. In specific embodiments, 2×10¹¹ to 1×10¹² genome copies are administered. In a specific embodiment, about 3×10⁹ genome copies are administered (which corresponds to about 1.2×10¹⁰ genome copies per ml in a volume of 250 μl). In another specific embodiment, about 1×10¹⁰ genome copies are administered (which corresponds to about 4×10¹⁰ genome copies per ml in a volume of 250 μl). In another specific embodiment, about 6×10¹⁰ genome copies are administered (which corresponds to about 2.4×10¹¹ genome copies per ml in a volume of 250 μl). In another specific embodiment, about 1.6×10¹¹ genome copies are administered (which corresponds to about 6.2×10¹¹ genome copies per ml in a volume of 250 μl). In another specific embodiment, about 1.6×10¹¹ genome copies are administered (which corresponds to about 6.4×10¹¹ genome copies per ml in a volume of 250 μl). In another specific embodiment, about 2.5×10¹¹ genome copies (which corresponds to about 2.5×10¹⁰ in a volume of 250 μl) are administered.

As used herein and unless otherwise specified, the term “about” means within plus or minus 10% of a given value or range.

### 5.3.3 Sampling and Monitoring of Efficacy

Effects of the methods of treatment provided herein on visual deficits may be measured by BCVA (Best-Corrected Visual Acuity), intraocular pressure, slit lamp biomicroscopy, and/or indirect ophthalmoscopy.

Effects of the methods of treatment provided herein on physical changes to eye/retina may be measured by SD-OCT (SD-Optical Coherence Tomography).

Efficacy may be monitored as measured by electroretinography (ERG).

Effects of the methods of treatment provided herein may be monitored by measuring signs of vision loss, infection, inflammation and other safety events, including retinal detachment.

Retinal thickness may be monitored to determine efficacy of the treatments provided herein. Without being bound by any particular theory, thickness of the retina may be used as a clinical readout, wherein the greater reduction in retinal thickness or the longer period of time before thickening of the retina, the more efficacious the treatment. Retinal function may be determined, for example, by ERG. ERG is a non-invasive electrophysiologic test of retinal function, approved by the FDA for use in humans, which examines the light sensitive cells of the eye (the rods and cones), and their connecting ganglion cells, in particular, their response to a flash stimulation. Retinal thickness may be determined, for example, by SD-OCT. SD-OCT is a three-dimensional imaging technology which uses low-coherence interferometry to determine the echo time delay and magnitude of backscattered light reflected off an object of interest. OCT can be used to scan the layers of a tissue sample (e.g., the retina) with 3 to 15 μm axial resolution, and SD-OCT improves axial resolution and scan speed over previous forms of the technology (Schuman, 2008, Trans. Am. Opthamol. Soc. 106:426-458).

### 5.4 Combination Therapies

The methods of treatment provided herein may be combined with one or more additional therapies. In one aspect, the methods of treatment provided herein are administered with laser photocoagulation. In one aspect, the methods of treatment provided herein are administered with photodynamic therapy with verteporfin.

In one aspect, the methods of treatment provided herein are administered with intravitreal (IVT) injections with anti-VEGF agents, including but not limited to HuPTMFab VEGFi, e.g., HuGlyFab VEGFi produced in human cell lines (Dumont et al., 2015, supra), or other anti-VEGF agents such as pegaptanib, ranibizumab, aflibercept, or bevacizumab.

The additional therapies may be administered before, concurrently or subsequent to the gene therapy treatment.

The efficacy of the gene therapy treatment may be indicated by the elimination of or reduction in the number of rescue treatments using standard of care, for example, intravitreal injections with anti-VEGF agents, including but not limited to HuPTMFab VEGFi, e.g., HuGlyFab VEGFi produced in human cell lines, or other anti-VEGF agents such as pegaptanib, ranibizumab, aflibercept, or bevacizumab.

TABLE 3
TABLE OF SEQUENCES

| SEQ | | |
|---|---|---|
| ID | | |
| NO: | Description | Sequence |
| 1 | Ranibizumab | DIQLTQSPSSLSASVGDRVTITCSASQDISNYLNWYQQKPGKAPKVLIYFTSSLH |
| | Fab Amino | SGVPSRFSGSGSGTDFTLTISSLQPEDFATYYCQQYSTVPWTFGQGTKVEIKRTV |
| | Acid Sequence | AAPSVFIFPPSDEQLKSGTASVVCLLNNFYPREAKVQWKVDNALQSGNSQESVTE |
| | (Light chain) | QDSKDSTYSLSSTLTLSKADYEKHKVYACEVTHQGLSSPVTKSFNRGEC |
| 2 | Ranibizumab | EVQLVESGGGLVQPGGSLRLSCAASGYDFTHYGMNWVRQAPGKGLEWVGWINTYT |
| | Fab Amino | GEPTYAADFKRRFTFSLDTSKSTAYLQMNSLRAEDTAVYYCAKYPYYYGTSHWYF |
| | Acid Sequence | DVWGQGTLVTVSSASTKGPSVFPLAPSSKSTSGGTAALGCLVKDYFPEPVTVSWN |
| | (Heavy chain) | SGALTSGVHTFPAVLQSSGLYSLSSVVTVPSSSLGTQTYICNVNHKPSNTKVDKK |
| | | VEPKSCDKTHL |
| 3 | Bevacizumab | DIQMTQSPSSLSASVGDRVTITCSASQDISNYLNWYQQKPGKAPKVLIYFTSSLH |
| | Fab Amino | SGVPSRFSGSGSGTDFTLTISSLQPEDFATYYCQQYSTVPWTFGQGTKVEIKRTV |
| | Acid Sequence | AAPSVFIFPPSDEQLKSGTASVVCLLNNFYPREAKVQWKVDNALQSGNSQESVTE |
| | (Light chain) | QDSKDSTYSLSSTLTLSKADYEKHKVYACEVTHQGLSSPVTKSFNRGEC |
| 4 | Bevacizumab | EVQLVESGGGLVQPGGSLRLSCAASGYTFTNYGMNWVRQAPGKGLEWVGWINTYT |
| | Fab Amino | GEPTYAADFKRRFTFSLDTSKSTAYLQMNSLRAEDTAVYYCAKYPHYYGSSHWYF |
| | Acid Sequence | DVWGQGTLVTVSSASTKGPSVFPLAPSSKSTSGGTAALGCLVKDYFPEPVTVSWN |
| | (Heavy chain) | SGALTSGVHTFPAVLQSSGLYSLSSVVTVPSSSLGTQTYICNVNHKPSNTKVDKK |
| | | VEPKSCDKTHL |
| 5 | VEGF-A signal | MNFLLSWVHW SLALLLYLHH AKWSQA |
| | peptide | |
| 6 | Fibulin-1 | MERAAPSRRV PLPLLLLGGL ALLAAGVDA |
| | signal peptide | |
| 7 | Vitronectin | MAPLRPLLIL ALLAWVALA |
| | signal peptide | |
| 8 | Complement | MRLLAKIICLMLWAICVA |
| | Factor H signal | |
| | peptide | |
| 9 | Opticin signal | MRLLAFLSLL ALVLQETGT |
| | peptide | |
| 10 | Bevacizumab | gctagcgcca ccatgggctg gtcctgcatc atcctgttcc tggtggccac |
| | cDNA | cgccaccggc gtgcactccg acatccagat gacccagtcc ccctcctccc |
| | (Light chain) | tgtccgcctc cgtgggcgac cgggtgacca tcacctgctc cgcctcccag |
| | | gacatctcca actacctgaa ctggtaccag cagaagcccg gcaaggcccc |
| | | caaggtgctg atctacttca cctcctccct gcactccggc gtgccctccc |
| | | ggttctccgg ctccggctcc ggcaccgact tcaccctgac catctcctcc |
| | | ctgcagcccg aggacttcgc cacctactac tgccagcagt actccaccgt |
| | | gccctggacc ttcggccagg gcaccaaggt ggagatcaag cggaccgtgg |
| | | ccgccccctc cgtgttcatc ttccccccct ccgacgagca gctgaagtcc |
| | | ggcaccgcct ccgtggtgtg cctgctgaac aacttctacc cccgggaggc |
| | | caaggtgcag tggaaggtgg acaacgccct gcagtccggc aactcccagg |
| | | agtccgtgac cgagcaggac tccaaggact ccacctactc cctgtcctcc |
| | | accctgaccc tgtccaaggc cgactacgag aagcacaagg tgtacgcctg |
| | | cgaggtgacc caccagggcc tgtcctcccc cgtgaccaag tccttcaacc |
| | | ggggcgagtg ctgagcggcc gcctcgag |
| 11 | Bevacizumab | gctagcgcca ccatgggctg gtcctgcatc atcctgttcc tggtggccac |
| | cDNA (Heavy | cgccaccggc gtgcactccg aggtgcagct ggtggagtcc ggcggcggcc |
| | chain) | tggtgcagcc cggcggctcc ctgcggctgt cctgcgccgc ctccggctac |
| | | accttcacca actacggcat gaactgggtg cggcaggccc ccggcaaggg |
| | | cctggagtgg gtgggctgga tcaacaccta caccggcgag cccacctacg |
| | | ccgccgactt caagcggcgg ttcaccttct ccctggacac ctccaagtcc |
| | | accgcctacc tgcagatgaa ctccctgcgg gccgaggaca ccgccgtgta |
| | | ctactgcgcc aagtaccccc actactacgg ctcctcccac tggtacttcg |
| | | acgtgtgggg ccagggcacc ctggtgaccg tgtcctccgc ctccaccaag |
| | | ggcccctccg tgttccccct ggccccctcc tccaagtcca cctccggcgg |
| | | caccgccgcc ctgggctgcc tggtgaagga ctacttcccc gagcccgtga |
| | | ccgtgtcctg gaactccggc gccctgacct ccggcgtgca caccttcccc |
| | | gccgtgctgc agtcctccgg cctgtactcc ctgtcctccg tggtgaccgt |
| | | gccctcctcc tccctgggca cccagaccta catctgcaac gtgaaccaca |
| | | agccctccaa caccaaggtg gacaagaagg tggagcccaa gtcctgcgac |
| | | aagacccaca cctgcccccc ctgccccgcc cccgagctgc tgggcggccc |
| | | ctccgtgttc ctgttccccc ccaagcccaa ggacaccctg atgatctccc |
| | | ggacccccga ggtgacctgc gtggtggtgg acgtgtccca cgaggacccc |
| | | gaggtgaagt tcaactggta cgtggacggc gtggaggtgc acaacgccaa |
| | | gaccaagccc cgggaggagc agtacaactc cacctaccgg gtggtgtccg |
| | | tgctgaccgt gctgcaccag gactggctga acggcaagga gtacaagtgc |
| | | aaggtgtcca acaaggccct gcccgccccc atcgagaaga ccatctccaa |
| | | ggccaagggc cagccccggg agccccaggt gtacaccctg cccccctccc |
| | | gggaggagat gaccaagaac caggtgtccc tgacctgcct ggtgaagggc |
| | | ttctacccct ccgacatcgc cgtggagtgg gagtccaacg gccagcccga |
| | | gaacaactac aagaccaccc cccccgtgct ggactccgac ggctccttct |
| | | tcctgtactc caagctgacc gtggacaagt cccggtggca gcagggcaac |
| | | gtgttctcct gctccgtgat gcacgaggcc ctgcacaacc actacaccca |
| | | gaagtccctg tccctgtccc ccggcaagtg agcggccgcc |
| 12 | Ranibizumab | gagctccatg gagtttttca aaaagacggc acttgccgca ctggttatgg |
| | cDNA (Light | gttttagtgg tgcagcattg gccgatatcc agctgaccca gagcccgagc |
| | chain | agcctgagcg caagcgttgg tgatcgtgtt accattacct gtagcgcaag |
| | comprising a | ccaggatatt agcaattatc tgaattggta tcagcagaaa ccgggtaaag |
| | signal | caccgaaagt tctgatttat tttaccagca gcctgcatag cggtgttccg |
| | sequence) | agccgtttta gcggtagcgg tagtggcacc gattttaccc tgaccattag |
| | | cagcctgcag ccggaagatt ttgcaaccta ttattgtcag cagtatagca |
| | | ccgttccgtg gacctttggt cagggcacca aagttgaaat taaacgtacc |
| | | gttgcagcac cgagcgtttt tatttttccg cctagtgatg aacagctgaa |
| | | aagcggcacc gcaagcgttg tttgtctgct gaataatttt tatccgcgtg |
| | | aagcaaaagt gcagtggaaa gttgataatg cactgcagag cggtaatagc |
| | | caagaaagcg ttaccgaaca ggatagcaaa gatagcacct atagcctgag |
| | | cagcaccctg accctgagca aagcagatta tgaaaaacac aaagtgtatg |
| | | cctgcgaagt tacccatcag ggtctgagca gtccggttac caaaagtttt |
| | | aatcgtggcg aatgctaata gaagcttggt acc |
| 13 | Ranibizumab | gagctcatat gaaatacctg ctgccgaccg ctgctgctgg tctgctgctc |
| | cDNA (Heavy | ctcgctgccc agccggcgat ggccgaagtt cagctggttg aaagcggtgg |
| | chain | tggtctggtt cagcctggtg gtagcctgcg tctgagctgt gcagcaagcg |
| | comprising a | gttatgattt tacccattat ggtatgaatt gggttcgtca ggcaccgggt |
| | signal | aaaggtctgg aatgggttgg ttggattaat acctataccg gtgaaccgac |
| | sequence) | ctatgcagca gattttaaac gtcgttttac ctttagcctg gataccagca |
| | | aaagcaccgc atatctgcag atgaatagcc tgcgtgcaga agataccgca |
| | | gtttattatt gtgccaaata tccgtattac tatggcacca gccactggta |
| | | tttcgatgtt tggggtcagg gcaccctggt taccgttagc agcgcaagca |
| | | ccaaaggtcc gagcgttttt ccgctggcac cgagcagcaa aagtaccagc |
| | | ggtggcacag cagcactggg ttgtctggtt aaagattatt ttccggaacc |
| | | ggttaccgtg agctggaata gcggtgcact gaccagcggt gttcatacct |
| | | ttccggcagt tctgcagagc agcggtctgt atagcctgag cagcgttgtt |
| | | accgttccga gcagcagcct gggcacccag acctatattt gtaatgttaa |
| | | tcataaaccg agcaatacca aagtggataa aaaagttgag ccgaaaagct |
| | | gcgataaaac ccatctgtaa tagggtacc |
| 14 | Bevacizumab | SASQDISNYLN |
| | and | |
| | Ranibizumab | |
| | Light Chain | |
| | CDR1 | |
| 15 | Bevacizumab | FTSSLHS |
| | and | |
| | Ranibizumab | |
| | Light Chain | |
| | CDR2 | |
| 16 | Bevacizumab | QQYSTVPWT |
| | and | |
| | Ranibizumab | |
| | Light Chain | |
| | CDR3 | |
| 17 | Bevacizumab | GYTFTNYGMN |
| | Heavy Chain | |
| | CDR1 | |
| 18 | Bevacizumab | WINTYTGEPTYAADFKR |
| | and | |
| | Ranibizumab | |
| | Heavy Chain | |
| | CDR2 | |
| 19 | Bevacizumab | YPHYYGSSHWYFDV |
| | Heavy Chain | |
| | CDR3 | |
| 20 | Ranibizumab | GYDFTHYGMN |
| | Heavy Chain | |
| | CDR1 | |
| 21 | Ranibizumab | YPYYYGTSHWYFDV |
| | Heavy Chain | |
| | CDR3 | |
| 22 | Albumin signal | MKWVTFISLLFLFSSAYS |
| | peptide | |
| 23 | Chymotrypsino | MAFLWLLSCWALLGTTFG |
| | gen signal | |
| | peptide | |
| 24 | Interleukin-2 | MYRMQLLSCIALILALVTNS |
| | signal peptide | |
| 25 | Trypsinogen-2 | MNLLLILTFVAAAVA |
| | signal peptide | |
| 26 | F2A site | LLNFDLLKLAGDVESNPGP |
| 27 | T2A site | (GSG)EGRGSLLTCGDVEENPGP |
| 28 | P2A site | (GSG)ATNFSLLKQAGDVEENPGP |
| 29 | E2A site | (GSG)QCTNYALLKLAGDVESNPGP |
| 30 | F2A site | (GSG)VKQTLNFDLLKLAGDVESNPGP |
| 31 | Furin linker | RKRR |
| 32 | Furin linker | RRRR |
| 33 | Furin linker | RRKR |
| 34 | Furin linker | RKKR |
| 35 | Furin linker | R-X-K/R-R |
| 36 | Furin linker | RXKR |
| 37 | Furin linker | RXRR |
| 38 | Ranibizumab | MDIQLTQSPSSLSASVGDRVTITCSASQDISNYLNWYQQKPGKAPKVLIYFTSSLHSGV |
| | Fab amino acid | PSRFSGSGSGTDFTLTISSLQPEDFATYYCQQYSTVPWTFGQGTKVEIKRTVAAPSVFI |
| | sequence (Light | FPPSDEQLKSGTASVVCLLNNFYPREAKVQWKVDNALQSGNSQESVTEQDSKDSTYSLS |
| | chain) | STLTLSKADYEKHKVYACEVTHQGLSSPVTKSFNRGEC |
| 39 | Ranibizumab | MEVQLVESGGGLVQPGGSLRLSCAASGYDFTHYGMNWVRQAPGKGLEWVGWINTYTGEP |
| | Fab amino acid | TYAADFKRRFTFSLDTSKSTAYLQMNSLRAEDTAVYYCAKYPYYYGTSHWYFDVWGQGT |
| | sequence | LVTVSSASTKGPSVFPLAPSSKSTSGGTAALGCLVKDYFPEPVTVSWNSGALTSGVHTF |
| | (Heavy chain) | PAVLQSSGLYSLSSVVTVPSSSLGTQTYICNVNHKPSNTKVDKKVEPKSCDKTHLRKRR |
| 40 | Ranibizumab | MEVQLVESGGGLVQPGGSLRLSCAASGYDFTHYGMNWVRQAPGKGLEWVGWINTYTGEP |
| | Fab amino acid | TYAADFKRRFTFSLDTSKSTAYLQMNSLRAEDTAVYYCAKYPYYYGTSHWYFDVWGQGT |
| | sequence | LVTVSSASTKGPSVFPLAPSSKSTSGGTAALGCLVKDYFPEPVTVSWNSGALTSGVHTF |
| | (Heavy chain) | PAVLQSSGLYSLSSVVTVPSSSLGTQTYICNVNHKPSNTKVDKKVEPKSCDKTHL |
| 41 | AAV1 | MAADGYLPDWLEDNLSEGIREWWDLKPGAPKPKANQQKQDDGRGLVLPGYKYLGPFNGL |
| | | DKGEPVNAADAAALEHDKAYDQQLKAGDNPYLRYNHADAEFQERLQEDTSFGGNLGRAV |
| | | FQAKKRVLEPLGLVEEGAKTAPGKKRPVEQSPQEPDSSSGIGKTGQQPAKKRLNFGQTG |
| | | DSESVPDPQPLGEPPATPAAVGPTTMASGGGAPMADNNEGADGVGNASGNWHCDSTWLG |
| | | DRVITTSTRTWALPTYNNHLYKQISSASTGASNDNHYFGYSTPWGYFDENREHCHFSPR |
| | | DWQRLINNNWGFRPKRLNFKLFNIQVKEVTTNDGVTTIANNLTSTVQVFSDSEYQLPYV |
| | | LGSAHQGCLPPFPADVFMIPQYGYLTLNNGSQAVGRSSFYCLEYFPSQMLRTGNNFTFS |
| | | YTFEEVPFHSSYAHSQSLDRLMNPLIDQYLYYLNRTQNQSGSAQNKDLLFSRGSPAGMS |
| | | VQPKNWLPGPCYRQQRVSKTKTDNNNSNFTWTGASKYNLNGRESIINPGTAMASHKDDE |
| | | DKFFPMSGVMIFGKESAGASNTALDNVMITDEEEIKATNPVATEREGTVAVNFQSSSTD |
| | | PATGDVHAMGALPGMVWQDRDVYLQGPIWAKIPHTDGHFHPSPLMGGFGLKNPPPQILI |
| | | KNTPVPANPPAEFSATKFASFITQYSTGQVSVEIEWELQKENSKRWNPEVQYTSNYAKS |
| | | ANVDFTVDNNGLYTEPRPIGTRYLTRPL |
| 42 | AAV2 | MAADGYLPDWLEDTLSEGIRQWWKLKPGPPPPKPAERHKDDSRGLVLPGYKYLGPFNGL |
| | | DKGEPVNEADAAALEHDKAYDRQLDSGDNPYLKYNHADAEFQERLKEDTSFGGNLGRAV |
| | | FQAKKRVLEPLGLVEEPVKTAPGKKRPVEHSPVEPDSSSGTGKAGQQPARKRLNFGQTG |
| | | DADSVPDPQPLGQPPAAPSGLGTNTMATGSGAPMADNNEGADGVGNSSGNWHCDSTWMG |
| | | DRVITTSTRTWALPTYNNHLYKQISSQSGASNDNHYEGYSTPWGYFDENREHCHFSPRD |
| | | WQRLINNNWGFRPKRLNFKLFNIQVKEVTQNDGTTTIANNLTSTVQVFTDSEYQLPYVL |
| | | GSAHQGCLPPFPADVFMVPQYGYLTLNNGSQAVGRSSFYCLEYFPSQMLRTGNNFTFSY |
| | | TFEDVPFHSSYAHSQSLDRLMNPLIDQYLYYLSRTNTPSGTTTQSRLQFSQAGASDIRD |
| | | QSRNWLPGPCYRQQRVSKTSADNNNSEYSWTGATKYHLNGRDSLVNPGPAMASHKDDEE |
| | | KFFPQSGVLIFGKQGSEKTNVDIEKVMITDEEEIRTTNPVATEQYGSVSTNLQRGNRQA |
| | | ATADVNTQGVLPGMVWQDRDVYLQGPIWAKIPHTDGHFHPSPLMGGFGLKHPPPQILIK |
| | | NTPVPANPSTTFSAAKFASFITQYSTGQVSVEIEWELQKENSKRWNPEIQYTSNYNKSV |
| | | NVDFTVDTNGVYSEPRPIGTRYLTRNL |
| 43 | AAV3-3 | MAADGYLPDWLEDNLSEGIREWWALKPGVPQPKANQQHQDNRRGLVLPGYKYLGPGNGL |
| | | DKGEPVNEADAAALEHDKAYDQQLKAGDNPYLKYNHADAEFQERLQEDTSFGGNLGRAV |
| | | FQAKKRILEPLGLVEEAAKTAPGKKGAVDQSPQEPDSSSGVGKSGKQPARKRLNFGQTG |
| | | DSESVPDPQPLGEPPAAPTSLGSNTMASGGGAPMADNNEGADGVGNSSGNWHCDSQWLG |
| | | DRVITTSTRTWALPTYNNHLYKQISSQSGASNDNHYEGYSTPWGYFDENREHCHFSPRD |
| | | WQRLINNNWGFRPKKLSFKLFNIQVRGVTQNDGTTTIANNLTSTVQVFTDSEYQLPYVL |
| | | GSAHQGCLPPFPADVFMVPQYGYLTLNNGSQAVGRSSFYCLEYFPSQMLRTGNNFQFSY |
| | | TFEDVPFHSSYAHSQSLDRLMNPLIDQYLYYLNRTQGTTSGTTNQSRLLFSQAGPQSMS |
| | | LQARNWLPGPCYRQQRLSKTANDNNNSNFPWTAASKYHLNGRDSLVNPGPAMASHKDDE |
| | | EKFFPMHGNLIFGKEGTTASNAELDNVMITDEEEIRTTNPVATEQYGTVANNLQSSNTA |
| | | PTTGTVNHQGALPGMVWQDRDVYLQGPIWAKIPHTDGHFHPSPLMGGFGLKHPPPQIMI |
| | | KNTPVPANPPTTFSPAKFASFITQYSTGQVSVEIEWELQKENSKRWNPEIQYTSNYNKS |
| | | VNVDFTVDTNGVYSEPRPIGTRYLTRNL |
| 44 | AAV4-4 | MTDGYLPDWLEDNLSEGVREWWALQPGAPKPKANQQHQDNARGLVLPGYKYLGPGNGLD |
| | | KGEPVNAADAAALEHDKAYDQQLKAGDNPYLKYNHADAEFQQRLQGDTSFGGNLGRAVF |
| | | QAKKRVLEPLGLVEQAGETAPGKKRPLIESPQQPDSSTGIGKKGKQPAKKKLVFEDETG |
| | | AGDGPPEGSTSGAMSDDSEMRAAAGGAAVEGGQGADGVGNASGDWHCDSTWSEGHVTTT |
| | | STRTWVLPTYNNHLYKRLGESLQSNTYNGESTPWGYFDENREHCHFSPRDWQRLINNNW |
| | | GMRPKAMRVKIFNIQVKEVTTSNGETTVANNLTSTVQIFADSSYELPYVMDAGQEGSLP |
| | | PFPNDVFMVPQYGYCGLVTGNTSQQQTDRNAFYCLEYFPSQMLRTGNNFEITYSFEKVP |
| | | FHSMYAHSQSLDRLMNPLIDQYLWGLQSTTTGTTLNAGTATTNETKLRPTNESNEKKNW |
| | | LPGPSIKQQGFSKTANQNYKIPATGSDSLIKYETHSTLDGRWSALTPGPPMATAGPADS |
| | | KFSNSQLIFAGPKQNGNTATVPGTLIFTSEEELAATNATDTDMWGNLPGGDQSNSNLPT |
| | | VDRLTALGAVPGMVWQNRDIYYQGPIWAKIPHTDGHFHPSPLIGGFGLKHPPPQIFIKN |
| | | TPVPANPATTFSSTPVNSFITQYSTGQVSVQIDWEIQKERSKRWNPEVQFTSNYGQQNS |
| | | LLWAPDAAGKYTEPRAIGTRYLTHHL |
| 45 | AAV5 | MSFVDHPPDWLEEVGEGLREFLGLEAGPPKPKPNQQHQDQARGLVLPGYNYLGPGNGLD |
| | | RGEPVNRADEVAREHDISYNEQLEAGDNPYLKYNHADAEFQEKLADDTSFGGNLGKAVF |
| | | QAKKRVLEPFGLVEEGAKTAPTGKRIDDHFPKRKKARTEEDSKPSTSSDAEAGPSGSQQ |
| | | LQIPAQPASSLGADTMSAGGGGPLGDNNQGADGVGNASGDWHCDSTWMGDRVVTKSTRT |
| | | WVLPSYNNHQYREIKSGSVDGSNANAYFGYSTPWGYFDENREHSHWSPRDWQRLINNYW |
| | | GFRPRSLRVKIFNIQVKEVTVQDSTTTIANNLTSTVQVFTDDDYQLPYVVGNGTEGCLP |
| | | AFPPQVFTLPQYGYATLNRDNTENPTERSSFFCLEYFPSKMLRTGNNFEFTYNFEEVPF |
| | | HSSFAPSQNLFKLANPLVDQYLYRFVSTNNTGGVQFNKNLAGRYANTYKNWFPGPMGRT |
| | | QGWNLGSGVNRASVSAFATTNRMELEGASYQVPPQPNGMTNNLQGSNTYALENTMIFNS |
| | | QPANPGTTATYLEGNMLITSESETQPVNRVAYNVGGQMATNNQSSTTAPATGTYNLQEI |
| | | VPGSVWMERDVYLQGPIWAKIPETGAHFHPSPAMGGFGLKHPPPMMLIKNTPVPGNITS |
| | | FSDVPVSSFITQYSTGQVTVEMEWELKKENSKRWNPEIQYTNNYNDPQFVDFAPDSTGE |
| | | YRTTRPIGTRYLTRPL |
| 46 | AAV6 | MAADGYLPDWLEDNLSEGIREWWDLKPGAPKPKANQQKQDDGRGLVLPGYKYLGPFNGL |
| | | DKGEPVNAADAAALEHDKAYDQQLKAGDNPYLRYNHADAEFQERLQEDTSFGGNLGRAV |
| | | FQAKKRVLEPFGLVEEGAKTAPGKKRPVEQSPQEPDSSSGIGKTGQQPAKKRLNFGQTG |
| | | DSESVPDPQPLGEPPATPAAVGPTTMASGGGAPMADNNEGADGVGNASGNWHCDSTWLG |
| | | DRVITTSTRTWALPTYNNHLYKQISSASTGASNDNHYFGYSTPWGYFDENREHCHFSPR |
| | | DWQRLINNNWGFRPKRLNFKLFNIQVKEVTTNDGVTTIANNLTSTVQVFSDSEYQLPYV |
| | | LGSAHQGCLPPFPADVFMIPQYGYLTLNNGSQAVGRSSFYCLEYFPSQMLRTGNNFTFS |
| | | YTFEDVPFHSSYAHSQSLDRLMNPLIDQYLYYLNRTQNQSGSAQNKDLLFSRGSPAGMS |
| | | VQPKNWLPGPCYRQQRVSKTKTDNNNSNFTWTGASKYNLNGRESIINPGTAMASHKDDK |
| | | DKFFPMSGVMIFGKESAGASNTALDNVMITDEEEIKATNPVATERFGTVAVNLQSSSTD |
| | | PATGDVHVMGALPGMVWQDRDVYLQGPIWAKIPHTDGHFHPSPLMGGFGLKHPPPQILI |
| | | KNTPVPANPPAEFSATKFASFITQYSTGQVSVEIEWELQKENSKRWNPEVQYTSNYAKS |
| | | ANVDFTVDNNGLYTEPRPIGTRYLTRPL |
| 47 | AAV7 | MAADGYLPDWLEDNLSEGIREWWDLKPGAPKPKANQQKQDNGRGLVLPGYKYLGPFNGL |
| | | DKGEPVNAADAAALEHDKAYDQQLKAGDNPYLRYNHADAEFQERLQEDTSFGGNLGRAV |
| | | FQAKKRVLEPLGLVEEGAKTAPAKKRPVEPSPQRSPDSSTGIGKKGQQPARKRLNFGQT |
| | | GDSESVPDPQPLGEPPAAPSSVGSGTVAAGGGAPMADNNEGADGVGNASGNWHCDSTWL |
| | | GDRVITTSTRTWALPTYNNHLYKQISSETAGSTNDNTYEGYSTPWGYFDENREHCHFSP |
| | | RDWQRLINNNWGFRPKKLRFKLFNIQVKEVTTNDGVTTIANNLTSTIQVFSDSEYQLPY |
| | | VLGSAHQGCLPPFPADVFMIPQYGYLTLNNGSQSVGRSSFYCLEYFPSQMLRTGNNFEF |
| | | SYSFEDVPFHSSYAHSQSLDRLMNPLIDQYLYYLARTQSNPGGTAGNRELQFYQGGPST |
| | | MAEQAKNWLPGPCFRQQRVSKTLDQNNNSNFAWTGATKYHLNGRNSLVNPGVAMATHKD |
| | | DEDRFFPSSGVLIFGKTGATNKTTLENVLMTNEEEIRPTNPVATEEYGIVSSNLQAANT |
| | | AAQTQVVNNQGALPGMVWQNRDVYLQGPIWAKIPHTDGNFHPSPLMGGFGLKHPPPQIL |
| | | IKNTPVPANPPEVFTPAKFASFITQYSTGQVSVEIEWELQKENSKRWNPEIQYTSNFEK |
| | | QTGVDFAVDSQGVYSEPRPIGTRYLTRNL |
| 48 | AAV8 | MAADGYLPDWLEDNLSEGIREWWALKPGAPKPKANQQKQDDGRGLVLPGYKYLGPFNGL |
| | | DKGEPVNAADAAALEHDKAYDQQLQAGDNPYLRYNHADAEFQERLQEDTSFGGNLGRAV |
| | | FQAKKRVLEPLGLVEEGAKTAPGKKRPVEPSPQRSPDSSTGIGKKGQQPARKRLNFGQT |
| | | GDSESVPDPQPLGEPPAAPSGVGPNTMAAGGGAPMADNNEGADGVGSSSGNWHCDSTWL |
| | | GDRVITTSTRTWALPTYNNHLYKQISNGTSGGATNDNTYFGYSTPWGYFDFNRFHCHFS |
| | | PRDWQRLINNNWGFRPKRLSFKLFNIQVKEVTQNEGTKTIANNLTSTIQVFTDSEYQLP |
| | | YVLGSAHQGCLPPFPADVFMIPQYGYLTLNNGSQAVGRSSFYCLEYFPSQMLRTGNNFQ |
| | | FTYTFEDVPFHSSYAHSQSLDRLMNPLIDQYLYYLSRTQTTGGTANTQTLGFSQGGPNT |
| | | MANQAKNWLPGPCYRQQRVSTTTGQNNNSNFAWTAGTKYHLNGRNSLANPGIAMATHKD |
| | | DEERFFPSNGILIFGKQNAARDNADYSDVMLTSEEEIKTTNPVATEEYGIVADNLQQQN |
| | | TAPQIGTVNSQGALPGMVWQNRDVYLQGPIWAKIPHTDGNFHPSPLMGGFGLKHPPPQI |
| | | LIKNTPVPADPPTTFNQSKLNSFITQYSTGQVSVEIEWELQKENSKRWNPEIQYTSNYY |
| | | KSTSVDFAVNTEGVYSEPRPIGTRYLTRNL |
| 49 | hu31 | MAADGYLPDWLEDTLSEGIRQWWKLKPGPPPPKPAERHKDDSRGLVLPGYKYLGPGNGL |
| | | DKGEPVNAADAAALEHDKAYDQQLKAGDNPYLKYNHADAEFQERLKEDTSFGGNLGRAV |
| | | FQAKKRLLEPLGLVEEAAKTAPGKKRPVEQSPQEPDSSAGIGKSGSQPAKKKLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYEGYSTPWGYFDENREHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGGQAVGRSSFYCLEYFPSQMLRTGNNFQF |
| | | SYEFENVPFHSSYAHSQSLDRLMNPLIDQYLYYLSKTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSAQAQ |
| | | AQTGWVQNQGILPGMVWQDRDVYLQGPIWAKIPHTDGNFHPSPLMGGFGMKHPPPQILI |
| | | KNTPVPADPPTAFNKDKLNSFITQYSTGQVSVEIEWELQKENSKRWNPEIQYTSNYYKS |
| | | NNVEFAVSTEGVYSEPRPIGTRYLTRNL |
| 50 | hu32 | MAADGYLPDWLEDTLSEGIRQWWKLKPGPPPPKPAERHKDDSRGLVLPGYKYLGPGNGL |
| | | DKGEPVNAADAAALEHDKAYDQQLKAGDNPYLKYNHADAEFQERLKEDTSFGGNLGRAV |
| | | FQAKKRLLEPLGLVEEAAKTAPGKKRPVEQSPQEPDSSAGIGKSGSQPAKKKLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYEGYSTPWGYFDENREHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSKTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSAQAQ |
| | | AQTGWVQNQGILPGMVWQDRDVYLQGPIWAKIPHTDGNFHPSPLMGGFGMKHPPPQILI |
| | | KNTPVPADPPTAFNKDKLNSFITQYSTGQVSVEIEWELQKENSKRWNPEIQYTSNYYKS |
| | | NNVEFAVNTEGVYSEPRPIGTRYLTRNL |
| 51 | AAV9 | MAADGYLPDWLEDNLSEGIREWWALKPGAPQPKANQQHQDNARGLVLPGYKYLGPGNGL |
| | | DKGEPVNAADAAALEHDKAYDQQLKAGDNPYLKYNHADAEFQERLKEDTSFGGNLGRAV |
| | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYEGYSTPWGYFDENREHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSKTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSAQAQ |
| | | AQTGWVQNQGILPGMVWQDRDVYLQGPIWAKIPHTDGNFHPSPLMGGFGMKHPPPQILI |
| | | KNTPVPADPPTAFNKDKLNSFITQYSTGQVSVEIEWELQKENSKRWNPEIQYTSNYYKS |
| | | NNVEFAVNTEGVYSEPRPIGTRYLTRNL |

### 6. EXAMPLES

### 6.1 Constructs

### 6.1.1 EXAMPLE 1: Bevacizumab Fab cDNA-Based Vector

A bevacizumab Fab cDNA-based vector is constructed comprising a transgene comprising bevacizumab Fab portion of the light and heavy chain cDNA sequences (SEQ ID NOs. 10 and 11, respectively). The transgene also comprises nucleic acids comprising a signal peptide chosen from the group listed in Table 1. The nucleotide sequences encoding the light chain and heavy chain are separated by IRES elements or 2A cleavage sites to create a bicistronic vector. Optionally, the vector additionally comprises a hypoxia-inducible promoter.

### 6.1.2 EXAMPLE 2: Ranibizumab cDNA-Based Vector

A ranibizumab Fab cDNA-based vector is constructed comprising a transgene comprising ranibizumab Fab light and heavy chain cDNAs (the portions of SEQ ID NOs. 12 and 13, respectively not encoding the signal peptide). The transgene also comprises nucleic acids comprising a signal peptide chosen from the group listed in Table 1. The nucleotide sequences encoding the light chain and heavy chain are separated by IRES elements or 2A cleavage sites to create a bicistronic vector. Optionally, the vector additionally comprises a hypoxia-inducible promoter.

### 6.1.3 EXAMPLE 3: Hyperglycosylated Bevacizumab Fab cDNA-Based Vector

A hyperglycosylated bevacizumab Fab cDNA-based vector is constructed comprising a transgene comprising bevacizumab Fab portion of the light and heavy chain cDNA sequences (SEQ ID NOs. 10 and 11, respectively) with mutations to the sequence encoding one or more of the following mutations: 1118N (heavy chain), E195N (light chain), or Q160N or Q160S (light chain). The transgene also comprises nucleic acids comprising a signal peptide chosen from the group listed in Table 1. The nucleotide sequences encoding the light chain and heavy chain are separated by IRES elements or 2A cleavage sites to create a bicistronic vector. Optionally, the vector additionally comprises a hypoxia-inducible promoter.

### 6.1.4 EXAMPLE 4: Hyperglycosylated Ranibizumab cDNA-based vector

A hyperglycosylated ranibizumab Fab cDNA-based vector is constructed comprising a transgene comprising ranibizumab Fab light and heavy chain cDNAs (the portions of SEQ ID NOs.12 and 13, respectively not encoding the signal peptide), with mutations to the sequence encoding one or more of the following mutations: L118N (heavy chain), E195N (light chain), or Q160N or Q160S (light chain). The transgene also comprises nucleic acids comprising a signal peptide chosen from the group listed in Table 1. The nucleotide sequences encoding the light chain and heavy chain are separated by IRES elements or 2A cleavage sites to create a bicistronic vector. Optionally, the vector additionally comprises a hypoxia-inducible promoter.

### 6.1.5 EXAMPLE 5: Ranibizumab Based HuGlyFabVEGFi

A ranibizumab Fab cDNA-based vector (see Example 2) is expressed in the PER.C6® Cell Line (Lonza) in the AAV8 background. The resultant product, ranibizumab-based HuGlyFab VEGFi is determined to be stably produced. N-glycosylation of the HuGlyFabVEGFi is confirmed by hydrazinolysis and MS/MS analysis. See, e.g., Bondt et al., Mol. & Cell. Proteomics 13.11:3029-3039. Based on glycan analysis, HuGlyFab VEGFi is confirmed to be N-glycosylated, with 2,6 sialic acid a predominant modification. Advantageous properties of the N-glycosylated HuGlyFab VEGFi are determined using methods known in the art. The HuGlyFab VEGFi can be found to have increased stability and increased affinity for its antigen (VEGF). See Sola and Griebenow, 2009, J Pharm Sci., 98(4): 1223-1245 for methods of assessing stability and Wright et al., 1991, EMBO J. 10:2717-2723 and Leibiger et al., 1999, Biochem. J. 338:529-538 for methods of assessing affinity.

### 6.2 Treatment with Constructs

### 6.2.1 EXAMPLE 6: Treatment of Wet AMD with Ranibizumab Based HuGly FabVEGFi

Based on determination of advantageous characteristics of ranibizumab-based HuGlyFabVEGFi (see Example 5), a ranibizumab Fab cDNA-based vector is deemed useful for treatment of wet AMD when expressed as a transgene. A subject presenting with wet AMD is administered AAV8 that encodes ranibizumab Fab at a dose sufficient to that a concentration of the transgene product at a Cₘᵢₙ of at least 0.330 μg/mL in the Vitreous humour for three months. Following treatment, the subject is evaluated for improvement in symptoms of wet AMD.

### 6.3 Mouse Studies

### 6.3.1 EXAMPLE 7: A Single Dose Subretinal Administration Reduces Retinal Neovascularization in Transgenic Rho/VEGF Mice

This study demonstrates the in vivo efficacy of a single dose of, an HuPTMFab VEGFi vector, as described in Section 5. 2, in juvenile transgenic Rho/VEGF mice (Tobe, 1998, IOVS 39(1): 180-188), a model for the neovascular changes in the retina of humans with nAMD. Rho/VEGF mice are transgenic mice in which the rhodopsin promoter constitutively drives expression of human VEGF165 in photoreceptors, causing new vessels to sprout from the deep capillary bed of the retina and grow into the subretinal space, starting at postnatal Day 10. The production of VEGF is sustained and therefore the new vessels continue to grow and enlarge and form large nets in the subretinal space similar to those seen in humans with neovascular age-related macular degeneration. (Tobe 1998, supra).

The vector used in this study (referred to herein as “Vector 1”) is a non-replicating AAV8 vector containing a gene cassette encoding a humanized mAb antigen-binding fragment that binds and inhibits human VEGF, flanked by AAV2 inverted terminal repeats (ITRs). Expression of heavy and light chains in Vector 1 is controlled by the CB7 promoter consisting of the chicken β-actin promoter and CMV enhancer, and the vector also comprises a chicken β-actin intron, and a rabbit β-globin polyA signal. In Vector 1, the nucleic acid sequences coding for the heavy and light chains of anti-VEGF Fab are separated by a self-cleaving furin (F)/F2A linker. Rho/VEGF mice were injected subretinally with either Vector 1 or control (n=10-17 per group) and one week later the amount of retinal neovascularization was quantitated.

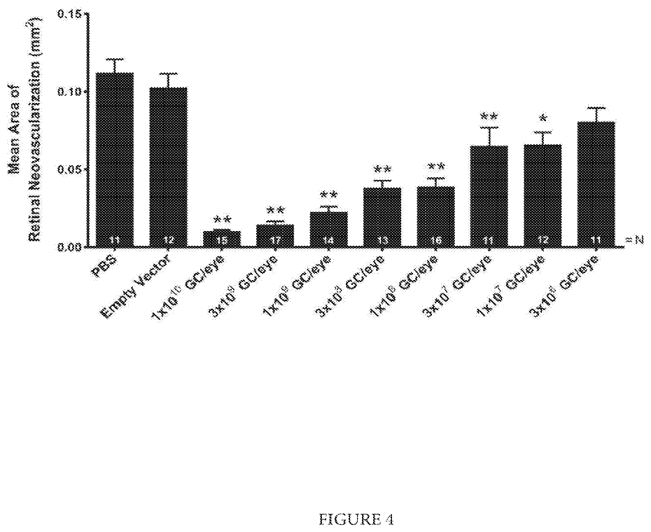

The total area of retinal neovascularization was significantly reduced (p<0.05) in Rho/VEGF mice receiving Vector 1 in a dose-dependent manner, as compared to mice receiving either phosphate buffered saline (PBS) or null AAV8 vector. The effectiveness criterion was set as a statistically significant reduction in the area of retinal neovascularization. With this criterion, a minimum dose of 1×10⁷ GC/eye of Vector 1 was determined to be efficacious for reduction of retinal neovascularization in the murine transgenic Rho/VEGF model for nAMD in human subjects (FIG. 4).

### 6.3.2 EXAMPLE 8: A Single Dose Subretinal Administration Reduces Retinal Detachment in Double Transgenic Tet/Opsin/VEGF Mice

This study demonstrates the in vivo efficacy of a single dose of the Vector 1, to prevent retinal detachment in a transgenic mouse model of ocular neovascular disease in human subjects—Tet/opsin/VEGF mice—in which inducible expression of VEGF causes severe retinopathy and retinal detachment (Ohno-Matsui, 2002 Am. J. Pathol. 160(2):711-719). Tet/opsin/VEGF mice are transgenic mice with doxycycline inducible expression of human VEGF 165 in photoreceptors. These transgenic mice are phenotypically normal until given doxycycline in drinking water. Doxycycline induces very high photoreceptor expression of VEGF, leading to massive vascular leakage, culminating in total exudative retinal detachment in 80-90% of mice within 4 days of induction.

Tet/opsin/VEGF mice (10 per group) were injected subretinally with Vector 1 or control. Ten days after injection, doxycycline was added to the drinking water to induce VEGF expression. After 4 days, the fundus of each eye was imaged and each retina was scored as either intact, partially detached, or totally detached by an individual who had no knowledge of treatment group.

These data (shown in FIG. 5) demonstrate that treatment with Vector 1 caused a reduction in the incidence and degree of retinal detachments in Tet/opsin/VEGF mice—an animal model for ocular neovascular disease in human subjects.

### 6.3.3 EXAMPLE 9: AAV8 Gene Therapy Expressing an Anti-VEGF Protein Strongly Suppresses Subretinal Neovascularization and Vascular Leakage in Mouse Models

In this example, the methods, results, and conclusions from the experiments described in Examples 7 and 8 are summarized.

Methods. Transgenic mice in which the rhodopsin promoter drives expression of VEGF 165 in photoreceptors (rho/VEGF mice) had a subretinal injection of Vector 1 with doses ranging from 3×10⁶-1×10¹⁰ genome copies (GC), 1×10¹⁰ GC of null vector, or PBS in one eye (n=10 per group) at post-natal day 14 (P14). At P21, the area of subretinal neovascularization (SNV) per eye was measured. Double transgenic mice with doxycycline (DOX)-inducible expression of VEGF165 in photoreceptors (Tet/opsin/VEGF mice) had a subretinal injection of 1×10⁸-1×10¹⁰ GC of Vector 1 in one eye and no injection in the fellow eye or 1×10¹⁰ GC of null vector in one eye and PBS in the fellow eye. Ten days after injection, 2 mg/ml of DOX was added to drinking water and after 4 days fundus photos were graded for presence of total, partial, or no retinal detachment (RD). Vector 1 transgene product levels were measured one week after subretinal injection of 1×10⁸-1×10¹⁰ GC of Vector 1 in adult mice by ELISA analyses of eye homogenates.

Results. Compared to eyes of rho/VEGF mice injected null vector, those injected with ≥1×10⁷ GC of Vector 1 had significant reduction in mean area of SNV, with modest reduction in eyes injected with ≤3×10⁷ and >50% reduction in eyes injected with ≥1×10⁸ GC. Eyes injected with 3×10⁹ or 1×10¹⁰ GC had almost complete elimination of SNV. In Tet/opsin/VEGF mice, compared to the null vector group in which 100% of eyes had total RD, there was significant reduction in exudative RD in eyes injected with ≥3×10⁸ GC of Vector 1 and reduction of total detachments by 70-80% in eyes injected with 3×10⁹ or 1×10¹⁰ GC. The majority of eyes injected with ≤1×10⁹ GC of Vector 1 had protein levels below the limit of detection, but all eyes injected with 3×10⁹ or 1×10¹⁰ GC had detectable levels with mean level per eye 342.7 ng and 286.2 ng.

Conclusions. Gene therapy by subretinal injection of Vector 1 caused dose dependent suppression of SNV in rho/VEGF mice with near complete suppression with doses of 3×10⁹ or 1×10¹⁰ GC. These same doses showed robust protein product expression and markedly reduced total exudative RD in Tet/opsin/VEGF mice.

### 6.4 Human Study

### 6.4.1 EXAMPLE 10: Gene Therapy for Neovascular AMD: A Dose-escalation Study to Evaluate the Safety and Tolerability of Gene Therapy With Vector 1 in Subjects With Neovascular AMD (nAMD)

Brief Summary of Study. Excessive vascular endothelial growth factor (VEGF) plays a key part in promoting neovascularization and edema in neovascular (wet) age-related macular degeneration (nAMD). VEGF inhibitors (anti-VEGF), including ranibizumab (LUCENTIS®, Genentech) and aflibercept (EYLEA®, Regeneron), have been shown to be safe and effective for treating nAMD and have demonstrated improvement in vision. However, anti-VEGF therapy is administered frequently via intravitreal injection and can be a significant burden to the patients. Vector 1 is a recombinant adeno-associated virus (AAV) gene therapy vector carrying a coding sequence for a soluble anti-VEGF protein. The long-term, stable delivery of this therapeutic protein following a one-time gene therapy treatment for nAMD could reduce the treatment burden of currently available therapies while maintaining vision with a favorable benefit:risk profile.

Detailed Description of Study. This dose-escalation study is designed to evaluate the safety and tolerability of Vector 1 gene therapy in subjects with previously treated nAMD. Three doses will be studied in approximately 18 subjects. Subjects who meet the inclusion/exclusion criteria and have an anatomic response to an initial anti VEGF injection will receive a single dose of Vector 1 administered by subretinal delivery. Vector 1 uses an AAV8 vector that contains a gene that encodes for a monoclonal antibody fragment which binds to and neutralizes VEGF activity. Safety will be the primary focus for the initial 24 weeks after Vector 1 administration (primary study period). Following completion of the primary study period, subjects will continue to be assessed until 104 weeks following treatment with Vector 1.

Dosing. Three doses will be used: 3×10⁹ GC of Vector 1, 1×10¹⁰ GC of Vector 1, and 6×10¹⁰ GC of Vector 1.

Outcome Measures. The Primary Outcome Measure will be safety—the incidence of ocular and non-ocular adverse events (AEs) and serious adverse events (SAEs)—over a time frame of 26 weeks.

Secondary Outcome Measures will include:

Safety—the incidence of ocular and non-ocular AEs and SAEs—over a time frame of 106 weeks.

Change in best corrected visual acuity (BCVA) over a time frame of 106 weeks.

Change in central retinal thickness (CRT) as measured by SD-OCT—over a time frame of 106 weeks.

Rescue injections—the mean number of rescue injections-over a time frame of 106 weeks.

Change in choroidal neovascularization (CNV) and lesion size and leakage area CNV changes, as measured by fluorescein angiography (FA)—over a time frame of 106 weeks.

Eligibility Criteria. The following eligibility criteria apply to the study:
Minimum Age: 50 years
Maximum Age: (none)
Sex: All
Gender Based: No
Accepts Healthy Volunteers: No
Inclusion Criteria:
Patients ≥50 years with a diagnosis of subfoveal CNV secondary to AMD in the study eye receiving prior intravitreal anti-VEGF therapy.
BCVA between ≤20/63 and ≥20/400 (≤63 and ≥19 Early Treatment Diabetic Retinopathy Study [ETDRS] letters) for the first patient in each cohort followed by BCVA between ≤20/20 and ≥20/400 (≤73 and ≥19 ETDRS letters) for the rest of the cohort.
History of need for and response to anti-VEGF therapy.
Response to anti-VEGF at trial entry (assessed by SD-OCT at week 1).
Must be pseudophakic (status post cataract surgery) in the study eye.
Aspartate aminotransferase/alanine aminotransferase (AST/ALT)<2.5× upper limit of normal (ULN); total bilirubin (TB)<1.5× ULN; prothrombin time (PT)<1.5× ULN; hemoglobin (Hb)>10 g/dL (males) and >9 g/dL (females); Platelets >100×10³/μL; estimated glomerular filtration rate (eGFR)>30 mL/min/1.73 m².
Must be willing and able to provide written, signed informed consent.

### Exclusion Criteria:

CNV or macular edema in the study eye secondary to any causes other than AMD.
Any condition preventing visual acuity improvement in the study eye, e.g., fibrosis, atrophy, or retinal epithelial tear in the center of the fovea.
Active or history of retinal detachment in the study eye.
Advanced glaucoma in the study eye.
History of intravitreal therapy in the study eye, such as intravitreal steroid injection or investigational product, other than anti-VEGF therapy, in the 6 months prior to screening.
Presence of an implant in the study eye at screening (excluding intraocular lens).
Myocardial infarction, cerebrovascular accident, or transient ischemic attacks within the past 6 months.
Uncontrolled hypertension (systolic blood pressure [BP]>180 mmHg, diastolic BP >100 mmHg) despite maximal medical treatment.

### 6.4.2 EXAMPLE 11: Protocol for Treating Human Subjects

This Example relates to a gene therapy treatment for patients with neovascular (wet) age-related macular degeneration (nAMD). This Example is an updated version of Example 10. In this example, Vector 1, a replication deficient adeno-associated viral vector 8 (AAV8) carrying a coding sequence for a soluble anti-VEGF Fab protein (as described in Example 7), is administered to patients with nAMD. The goal of the gene therapy treatment is to slow or arrest the progression of retinal degeneration and to slow or prevent loss of vision with minimal intervention/invasive procedures.

Dosing & Route of Administration. A volume of 250 μL of Vector 1 is administered as a single dose via subretinal delivery in the eye of a subject in need of treatment. The subject receives a dose of 3×10⁹ GC/eye, 1×10¹⁰ GC/eye, or 6×10¹⁰ GC/eye.

Subretinal delivery is performed by a retinal surgeon with the subject under local anesthesia. The procedure involves a standard 3-port pars plana vitrectomy with a core vitrectomy followed by subretinal delivery of Vector 1 into the subretinal space by a subretinal cannula (38 gauge). The delivery is automated via the vitrectomy machine to deliver 250 μL to the subretinal space. The injection and resulting bleb is documented by video recording and by a drawn representation by the surgeon.

Gene therapy can be administered in combination with one or more therapies for the treatment of wetAMD. For example, gene therapy is administered in combination with laser coagulation, photodynamic therapy with verteporfin, and intravitreal with anti-VEGF agent, including but not limited to pegaptanib, ranibizumab, aflibercept, or bevacizumab.

Starting at about 4 weeks post-Vector 1 administration, a patient may receive intravitreal ranibizumab rescue therapy at the treating physician's discretion in the affected eye.

Patient Subpopulations. Suitable patients may include those:
Having a diagnosis of nAMD;
Responsive to anti-VEGF therapy;
Requiring frequent injections of anti-VEGF therapy;
Males or females aged 50 years or above;
Having a BCVA ≤20/63 and ≥20/400 (≤63 and ≥19 ETDRS letters) in the affected eye;
Having a BCVA between ≤20/20 and ≥20/400 (≤73 and ≥19 ETDRS letters);
Having a documented diagnosis of subfoveal CNV secondary to AMD in the affected eye;
Having CNV lesion characteristics as follows: lesion size less than 10 disc areas (typical disc area is 2.54 mm²), blood <50% of the lesion size;
Having received at least 4 intravitreal injections of an anti-VEGF agent for treatment of nAMD in the affected eye in approximately 8 months (or less) prior to treatment, with anatomical response documented on SD-OCT; and/or
Having subretinal or intraretinal fluid present in the affected eye, evidenced on SD-OCT.

Prior to treatment, patients are screened and one or more of the following criteria may indicate this therapy is not suitable for the patient:
CNV or macular edema in the affected eye secondary to any causes other than AMD;
Blood occupying ≥50% of the AMD lesion or blood >1.0 mm2 underlying the fovea in the affected eye;
Any condition preventing VA improvement in the affected eye, e.g., fibrosis, atrophy, or retinal epithelial tear in the center of the fovea;
Active or history of retinal detachment in the affected eye;
Advanced glaucoma in the affected eye;
Any condition in the affected eye that may increase the risk to the subject, require either medical or surgical intervention to prevent or treat vision loss, or interfere with study procedures or assessments;
History of intraocular surgery in the affected eye within 12 weeks prior to screening (Yttrium aluminum garnet capsulotomy may be permitted if performed >10 weeks prior to the screening visit.);
History of intravitreal therapy in the affected eye, such as intravitreal steroid injection or investigational product, other than anti-VEGF therapy, in the 6 months prior to screening;
Presence of an implant in the affected eye at screening (excluding intraocular lens).
History of malignancy requiring chemotherapy and/or radiation in the 5 years prior to screening (Localized basal cell carcinoma may be permitted.);
History of therapy known to have caused retinal toxicity, or concomitant therapy with any drug that may affect visual acuity or with known retinal toxicity, e.g., chloroquine or hydroxychloroquine;
Ocular or periocular infection in the affected eye that may interfere with the surgical procedure;
Myocardial infarction, cerebrovascular accident, or transient ischemic attacks within the past 6 months of treatment;
Uncontrolled hypertension (systolic blood pressure [BP]>180 mmHg, diastolic BP >100 mmHg) despite maximal medical treatment;
Any concomitant treatment that may interfere with ocular surgical procedure or healing process;
Known hypersensitivity to ranibizumab or any of its components or past hypersensitivity to agents like Vector 1;
Any serious or unstable medical or psychological condition that, in the opinion of the Investigator, would compromise the subject's safety or successful participation in the study.
Aspartate aminotransferase (AST)/alanine aminotransferase (ALT)>2.5 x upper limit of normal (ULN)
Total bilirubin >1.5× ULN unless the subject has a previously known history of Gilbert's syndrome and a fractionated bilirubin that shows conjugated bilirubin <35% of total bilirubin
Prothrombin time (PT)>1.5× ULN
Hemoglobin <10 g/dL for male subjects and <9 g/dL for female subjects
Platelets <100×10³/μL
Estimated glomerular filtration rate (GFR)<30 mL/min/1.73 m²

Starting at about 4 weeks post-gene therapy administration, a patient may receive intravitreal ranibizumab rescue therapy at the treating physician's discretion in the affected eye for disease activity if 1 or more of the following rescue criteria apply:
Vision loss of >5 letters (per Best Corrected Visual Acuity [BCVA]) associated with accumulation of retinal fluid on Spectral Domain Optical Coherence Tomography (SD-OCT)
Choroidal neovascularization (CNV)-related increased, new, or persistent subretinal or intraretinal fluid on SD-OCT
New ocular hemorrhage

Further rescue injections may be deferred per the treating physician's discretion if one of the following sets of findings occur:
Visual acuity is 20/20 or better and central retinal thickness is “normal” as assessed by SD-OCT, or
Visual acuity and SD-OCT are stable after 2 consecutive injections.

If injections are deferred, they will be resumed if visual acuity or SD-OCT get worse per the criteria above.

Measuring Clinical Objectives. Primary clinical objectives include slowing or arresting the progression of retinal degeneration and slowing or preventing loss of vision. Clinical objectives are indicated by the elimination of or reduction in the number of rescue treatments using standard of care, for example, intravitreal injections with anti-VEGF agents, including but not limited to pegaptanib, ranibizumab, aflibercept, or bevacizumab. Clinical objectives are also indicated by a decrease or prevention of vision loss and/or a decrease or prevention of retinal detachment.

Clinical objectives are determined by measuring BCVA (Best-Corrected Visual Acuity), intraocular pressure, slit lamp biomicroscopy, indirect ophthalmoscopy, and/or SD-OCT (SD-Optical Coherence Tomography). In particular, clinical objectives are determined by measuring mean change from baseline in BCVA over time, measuring the gain or loss of ≥15 letters compared to baseline as per BCVA, measuring mean change from baseline in CRT as measured by SD-OCT over time, measuring mean number of ranibizumab rescue injections over time, measuring time to 1^{st} rescue ranibizumab injection, measuring mean change from baseline in CNV and lesion size and leakage area based on FA over time, measuring mean change from baseline in aqueous aVEGF protein over time, performing vector shedding analysis in serum and urine, and/or measuring immunogenicity toVector 1, i.e., measuring Nabs to AAV, measuring binding antibodies to AAV, measuring antibodies to aVEGF, and/or performing ELISpot.

Clinical objectives are also determined by measuring the mean change from baseline over time in area of geographic atrophy per fundus autofluorescence (FAF), measuring the incidence of new area of geographic atrophy by FAF (in subjects with no geographic atrophy at baseline, measuring the proportion of subjects gaining or losing ≥5 and ≥10 letters, respectively, compared with baseline as per BCVA, measuring the proportion of subjects who have a reduction of 50% in rescue injections compared with previous year, measuring the proportion of subjects with no fluid on SD-OCT.

Improvement/efficacy resulting from Vector 1 administration can be assessed as a defined mean change in baseline in visual acuity at about 4 weeks, 12 weeks, 6 months, 12 months, 24 months, 36 months, or at other desired timepoints. Treatment with Vector 1 can result in a 5%, 10%, 15%, 20%, 30%, 40%, 50% or more increase in visual acuity from baseline. Improvements/efficacy can be assessed as mean change from baseline in central retinal thickness (CRT) as measured by spectral domain optical coherence tomography (SD-OCT) at 4 weeks, 12 weeks, 6 months, 12 months, 24 months and 36 months. Treatment with Vector 1 can result in a 5%, 10%, 15%, 20%, 30%, 40%, 50% or more increase central retinal thickness from baseline.

### 6.4.3 EXAMPLE 12: Gene Therapy for Neovascular AMD: A Dose-escalation Study to Evaluate the Safety and Tolerability of Gene Therapy With Vector 1 in Subjects With Neovascular AMD (nAMD)

Brief Summary of Study. Excessive vascular endothelial growth factor (VEGF) plays a key part in promoting neovascularization and edema in neovascular (wet) age-related macular degeneration (nAMD). VEGF inhibitors (anti-VEGF), including ranibizumab (LUCENTIS®, Genentech) and aflibercept (EYLEA®, Regeneron), have been shown to be safe and effective for treating nAMD and have demonstrated improvement in vision. However, anti-VEGF therapy is administered frequently via intravitreal injection and can be a significant burden to the patients. Vector 1 is a recombinant adeno-associated virus (AAV) gene therapy vector carrying a coding sequence for a soluble anti-VEGF protein. The long-term, stable delivery of this therapeutic protein following a one-time gene therapy treatment for nAMD could reduce the treatment burden of currently available therapies while maintaining vision with a favorable benefit:risk profile.

Detailed Description of Study. This dose-escalation study is designed to evaluate the safety and tolerability of Vector 1 gene therapy in subjects with previously treated nAMD. Five doses will be studied in approximately 30 subjects. Additional subjects may be enrolled if subject(s) does not receive a full 250 μL dose in the subretinal space. Subjects who meet the inclusion/exclusion criteria and have an anatomic response to an initial anti VEGF injection will receive a single dose of Vector 1 administered by subretinal delivery. Subretinal delivery in this study will be targeted to the area superior to the fovea within the vascular arcades, which will avoid the macula. Vector 1 uses an AAV8 vector that contains a gene that encodes for a monoclonal antibody fragment which binds to and neutralizes VEGF activity. Safety will be the primary focus for the initial 24 weeks after Vector 1 administration (primary study period). Following completion of the primary study period, subjects will continue to be assessed until 104 weeks following treatment with Vector 1.

Dosing. Five doses will be used in a 250 μL volume of Vector 1: 3×10⁹ GC of Vector 1 (1.2×10¹⁰ GC/mL), 1×10¹⁰ GC of Vector 1 (4×10¹⁰ GC/mL), 6×10¹⁰ GC of Vector 1 (2.4×10¹¹ GC/mL), 1.6×10¹¹ GC of Vector 1 (6.2×10¹¹ GC/mL), and 2.5×10¹¹ GC of Vector 1 (1×10¹² GC/mL).

Outcome Measures. The Primary Outcome Measure will be safety—the incidence of ocular and non-ocular adverse events (AEs) and serious adverse events (SAEs)—over a time frame of 26 weeks.

Secondary Outcome Measures will include:

Safety—the incidence of ocular and non-ocular AEs and SAEs—over a time frame of 106 weeks.

Change in best corrected visual acuity (BCVA)—over a time frame of 106 weeks.

Change in central retinal thickness (CRT) as measured by SD-OCT—over a time frame of 106 weeks.

Rescue injections—the mean number of rescue injections—over a time frame of 106 weeks.

Change in choroidal neovascularization (CNV) and lesion size and leakage area CNV changes, as measured by fluorescein angiography (FA)—over a time frame of 106 weeks.

Eligibility Criteria. The following eligibility criteria apply to the study:
Minimum Age: 50 years
Maximum Age: 89 years
Sex: All
Gender Based: No
Accepts Healthy Volunteers: No

Inclusion Criteria:
Patients ≥50 years and ≤89 years with a diagnosis of subfoveal CNV secondary to AMD in the study eye receiving prior intravitreal anti-VEGF therapy.
BCVA between ≤20/63 and ≥20/400 (≤63 and ≥19 Early Treatment Diabetic Retinopathy Study [ETDRS] letters) for the first patient in each cohort followed by BCVA between ≤20/20 and ≥20/400 (≤73 and ≥19 ETDRS letters) for the rest of the cohort.
History of need for and response to anti-VEGF therapy.
Response to anti-VEGF at trial entry (assessed by SD-OCT at week 1).
Must be pseudophakic (status post cataract surgery) in the study eye.
Aspartate aminotransferase/alanine aminotransferase (AST/ALT)<2.5× upper limit of normal (ULN); total bilirubin (TB)<1.5 x ULN; prothrombin time (PT)<1.5× ULN; hemoglobin (Hb)>10 g/dL (males) and >9 g/dL (females); Platelets >100×10³/μL; estimated glomerular filtration rate (eGFR)>30 mL/min/1.73 m².
Must be willing and able to provide written, signed informed consent.

Exclusion Criteria:
CNV or macular edema in the study eye secondary to any causes other than AMD.
Any condition preventing visual acuity improvement in the study eye, e.g., fibrosis, atrophy, or retinal epithelial tear in the center of the fovea.
Active or history of retinal detachment in the study eye.
Advanced glaucoma in the study eye.
History of intravitreal therapy in the study eye, such as intravitreal steroid injection or investigational product, other than anti-VEGF therapy, in the 6 months prior to screening.
Presence of an implant in the study eye at screening (excluding intraocular lens).
Myocardial infarction, cerebrovascular accident, or transient ischemic attacks within the past 6 months.
Uncontrolled hypertension (systolic blood pressure [BP]>180 mmHg, diastolic BP>100 mmHg) despite maximal medical treatment.

### 6.4.4 EXAMPLE 13: Protocol for Treating Human Subjects

This Example relates to a gene therapy treatment for patients with neovascular (wet) age-related macular degeneration (nAMD). This Example is an updated version of Example 12. In this example, Vector 1, a replication deficient adeno-associated viral vector 8 (AAV8) carrying a coding sequence for a soluble anti-VEGF Fab protein (as described in Example 7), is administered to patients with nAMD. The goal of the gene therapy treatment is to slow or arrest the progression of retinal degeneration and to slow or prevent loss of vision with minimal intervention/invasive procedures.

Dosing & Route of Administration. A volume of 250 μL of Vector 1 is administered as a single dose via subretinal delivery in the eye of a subject in need of treatment. The subject receives a dose of 3×10⁹ GC (1.2×10¹⁰ GC/mL), 1×10¹⁰ GC (4×10¹⁰ GC/mL), 6×10¹⁰ GC (2.4×10¹¹ GC/mL), 1.6×10¹¹ GC (6.2×10¹¹ GC/mL), or 2.5×10¹¹ GC (1×10¹² GC/mL).

Subretinal delivery is performed by a retinal surgeon with the subject under local anesthesia. The procedure involves a standard 3-port pars plana vitrectomy with a core vitrectomy followed by subretinal delivery of Vector 1 into the subretinal space by a subretinal cannula (38 gauge). The delivery is automated via the vitrectomy machine to deliver 250 μL to the subretinal space. The injection and resulting bleb is documented by video recording and by a drawn representation by the surgeon. Subretinal delivery is targeted to the area superior to the fovea within the vascular arcades, which avoids the macula.

Gene therapy can be administered in combination with one or more therapies for the treatment of wetAMD. For example, gene therapy is administered in combination with laser coagulation, photodynamic therapy with verteporfin, and intravitreal with anti-VEGF agent, including but not limited to pegaptanib, ranibizumab, aflibercept, or bevacizumab.

Starting at about 4 weeks post-Vector 1 administration, a patient may receive intravitreal ranibizumab rescue therapy at the treating physician's discretion in the affected eye. The rescue therapy may be changed from ranibizumab to aflibercept at the treating physician's discretion.

Patient Subpopulations. Suitable patients may include those:
Having a diagnosis of nAMD;
Responsive to anti-VEGF therapy;
Requiring frequent injections of anti-VEGF therapy;
Males or females aged 50 years or above and 89 years or younger;
Having a BCVA≤20/63 and ≥20/400 (≤63 and ≥19 ETDRS letters) in the affected eye;
Having a BCVA between ≤20/20 and ≥20/400 (≤73 and ≥19 ETDRS letters);
Having a documented diagnosis of subfoveal CNV secondary to AMD in the affected eye;
Having CNV lesion characteristics as follows: lesion size less than 10 disc areas (typical disc area is 2.54 mm²), blood <50% of the lesion size;
Having received at least 4 intravitreal injections of an anti-VEGF agent for treatment of nAMD in the affected eye in approximately 8 months (or less) prior to treatment, with anatomical response documented on SD-OCT; and/or
Having subretinal or intraretinal fluid present in the affected eye, evidenced on SD-OCT.

Prior to treatment, patients are screened and one or more of the following criteria may indicate this therapy is not suitable for the patient:
CNV or macular edema in the affected eye secondary to any causes other than AMD;
Blood occupying ≥50% of the AMD lesion or blood >1.0 mm2 underlying the fovea in the affected eye;
Any condition preventing VA improvement in the affected eye, e.g., fibrosis, atrophy, or retinal epithelial tear in the center of the fovea;
Active or history of retinal detachment in the affected eye;
Advanced glaucoma in the affected eye;
Any condition in the affected eye that may increase the risk to the subject, require either medical or surgical intervention to prevent or treat vision loss, or interfere with study procedures or assessments;
History of intraocular surgery in the affected eye within 12 weeks prior to screening (Yttrium aluminum garnet capsulotomy may be permitted if performed >10 weeks prior to the screening visit.);
History of intravitreal therapy in the affected eye, such as intravitreal steroid injection or investigational product, other than anti-VEGF therapy, in the 6 months prior to screening;
Presence of an implant in the affected eye at screening (excluding intraocular lens).
History of malignancy requiring chemotherapy and/or radiation in the 5 years prior to screening (Localized basal cell carcinoma may be permitted.);
History of therapy known to have caused retinal toxicity, or concomitant therapy with any drug that may affect visual acuity or with known retinal toxicity, e.g., chloroquine or hydroxychloroquine;
Ocular or periocular infection in the affected eye that may interfere with the surgical procedure;
Myocardial infarction, cerebrovascular accident, or transient ischemic attacks within the past 6 months of treatment;
Uncontrolled hypertension (systolic blood pressure [BP]>180 mmHg, diastolic BP >100 mmHg) despite maximal medical treatment;
Any concomitant treatment that may interfere with ocular surgical procedure or healing process;
Known hypersensitivity to ranibizumab or any of its components or past hypersensitivity to agents like Vector 1;
Any serious, chronic, or unstable medical or psychological condition that, in the opinion of the Investigator, may compromise the subject's safety or ability to complete all assessments and follow-up in the study.
Aspartate aminotransferase (AST)/alanine aminotransferase (ALT)>2.5 x upper limit of normal (ULN)
Total bilirubin >1.5× ULN unless the subject has a previously known history of Gilbert's syndrome and a fractionated bilirubin that shows conjugated bilirubin <35% of total bilirubin
Prothrombin time (PT)>1.5× ULN
Hemoglobin <10 g/dL for male subjects and <9 g/dL for female subjects
Platelets <100×10³/μL
Estimated glomerular filtration rate (GFR)<30 mL/min/1.73 m²

Starting at about 4 weeks post-gene therapy administration, a patient may receive intravitreal ranibizumab rescue therapy at the treating physician's discretion in the affected eye for disease activity if 1 or more of the following rescue criteria apply:
Vision loss of ≥5 letters (per Best Corrected Visual Acuity [BCVA]) associated with accumulation of retinal fluid on Spectral Domain Optical Coherence Tomography (SD-OCT)
Choroidal neovascularization (CNV)-related increased, new, or persistent subretinal or intraretinal fluid on SD-OCT
New ocular hemorrhage

Further rescue injections may be deferred per the treating physician's discretion if one of the following sets of findings occur:
Visual acuity is 20/20 or better and central retinal thickness is “normal” as assessed by SD-OCT, or
Visual acuity and SD-OCT are stable after 2 consecutive injections.

If injections are deferred, they will be resumed if visual acuity or SD-OCT get worse per the criteria above. The rescue therapy may be changed from ranibizumab to aflibercept at the treating physician's discretion.

Measuring Clinical Objectives. Primary clinical objectives include slowing or arresting the progression of retinal degeneration and slowing or preventing loss of vision. Clinical objectives are indicated by the elimination of or reduction in the number of rescue treatments using standard of care, for example, intravitreal injections with anti-VEGF agents, including but not limited to pegaptanib, ranibizumab, aflibercept, or bevacizumab. Clinical objectives are also indicated by a decrease or prevention of vision loss and/or a decrease or prevention of retinal detachment.

Clinical objectives are determined by measuring BCVA (Best-Corrected Visual Acuity), intraocular pressure, slit lamp biomicroscopy, indirect ophthalmoscopy, and/or SD-OCT (SD-Optical Coherence Tomography). In particular, clinical objectives are determined by measuring mean change from baseline in BCVA over time, measuring the gain or loss of ≥15 letters compared to baseline as per BCVA, measuring mean change from baseline in CRT as measured by SD-OCT over time, measuring mean number of ranibizumab rescue injections over time, measuring time to 1st rescue ranibizumab injection, measuring mean change from baseline in CNV and lesion size and leakage area based on FA over time, measuring mean change from baseline in aqueous aVEGF protein over time, performing vector shedding analysis in serum and urine, and/or measuring immunogenicity toVector 1, i.e., measuring Nabs to AAV, measuring binding antibodies to AAV, measuring antibodies to aVEGF, and/or performing ELISpot.

Clinical objectives are also determined by measuring the mean change from baseline over time in area of geographic atrophy per fundus autofluorescence (FAF), measuring the incidence of new area of geographic atrophy by FAF (in subjects with no geographic atrophy at baseline, measuring the proportion of subjects gaining or losing ≥5 and ≥10 letters, respectively, compared with baseline as per BCVA, measuring the proportion of subjects who have a reduction of 50% in rescue injections compared with previous year, measuring the proportion of subjects with no fluid on SD-OCT.

Improvement/efficacy resulting from Vector 1 administration can be assessed as a defined mean change in baseline in visual acuity at about 4 weeks, 12 weeks, 6 months, 12 months, 24 months, 36 months, or at other desired timepoints. Treatment with Vector 1 can result in a 5%, 10%, 15%, 20%, 30%, 40%, 50% or more increase in visual acuity from baseline. Improvements/efficacy can be assessed as mean change from baseline in central retinal thickness (CRT) as measured by spectral domain optical coherence tomography (SD-OCT) at 4 weeks, 12 weeks, 6 months, 12 months, 24 months and 36 months. Treatment with Vector 1 can result in a 5%, 10%, 15%, 20%, 30%, 40%, 50% or more increase central retinal thickness from baseline.

### 6.5 EXAMPLE 14: AAV8-antiVEGFfab for Neovascular AMD

### 6.5.1 Brief Summary of Study

In this study, expression of an anti-human VEGF antibody fragment (antiVEGFfab) after subretinal injection of AAV8-antiVEGFfab is demonstrated. In transgenic mice expressing human VEGF in the retina (rho/VEGF mice), a model for type 3 choroidal neovascularization (NV) in humans, compared to eyes injected with null vector, those injected with ≥1×10⁷ gene copies (GC) of AAV8-antiVEGFfab had significant reduction in mean area of NV. A dose-dependent response was observed with modest reduction of NV with ≤3×10⁷, >50% reduction with ≥1×10⁸ GC, and almost complete elimination of NV with 3×10⁹ or 1×10¹⁰ GC. In Tet/opsin/VEGF mice, in which doxycycline-induced high expression of VEGF leads to severe vascular leakage and exudative retinal detachment (RD), reduction of total RD by 70-80% occurred with 3×10⁹ or 1×10¹⁰ GC of AAV8-antiVEGFfab. These data strongly support initiating clinical trials testing subretinal injection of AAV8-antiVEGFfab in patients with NVAMD.

### 6.5.2 Introduction

Age-related macular degeneration (AMD) is a highly prevalent neurodegenerative disease in which death of photoreceptors and retinal pigmented epithelial (RPE) cells results in gradual loss of central vision. A subgroup of 10-15% of patients with AMD develop subretinal neovascularization (NV) resulting in relatively rapid reduction in visual acuity due to leakage of plasma from incompetent new vessels and collection of fluid within and under the retina which compromises retinal function. This subgroup is said to have neovascular AMD (NVAMD).

Vascular endothelial growth factor (VEGF) plays a central role in the development of subretinal NV and excessive leakage from the NV causing subretinal and/or intraretinal fluid in the macula that decreases vision. Intraocular injections of VEGF-neutralizing proteins reduce leakage allowing fluid reabsorption and improvement in visual acuity (see, e.g., Rosenfeld et al., 2006, Ranibizumab for Neovascular Age-Related Macular Degeneration, N Eng J Med 355: 1419-31; Brown et al., 2006, Ranibizumab Versus Verteporfin for Neovascular Age-Related Macular Degeneration, N Eng J Med 355: 1432-44; each of which is incorporated by reference herein in its entirety); however, the production of VEGF is chronic so that leakage and NV growth recur when vitreous levels of a VEGF-neutralizing protein drop below therapeutic levels. In initial clinical trials, treatment-naïve subjects with recent onset of NVAMD were given monthly intravitreous injections of a VEGF-neutralizing protein and 34-40% of subjects experienced at least a 15 letter improvement in best-corrected visual acuity (BCVA), a large and clinically meaningful benefit, that was maintained for 2 years. In an extension study in which subjects were seen and treated as infrequently as every 3 months, almost all of the visual benefits were lost (see, e.g., Singer et al., 2012, Horizon: An Open-Label Extension Trial of Ranibizumab for Choroidal Neovascularization Secondary to Age-Related Macular Degeneration, Ophthalmology 119: 1175-83; which is incorporated by reference herein in its entirety). In a subsequent study in subjects with NVAMD, the mean improvement from baseline BCVA was significantly greater in those given monthly injections of a VEGF-neutralizing protein compared to those who had monthly visits with injections only when intraretinal or subretinal fluid was present in the macula (see, e.g., Martin et al., 2011, Ranibizumab and Bevacizumab for Neovascular Age-Related Macular Degeneration, N Eng J Med 364: 1897-908; which is incorporated by reference herein in its entirety). Visual benefits were maintained for 2 years with these treatment regimens, after which subjects were treated at the discretion of their physician and after 3 years, visual benefits were lost and mean BCVA in all groups was worse than baseline (see, e.g., Maguire et al., 2016, Five-Year Outcomes with Anti-Vascular Endothelial Growth Factor Treatment of Neovascula Age-Related Macular Degeneration: The Comparison of Age-Related Macular Degeneration Treatment Trials, Ophthalmology 123: 1751-61; which is incorporated by reference herein in its entirety). Thus, frequent injections with sustained suppression of VEGF are required in patients with NVAMD to maximize and maintain visual benefits.

One strategy to provide long-term benefits in patients with NVAMD is ocular gene transfer to continuously express an anti-angiogenic protein within the retina. This approach strongly suppresses retinal or subretinal NV in animal models (see, e.g., Honda et al., 2000, Experimental Subretinal Neovascularization is Inhibited by Adenovirus-Mediated Soluble VEGF.flt-1 Receptor Gene Rransfection, a Role of VEGF and Possible Treatment for SRN in Age-Related Macular Degeneration, Gene Ther 7: 978-85; Mori et al., 2001, Pigment Epithelium-Derived Factor Inhibits Retinal and Choroidal Neovascularization, J Cell Physiol 188: 253-63; Lai et al., 2001, Suppression of Choroidal Neovascularization by Adeno-Associated Virus Vector Expressing Angiostatin, Invest Ophthalmol Vis Sci 42: 2401-7; Mori et al., 2002, AAV-Mediated Gene Transfer of Pigment Epithelium-Derived Factor Inhibits Choroidal Neovascularization, Invest Ophthalmol Vis Sci 43: 1994-2000; Bainbridge et al., 2002, Inhibition of Retinal Neovascularization by Gene Transfer of Soluble VEGF Receptor sFlt-1, Gene Ther 9: 320-6; Takahashi et al., 2003, Intraocular Expression of Endostatin Reduces VEGF-Induced Retinal Vascular Permeability, Neovascularization, and Retinal Detachment, FASEB J 17: 896-8; Gehlbach et al., 2003, Periocular Injection of an Adenoviral Vector Encoding Pigment Epithelium-Derived Factor Inhibits Choroidal Neovascularization, Gene Ther 10: 637-46; Rota et al., 2004, Marked Inhibition of Retinal Neovascularization in Rats Following Soluble-flt-1 Gene Transfer, J Gene Med 6: 992-1002; Lai et al., 2005, Long-Term Evaluation of AAV-Mediated sFlt-1 Gene Therapy for Ocular Neovascularization in Mice and Monkeys, Mol Ther 12: 659-68; each of which is incorporated by reference herein in its entirety). In subjects with advanced NVAMD, intravitreous injection of an adenoviral vector expressing pigment epithelium-derived factor caused reabsorption of subretinal hemorrhage and fluid in several patients with advanced NVAMD providing proof of concept for this approach (see, e.g., Campochiaro, et al., 2006, Adenoviral Vector-Delivered Pigment Epithelium-Derived Factor For Neovascular Age-Related Macular Degeneration, Results of a Phase I Clinical Trial, Hum Gene Ther 17: 167-76; which is incorporated by reference herein in its entirety). AAV vectors are an appealing platform because they provide long term transgene expression. A recent phase 1 clinical trial in subjects with advanced NVAMD tested intravitreous injection of an AAV2 vector containing a chicken beta actin (CBA) promoter driving expression of a VEGF neutralizing protein consisting of domain 2 of Flt-1 (VEGFR1) linked by a polyglycine 9-mer to human IgG1-Fc (sFLT01) (see, e.g., Heier, et al., 2017, Intravitreous Injection of AAV2-sFIT01 in Patients with Advanced Neovascular Age-Related Macular Degeneration, a Phase 1, Open-Label Trial, The Lancet 389: May 17; which is incorporated by reference herein in its entirety). Transgene expression was detected in 5 of 10 eyes injected with the highest dose, 2×10¹⁰ genome copies (GC), and none of the eyes with <2×10¹⁰ GC. In 11 of 19 patients with intraretinal or subretinal fluid at baseline judged to be reversible, six showed substantial fluid reduction and improvement in vision, whereas five showed no fluid reduction. Thus while there was some evidence of biologic activity, there was considerable heterogeneity among patients with regard to response. In general, compared with intravitreous injections of AAV2 vectors, subretinal injections provide substantially higher transgene expression and a phase 1 trial testing subretinal injection of an AAV2 vector in which the cytomegalovirus (CMV) promoter drives expression of native soluble VEGFR1 (AAV2-sFlt-1) in subjects with NVAMD showed good safety and 3 of 6 subjects given a subretinal injection of 1×10¹⁰ or 1×10¹¹ GC of AAV2.sFlt-1 showed some reduction of intraretinal fluid (see, e.g., Rakoczy et al., 2015, Gene Therapy with Recombinant Adeno-Associated Vectors for Neovascular Age-Related Macular Degeneration, 1 year Follow-Up of a Phase 1 Randomized Clinical Trial, Lancet 386: 2395-403; which is incorporated by reference herein in its entirety). In a phase 2 trial of 32 subjects with NVAMD, 21 were randomized to subretinal injection of 100 μl containing 1×10¹¹ GC of AAV2.sFlt-1 and 11 were randomized to ranibizumab injections only as needed for recurrent intraretinal or subretinal fluid (see, e.g., Constable, et al., 2016, Phase 2a Randomized Clinical Trial: Safety and Post Hoc Analysis of Subretinal rAAV.sFLT-1 for Wet Age-Related Macular Degeneration, EBioMedicine 14: 168-75; which is incorporated by reference herein in its entirety). All subjects received ranibizumab injections at baseline and week 4 and thereafter according to prespecified criteria. The study failed to demonstrate sufficient efficacy to continue development of AAV2.sFlt-1. Levels of sFlt-1 were not reported and therefore lack of sufficient sFlt-1 expression cannot be ruled out as a potential factor in the lack of response seen in the study.

Screening of tissues from rhesus monkeys by PCR for sequence homologies to known AAV serotypes led to the identification of AAV7 and AAV8 (see, e.g., Gao et al., 2002, Novel Adeno-Associated Viruses From Rhesus Monkeys as Vectors for Human Gene Therapy, Proc Natl Acad Sci USA 99: 11854-9; which is incorporated by reference herein in its entirety). Vectors with AAV8 capsids were generated and in liver, transgene expression was 10-100 fold higher than with AAV vectors with a capsid of another serotype such as AAV2. Neutralizing antibodies to AAV8 were rare in human serum and antibodies to other AAV serotypes did not reduce AAV8 vector-mediated expression (see, e.g., Gao et al., 2002, Novel Adeno-Associated Viruses From Rhesus Monkeys as Vectors for Human Gene Therapy, Proc Natl Acad Sci USA 99: 11854-9). AAV2 and AAV8 both transduce RPE cells after subretinal injection of moderate or low doses, but AAV8 is more efficient at transducing photoreceptors and therefore results in overall higher expression levels (Vandenberghe et al., 2011, Dosage Thresholds for AAV2 and AAV8 Photorecepotr Gene Therapy in Monkey, Sci Trans Med 3: 1-9; which is incorporated by reference herein in its entirety). In this study, transgenic mouse models were used in which human VEGF165 is expressed in photoreceptors to test the efficacy of subretinal injection of a wide range of doses of an AAV8 vector containing an expression cassette for a humanized antibody fragment that binds human VEGF.

### 6.5.3 Materials and Methods

Construction of AAV8-antiVEGFfab.

AAV8-antiVEGFfab is a non-replicating AAV8 vector containing a gene cassette encoding a humanized monoclonal antigen binding fragment that binds and inhibits human VEGF, flanked by AAV2 inverted terminal repeats (ITRs). Expression of heavy and light chains is controlled by the CB7 promoter consisting of the chicken β-actin promoter and CMV enhancer, a chicken β-actin intron, and a rabbit β-globin poly A signal. The nucleic acid sequences coding for the heavy and light chains of anti VEGFfab are separated by a self-cleaving furin (F)/F2A linker. The expressed protein product is similar, but not identical, to ranibizumab. Due to the mechanism of furin-mediated cleavage, the vector-expressed antiVEGFfab may contain none, one, or more additional amino acid residues in the last position of the heavy chain in addition to all the amino acids normally found in ranibizumab.

### Mice.

All mice were treated in accordance with the Association for Research in Vision and Ophthalmology Statement for Use of Animals in Ophthalmic and Vision Research and protocols were reviewed and approved by the Johns Hopkins University Animal Care and Use Committee. Transgenic mice in which the rhodopsin promoter drives expression of VEGF165 in photoreceptors (rho VEGF mice) and double transgenic mice with inducible expression of VEGF in photoreceptors (Tet opsin VEGF mice) have been previously described. All transgenic mice were in C57BL/6 background and were genotyped to confirm the presence of transgenes prior to use in experiments. Wild type C57BL/6 mice were purchased from Charles River (Frederick, MD, USA).

Subretinal Injections of Vector.

Mice were anesthetized and eyes were visualized with a Zeiss Stereo Dissecting Microscope. A 30-gauge needle on an insulin syringe was used to create a small partial thickness opening in the sclera and a 33-gauge needle on a Hamilton syringe was inserted into the scleral puncture and slowly advanced through the remaining scleral fibers into the subretinal space and 1 μL of vehicle containing AAV8-antiVEGFfab or empty AAV8 vector was injected. A cotton swab was applied to the injection site as the needle was removed to prevent reflux.

Expression of antiVEGFfab Protein in the Retina.

Wild type C57BL/6 mice received a single subretinal injection in each eye of 1×10⁸, 3×10⁸, 1×10⁹, 3×10⁹, 1×10¹⁰ GC of AAV8-antiVEGFfab or 1×10¹⁰ GC of empty vector. Fourteen days after injection, mice were euthanized, eyes were removed and frozen. Eyes were homogenized in 200 μl RIPA buffer using Qiagen Tissue Lyser. The concentration of anti VEGFfab protein was measured by ELISA using known concentrations of ranibizumab to generate a standard curve. Briefly, ELISA plates were coated with 1 μg/mL of human VEGF 165 overnight at 4° C. Wells were blocked with 200 uI. 1% BSA for 1 hour at room temperature. Samples were diluted 1:80 and 100 μL was added to duplicate wells, incubated for 1 hour at 37° C., and followed by a second blocking buffer incubation. After washing, wells were incubated for 1 hour at room temperature in 100 μL of a cocktail of 1 mg/mL goat anti-human IgG heavy chain and 0.5 mg/mL goat anti-human IgG light chain, both labeled with biotin and pre-absorbed. After washing, wells were incubated for 1 hour at room temperature in 100 μL of a 1:30,000 dilution of Streptavidin-HRP, washed, and incubated in 150 μL TMB detection solution consisting of 0.1M NaOAc citrate buffer (pH 6.0), 30% hydrogen peroxide, 3,3′,5,5′-tetramethylbenzidine ≥99% at room temperature in the dark for 30 minutes after which 50 μL of stop solution (2N H₂SO₄) was added to each well and the plate was read at 450 nm-540 nm.

Assessment of Effects on Type 3 Choroidal NV in Rho/VEGF Mice.

At postnatal day (P) 14, rho/VEGF mice were given a single subretinal injection in one eye of 3×10⁶, 1×10⁷, 3×10⁷, 1×10⁸, 3×10⁸, 1×10⁹, 3×10⁹, 1×10¹⁰ GC of AAV8-anti VEGFfab or 1×10¹⁰ GC of empty vector or PBS. At P21, mice were euthanized, eyes removed, retinas were dissected intact, stained with FITC-conjugated GSA lectin (Vector Laboratories, Burlingame, CA), and flat-mounted with photoreceptor side facing up. Fluorescent images were obtained with a Zeiss Axioskop fluorescence microscope and the area of type 3 choroidal NV per retina was measured by image analysis using ImagePro Plus software with the investigator masked with regard to treatment group.

Assessment of Effects on Severe VEGF-Induced Vascular Leakage.

Ten week old Tet opsin VEGF mice were given a single subretinal injection in one eye of 1×10⁸, 3×10⁸, 1×10⁹, 3×10⁹, 1×10¹⁰ GC of AAV8-antiVEGFfab or 1×10¹⁰ GC of empty vector or PBS. Ten days after injection, 2 mg/mL of doxycycline was added to drinking water and after 4 days, mice were anesthetized, pupils dilated, and fundus photographs were obtained with a Micron III Retinal Imaging Microscope. Images were examined by a masked investigator and were determined to show no, partial, or total exudative retinal detachment. The total retinal area and area of detached retina were measured by image analysis using ImagePro Plus software with the investigator masked with regard to treatment group. The percentage retinal detachment was calculated as area of detached retina/total retina. In a small number of eyes sharp fundus images could not be obtained due to lack of cornea or lens clarity and in those cases, mice were euthanized, eyes were removed and frozen, and 10 μm serial sections were cut. Sections were post-fixed in 4% paraformaldehyde, stained with Hoechst and examined by light microscopy to determine the presence and extent of exudative retinal detachment.

To investigate long term effects, Tet opsin VEGF mice were given a single subretinal injection of 3×10⁹ GC of AAV8-antiVEGFfab or 3×10⁹ GC of empty vector in one eye. The fellow eye served as an untreated control. One month after injection, 2 mg/mL of doxycycline was added to drinking water and after 4 days, fundus photographs were obtained and graded as described above.

### Statistical Comparisons

Student's t-tests were carried out to compare the outcome measures between two experiment groups. For comparisons among three or more experiment groups, one-way analysis of variance (ANOVA) adjusting for multiple comparison using Bonferroni multiple-comparison correction were performed. For comparing the types of detachment between the groups with different doses versus the empty vector group, the p values were calculated using the Fisher's exact tests. All statistical tests were conducted at 5% statistical significance. Statistical analyses were performed using Stata version 14.2 (College Station, Texas 77845).

### 6.5.4 Results

Selection of VEGF neutralizing protein.

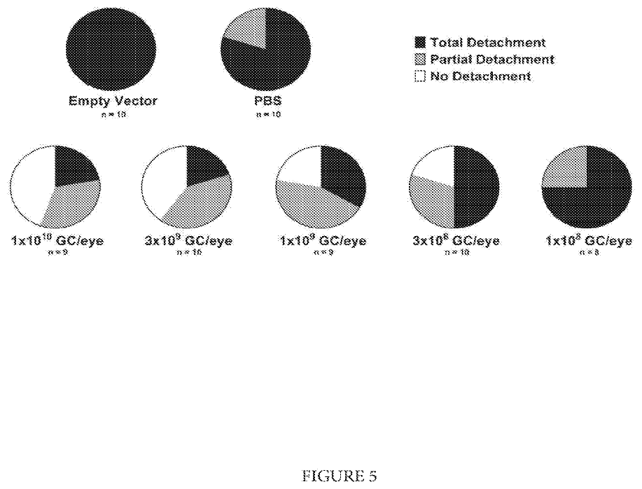

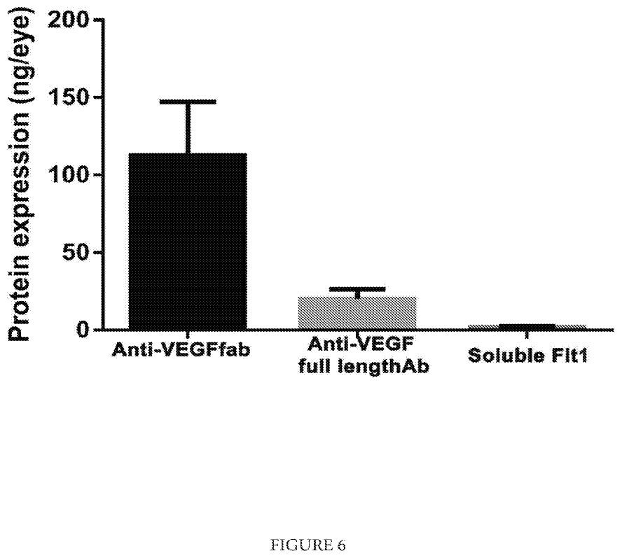

In initial experiments, cDNAs were generated for a full length anti-VEGF antibody, an anti-VEGF antibody fragment (anti-VEGFfab), and soluble VEGF receptor-1 (sFlt-1) and they were inserted into an expression cassette containing a CMV promoter and packaged in AAV8. Fourteen days after subretinal injection of 3×10⁹ GC of each vector, the total amount of each of the transgenes per eye was measured by ELISA. Eyes injected with AAV8-CMV.anti-VEGFfab had high levels of anti-VEGFfab protein, while those injected with AAV8-CMV.anti-VEGF full lenth Ab had relatively low levels of full length anti-VEGF Ab and eyes injected with AAV8-CMV.sFlt1 had very low or undetectable levels of sFlt1 (FIG. 6). Therefore Anti VEGFfab was selected as the anti-VEGF neutralizing protein for subsequent experiments.

Construction and in vitro testing of AAV8-antiVEGFfab.

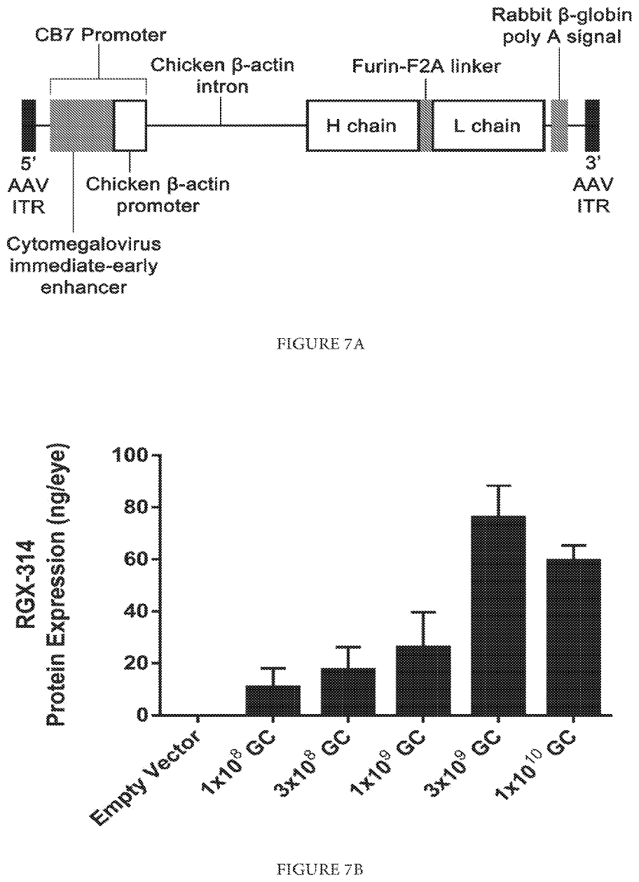

The cDNA for anti-VEGFfab was inserted into an expression cassette containing a CB7 promoter. A schematic of the genome of AAV8-anti VEGFfab (RGX-314) is shown in FIG. 7A. The CB7 promoter drives expression of the heavy and light chain of anti VEGFfab and a Furin-F2A linker resulting in post-translational assembly of antiVEGFfab.

Transgene expression after subretinal injection of AAV8-antiVEGFfab in mice.

One week after subretinal injection of 1 μl of doses ranging from 1×10⁸-1×10¹⁰ GC of AAV8-anti VEGFfab the level of anti VEGFfab protein was measured in eye homogenates. As vector dose was increased, there was an increase in antiVEGFfab with peak levels obtained at doses of 3×10⁹ and 1×10¹⁰ GC (FIG. 7B).

Subretinal injection of AAV8-antiVEGFfab suppression of subretinal NV in rho VEGF mice.

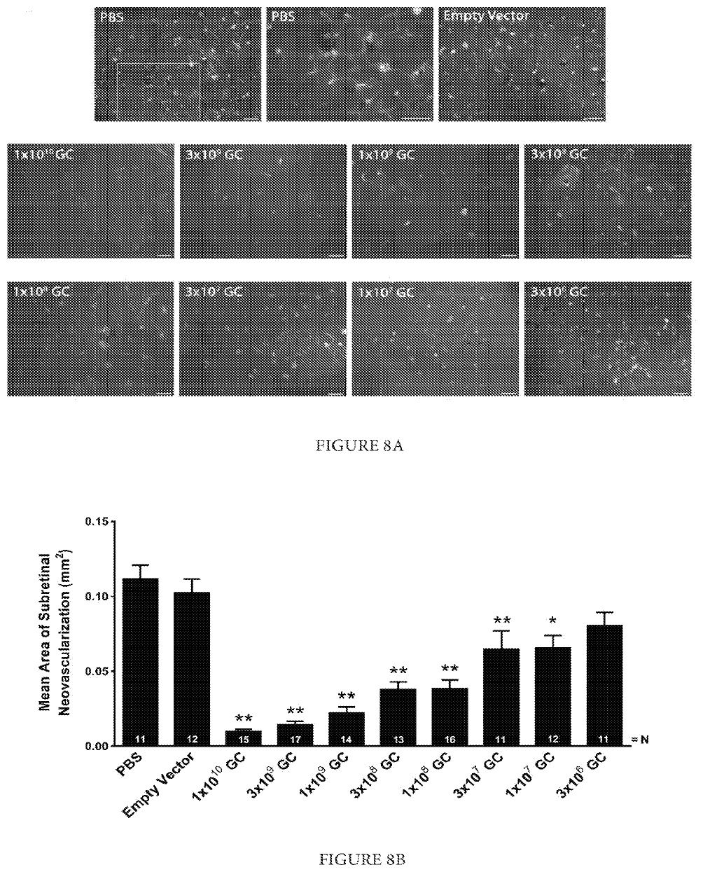

Transgenic mice in which the rhodopsin promoter drives expression of human VEGF 165 sprout new vessels from the deep capillary bed starting around postnatal day (P) 14 and have extensive subretinal NV by P21 (see e.g., Okamoto et al., 1998, Evolution of Neovascularization in Mice with Overexpression of Vascular Endothelial Growth Factor in Photoreceptors, Invest Ophthalmol Vis Sci 39: 180-8; Tobe et al., 1998, Evolution of Neovascularization in Mice with Overexpression of Vascular Endothelial Growth Factor in Photoreceptors, Invest Ophthalmol Vis Sci 39: 180-8; each of which is incorporated by reference herein in its entirety). These mice provide a model for retinal angiomatous proliferation also known as type 3 choroidal NV in humans (see e.g., Yannuzzi, et al., 2001, Retinal Angiomatous Proliferation in Age-Related Macular Degeneration, Retina 21: 416-34; which is incorporated by reference herein in its entirety). After subretinal injection of PBS at P14, retinas of rho/VEGF mice stained with FITC-labeled GSA lectin which selectively stains vascular cells and flat mounted with the photoreceptor side facing upward showed numerous hyperfluorescent spots (FIG. 8A, top row, left column). At higher magnification, feeder vessels were seen extending from the deep capillary bed in the background to the buds of subretinal NV partially surrounded by dark black retinal pigmented epithelial cells (FIG. 8B, top row, middle column). Mice injected with 1×10¹⁰ GC of empty AAV8 vector at P14 showed comparable amounts of subretinal NV at P21 as that seen in PBS-injected eyes (FIG. 8A, top row, right column). Rho VEGF mice given a subretinal injection of 1×10¹⁰, 3×10⁹, or 1×10⁹ GC of AAV8-antiVEGFfab showed very little subretinal NV at P21 (FIG. 8A, middle row), while those injected with 3×10⁸ or 1×10⁸ GC (FIG. 8A, middle and bottom rows) showed somewhat more, but still considerably less than mice injected with empty vector. An intermediate amount of NV was seen in mice injected with 3×10⁷ and 1×10⁷ GC and those injected with 3×10⁶ GC appeared similar to those injected with empty vector (FIG. 8A, bottom row).

Now referring to FIG. 8B, measurement of the mean area of NV per retina by image analysis showed a dose-response that paralleled what visual inspection of the retinal flat mounts suggested with mean area of NV per retina significantly less in eyes injected with doses of AAV8-antiVEGFfab between 1×10¹⁰ and 1×10⁷ GC than in empty vector-injected eyes.

Subretinal injection of AAV8-antiVEGFfab blocks VEGF-induced vascular leakage.

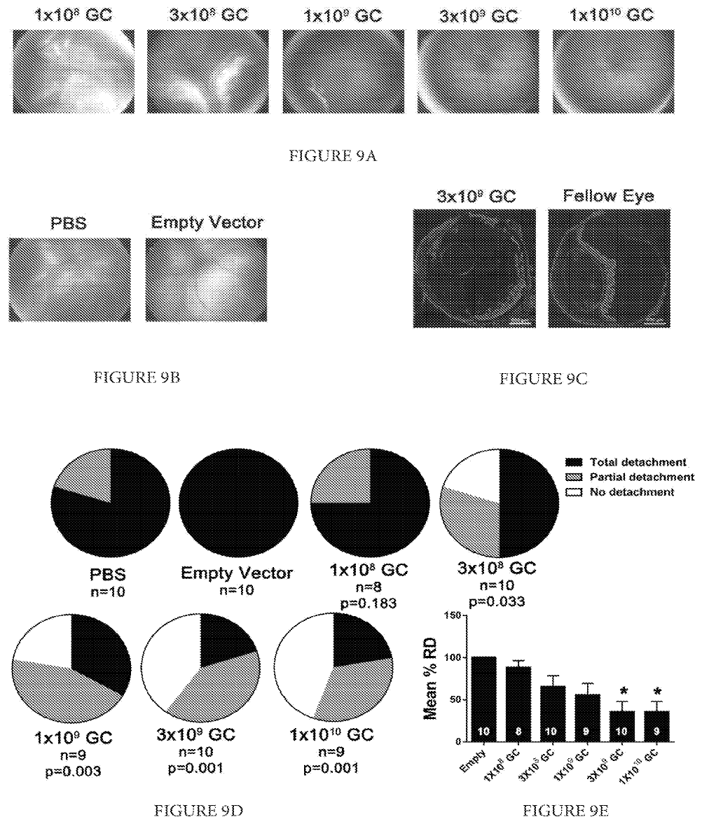

Tet/opsin VEGF double transgenic mice in which the tet-on system and the rhodopsin promoter provide doxycycline-inducible expression of VEGF₁₆₅ at levels 10-fold higher than those present in the retinas of rho VEGF mice resulting in exudative retinal detachment within 4 days of starting doxycycline 2 mg/ml in drinking water (see e.g., Ohno-Matsui et al., 2002, Inducible Expression of Vascular Endothelial Growth Factor in Photoreceptors of Adult Mice Causes Severe Proliferative Retinopathy and Retinal Detachment, Am J Pathol 160: 711-9; which is incorporated by reference herein in its entirety). Ten days after subretinal injection of 1×10⁸ or 3×10⁸ GC of AAV8-anti VEGFfab, 75% and 50% developed total exudative retinal detachments 4 days after starting 2 mg/ml doxycycline in drinking water (FIG. 9A, left two panels) similar to those seen in doxycycline-treated Tet opsin VEGF mice that had been given a subretinal injection of PBS or 1×10¹⁰ GC of empty vector (FIG. 9B). FIG. 9C shows an ocular section from an eye injected with 3×10⁹ GC of AAV8-anti VEGFfab showing no exudative retinal detachment and an uninjected fellow eye with a total retinal detachment. Ten of 10 eyes given a subretinal injection of empty vector had a total retinal detachment 4 days after starting doxycycline, and 10 of 10 eyes given a subretinal injection of 1×10¹⁰ GC of empty vector had total (7 eyes) or partial (3 eyes) retinal detachment (FIG. 9D). Compared with mice injected with empty vector, the percentage of eyes with total retinal detachment was significantly less in doxycycline-treated Tet/opsin/VEGF mice that had been given a subretinal injection of 3×10⁸ (50% less), 1×10⁹ (67% less), 3×10⁹ (80% less), or 1×10¹⁰ (78% less) GC of AAV8-antiVEGFfab. The percentage of the retina that was detached was measured by image analysis and compared with eyes injected with empty vector, the mean percentage detachment was significantly less in eyes injected with 3×10⁹ GC or 1×10¹⁰ GC of AAV8-antiVEGFfab (FIG. 9E).

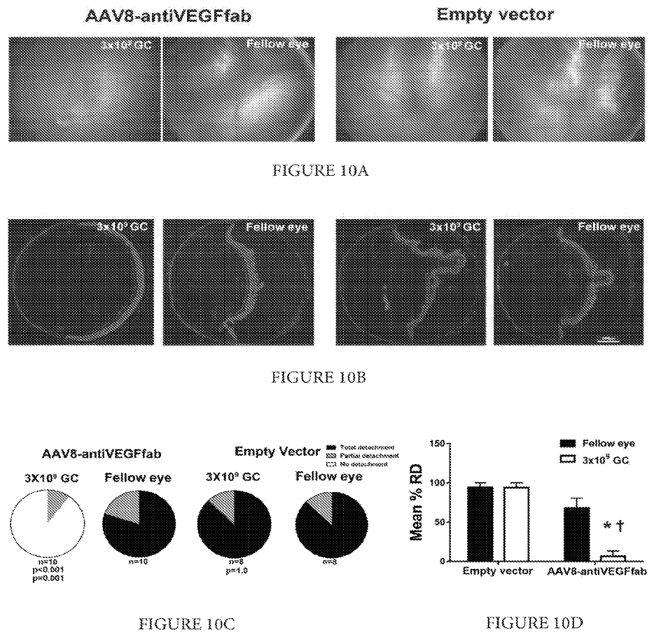

To assess long term effects, Tet/opsin/VEGF mice were treated with 2 mg/ml doxycycline 1 month after subretinal injection of 3×10⁹ GC of AAV8-antiVEGFfab or empty vector in one eye. Representative mice showed no detachment in the AAV8-antiVEGFfab-injected eye and total detachment in the fellow eye (FIG. 10A, left side), and total detachment in both the empty vector-injected eye and fellow eye (FIG. 10A, right side). In other mice from the 2 groups, Hoecht-stained ocular sections showed no detachment in an AAV8-antiVEGFfab-injected eye and total detachment in the fellow eye, and total detachment in an empty vector-injected eye and fellow eye (FIG. 10B). Nine of 10 eyes injected with AAV8-antiVEGFfab had no retinal detachment which was significantly different from uninjected fellow eyes in the same mice for which 8 eyes had total detachment and 2 eyes had partial detachment (FIG. 10C). Compared with 8 eyes injected with empty vector in which there were 7 total and 1 partial detachments, those injected with AAV8-antiVEGFfab showed significantly fewer detachments. Also, the mean percentage retinal detachment in eyes injected with AAV8-antiVEGFfab was significantly less than that in uninjected fellow eyes in the same mice or eyes injected with empty vector (FIG. 10D).

### 6.5.5 Discussion

Sustained suppression of VEGF is needed in most patients with NVAMD to maximally improve visual acuity and prevent disease progression and vision loss over time. Several strategies designed to achieve this goal have been tested. Surgical implantation of a refillable reservoir that slowly releases a VEGF-neutralizing protein into the eye is evaluated in a phase 2 clinical trial in patients with NVAMD (Study of the Efficacy and Safety of the Ranibizumab Port Delivery System for Sustained Delivery of Ranibizumab in Participants With Subfoveal Neovascular Age-Related Macular Degeneration, ClinicalTrials.gov identifier: NCT02510794). Another approach is to incorporate a small molecule inhibitor of HIF-1, which suppresses expression of VEGF, into a biodegradable polymer, formulate microparticles that allow sustained release of the inhibitor, and inject them into the eye (see e.g., Iwase et al., 2013, Sustained Delivery of a HIF-1 Antagonist for Ocular Neovascularization, J Control Release 172: 625-33; which is incorporated by reference herein in its entirety). A third approach is ocular gene transfer to express a VEGF-neutralizing protein or other antiangiogenic protein in the eye and, while clinical trials have shown some encouraging signals (see, e.g., Campochiaro, et al., 2006, Adenoviral Vector-Delivered Pigment Epithelium-Derived Factor For Neovascular Age-Related Macular Degeneration, Results of a Phase I Clinical Trial, Hum Gene Ther 17: 167-76; Heier, et al., 2017, Intravitreous Injection of AAV2-sFLTO1 in Patients with Advanced Neovascular Age-Related Macular Degeneration, a Phase 1, Open-Label Trial, The Lancet 389: May 17; Rakoczy et al., 2015, Gene Therapy with Recombinant Adeno-Associated Vectors for Neovascular Age-Related Macular Degeneration, 1 year Follow-Up of a Phase 1 Randomized Clinical Trial, Lancet 386: 2395-403; Constable, et al., 2016, Phase 2a Randomized Clinical Trial: Safety and Post Hoc Analysis of Subretinal rAAV.sFLT-1 for Wet Age-Related Macular Degeneration, EBioMedicine 14: 168-75), demonstration of consistent, sustained transgene expression with strong antipermeability and antiangiogenic activity is lacking.

In this study, it was found that subretinal injection of 1×10⁹ to 1×10¹⁰ GC of AAV8-anti VEGFfab strongly suppressed type 3 choroidal NV in rho/VEGF mice. The minimally effective subretinal dose of AAV8-anti VEGFfab which significantly reduced mean area of subretinal NV per retina compared with subretinal injection of empty vector was 1×10⁷ GC. In Tet/opsin/VEGF double transgenic mice with doxycycline-inducible expression of at least 10-fold higher doses of VEGF compared with that in rho/VEGF mice, 10 days after subretinal injection of AAV8-antiVEGFfab doses as low as 3×10⁸ GC, there was significant reduction in the incidence and severity of exudative retinal detachments. Leakage suppression was particularly good 10 days after injection of 3×10⁹ or 1×10¹⁰ GC which showed a significant reduction in mean percentage retinal detachment, and effect that lasted at least 1 month, the longest time point examined. Most eyes injected with 1×10⁸ GC or greater had detectable levels of anti VEGFfab with peak levels of 60-80 ng per eye after injection of 3×10⁹ or 1×10¹⁰ GC

These data show that subretinal injection of AAV8-antiVEGFfab may help to overcome some of the problems encountered in prior gene transfer clinical trials for NVAMD. In the AAV2-sFLT01 study, aqueous humor samples were assayed for sFLT01 and all samples from subjects injected with 2×10⁸, 2×10⁹, or 6×10⁹ GC were below the lower limit of detection at all time points, but 5 of 10 subjects injected with 2×10¹⁰ GC had detectable levels that peaked at 32.7 to 112.0 ng/ml (mean 73.7 ng/ml) by week 26 with a slight decrease to a mean of 53.2 ng/ml at week 52 (see, e.g., Heier, et al., 2017, Intravitreous Injection of AAV2-sFLT01 in Patients with Advanced Neovascular Age-Related Macular Degeneration, a Phase 1, Open-Label Trial, The Lancet 389: May 17). Pre-existent neutralizing antibodies have been found to neutralize intravitreal gene therapy in nonhuman primates (see e.g., Kotterman et al., 2014, Antibody Neutralization Poses a Barrier to Intravitreal Adeno-Associated Viral Vector Gene Delivery to Non-Human Primates, Gene Ther 22: 116-26; which is incorporated by reference herein in its entirety) and anti-AAV2 serum antibodies might explain this variability in expression in this trial. Four of the 5 subjects with detectable sFI.TO1 levels were negative for anti-AAV2 antibodies at baseline and the fifth had a 1:100 titer, whereas 4 of the 5 high-dose subjects with undetectable sFLT01 levels had titers ≥1:400. The incidence of anti-AAV8 serum antibodies in the general population is far less than the incidence of anti-AAV2 antibodies (see e.g., Calcedo, et al., 2009, Worldwide Epidemiology of Neutralizing Antibodies to Adeno-Associated Viruses, J Infectious Dis 199: 381-90; which is incorporated by reference herein in its entirety). In addition, subretinal injection of AAV8 is likely to provide an additional factor that may mitigate layer of protection from vector inactivation, because anti-AAV2 serum antibodies do not appear to prevent transgene expression after subretinal injections of AAV2 vectors like they do with intravitreal delivery (see e.g., Kotterman et al., 2014, Antibody Neutralization Poses a Barrier to Intravitreal Adeno-Associated Viral Vector Gene Delivery to Non-Human Primates, Gene Ther 22: 116-26). Also, transgene expression is substantially higher after subretinal versus intravitreous injection of AAV vectors, and at equivalent doses, transgene expression is greater with subretinal injection of AAV8 versus AAV2 vectors (see e.g., Okamoto et al., 1998, Evolution of Neovascularization in Mice with Overexpression of Vascular Endothelial Growth Factor in Photoreceptors, Invest Ophthalmol Vis Sci 39: 180-8).

### 6.6 EXAMPLE 15: Intact Mass Analysis, Gel-Based Peptide Mapping and Solution-Based Peptide Mapping on Sample Control and Sample Retinal Cell Line

### 6.6.1 Samples

| | Expression | Heavy Chain C- |
|---|---|---|
| Identifier | Species | terminus |
| Control | E. Coli | L |
| Retnal Cell Line | Human | LRKRR |

### 6.6.2 Objective

Intact mass analysis and gel-based peptide mapping was performed on both samples. Solution-based peptide mapping was performed on Control. Target sequences (SEQ ID NO. 38 and SEQ ID NO. 39) are illustrated in FIG. 11.

### 6.6.3 Experimental Methods

(a) Sample Preparation

Peptide Mapping—Gel

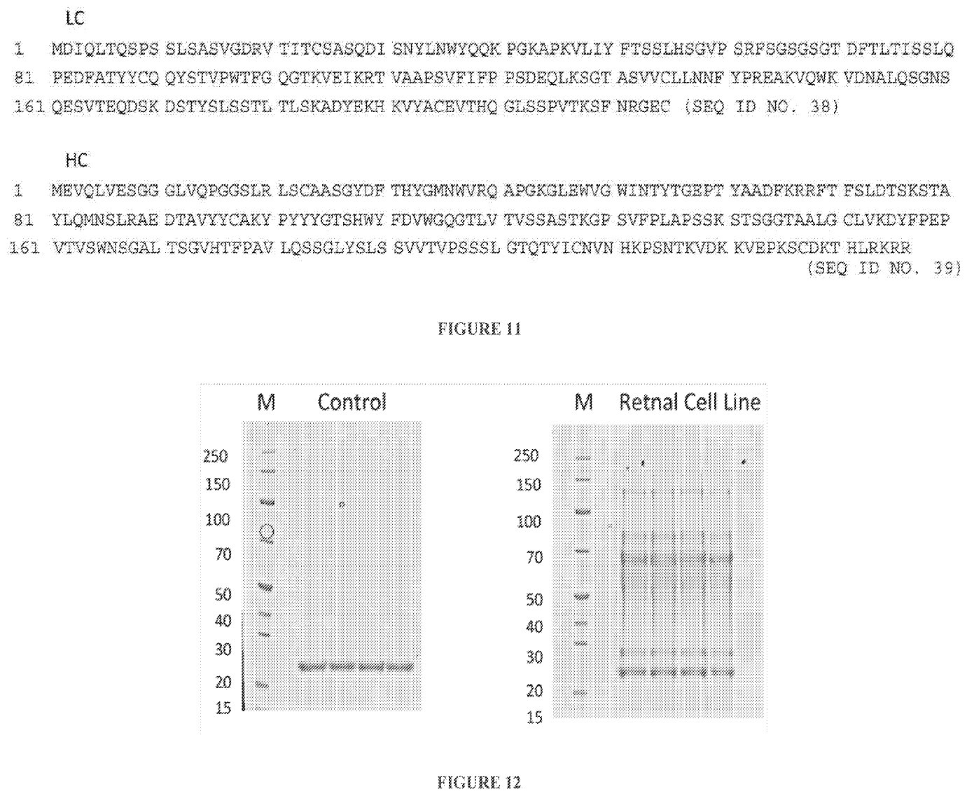

4×2.5 μg of samples Control and Retinal Cell Line were separated using SDS-PAGE. The bands at ˜25 kD were excised as illustrated in FIG. 12.

Trypsin digestion was performed using a robot (ProGest, DigiLab) with the following protocol:
Washed with 25 mM ammonium bicarbonate followed by acetonitrile.
Reduced with 10 mM dithiothreitol at 60° C. followed by alkylation with 50 mM iodoacetamide at RT.
Digested with trypsin (Promega) at 37° C. for 4h.
Quenched with formic acid and the supernatant was analyzed directly without further processing

Chymotrypsin and elastase digestion was performed manually with the following protocol:
Washed with 25 mM ammonium bicarbonate followed by acetonitrile.
Reduced with 10 mM dithiothreitol at 60° C. followed by alkylation with 50 mM iodoacetamide at RT.
Digested with chymotrypsin/elastase (Promega) at 37° C. for 12h.
Quenched with formic acid and the supernatant was analyzed directly without further processing.

Peptide Mapping—Solution

The balance of Control was subjected to TCA precipitation, washed, and resuspended in 55 μL of 8M Urea, 50 mM Tris HCl, pH 8.0. The TCA precipitated sample was quantified by Qubit fluorometry which reported the following value:

$$0.78 μg / μL \times 50 μL = 39 μg recovery .$$

10 μg of the sample was aliquoted in triplicate. Each triplicate was reduced in 11 mM DTT for 1h at RT, alkylated in 12 mM Iodoacetamide for 1h, and digested with trypsin, chymotrypsin, and elastase at 37C overnight. Samples were subjected to SPE on an Empore C18 SD plate.

(b) Mass Spectrometry

Peptide Mapping

The gel digests were analyzed by nano LC/MS/MS with a Waters NanoAcquity HPLC system interfaced to a ThermoFisher Q Exactive. Peptides were loaded on a trapping column and eluted over a 75 μm analytical column at 350 nL/min; both columns were packed with Luna C18 resin (Phenomenex). The mass spectrometer was operated in data-dependent mode, with MS and MS/MS performed in the Orbitrap at 70,000 FWHM and 17,500 FWHM resolution, respectively. The fifteen most abundant ions were selected for MS/MS.

Intact Mass

10 pmol was analyzed by LC/MS using a C4 column (Waters Symmetry C4 3.5 μm, 2.1 mm×50 mm) interfaced to a Q Exactive mass spectrometer. Data was acquired from m/z 600-2500 at resolution 17,500 FWHM (at m/z 400) with three microscans per spectrum.

(c) Data Processing

Peptide Mapping

Data were searched using a local copy of Mascot with the following parameters:
Enzyme: Semi-Trypsin or None (for elastase and chymotrypsin)
Database: Swissprot Human (forward and reverse appended with common contaminants and target sequence)
Fixed modification: Carbamidomethyl (C)
Variable modifications: Oxidation (M), Acetyl (Protein N-term), Deamidation (NQ), Pyro Glu (N-term E)
Mass values: Monoisotopic
Peptide Mass Tolerance: 10 ppm
Fragment Mass Tolerance: 0.02 Da
Max Missed Cleavages: 2

Mascot DAT files were parsed into the Scaffold software for validation, filtering and to create a nonredundant list per sample. Data were filtered using a minimum protein value of 99%, a minimum peptide value of 50% (Prophet scores) and requiring at least two unique peptides per protein.

Intact Mass

Intact mass data were processed using the MagTran v1.03 software.

### 6.6.4 Results

(a) Sample Control

Peptide Mapping—Gel

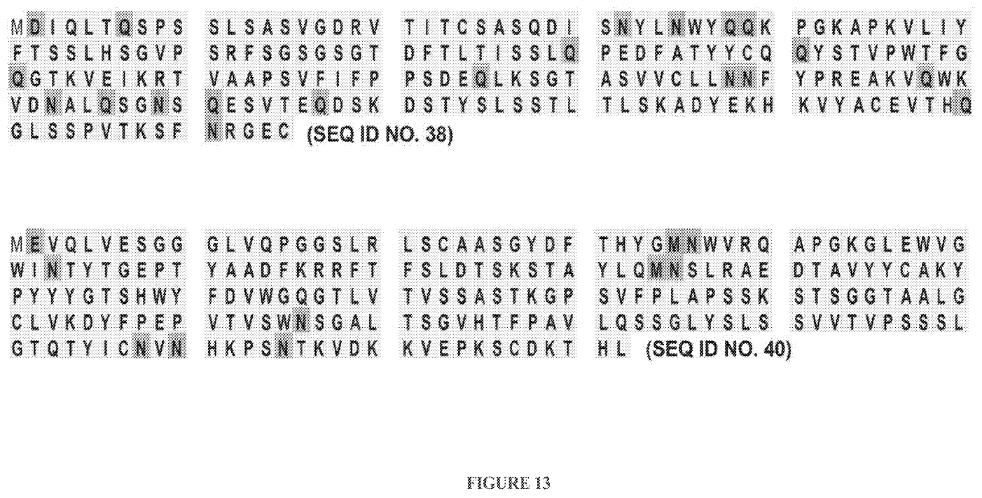

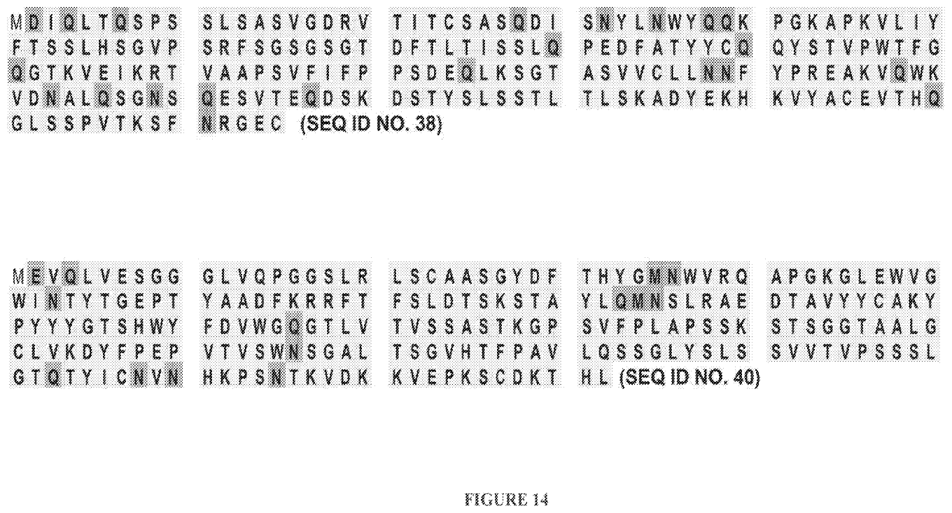

Data matched to both sequences (SEQ ID NO. 38 and SEQ ID NO. 40) as illustrated in FIG. 13.

Light chain (“LC”)—1430 total spectra, 552 unique peptides and 100% sequence coverage. Note there was no evidence of N-terminal Met. The N-terminus is present as both free amine and N-acetylated. There were 7 N-acetylated spectra and 40 non-acetylated spectra.

Heavy chain (“HC”)—1069 total spectra, 470 unique peptides and 100% sequence coverage. Note there was no evidence of N-terminal Met. The N-terminus is present as both free amine and N-acetylated. There were 7 N-acetylated spectra and 56 non-acetylated spectra.

HC C-Terminal Leu Cleavage—Peptides were detected corresponding to the H231 C-terminus and L232 C-terminus. The extracted ion chromatogram peak areas for two representative peptides are shown below along with the relative percentage. Note there is potential for error in this measurement based on difference in response factors for the two peptides:

| | | Peak | |
|---|---|---|---|
| Peptide | m/z | Area | % |
| ScDKTH | 374.1574 | 1.93E+07 | 0.01% |
| ScDKTHL | 430.7007 | 3.14E+11 | 99.99% |

### Peptide Mapping—Solution

Data matched to both sequences (SEQ ID NO. 38 and SEQ ID NO. 40) as illustrated in FIG. 14.

LC—1453 total spectra, 489 unique peptides and 100% sequence coverage. Note there was no evidence of N-terminal Met. The N-terminus is present as both free amine and N-acetylated. There were 3 N-acetylated spectra and 48 non-acetylated spectra.

HC—985 total spectra, 423 unique peptides and 100% sequence coverage. Note there was no evidence of N-terminal Met. The N-terminus was present as both free amine and Nacetylated. There were 7 N-acetylated spectra and 76 non-acetylated spectra.

HC C-Terminal Leu Cleavage—No peptides were detected corresponding to C-terminal Leu cleavage.

### Intact Mass

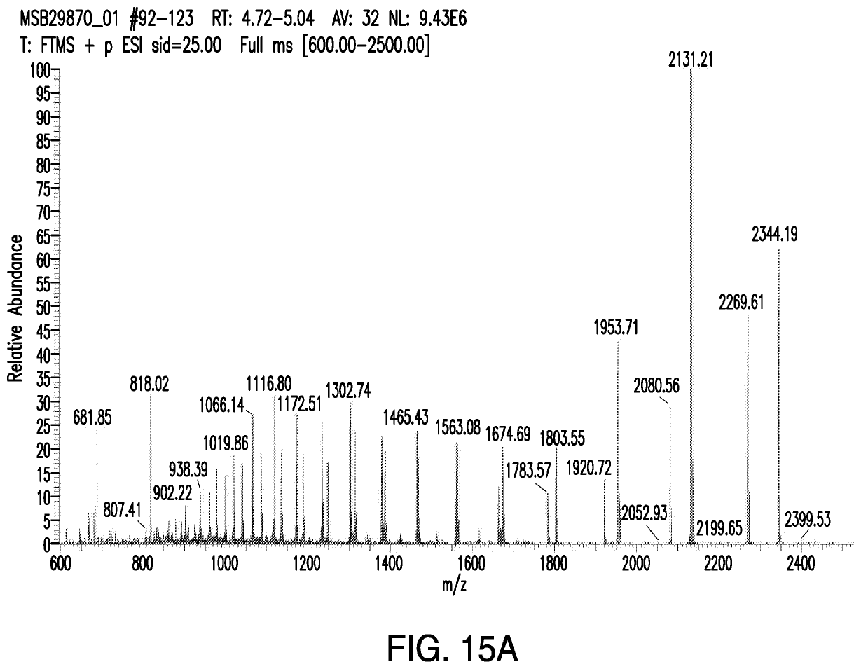

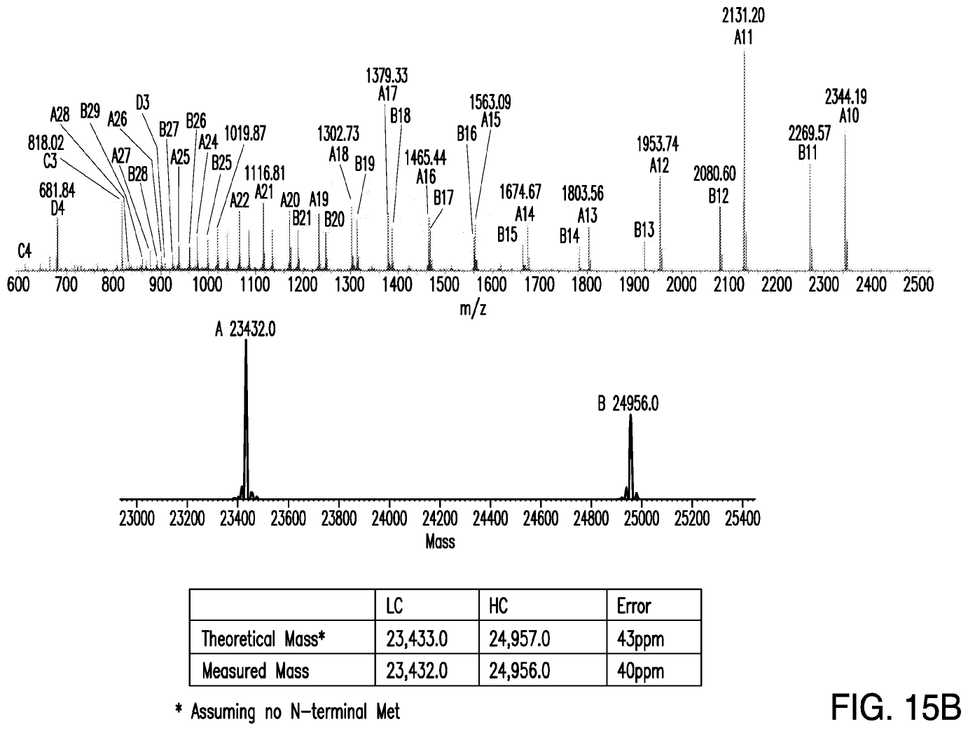

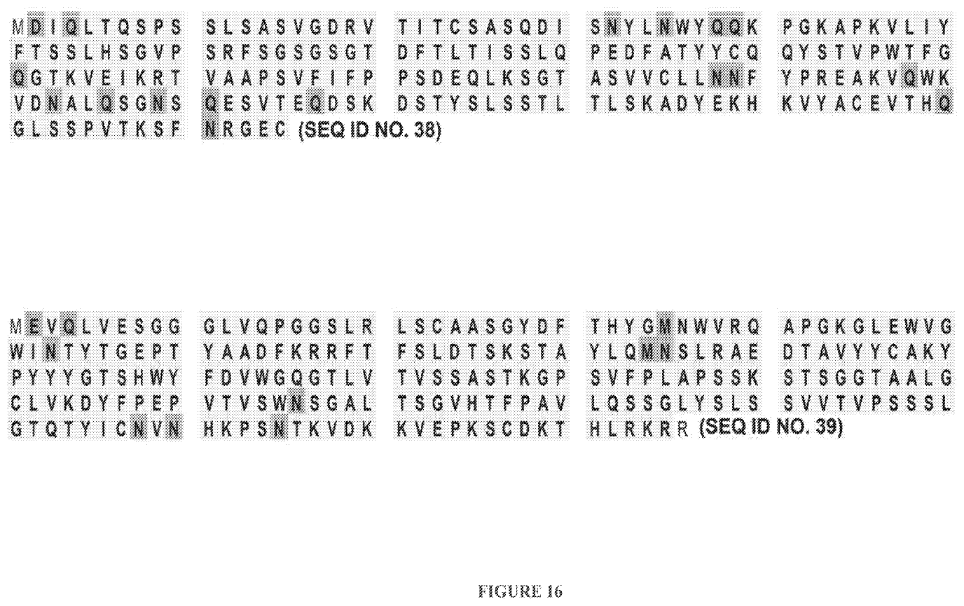

Referring to FIG. 15, the main peak in the observed chromatogram was summed to obtain a spectrum for deconvolution (FIG. 15A). The spectrum was deconvoluted to two components at 24,432.0 Da and 24,956.0 Da average mass. The deconvoluted spectrum and annotated raw data are illustrated in FIG. 15B.

(b) Sample Retinal Cell Line

### Peptide Mapping—Gel

Data matched to both sequences (SEQ ID NO. 38 and SEQ ID NO. 39) as illustrated in FIG. 16.

LC—1107 total spectra, 442 unique peptides and 100% sequence coverage. Note there was no evidence of N-terminal Met. The N-terminus is present as both free amine and N-acetylated. There were 6 N-acetylated spectra and 27 non-acetylated spectra.

HC—843 total spectra, 409 unique peptides and 98% sequence coverage. Note there was no evidence of N-terminal Met. The N-terminus is present as both free amine and N-acetylated. There was 1 N-acetylated spectra and 35 non-acetylated spectra.

HC C-Terminal Cleavage—Peptides were detected corresponding to the L232, R233, and R235 C-terminii. There was no evidence of R236. The extracted ion chromatogram peak areas for three representative peptides are shown below along with the relative percentage. Note there is potential for error in this measurement based on difference in response factors for the three peptides:

| | | Peak | |
|---|---|---|---|
| Peptide | m/z | Area | % |
| ScDKTHL\hat{ } | 430.7007 | 1.71E+10 | 99.36% |
| ScDKTHLR | 339.5026 | 1.09E+08 | 0.63% |
| ScDKTHLRKR* | 434.2332 | 6.56E+05 | 0.004% |

*Pentide present only in chymotrypsin digest, other peptides were taken from trypsin
\hat{ }Different charge state from other peptides

### Intact Mass

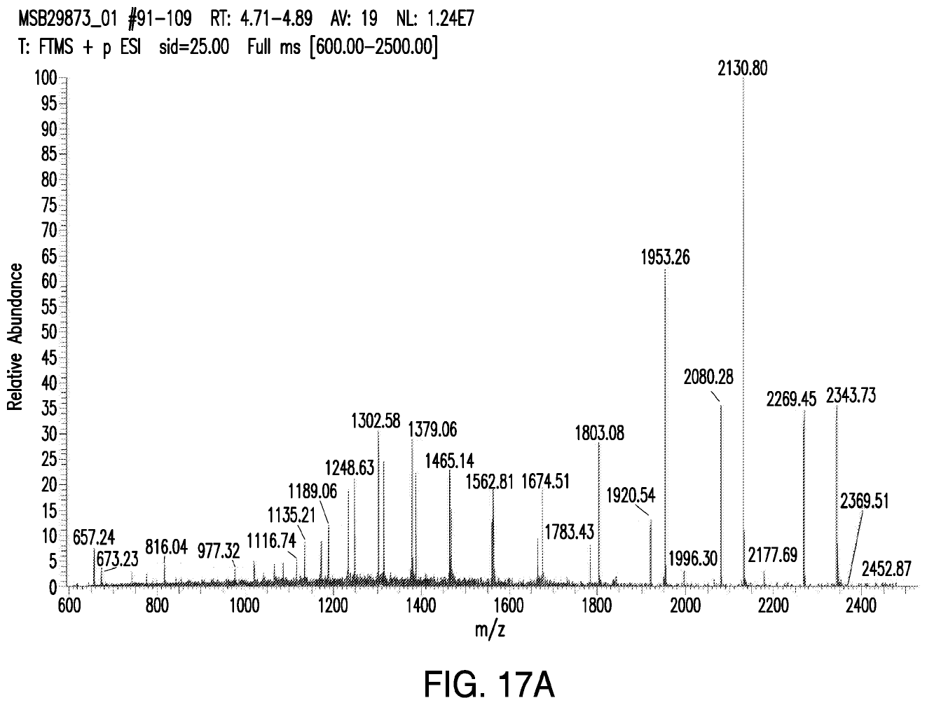

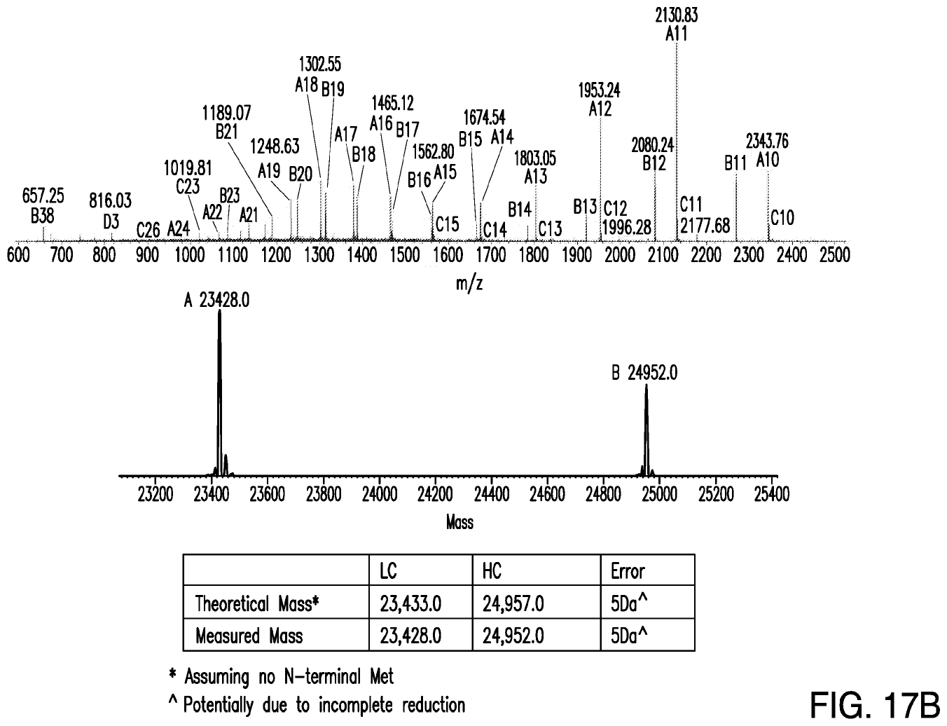

Referring to FIG. 17, the main peak in the observed chromatogram was summed to obtain a spectrum for deconvolution (FIG. 17A). The spectrum was deconvoluted to two components at 24,428.0 Da and 24,952.0 Da average mass. The deconvoluted spectrum and annotated raw data are illustrated in FIG. 17B.

### 6.7 EXAMPLE 16: Suprachoroidal Injection of AAV8.antiVEGFfab in Rat Model

### 6.7.1 Brief Summary of Study

The following studies were conducted to determine the levels of expression and cell types transduced in the eye after suprachoroidal injection of AAV8. The results showed widespread transgene expression throughout the circumference of the eye between one to two weeks after suprachoroidal injection of AAV8.CB7.GFP. Green fluorescent protein(GFP) expression was detected on retinal pigment epithelium(RPE)/choroid cells and all layers of the retina, including ganglion cells.

### 6.7.2 Methods

Norway Brown rats (N=40) received suprachoroidal or subretinal injection of 3 μl containing either 7.2×10⁸ or 2.85×10¹⁰ genome copies (GC) of AAV8.CB7.GFP in each eye. The injections were performed in two steps: first using a sharp needle to puncture through ¾ of the sclera (a sharp tip partial thickness sclerotomy) followed by injecting the same sclerotomy site with a blunt needle to inject just into the suprachoroidal space.

Five animals were euthanized at each of 1, 2, 4 and 8 weeks post injection. One eye was analyzed for GFP expression in homogenates by enzyme-linked immunosorbent assay (ELISA), and the other eye was used for ocular frozen sections that were analyzed by immunofluorescence.

### 6.7.3 Results

Immunofluorescence analysis of frozen sections showed widespread GFP expression 1 week after suprachoroidal injection. A 10 μm horizontal frozen section at the equator showed GFP expression in the retina and RPE/choroid extending more than half the circumference of the eye. At higher magnifications, GFP was detected in the choroid and outer nuclear layer in some regions of the eye, and predominantly in the inner nuclear layer of ganglion cell layer. An RPE/choroid flat mount showed GFP expression throughout about ¼ of the eyecup extending from the ciliary body posteriorly almost to the optic nerve. A retinal flat mount showed GFP from the anterior edge of the retina posterior to the equator throughout about ⅕ of the retina area.

The expression area and fluorescent intensity of GFP increased between 1 and 2 weeks after suprachoroidal expression. The immunofluorescence analysis of ocular sections revealed that by 2 weeks, GFP expression was detected in RPE/choroid cells and all cells of the retina, including ganglion cells. At 2 weeks, a 10 μm horizontal frozen section at the equator showed GFP in the retina and RPE/choroid extending around the entire circumference of the eye. Higher magnifications showed GFP in the choroid, outer segments, outer nuclear layer, inner nuclear layer and ganglion cell layer. An RPE/choroid flat mount showed GFP throughout about ⅓ of the eyecup extending from the ciliary body posteriorly almost to the optic nerve. Higher magnification of the region showed many more high GFP-expressing than low GFP-expressing RPE cells. A retinal flat mount showed GFP in about ¼ of the retina from the anterior edge posteriorly nearly to the optic nerve.

At 4 and 8 weeks, fluorescence intensity decreased, and the decrease is believed to be due to inflammatory reactions in response to the high level of protein produced.

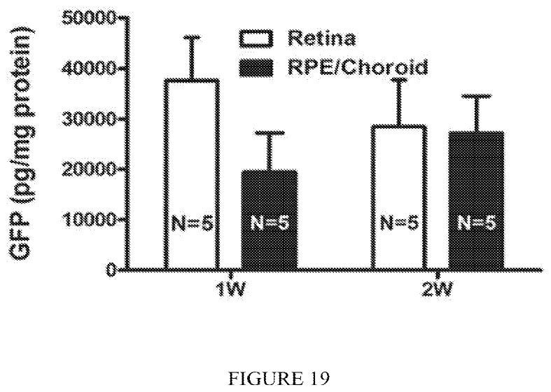

The mean expression level of GFP was high in homogenates of retina and RPE/choroid at 1 and 2 weeks after suprachoroidal injection (FIG. 19).

### 6.8 EXAMPLE 17: Comparison of Suprachoroidal and Subretinal Injection of AAV8.antiVEGFfab in Rat Model

### 6.8.1 Brief Summary of the Study

The following studies were conducted to compare the expression achieved by superchoroidal versus subretinal injection of AAV8.anti-VEGF, and to determine whether suprachoroidal injection of AAV8. antiVEGFfab can reduce VEGF-induced leakage and neovascularization in the eye and produce similar levels of ant-VEGF. Results showed that equally high levels of antiVEGFfab were detected in eyes injected with suprachoroidal or subretinal AAV8.antiVEGFfab, distribution of anti-VEGF protein was similar in the retina vs choroid and that suprachoroidal injection was equally effective at neutralizing VEGF-induced leakage and neovascularization to the subretinal delivery.

### 6.8.2 Methods

Norway Brown rats received suprachoroidal or subretinal injection of 3 μl containing 2.85×10¹⁰ GC per eye (concentration of 4×10¹⁰ GC/ml) of AAV8.CB7.antiVEGFfab in one eye, and a suprachoroidal or subretinal injection of 3 μl containing 7.2×10⁸ GC of AAV8.CB7.GFP in the other eye. After 2 weeks, 200 ng of VEGF was injected into the vitreous. In a subset of rats evaluated at 2 weeks, 100 ng of VEGF was injected.

### 6.8.3 Results

At 2 weeks, fundus photographs taken 24 hours after the VEGF injection showed normal retinas and retinal vessel caliber in the AAV8. antiVEGFfab-injected eyes, whereas the AAV8.GFP-injected eyes showed dilated vessels, evidence of edema, blurred optic disc margins and opalescent retina.

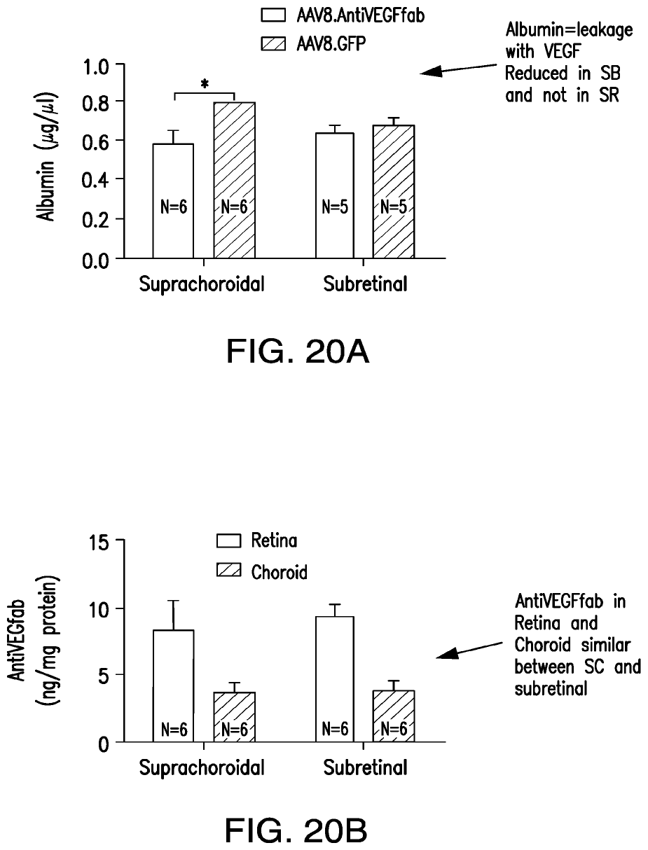

Vascular leakage was assessed by measurement of albumin in vitreous samples by ELISA showed significant reduction in eyes given suprachoroidal injection of AAV8. anti VEGFfab versus fellow eyes given suprachoroidal AAV8.GFP. However, there was no significant difference in vitreous albumin between eyes given subretinal AAV8.antiVEGFfab versus fellow eyes given subretinal AAV8.GFP (FIG. 20A).

Equally high levels of anti VEGFfab were detected in eyes injected with suprachoroidal or subretinal AAV8.antiVEGFfab (FIG. 20B) and the distribution in the retina vs choroida was similar.

### 6.8.4 EXAMPLE 18: Suprachoroidal AAV8-Vectored Gene Transfer Provides Widespread Transgene Expression in RPE and Retina

### 6.8.5 Brief Summary of the Study

There has been great progress in gene replacement for inherited retinal degenerations and gene delivery of VEGF-neutralizing proteins for neovascular age-related macular degeneration (NVAMD); however, delivery of viral vectors to the retinal pigmented epithelium (RPE) and retina can still be problematic. Transgene expression is limited after intravitreous injection of AAV vectors has limited to no expression in photoreceptors and RPE so that subretinal injection is the preferred route of delivery for most applications. However, subretinal injections separate the photoreceptors from the RPF and when the fovea is included, damage to cone photoreceptors can occur and limit visual potential. Also subretinal injections are done after vitrectomy in the operating room and carry a high risk of procedure-releated events, cataract formation and a 1-2% risk of retinal detachment. This study reports a novel approach that has important advantages for ocular gene transfer, suprachoroidal injection of AAV8 vectors. Two weeks after suprachoroidal injection of 3 μl containing 2.85 ×10¹⁰ gene copies (GC) of AAV8.GFP in rats, there was strong GFP fluorescence covering 23% of RPE/choroid flat mounts extending from the ora serrata posteriorly to the region adjacent to the optic nerve. Ocular sections showed GFP fluorescence around the entire circumference of the equator of the eye throughout the choroid, RPE, and all layers of the retina. Two suprachoroidal injections of 3 μl containing 2.85 ×10¹⁰ gene copies (GC) of AAV8.GFP, resulted in GFP fluorescence covering 42% of the area of RPE/choroid flat mounts. The mean level of anti VEGFfab in retina and RPE/choroid 2 weeks after suprachoroidal injection of 3 μl containing 2.85 ×10¹⁰ gene copies (GC) of AAV8.antiVEGFfab was 8.68 and 3.72 ng/mg protein, respectively, which was not significantly different from that seen after subretinal injection of the same amount of vector. Suprachoroidal and subretinal injection of AAV8.antiVEGF was equally effective preventing VEGF-induced retinal hemorrhages, dilation of retinal vessels, and vascular leakage at 2 and 7 weeks after vector injection. These data demonstrate that suprachoroidal injection may provide a preferred route for ocular gene transfer that can maximize efficacy and safety.

### 6.8.6 Background of the Study

Subretinal injection of AAV2.CMV-RPE65 has resulted in improved mobility in patients with Leber Congenital Amaurosis (LCA) due to mutation in the RPE65 gene (Maguire et al., 2008, N. Eng. J. Med. 358:2240-2248; Bainbridge et al., 2008, N. Eng. J. Med. 358: 2231-2239; Hauswirth et al., 2008, Hum. Gen. Ther. 19:979-990). Approval of this treatment by the Food and Drug Administration represents an important validation of current and future potential of ocular gene replacement. However, despite the overall benefit for the LCA study population, there were serious complications from injecting under the fovea in some study patients including endophthalmitis, macular hole, and reduced visual acuity (Jacobson et al., 2015, N. Eng. J. Med. 372: 1920-1926; Bennett et al., 2016, Lancet 388:661-672). Any intraocular injection or procedure can result in endophthalmitis, but the longer and more involved a procedure is, the greater the risk of endophthalmitis. Subretinal injections separate the photoreceptors from the retinal pigmented epithelium (RPE) which can compromise photoreceptors in a normal eye, but may be particularly hazardous in an eye with photoreceptors damaged from an inherited retinal degeneration (Hauswirth et al., 2008, Hum. Gen. Ther. 19:979-990). Such eyes have subretinal fibrosis that increases retinal-RPE adherence necessitating greater pressure to create a subretinal bleb and since the fovea is the thinnest part of the macula, pressurized subretinal fluid can escape through the fovea creating a macular hole and reducing vector in the subretinal space. After subretinal vector injection, infection is limited to cells within the bleb (the region where the photoreceptors and RPE are separated by the vector-containing fluid), so the size and location of the bleb is critical, but it is not always casy to control because the path of least resistance which determines the direction a bleb spreads is not predictable from inspection of the retina at the time of surgery. Sometimes a bleb extends out symmetrically from a subretinal injection site resulting in a circle and sometimes it spreads asymmetrically to the retinal periphery in one direction failing to involve an area of posterior retina that was targeted. Sometimes a bleb extends more along the z-axis than the x or y axes resulting in a high bleb involving a relatively small area of retina and RPE. This unpredictability can be a source of variability in location and amount of transgene expression resulting in variability in outcomes and potentially poorer outcomes in some patients. Multiple subretinal injections may help to expose targeted areas of retina and RPE to vector, but will increase the risk of complications.

Suprachoroidal injection has recently been demonstrated to provide a new route for ocular drug delivery. The suprachoroidal space is a potential space along the inner surface of the sclera that can be expanded by injection of fluid just inside the sclera. The development of microneedles with a length that approximates the thickness of the sclera has facilitated suprachoroidal injections (Patel et al., 2011, Pharm. Res. 28:166-176), but they can also be done using standard needles. Fluorescently labeled particles injected near the limbus flow circumferentially around the eye resulting in a broad area of exposure (Patel et al., 2012, Invest. Opthalmol. Vis. Sci. 53: 4433-4441). Most small molecules have a half-life of a few hours in the suprachoroidal space, but lipophilic molecules, such as triamcinolone acetonide, form precipitates that dissolve slowly providing sustained delivery to the retina (Patel et al., 2012, Invest. Opthalmol. Vis. Sci. 53: 4433-4441; Chen et al., 2015, J. Control. Release 203:109-117) Clinical trials have demonstrated prolonged improvement in macular edema in multiple disease processes after suprachoroidal injection of triamcinolone acetonide (Yeh et al., 2018 Aug. 15, Retina cpub: doi: 10.1097/IAE.0000000000002279). In this study, the potential value of suprachoroidal injection of AAV8 vectors was investigated for ocular gene transfer.

### 6.8.7 Materials and Methods

(a) AAV8 vectors

The AAV8 vectors were provided by REGENXBIO Inc. (Rockville, MD) AAV8.GFP is a non-replicating AAV8 vector containing a gene cassette encoding GFP utilizing the CB7 promoter. AAV8.antiVEGFfab is a non-replicating AAV8 vector containing a gene cassette encoding a humanized monoclonal antigen-binding fragment that neutralizes human VEGF utilizing the CB7 promoter consisting of the chicken β-actin promoter and CMV enhancer, a chicken β-acitn intron, and a rabbit β-globin poly A signal.

(b) Animals

All animals were treated in accordance with the Association for Research in Vision and Ophthalmology Statement for Use of Animals in Ophthalmic and Vision Research, and protocols were reviewed and approved by the Johns Hopkins University Animal Care and Use Committee. 8 weeks Norway Brown rats were purchased from Charles River (Frederick, MD, USA). Adult Dutch Belted rabbits were purchased from Robinson Services Inc, (Mocksville, NC, USA). Adult normal-sighted Rhesus macaques were housed and cared for at the animal facility at the Johns Hopkins University.

(c) Suprachoroidal Injection of Vector in Rats, Rabbits and Monkeys

Rats were anesthetized with Ketamine/Xylazine, and eyes were visualized with a Zeiss Stereo Dissecting Microscope with 10× magnification (Zeiss, Oberkochen, Germany). A 30-gauge needle on 1 ml syringe was used to generate a partial thickness opening in the sclera paralleled to the limbus and a 33-gauge 45 degree angle needle on a Hamilton syringe (Hamilton Company, Reno, NV) was inserted into the scleral puncture and slowly advanced through the remaining scleral fibers into the suprachoroidal space and 3 μl containing 2.85×10¹⁰ GC of vector was injected. After 30 seconds, the needle was withdrawn while holding a cotton tipped applicator over the injection site and antibiotic ointment (Moore Medical LLC, Farmington, CT) was administered to the ocular surface.

For the rabbit injection, Dutch Belted rabbits were anesthetized with Ketamine/Xylazine, and eyes were exposed under Zeiss Surgical Microscope. An insulin syringe with 27-gauge needle was inserted tangentially through sclera 6 mm behind the limbus. The needle was pushed in about 4-5 mm within the sclera when fingertip gained an empty feeling and 50 μl of vector was slowly injected into the suprachoroidal space. After 30 seconds, the needle was withdrawn while holding a cotton tipped applicator over the injection site and antibiotic ointment was administered to the ocular surface.

For monkey injection, three AAV8 neutralizing antibody (-) adult Rhesus macaques were sedated with ketamine hydrochloride (15-20 mg/kg) followed by topical anesthesia of both eyes using 0.5% Proparacaine (Akorn, IL, USA). 5% povidone iodine was administered to sterilize the ocular surface before the procedure. The procedure was same as in rabbit.

(d) Subretinal Injection of Vector in Rats

Rats were anesthetized and eyes were visualized with a Zeiss surgical Microscope (Zeiss, Oberkochen, Germany) and a 20D Fundus Laser Lens (Ocular Instruments Inc, WA, USA). First, a 30-gauge needle on an insulin syringe was inserted tangentially to punch a hole in the sclera. Then, a 33-gauge Hamilton needle on a 5 μl syringe (Hamilton Company, Reno, NV) was inserted into the scleral hole and gently pushed through the eyeball layers, vitreous cavity, and into the subretinal space of the other side, and 3 μl containing 2.85×10¹⁰ GC of vector was injected. Antibiotic ointment was administered to the ocular surface after the procedure.

(e) Tissue harvesting and histology

Blood samples were collected in monkeys after sedated at two different time points, before vector injection and at 3 weeks before euthanasia. Optic nerve and liver samples were obtained. Rat blood samples were also collected before euthanasia. Liver samples were obtained post-mortem.

After euthanasia, eyes were removed and fixed in 4% paraformaldehyde. One eye had the anterior segment and vitreous removed and then retina and RPE/choroid were isolated and flat mounted separately. The other eye was frozen in optimal cutting temperature media (Fisher Scientific Co.) and cryosectioned (10 μm). Hoechst staining (Vector Laboratories, Burlingame, CA) was done to visualize retinal layers and RPE. Both flat mount and ocular sections were examined by fluorescence microscopy.

After the eyes were removed, an incision was made in the abdominal wall to expose the organs in the peritoneal cavity. The liver was collected and frozen.

(f) Preparation of RPE/choroid and retinal homogenates

Rat retinal and eyecup samples were isolated under dissection microscope and put in RIPA buffer (Sigma Aldrich, Arlington, MA) added with protease inhibitor cocktail (Roche, 68298 Mannheim, Germany). Samples were sonicanized for 4-5 seconds (Sonic Dismembrator Model 300, Fisher Scientific, Walkersville, MD), put on ice bath for about 5 min, then centrifuged for 10 min at 14,000 rpm (Eppendorf, Germany). The supernatants were isolated, and stored at −80C°.

(g) Measurement of GFP in retina and RPE/choroid

GFP concentrations from rat retinal and choroidal samples were measured using GFP SimpleStep ELISA kit (ab171581, Abcam, Cambridge, MA). Briefly, GFP standard and samples were loaded to each well, along with GFP capture antibody. Plates were incubated at room temperature for 1 hour. After 5 time of wash, 100 μl of TMB substrate solution was added to each well and the plates were incubated in dark for 10 min and then 100 μl of stop solution was added to each well. 450 nm absorption was measured by Spectra Max Plus 384 Microplate Reader (Molecular Devices, San Jose, California). GFP concentrations were normalized by the total protein concentration of each sample.

(h) Measurement of antiVEGFfab in retina and RPE/choroid

After eyes were removed, retina and RPE/choroid were separated and homogenized as described above. Using the manufacturer's instructions, a lucentis (ranibizumab) Anti-VEGF ELISA kit (#200-880-LUG; Alpha Diagnostic Intl, San Antonio, TX) was used for quantitation of active lucentis in retina and RPE/choroid samples. The plate was read at 450 nm wavelength by Spectra Max Plus 384 Microplate Reader (Molecular Devices, San Jose, California).

(i) Measurement of albumin in vitreous samples

Vitreous humour was collected using insulin syringe. Using the manufacturer's instructions, a rat albumin ELISA kit (ab 108790; Abcam, Cambridge, MA) was used to measure albumin levels in 1 μL of diluted vitreous humour and albumin samples for standard curve generation. The plate was read at 450 nm and 570 nm by Spectra Max Plus 384 Microplate Reader (Molecular Devices, San Jose, California).

### 6.8.8 Results

(a) Suprachoroidal injection of AAV8.GFP results in GFP expression throughout the retina, RPE, and choroid

One week after suprachoroidal injection of 3 μl containing 2.85 ×10¹⁰ gene copies (GC) of AAV8.GFP 1 mm posterior to the limbus in Brown Norway rats, a 10 μm horizontal frozen section through the equator of the globe showed green fluorescence in the choroid, RPE, and outer retina extending nearly half the circumference of the eye. Higher magnification views showed strong fluorescence in the choroid, RPE, and outer nuclear layer (ONL) of the retina in the quadrant of the injection and more remote from the injection site fluorescence in the retina was primarily in photoreceptor inner segments. After removal of the retina, a flat mount of the eyecup, containing the sclera, choroid, and RPE, showed green fluorescence signal in about 25% of the RPE extending from the border with the ciliary body posteriorly to a region adjacent to the optic nerve. High magnification views showed variable GFP expression in binucleate RPE cells with some showing intense fluorescence obscuring Hoechst-stained nuclei and others showing no detectable fluorescence. Retinal flat mounts showed fluorescence from anterior edge of retina posterior to the equator throughout an area about ⅕ of the retina, somewhat smaller than that seen in the RPE. High magnification views showed poor resolution of fluorescent cells due to the multiple cell layers within the retina.

Two weeks after suprachoroidal injection of 3 μl containing 2.85 ×10¹⁰ GC of AAV8.GFP 1 mm posterior to the limbus in Brown Norway rats, a 10 μm horizontal frozen section through the equator of the globe showed green fluorescence in the choroid, RPE, and outer retina extending around the entire circumference of the cyc. High magnification view showed green fluorescence in the choroid, RPE, outer nuclear layer (ONL), inner nuclear layer (INL), and ganglion cell layer (GCL). Flat mount of the eyecup showed green fluorescence covering about ⅓ of the area of the RPE extending from the anterior border adjacent to the ciliary body posteriorly nearly to the optic nerve. Higher magnification views showed considerable heterogeneity in GFP expression, but a greater percentage of high-expressing RPE cells than low-expressing RPE cells. A retinal flat mount showed intense green fluorescence throughout about ¼ of the retina. As shown in FIGS. 19, at 1 and 2 weeks after suprachoroidal injection of 3 μl containing 2.85 ×10¹⁰ GC of AAV8.GFP, the expression levels of GFP protein in homogenates of RPE/choroid or retina were very high, in the range of 20-40 ng/mg protein.(b) Enhancement of GFP expression by 2 suprachoroidal injections of AAV8.GFP

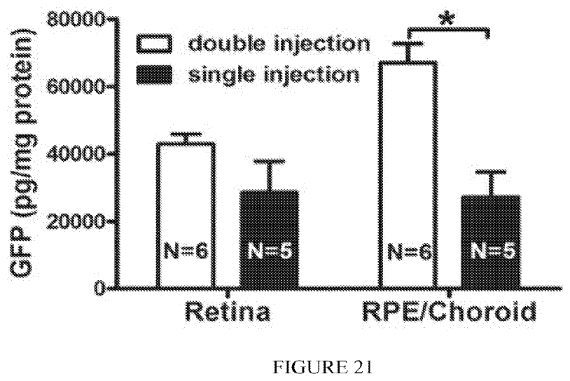

In order to determine if a larger area of the RPE/choroid and retina could be infected by multiple suprachoroidal injections, rats were given a single suprachoroidal injection of 3 μl containing 2.85 ×10¹⁰ GC of AAV8.GFP or two injections of 3 μl containing 2.85 ×10¹⁰ GC of AAV8.GFP three days apart. Two weeks after the first injection, 42.2% of a RPE/choroid flat mount from an eye that received 2 injections showed green fluorescence compared with 22.9% of a RPE/choroid flat mount from an eye 2 weeks after a single injection. The GFP protein level was significantly greater in RPE/choroid homogenates from eyes that had received two suprachoroidal injections compared with those that had received one (FIG. 21).

(c) Suprachoroidal injection of AAV8.antiVEGFfab suppresses VEGF-induced vasodilation and vascular leakage

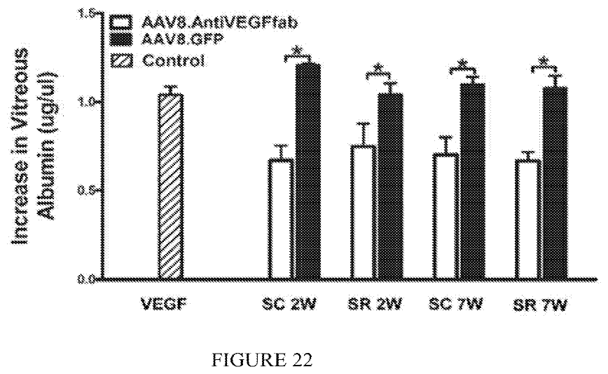

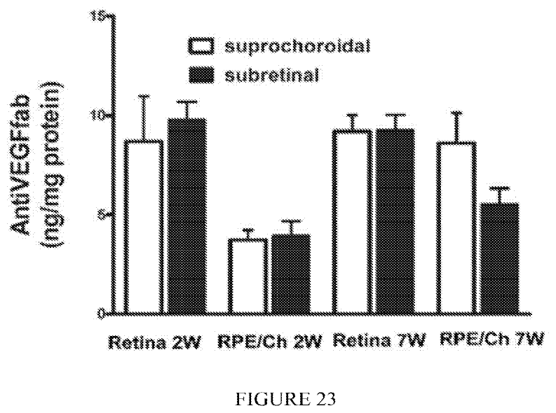

The above experiements with AAV8.GFP show that suprachoroidal injections of AAV8 vectors can provide widespread transgene expression in the RPE/choroid and retina, but provide no information on potential biological effects. Therefore, experiments were performed with AAV8.antiVEGFfab which expresses a VEGF-neutralizing protein that has the potential for therapeutic effects in retinal/choroidal vascular diseases in which overexpression of VEGF is the major driver of excessive vascular leakage and neovascularization (Liu et al., 2018, Mol. Ther. 26:542-549). Rats were given a suprachoroidal or a subretinal injection of 3 μl containing 2.85×10¹⁰ GC of AAV8. antiVEGFfab in one eye and 3 μl containing 2.85×10¹⁰ GC of AAV8.GFP in the fellow eye. After 2 weeks they were given an intravitreous injection of 200 ng of recombinant VEGF₁₆₅ (VEGF) in each eye and for comparison a treatment-naïve rat was given an injection of 200 ng of recombinant VEGF 165 as well. Twenty-four hours later, the VEGF-injected eye that had not received any prior treatment showed dilated blood vessels and a hemorrhage. In contrast, eyes that had previously been given a suprachoroidal injection of AAV8. anti VEGFfab had normal retinal vessels and retinas, while fellow eyes that had received a suprachoroidal injection of AAV8.GFP showed dilated vessels and hemorrhages, which are typical VEGF-induced phenotypes. Similarly, eyes that had previously been given a subretinal injection of AAV8.antiVEGFfab had normal looking fundi, while eyes that had been given subretinal injection of AAV8.GFP had dilated, tortuous vessels. To assess longer term effects, an intravitreous injection of 100 ng of VEGF was done 7 weeks after suprachoroidal or subretinal vector injection. Twenty-four hours after VEGF injection in a previously untreated rat, there were dilated vessels and hemorrhages in eyes. Similar to the earlier time point, eyes that had been given suprachoroidal or subretinal injection of AAV8.anti VEGFfab had normal fundi and those that had been given suprachoroidal or subretinal injection of AAV8.GFP had dilated vessels and hemorrhages. Measurement of serum albumin that leaks into the vitreous provides a valuable technique to quantify retinal vascular leakage (Fortmann et al., 2018, Sci. Rep. 8:6371). The mean albumin level in the vitreous of untreated rats was 0.6 (±0.49) which is consistent with previous reports. The mean increase in vitreous albumin 24 hours after intravitreous injection of 200 ng of VEGF was 1.04 (10.12) and vitreous albumin was similarly increased in eyes that received VEGF injection 2 or 7 weeks after suprachoroidal or subretinal injection of AAV8.GFP, but the increase in albumin was significantly and comparably reduced in eyes that had received suprachoroidal or subretinal injection of of AAV8.antiVEGF 2 or 7 weeks before(FIG. 22). The mean levels of antiVEGFfab protein in retina and RPE/choroid were similar after suprachoroidal or subretinal injection of AAV8.antiVEGFfab at both time points (FIG. 23).

(d) Suprachoroidal injection in Rabbits

Rabbits were injected with 50 μl containing 4.75 ×10¹¹ GC of AAV8.GFP. After 1 or 2 weeks, RPE/choroid and retinal flat mounts were performed. High GFP expression was observed in some RPE cells and low GFP expression was observed in others. The pattern of heterogeneous levels of expression was similar to what was observed in rats, but more pronounced. The area of high expression extended from the far periphery to the mid-periphery, and was not as posterior as that seen in rats. Retinal flat mounts showed strongest GFP expression in the myelinated streak surrounding the optic nerve which consist of glial cells, suggesting that transduction of glial cells may be particularly effecient. Although the highest expression was observed in the myelinated streak, sections showed that there was expression in neuronal retinal cells spanning the entire thickness of the retina in the mid-periphery.

(e) Suprachoroidal injection in Monkeys

Rhesus monkeys were given a suprachoroidal injection of 50 μl containing 4.75×10¹¹ GC of AAV8.GFP in each eye and after 3 weeks flat mounts from one eye and frozen ocular sections from the fellow eye were examined by fluorescence microscopy. Fluorescence microscopy of a RPE/choroid flat mount 3 weeks after suprachoroidal injection showed strong expression of GFP throughout approximately ⅓ of the mid-peripheral.

The 3 eyes used for flat mounts showed very similar results. Three weeks after suprachoroidal injection, there was GFP expression seen on high magnification views of RPE/choroid flat mounts in the quadrant of the injection all the way posterior to the optic nerve. The level of expression was heterogeneous (very high is some RPE cells, low in others), similar to what was seen in rats. A RPE/choroid flat mount from the mid-periphery in the quadrant of the injection showed heterogeneous GFP expression with intense fluorescence in some cells and little in others (this was better seen at higher magnification which also shows the hexagonal shape of RPE cells). A RPE/choroid flat mount from the posterior retina in the quadrant of the injection showed less intense, but more uniform GFP fluorescence extending almost to the border of the optic nerve (ON) which was outlined by fluorescence. Retinal flat mounts also showed GFP expression extending posteriorly to the cut edge of retina where it had been cut free from the optic nerve all the way posterior to the optic nerve, and at higher magnification fluorescence was seen within a variety of cells of the multilayered retina. The mid-periphery of a scleral flat mount showed strong fluorescence within scleral fibroblasts and a flat mount of the ciliary body showed strong fluorescence within spindle-shaped cells. A section through the ciliary body showed strong fluorescence throughout including the ciliary processes. Retinal sections showed strong GFP expression in all cells spanning the entire thickness of the retina from choroid to ganglion cells and nerve fiber layer. Ocular sections from the mid-periphery and posterior retina showed strong fluorescence in all cells from the outer to inner border of the retina with strong fluorescence in the wall of a retinal vessel in the inner retina. A section of the optic nerve shows GFP expression in sheath around the border of the nerve and in septae separating nerve bundles.

### 6.8.9 Discussion

The eye is a relatively confined space which is advantageous for gene transfer because only small amounts of vector are needed and exposure to the remainder of the body is limited. Two major applications for ocular gene transfer are replacement of mutant genes that cause retinal degeneration and sustained expression of therapeutic proteins, and considerable progress has been made in each of these areas (Maguire et al., 2008, N. Eng. J. Med. 358:2240-2248; Bainbridge et al., 2008, N. Eng. J. Med. 358: 2231-2239; Hauswirth et al., 2008, Hum. Gen. Ther. 19:979-990; MacLaren et al., 2014, Lancet 383: 1129-1137; Campochiaro et al., 2006, Hum. Gen. Ther. 17:167-176; Campochiaro et al., 2016, Hum. Gen. Ther. 28:99-111; Heier et al., 2017, The Lancet 389: 50-61). AAV vectors have emerged as the most widely used vectors for ocular gene transfer and 2 routes of delivery have been studied, intravitreous injection and subretinal injection. Intravitreous injection can be done in an outpatient clinic and exposes all cells lining the vitreous cavity to vector, but expression in the retina is limited to a small population of ganglion cells surrounding the fovea and transitional epithelium of the pars plana. This precludes intravitreous delivery for gene replacement in photoreceptors and severely compromises its use for long term expression of therapeutic proteins. Subretinal injection of AAV vectors results in strong transgene expression in RPE and photoreceptors within the retinal detachment caused by the injection. This provides the ability to replace of mutant genes in photoreceptors or RPE in the area of the detachment or strongly express soluble therapeutic proteins that can access the entire retina. The disadvantages of subretinal injection of vector are: 1) it requires going to the operating room and performing vitrectomy which induces cataract in the majority of patients and is complicated by retinal detachment in a small percentage, 2) gene delivery is limited to a relatively small area of the retina and RPE that borders the retinal detachment requiring prioritization of the most important region to be targeted for gene replacement and sacrificing the remainder of the retina and RPE, 3) since the fovea has the highest visual potential, it is high priority for gene replacement, but separation of already compromised photoreceptors and RPE by retinal detachment from subretinal injection of vector may cause permanent damage that reduces vision providing a quandary (Hauswirth et al., 2008, Hum. Gen. Ther. 19:979-990).

In this study, a new route of delivery was demonstrated for AAV8 vectors-suprachoroidal injection, which has important advantages over other routes of delivery. It can be done in an outpatient setting like intravitreous injection thereby avoiding the inconvenience of going to the operating room, but more importantly avoiding the risk of cataract and retinal detachment that accompanies vitrectomy. It also eliminates the risks of separating photoreceptors from the RPE in the fovea, a major concern of subretinal injections for gene replacement targeting the central retina. Compared with spread of vector in the subretinal space, there is much greater spread in the suprachoroidal space allowing expression throughout a very large area of RPE/choroid and retina. Fluorescence microscopy of retinal sections, the most sensitive way of visualizing GFP, showed expression extending around the entire circumference of the eye at the equator 2 weeks after a single suprachoroidal injection of 3 μl containing 2.85 ×10¹⁰ GC of AAV8.GFP. Only high levels of GFP can be visualized by fluorescence microscopy of RPE/choroid or retinal flat mounts and about 25-30% of each showed high expression. The area of high GFP expression was increased by a second injection 3 days after the first. This suggests that using multiple suprachoroidal injections of AAV8 vector, it would be possible to achieve high level expression throughout most or all of the RPE/choroid and retina. This could be achieved during a single clinic visit by performing suprachoroidal injections in each of the 4 quadrants of the eye, waiting for the intraocular pressure to normalize after each injection or speeding that normalization by anterior chamber taps.

Gene replacement is a potentially curative treatment for relatively rare inherited retinal degenerations and therefore has tremendous potential for patients with that uncommon condition. Gene delivery of therapeutic proteins has the potential to revolutionize the management of millions of patients with common retinal and choroidal vascular diseases. Vascular endothelial growth factor is a critical stimulus in neovascular age-related macular degeneration (NVAMD), diabetic macular edema (DME), and macular edema secondary to retinal vein occlusion (RVO) (Campochiaro et al., 2016, Ophthalmology 123:S78-S88). The current approach in each of these conditions is to do intravitreous injections of VEGF neutralizing proteins which reduces vascular leakage and improves vision, but these diseases are chronic with sustained overexpression of VEGF requiring frequent, repeated injections in most patients. It is difficult for patients and physicians to maintain the optimal injection frequency over many years so that in patients with NVAMD treated outside clinical trials, long term visual outcomes are substantially worse than those reported in clinical trials (HORIZON, SEVEN-UP, CATT 5 Year, Holz et al., 2015, Br. J. Ophthalmol. 99:220-226; Cohen et al., 2013, Retina 33:474-481; Finger et al., 2013, Acta Ophthalmol). Gene transfer of expression constructs coding for a VEGF-neutralizing protein provides a good strategy to provide reliable, long-term suppression of the chronically over-expressed VEGF. A clinical trial testing the effect of intravitreous injection of AAV2.sFLT01 showed detectable expression in patients without AAV antibodies with NVAMD given the highest dose and suppressed leakage reducing the need for anti-VEGF injection in some patients, but was insufficient to provide stability in the majority of patients (Heier et al., 2017, The Lancet 389: 50-61). Preclinical studies have shown reliable, high level expression of an antibody fragment that binds VEGF (antiVEGFfab) after subretinal injection of AAV8.antiVEGFfab that resulted in impressive efficacy in models relevant to NVAMD (Liu et al., 2018, Mol. Ther. 26:542-549) and this is currently being tested in a clinical trial (ClinicalTrials.gov Identifier: NCT03066258). In this study, it was demonstrated in rats that, compared with subretinal injection of 3 μl containing 2.85 ×10¹⁰ GC of AAV8.antiVEGFfab, suprachoroidal injection of the same amount of AAV8.antiVEGFfab provided similar expression of anti VEGFfab and similar suppression of VEGF-induced vascular leakage. Pre-existent anti-AAV antibodies do not compromise the efficacy of subretinal injection of AAV vectors as they do with intravitreal delivery. The effect of these antibodies is unknown in suprachoroidal. Suprachoroidal injection of AAV8.antiVEGFfab could be considered as a less invasive approach in patients with retinal or choroidal vascular diseases in patients who lack anti-AAV8 antibodies.

Intravitreous injections, like suprachoroidal injections, are relatively noninvasive and can be done in an outpatient setting. Infection and expression are limited after intravitreous injection of AAV2, AAV8, or other wild type AAV vectors for which it has been tested because the internal limiting membrane (ILM) provides a physical barrier and also binds AAV vectors. With an intact ILM, only the cells within the fovea are able to be transduced by this route although newed vectors may be able to circumvent the ILM blockage. The ILM is thin throughout the entire retina in rodents allowing infection of a wide area of retinal cells extending deep into the retina after intravitreous injection of AAV2. However, the ILM is much more substantial in primates and after intravitreous injection of AAV2.GFP, GFP expression is limited to ganglion cells surrounding the fovea and occasionally along blood vessels (Pechan et al., 2009, Gen. Ther. 16:10-16). Mutant AAV vectors in which surface tyrosine residues involved in ubiquitination are replaced with phenylalanines have reduced vector degradation and increased transgene expression at lower vector doses, increasing effectiveness of small amounts of vector that penetrate the ILM (Zhong et al., 2008, Proc. Natl. Acad. Sci. USA 105:7827-7832; Mowat et al., 2014, Gen. Ther. 21:96-105). Creation of diverse mutant AAV vector libraries and in vivo selection protocols has resulted in identification of vectors that may provide more extensive infection of retinal cells after intravitreous injection in primates which may be useful in the future (Santiago-ORtiz et al., 2011, Gene Ther. 22:934-946). It is important to continue to improve vectors to expand the applications of intravitreous injections of vectors while at the same time exploring suprachoroidal injection of vectors to take advantage of the emerging benefits of gene transfer in a variety of ocular diseases.

### 6.9 EXAMPLE 18: Suprachoroidal Injection

A patient presents with neovascular (wet) age-related macular degeneration (nAMD). A replication deficient adeno-associated viral vector 8 (AAV8) carrying a coding sequence for a soluble anti-VEGF Fab protein (as described in Example 7) is administered to the suprachoroidal space in the eye of the patient via a suprachoroidal drug delivery device (as shown in FIG. 24). The patient is monitored before, during, and after the administration for response by clinical assessments such as retina thickness on OCT, visual acuity and need for additional injections

### 6.10 EXAMPLE 19: Subretinal Administration Via Suprachoroidal Space

A patient presents with neovascular (wet) age-related macular degeneration (nAMD). A replication deficient adeno-associated viral vector 8 (AAV8) carrying a coding sequence for a soluble anti-VEGF Fab protein (as described in Example 7) is administered to the subretinal space in the eye of the patient via the suprachoroidal space in the eye of the patient by the use of a subretinal drug delivery device comprising a catheter that can be inserted and tunneled through the suprachoroidal space toward the posterior pole, where a small needle injects into the subretinal space (as shown in FIG. 25). The patient is monitored before, during, and after the administration for response by clinical assessments such as retina thickness on OCT, visual acuity and need for additional injections.

### 6.11 EXAMPLE 20: Injection Via The Posterior Juxtascleral Depot Procedure

A patient presents with neovascular (wet) age-related macular degeneration (nAMD). A replication deficient adeno-associated viral vector 8 (AAV8) carrying a coding sequence for a soluble anti-VEGF Fab protein (as described in Example 7) is administered to the the outer surface of the sclera in the eye of the patient via a posterior juxtascleral depot procedure (as shown in FIG. 26). The patient is monitored before, during, and after the administration for response by clinical assessments such as retina thickness on OCT, visual acuity and need for additional injections.

### EQUIVALENTS

Although the invention is described in detail with reference to specific embodiments thereof, it will be understood that variations which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims. Those skilled in the art will recognize, or be able to ascertain using no more than routine experimentation, many equivalents to the specific embodiments of the invention described herein. Such equivalents are intended to be encompassed by the following claims

All publications, patents and patent applications mentioned in this specification are herein incorporated by reference into the specification to the same extent as if each individual publication, patent or patent application was specifically and individually indicated to be incorporated herein by reference in their entireties.

1. A method of treating a human subject diagnosed with neovascular age-related macular degeneration (nAMD), comprising administering to the suprachoroidal space of an eye of said human subject an AAV8 expression vector encoding an anti-human vascular endothelial growth factor (hVEGF) antibody or antigen-binding fragment (Fab), wherein the expression vector comprises an expression cassette flanked by AAV2 inverted terminal repeats, and wherein the expression cassette comprises a nucleotide sequence encoding:
(a) heavy chain CDRs 1-3 of SEQ ID NOs: 20, 18, and 21; or a heavy chain variable region comprising amino acids 1-123 of SEQ ID NO: 2; or a heavy chain comprising the amino acid sequence of SEQ ID NO: 2; and
(b) light chain CDRs 1-3 of SEQ ID NOs: 14-16; or a light chain variable region comprising amino acids 1-107 of SEQ ID NO: 1; or a light chain comprising the amino acid sequence of SEQ ID NO: 1.

2. The method of claim 1, wherein the administering is by injecting the expression vector into the suprachoroidal space using a suprachoroidal drug delivery device.

3. The method of claim 2, wherein the suprachoroidal drug delivery device is a microinjecor.

4. A method of treating a human subject diagnosed with nAMD, comprising administering to the subretinal space of an eye of said human subject an AAV8 expression vector encoding an anti-hVEGF antibody or Fab via the suprachoroidal space in the eye of said human subject, wherein the expression vector comprises an expression cassette flanked by AAV2 inverted terminal repeats, and wherein the expression cassette comprises a nucleotide sequence encoding:
(a) heavy chain CDRs 1-3 of SEQ ID NOs: 20, 18, and 21; or a heavy chain variable region comprising amino acids 1-123 of SEQ ID NO: 2; or a heavy chain comprising the amino acid sequence of SEQ ID NO: 2; and
(b) light chain CDRs 1-3 of SEQ ID NOs: 14-16; or a light chain variable region comprising amino acids 1-107 of SEQ ID NO: 1; or a light chain comprising the amino acid sequence of SEQ ID NO: 1.

5. The method of claim 4, wherein the administering is by use of a subretinal drug delivery device comprising a catheter that can be inserted and tunneled through the suprachoroidal space toward the posterior pole, where a small needle injects into the subretinal space.

6. The method of claim 5, wherein the administering comprises inserting and tunneling the catheter of the subretinal drug delivery device through the suprachoroidal space.

7. A method of treating a human subject diagnosed with nAMD, comprising administering to the outer surface of the sclera in the eye of said human subject an AAV8 expression vector encoding an anti-hVEGF antibody or Fab, wherein the expression vector comprises an expression cassette flanked by AAV2 inverted terminal repeats, and wherein the expression cassette comprises a nucleotide sequence encoding:
(a) heavy chain CDRs 1-3 of SEQ ID NOs: 20, 18, and 21; or a heavy chain variable region comprising amino acids 1-123 of SEQ ID NO: 2; or a heavy chain comprising the amino acid sequence of SEQ ID NO: 2; and
(b) light chain CDRs 1-3 of SEQ ID NOs: 14-16; or a light chain variable region comprising amino acids 1-107 of SEQ ID NO: 1; or a light chain comprising the amino acid sequence of SEQ ID NO: 1.

8. The method of claim 7, wherein the administering is by use of a juxtascleral drug delivery device that comprises a cannula whose tip can be inserted and kept in direct apposition to the scleral surface.

9. The method of claim 8, wherein the administering comprises inserting and keeping the tip of the cannula in direct apposition to the scleral surface.

10. The method of claim 1, wherein the administering delivers a therapeutically effective amount of the anti-hVEGF antibody or Fab to the retina of said human subject.

11. The method of claim 10, wherein the therapeutically effective amount of the anti-hVEGF antibody or Fab is produced by human retinal cells of said human subject.

12. The method of claim 10, wherein the therapeutically effective amount of the anti-hVEGF antibody or Fab is produced by human photoreceptor cells, horizontal cells, bipolar cells, amacrine cells, retina ganglion cells, and/or retinal pigment epithelial cells in the external limiting membrane of said human subject.

13. The method of claim 12, wherein the human photoreceptor cells are cone cells and/or rod cells.

14. The method of claim 12, wherein the retina ganglion cells are midget cells, parasol cells, bistratified cells, giant retina ganglion cells, photosensitive ganglion cells, and/or Müller glia.

15. The method of claim 1, wherein the human subject has a Best-Corrected Visual Acuity (BCVA) that is ≤20/20 and ≥20/400.

16. The method of claim 1, wherein the human subject has a BCVA that is ≤20/63 and ≥20/400.

17. The method of claim 15, wherein the BCVA is the BCVA in the eye to be treated in the human subject.

18. The method of claim 1, wherein the expression vector encodes an anti-hVEGF Fab.

19. (canceled)

20. (canceled)

21. (canceled)

22. The method of claim 1, wherein the nucleotide sequence encodes a heavy chain comprising the amino acid sequence of SEQ ID NO. 2, and a light chain comprising the amino acid sequence of SEQ ID NO. 1.

23. The method of claim 1, wherein the nucleotide sequence encodes light chain CDRs 1-3 of SEQ ID NOs: 14-16 and heavy chain CDRs 1-3 of SEQ ID NOs: 20, 18, and 21.

24. The method of claim 23, wherein the second amino acid residue of the light chain CDR3 does not carry one or more of the following chemical modifications: oxidation, acetylation, deamidation, and pyroglutamation (pyro Glu).

25. The method of claim 23, wherein the second amino acid residue of the light chain CDR3 is not acetylated.

26. The method of claim 23, wherein the eighth and eleventh amino acid residues of the light chain CDR1 each carries one or more of the following chemical modifications: oxidation, acetylation, deamidation, and pyroglutamation (pyro Glu).

27. The method of claim 22, wherein the anti-hVEGF antibody comprises a heavy chain CDR1 of SEQ ID NO: 20 and wherein the last amino acid residue of the heavy chain CDR1 does not carry one or more of the following chemical modifications: oxidation, acetylation, deamidation, and pyroglutamation (pyro Glu).

28. The method of claim 26, wherein the last amino acid residue of the heavy chain CDR1 is not acetylated.

29. The method of claim 26, wherein the ninth amino acid residue of the heavy chain CDR1 carries one or more of the following chemical modifications: acetylation, deamidation, and pyroglutamation (pyro Glu), the third amino acid residue of the heavy chain CDR2 carries one or more of the following chemical modifications: acetylation, deamidation, and pyroglutamation (pyro Glu).

30. (canceled)

31. (canceled)


SEQUENCE LISTING (51 sequences)

SEQ ID NO: 1 - protein, 214 aa, synthetic construct
  source 1..214: note = Ranibizumab Fab Amino Acid Sequence - light chain
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 231 aa, synthetic construct
  source 1..231: note = Ranibizumab Fab Amino Acid Sequence - heavy chain
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 3 - protein, 214 aa, synthetic construct
  source 1..214: note = Bevacizumab Fab Amino Acid Sequence - Light chain
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 231 aa, synthetic construct
  source 1..231: note = Bevacizumab Fab Amino Acid Sequence - Heavy chain
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 5 - protein, 26 aa, synthetic construct
  source 1..26: note = VEGF-A signal peptide
MNFLLSWVHWSLALLLYLHHAKWSQA

SEQ ID NO: 6 - protein, 29 aa, synthetic construct
  source 1..29: note = Fibulin-1 signal peptide
MERAAPSRRVPLPLLLLGGLALLAAGVDA

SEQ ID NO: 7 - protein, 19 aa, synthetic construct
  source 1..19: note = Vitronectin signal peptide
MAPLRPLLILALLAWVALA

SEQ ID NO: 8 - protein, 18 aa, synthetic construct
  source 1..18: note = Complement Factor H signal peptide
MRLLAKIICLMLWAICVA

SEQ ID NO: 9 - protein, 19 aa, synthetic construct
  source 1..19: note = Opticin signal peptide
MRLLAFLSLLALVLQETGT

SEQ ID NO: 10 - DNA, 728 bp, synthetic construct
  source 1..728: mol_type = other DNA; note = Bevacizumab cDNA - Light chain
gctagcgccaccatgggctggtcctgcatcatcctgttcctggtggccaccgccaccggcgtgcactccgacatccagatgacccagtccccctcctccctgtccgcctccgtgggcgaccgggtgaccatcacctgctccgcctcccaggacatctccaactacctgaactggtaccagcagaagcccggcaaggcccccaaggtgctgatctacttcacctcctccctgcactccggcgtgccctcccggttctccggctccggctccggcaccgacttcaccctgaccatctcctccctgcagcccgaggacttcgccacctactactgccagcagtactccaccgtgccctggaccttcggccagggcaccaaggtggagatcaagcggaccgtggccgccccctccgtgttcatcttccccccctccgacgagcagctgaagtccggcaccgcctccgtggtgtgcctgctgaacaacttctacccccgggaggccaaggtgcagtggaaggtggacaacgccctgcagtccggcaactcccaggagtccgtgaccgagcaggactccaaggactccacctactccctgtcctccaccctgaccctgtccaaggccgactacgagaagcacaaggtgtacgcctgcgaggtgacccaccagggcctgtcctcccccgtgaccaagtccttcaaccggggcgagtgctgagcggccgcctcgag

SEQ ID NO: 11 - DNA, 1440 bp, synthetic construct
  source 1..1440: mol_type = other DNA; note = Bevacizumab cDNA - heavy chain
gctagcgccaccatgggctggtcctgcatcatcctgttcctggtggccaccgccaccggcgtgcactccgaggtgcagctggtggagtccggcggcggcctggtgcagcccggcggctccctgcggctgtcctgcgccgcctccggctacaccttcaccaactacggcatgaactgggtgcggcaggcccccggcaagggcctggagtgggtgggctggatcaacacctacaccggcgagcccacctacgccgccgacttcaagcggcggttcaccttctccctggacacctccaagtccaccgcctacctgcagatgaactccctgcgggccgaggacaccgccgtgtactactgcgccaagtacccccactactacggctcctcccactggtacttcgacgtgtggggccagggcaccctggtgaccgtgtcctccgcctccaccaagggcccctccgtgttccccctggccccctcctccaagtccacctccggcggcaccgccgccctgggctgcctggtgaaggactacttccccgagcccgtgaccgtgtcctggaactccggcgccctgacctccggcgtgcacaccttccccgccgtgctgcagtcctccggcctgtactccctgtcctccgtggtgaccgtgccctcctcctccctgggcacccagacctacatctgcaacgtgaaccacaagccctccaacaccaaggtggacaagaaggtggagcccaagtcctgcgacaagacccacacctgccccccctgccccgcccccgagctgctgggcggcccctccgtgttcctgttcccccccaagcccaaggacaccctgatgatctcccggacccccgaggtgacctgcgtggtggtggacgtgtcccacgaggaccccgaggtgaagttcaactggtacgtggacggcgtggaggtgcacaacgccaagaccaagccccgggaggagcagtacaactccacctaccgggtggtgtccgtgctgaccgtgctgcaccaggactggctgaacggcaaggagtacaagtgcaaggtgtccaacaaggccctgcccgcccccatcgagaagaccatctccaaggccaagggccagccccgggagccccaggtgtacaccctgcccccctcccgggaggagatgaccaagaaccaggtgtccctgacctgcctggtgaagggcttctacccctccgacatcgccgtggagtgggagtccaacggccagcccgagaacaactacaagaccaccccccccgtgctggactccgacggctccttcttcctgtactccaagctgaccgtggacaagtcccggtggcagcagggcaacgtgttctcctgctccgtgatgcacgaggccctgcacaaccactacacccagaagtccctgtccctgtcccccggcaagtgagcggccgcc

SEQ ID NO: 12 - DNA, 733 bp, synthetic construct
  source 1..733: mol_type = other DNA; note = Ranibizumab cDNA (Light chain comprising a signal sequence)
gagctccatggagtttttcaaaaagacggcacttgccgcactggttatgggttttagtggtgcagcattggccgatatccagctgacccagagcccgagcagcctgagcgcaagcgttggtgatcgtgttaccattacctgtagcgcaagccaggatattagcaattatctgaattggtatcagcagaaaccgggtaaagcaccgaaagttctgatttattttaccagcagcctgcatagcggtgttccgagccgttttagcggtagcggtagtggcaccgattttaccctgaccattagcagcctgcagccggaagattttgcaacctattattgtcagcagtatagcaccgttccgtggacctttggtcagggcaccaaagttgaaattaaacgtaccgttgcagcaccgagcgtttttatttttccgcctagtgatgaacagctgaaaagcggcaccgcaagcgttgtttgtctgctgaataatttttatccgcgtgaagcaaaagtgcagtggaaagttgataatgcactgcagagcggtaatagccaagaaagcgttaccgaacaggatagcaaagatagcacctatagcctgagcagcaccctgaccctgagcaaagcagattatgaaaaacacaaagtgtatgcctgcgaagttacccatcagggtctgagcagtccggttaccaaaagttttaatcgtggcgaatgctaatagaagcttggtacc

SEQ ID NO: 13 - DNA, 779 bp, synthetic construct
  source 1..779: mol_type = other DNA; note = Ranibizumab cDNA (Heavy chain comprising a signal sequence)
gagctcatatgaaatacctgctgccgaccgctgctgctggtctgctgctcctcgctgcccagccggcgatggccgaagttcagctggttgaaagcggtggtggtctggttcagcctggtggtagcctgcgtctgagctgtgcagcaagcggttatgattttacccattatggtatgaattgggttcgtcaggcaccgggtaaaggtctggaatgggttggttggattaatacctataccggtgaaccgacctatgcagcagattttaaacgtcgttttacctttagcctggataccagcaaaagcaccgcatatctgcagatgaatagcctgcgtgcagaagataccgcagtttattattgtgccaaatatccgtattactatggcaccagccactggtatttcgatgtttggggtcagggcaccctggttaccgttagcagcgcaagcaccaaaggtccgagcgtttttccgctggcaccgagcagcaaaagtaccagcggtggcacagcagcactgggttgtctggttaaagattattttccggaaccggttaccgtgagctggaatagcggtgcactgaccagcggtgttcatacctttccggcagttctgcagagcagcggtctgtatagcctgagcagcgttgttaccgttccgagcagcagcctgggcacccagacctatatttgtaatgttaatcataaaccgagcaataccaaagtggataaaaaagttgagccgaaaagctgcgataaaacccatctgtaatagggtacc

SEQ ID NO: 14 - protein, 11 aa, synthetic construct
  source 1..11: note = Bevacizumab and Ranibizumab Light Chain CDR1
SASQDISNYLN

SEQ ID NO: 15 - protein, 7 aa, synthetic construct
  source 1..7: note = Bevacizumab and Ranibizumab Light Chain CDR2
FTSSLHS

SEQ ID NO: 16 - protein, 9 aa, synthetic construct
  source 1..9: note = Bevacizumab and Ranibizumab Light Chain CDR3
QQYSTVPWT

SEQ ID NO: 17 - protein, 10 aa, synthetic construct
  source 1..10: note = Bevacizumab Heavy Chain CDR1
GYTFTNYGMN

SEQ ID NO: 18 - protein, 17 aa, synthetic construct
  source 1..17: note = Bevacizumab Heavy Chain CDR2
WINTYTGEPTYAADFKR

SEQ ID NO: 19 - protein, 14 aa, synthetic construct
  source 1..14: note = Bevacizumab Heavy Chain CDR3
YPHYYGSSHWYFDV

SEQ ID NO: 20 - protein, 10 aa, synthetic construct
  source 1..10: note = Ranibizumab Heavy Chain CDR1
GYDFTHYGMN

SEQ ID NO: 21 - protein, 14 aa, synthetic construct
  source 1..14: note = Ranibizumab Heavy Chain CDR3
YPYYYGTSHWYFDV

SEQ ID NO: 22 - protein, 18 aa, synthetic construct
  source 1..18: note = Albumin signal peptide
MKWVTFISLLFLFSSAYS

SEQ ID NO: 23 - protein, 18 aa, synthetic construct
  source 1..18: note = Chymotrypsinogen signal peptide
MAFLWLLSCWALLGTTFG

SEQ ID NO: 24 - protein, 20 aa, synthetic construct
  source 1..20: note = Interleukin-2 signal peptide
MYRMQLLSCIALILALVTNS

SEQ ID NO: 25 - protein, 15 aa, synthetic construct
  source 1..15: note = Trypsinogen-2 signal peptide
MNLLLILTFVAAAVA

SEQ ID NO: 26 - protein, 19 aa, synthetic construct
  source 1..19: note = F2A site
LLNFDLLKLAGDVESNPGP

SEQ ID NO: 27 - protein, 21 aa, synthetic construct
  source 1..21: note = Linker T2A
GSGEGRGSLLTCGDVEENPGP

SEQ ID NO: 28 - protein, 22 aa, synthetic construct
  source 1..22: note = Linker P2A
GSGATNFSLLKQAGDVEENPGP

SEQ ID NO: 29 - protein, 23 aa, synthetic construct
  source 1..23: note = Linker E2A
GSGQCTNYALLKLAGDVESNPGP

SEQ ID NO: 30 - protein, 25 aa, synthetic construct
  source 1..25: note = Linker F2A
GSGVKQTLNFDLLKLAGDVESNPGP

SEQ ID NO: 31 - protein, 4 aa, synthetic construct
  source 1..4: note = Furin linker
RKRR

SEQ ID NO: 32 - protein, 4 aa, synthetic construct
  source 1..4: note = Furin linker
RRRR

SEQ ID NO: 33 - protein, 4 aa, synthetic construct
  source 1..4: note = Furin linker
RRKR

SEQ ID NO: 34 - protein, 4 aa, synthetic construct
  source 1..4: note = Furin linker
RKKR

SEQ ID NO: 35-37 - skipped (3 entries)

SEQ ID NO: 38 - protein, 215 aa, synthetic construct
  source 1..215: note = Ranibizumab Fab Amino Acid Sequence - light chain
MDIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 236 aa, synthetic construct
  source 1..236: note = Ranibizumab Fab Amino Acid Sequence - heavy chain
MEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHLRKRR

SEQ ID NO: 40 - protein, 232 aa, synthetic construct
  source 1..232: note = Ranibizumab Fab Amino Acid Sequence - heavy chain
MEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 41 - protein, 736 aa, synthetic construct
  source 1..736: note = AAV1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 42 - protein, 735 aa, synthetic construct [sequence omitted]

SEQ ID NO: 43 - protein, 736 aa, synthetic construct
  source 1..736: note = AAV3-3
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 44 - protein, 734 aa, synthetic construct
  source 1..734: note = AAV4-4
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 45 - protein, 724 aa, synthetic construct
  source 1..724: note = AAV5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 46 - protein, 736 aa, synthetic construct
  source 1..736: note = AAV6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 47 - protein, 737 aa, synthetic construct
  source 1..737: note = AAV7
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 48 - protein, 738 aa, synthetic construct
  source 1..738: note = AAV8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 49 - protein, 736 aa, synthetic construct
  source 1..736: note = hu31
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGGQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVSTEGVYSEPRPIGTRYLTRNL

SEQ ID NO: 50 - protein, 736 aa, synthetic construct
  source 1..736: note = hu32
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 51 - protein, 736 aa, synthetic construct
  source 1..736: note = AAV9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL